(19) United States
(12) Patent Application Publication
(10) Pub. No.: US 2020/0397860 A1
(43) Pub. Date: Dec. 24, 2020

(54) SLIT AND BONE GROWTH MODULATION

(71) Applicant: Cornell University, Ithaca, NY (US)

(72) Inventors: Matthew Greenblatt et al., New York, NY (US); Ren Xu, New York, NY (US); Lauri H. Glimcher, Boston, MA (US)

(21) Appl. No.: 16/969,155

(22) PCT Filed: Feb. 14, 2019
(86) PCT No.: PCT/US2019/018115

§ 371 Date: Aug. 11, 2020

Related U.S. Application Data
(60) Provisional application No. 62/630,557, filed on Feb. 14, 2018.

(51) Int. Cl. A61K 38/17; A61P 19/08; A61K 47/42; A61L 27/22; A61L 27/24; A61L 27/56; A61L 27/54; C12N 15/113; A61K 31/519; C07K 16/18

(52) U.S. Cl. CPC A61K 38/1709; A61L 2300/602; A61K 47/42; A61L 27/222; A61L 27/24; A61L 27/56; A61L 27/54; C12N 15/113; A61K 31/519; C07K 16/18; A61L 2430/02; A61L 2300/412; C12N 2310/14; C07K 2317/76; A61L 2300/256; A61L 2300/258; A61P 19/08

(57) ABSTRACT

Methods and compositions are described herein that promote bone formation. Such methods and compositions include SLIT3 or SLIT2 agents that can be administered to a subject (e.g., one in need thereof). Methods are also described herein that reduce or prevent unwanted bone formation. Such methods can involve administering an inhibitor of SLIT3 or SLIT2 to a subject.

This application claims benefit of priority to the filing date of U.S. Provisional Application Ser. No. 62/630,557, filed Feb. 14, 2018, the contents of which are specifically incorporated by reference herein in their entirety.

### BACKGROUND

There is an urgent need to develop improved methods to treat osteoporosis and other disorders of low bone mass. One in two women and one in four men will experience a fracture due to osteoporosis during their lifetimes. In total, osteoporosis kills as many women each year as breast cancer. Moreover, medical systems are vastly undertreating osteoporosis, and one factor in this undertreatment is a concern regarding the highly publicized side effects of existing drugs, such as the atypical femoral fractures or osteoporosis of the jaw occurring with bisphosphonate, the most commonly used class of anti-osteoporosis drugs.

Drugs for the treatment of osteoporosis typically fall into one of two categories. They function either to block bone resorption by osteoclasts or to augment bone formation by osteoblasts. Such different categories of anti-osteoporosis medications can have distinct uses. For instance, augmenting bone formation during the repair of bones, as may occur after a traumatic fracture, tumor resection, or after surgical management of other orthopedic problems, is desirable, as substantial secondary morbidity can be associated with prolonged convalescence from these injuries. Complete failure of bone healing is a common occurrence in patients with other comorbid conditions including vascular diseases such as diabetes or disorders causing ongoing inflammation. Impaired healing is commonly noted when optimal physical stabilization of the injury site cannot be achieved by orthopedic surgical approaches or when the injury size is too large (so-called critical-sized defects). However, so far, no existing anabolic agent has demonstrated clear activity in clinical studies to enhance bone healing across any of these indications.

It is increasingly appreciated that the ancillary cell types present in bone tissue actively participate in osteogenesis. For example, a subset of CD31^{hi}, endomucin^{hi} (EMCN^{hi}) vascular endothelium has been recently identified as residing in the bone marrow (BM) near the growth plate. CD31^{hi}EMCN^{hi} endothelium, also known as “type H endothelium”, is believed to actively direct bone formation, as alterations in CD31^{hi}EMCN^{hi} endothelium impact bone architecture, bone formation, and numbers of osteoprogenitors present in the marrow (Kusumbe, Ramasamy, and Adams 2014, Ramasamy et al. 2014). CD31^{hi}EMCN^{hi} endothelium levels can be altered by platelet-derived growth factor type BB (PDGF-BB), providing a potential link between bone resorption activity by osteoclasts and CD31^{hi}EMCN^{hi} endothelium (Xie et al. 2014). However, it is currently unclear if and how levels of CD31^{hi} EMCN^{hi} endothelium are coupled with the physiologic need for bone formation and whether osteoblasts participate in this regulation.

The inventors and coworkers have previously shown that the adaptor protein Schnurri3 (SHN3) is a suppressor of osteoblast activity, as mice lacking SHN3 display a progressive increase in postnatal bone mass due to augmented bone formation (Shim et al. 2013, Jones et al. 2006). As deletion of Shn3 in osteoblast-lineage cells is sufficient to greatly enhance bone formation, it appears that osteoblasts can coordinate the various tissue activities beyond osteoblast-mediated matrix secretion needed to form mature bone.

### SUMMARY

Described herein are methods for prevention of bone loss or for promoting bone growth, bone-strengthening, or bone healing in a subject in need thereof, comprising administering a composition that included a SLIT3 or SLIT2 agent to said subject. It may be in the form of a protein or an expression vector that can express the SLIT3 or SLIT2 protein. The composition can be delivered locally and may be targeted to bone. It may be delivered by injection or in combination with a carrier or medical device. The bone formation may involve promoting the formation or growth of CD31^{hi}EMCN^{hi} endothelium.

Also described herein are methods of preventing bone growth in a subject in need thereof, comprising administering a SLIT3 or SLIT2 inhibiting agent to said subject. The SLIT3 or SLIT2 inhibiting agent may be targeted to bone or delivered locally. The SLIT3 or SLIT2 inhibiting agent is a small interfering RNA or an antibody.

As shown herein, CD31^{hi}EMCN^{hi} skeletal vasculature is increased in mice with an inducible deletion of Shn3 in osteoblasts and CD31^{hi}EMCN^{hi} endothelium is regulated by osteoblasts. More significantly, the secreted ligand SLIT3 as a novel SHN3-controlled, osteoblast-derived regulator of CD31^{hi}EMCN^{hi} endothelium levels. SLIT3 belongs to a conserved family of SLIT ligands that were initially discovered in the context of CNS development, where they mediate axonal guidance through ROBO receptors (Robo1-4) (Nguyen Ba-Charvet et al. 1999, Jaworski and Tessier-Lavigne 2012). Subsequent studies showed SLITs are widely expressed, and the SLIT/ROBO pathway has been implicated in multiple physiological functions outside of the nervous system such as angiogenesis/vasculogenesis (Jones et al. 2008, Rama et al. 2015, Vasam et al. 2017), stem cell regulation (Geutskens et al. 2012, Zhou et al. 2013) and cancer development (Mehlen, Delloye-Bourgeois, and Chedotal 2011). Additionally, SLIT3 has been identified as a proangiogenic factor in mouse models and human engineered tissue (Zhang et al. 2009. Paul et al. 2013). However, the role of SLIT3 in bone metabolism was largely unclear until this work. Herein, we used genetic models to demonstrate that osteoblast-derived SLIT3 controls levels of skeletal CD31^{hi}EMCN^{hi} endothelium and bone mass accrual. Furthermore, we provide proof-of principle that exogenous SLIT3 administration is a novel class of vascular-targeted therapeutics for the treatment of disorders of low bone mass or for enhancing fracture healing.

### BRIEF DESCRIPTION OF THE DRAWINGS

FIG. 1A-1F illustrate that Shn3 mice have increased skeletal vasculature and CD31^{hi}EMCN^{hi} endothelium. FIG. 1A shows representative images of 2-week-old mouse femurs stained with CD31 and 4′,6-diamidino-2-phenylindole (DAPI) (n=4 per group) (Top, lower power; Bottom, higher power). Growth plate (GP) and cortical bone (CB) ar marked. Scale bars, 300 μm (lower power) and 100 μm (higher power). FIG. 1B shows representative images of 2-week-old mouse femurs stained with Endomucin (EMCN) and 4′,6-diamidino-2-phenylindole (DAPI) (n=4 per group) (Top, lower power; Bottom, higher power). FIG. 1C-1 graphically illustrates relative CD31-positive in the bone marrow cavity (n=4 per group). FIG. 1C-2 graphically illustrates relative EMCN-positive vessel area in the bone marrow cavity (n=4 per group). FIG. 1D shows representative image of CD31 and EMCN dual-immunostained femur sections from the femur in 2-week-old mice. Scale bars, 100 μm, n=3 per group. FIG. 1E shows representative flow cytometry plots of CD31^{hi}EMCN^{hi} endothelial cells in Ter119⁻ and CD45⁻ cell population from the femur in 2-week-old mice (n=4 per group). FIG. 1F illustrates the relative frequency of CD31^{hi}EMCN^{hi} endothelial cells in Ter119⁻ and CD45⁻ cell population from the femur in 2-week-old mice (n=4 per group). Values represent mean±s.e.m.; **P<0.01 and ***P<0.001 by an unpaired two-tailed Student's t-test in all panels.

FIG. 2A-2L illustrate that ablation of Shn3 in osteoblasts selectively enhances osteogenesis and angiogenesis in vivo. FIG. 2A-I shows representative micro-CT (μCT) images of trabecular bone in the distal femurs of Shn3^{f/f} (n=7) and Shn3 conditional knockout Shn3^{dmp1} mice (n=8) at 8-weeks of age. Scale bars, 1 mm. FIG. 2A-2 graphically illustrates relative quantitative analysis of bone volume/total volume (BV/TV) in Shn3^{f/f} (n=7) and Shn3^{dmp1} mice (n=8) at 8-weeks of age. Scale bars, 1 mm. FIG. 2B-1 shows representative histomorphometry images of the L3 vertebrae in Shn3^{f/f} (n=6) and Shn3^{dmp1} mice (n=5) at 8-weeks of age. Scale bars, 500 μm. FIG. 2B-2 graphically illustrates quantitative analysis of bone volume/total volume in Shn3^{f/f} (n=6) and Shn3^{dmp1} mice (n=5) at 8-weeks of age. FIG. 2C shows representative calcein double labeling images of the L3 vertebrae in Shn3^{f/f} mice (n=6) and Shn3^{dmp1} mice (n=6) at 8-weeks of age. FIG. 2D graphically illustrates quantification of histomorphometric parameters of the L3 vertebrae in Shn3^{f/f} mice (n=6) and Shn3^{dmp1} mice (n=6) at 8-weeks of age. Trabecular mineral apposition rate (MAR, mm day⁻¹), bone formation rate/bone surface (BFR/BS, mm³ mm⁻² yr⁻¹), osteoclast number/bone perimeter (No. Oc./B. Pm) and osteoblast surface/bone surface (Ob.S/BS, %). FIG. 2E shows representative confocal images of femurs from 2-week old mice with EMCN and CD31 n=3, per group. GP, growth plate. Scale bars, 100 μm. FIG. 2F shows representative flow cytometry plots of CD31^{hi}EMCN^{hi} endothelial cells from the femur of 2-week-old Shn3^{dmp1} (n=6) and Shn3^{f/f} littermate control (n=5) mice. FIG. 2G graphically illustrates relative frequency of CD31^{hi}EMCN^{hi} endothelial cells in the femur of 2-week-old Shn3^{dmp1} (n=6) and Shn3^{f/f} littermate control (n=5) mice. FIG. 2H shows representative μCT images of the trabecular bone in the distal femur metaphysis of Shn3^{f/f} (oil=7; tamoxifen=7) and in mice exhibiting tamoxifen-inducible inhibition of Shn3 (Shn3^{ocn-ert2} mice) (oil=6; tamoxifen=8) twelve-weeks after tamoxifen injection or corn oil injection at 4-weeks of age. FIG. 2I graphically illustrates relative quantitative analysis of bone volume/total volume in Shn3^{f/f} mice (oil=7; tamoxifen=7) and in mice exhibiting tamoxifen-inducible inhibition of Shn3 (Shn3^{ocn-ert2} mice) (oil=6; tamoxifen=8) twelve-weeks after tamoxifen injection or corn oil injection at 4-weeks of age. FIG. 2J shows representative confocal images of endomucin (red) immunostained femur sections from Shn3^{f/f} (n=3) and Shn3^{ocn-ert2} mice (n=3) at 4-weeks after tamoxifen injection into 4-week old mice. GP, growth plate. Scale bars, 300 μm. FIG. 2K shows representative flow cytometry plots of CD31^{hi}Emcn^{hi} endothelial cells from the femurs of Shn3^{f/f} (n=5) and Shn3^{ocn-ert2} mice (n=5) 6-weeks after tamoxifen injection at 4-weeks of age. FIG. 2L graphically illustrates relative frequency of CD31^{hi}Emcn^{hi} endothelial cells from the femurs of Shn3^{f/f} (n=5) and Shn3^{ocn-ert2} mice (n=5) 6-weeks after tamoxifen injection at 4-weeks of age. Values represent mean±s.e.m.; n.s., not significant and *, P<0.05; **, P<0.01; ***, P<0.001 all by an unpaired two-tailed Student's-test or by one-way ANOVA followed by a Tukey's posttest in all panels.

FIG. 3A-3M illustrate that inhibition of Shn3 enhances Slit3 expression in osteoblasts. FIG. 3A shows representative images of bone marrow-derived endothelial progenitor outgrowth cells (EPOCs) from a transwell migration assay. FIG. 3B graphically illustrates migrated cells per field from a transwell migration assay of bone marrow-derived endothelial progenitor outgrowth cells (EPOCs). Basal medium, (BM); Conditioned medium, (CM). n=5 per group. FIG. 3C shows representative images of tube branches from a Matrigel tube formation assay of EPOCs. FIG. 3D graphically illustrates relative quantification of tube branch numbers from a Matrigel tube formation assay with EPOCs. n=5 per group. FIG. 3E illustrates gene ontology (GO) enrichment analysis of differentially expressed genes between Shn3^{-/-} mice and littermate control mice. Each bar is colored and labeled according to p-value of enrichment analysis, where darker colors indicate greater differential expression than lighter colors. FIG. 3F illustrates a heatmap of proangiogenic gene expression from three independently derived sets of primary osteoblasts from Shn3^{+/+} and Shn3^{-/-} mice. FIG. 3G graphically illustrates relative RNA expression as detected by real-time PCR analysis of Slit3 expression in Shn3^{+/+} and Shn3^{-/-} osteoblasts. FIG. 3H-1 graphically illustrates Slit3 mRNA expression in human mesenchymal stromal cells (hMSCs) expressing a control vector or Shn3 shRNAs cultured under osteogenic conditions. n=4 per group. FIG. 3H-2 shows a protein blot illustrating protein levels of Slit3 in human mesenchymal stromal cells (hMSCs) expressing a control vector or Shn3 shRNAs cultured under osteogenic conditions. n=4 per group. FIG. 3I-1 graphically illustrates mRNA levels of Slit3 in hMSCs transgenically expressing Shn3 and mRNA levels of Slit3 in hMSCs transformed with a control vector where the hMSCs were cultured in osteogenic conditions. n=4, per group. FIG. 3I-2 shows a protein blot illustrating protein levels of Slit3 in hMSCs having a control vector or a vector encoding Shn3 when the hMSCs were cultured under osteogenic conditions. n=4, per group. FIG. 3J graphically illustrates results of an ELISA for SLIT3 in osteoblast conditioned media (CM). n=6 per group. FIG. 3K graphically illustrates results of quantitative real-time PCR analysis of Slit3 in Shn3^{+/+} osteoblasts and in osteoblasts expressing a defective Shn3 (resulting from knock-in of a mutation in 3 amino acids within the ERK interacting motif of Shn3. Shn3^{KI/KI}) osteoblasts. FIG. 3L graphically illustrates results of quantitative real-time PCR analysis of Slit3 in hMSCs treated with the indicated doses of trametinib (TTNB). n=4 per group. FIG. 3M shows representative confocal images of CD31 and endomucin (red) dual immunostained sections from the femur of 2-week-old Shn3^{KI/KI} mice or controls n=3 per group. GP, growth plate. Scale bars, 100 μm. Values represent mean±s.e.m.; n.s., not significant, *F<0.05, **P<0.01, ***P<0.001 and ****P<0.0001 by an unpaired two-tailed Student's t-test or one-way ANOVA followed by a Tukey's posttest in all panels.

FIG. 4A-4L illustrate that Slit3^{-/-} mice have decreased skeletal vasculature and bone mass. FIG. 4A shows representative images of tube branches formed in a Matrigel tube formation assay of endothelial progenitor outgrowth cells (EPOCs) in varying concentrations of Slit3. Scale bars, 200 μm. FIG. 4B graphically illustrates that the quantity of tube branch numbers increases with increased Slit3 as measured in a Matrigel tube formation assay with endothelial progenitor outgrowth cells (EPOCs). n=5 per group. FIG. 4C shows representative images of endothelial progenitor outgrowth cells (EPOCs) of various genotypes cultured under different conditions in a Matrigel tube formation assay. Where indicated, a SLIT3-neutralizing antibody or a related isotype control was added. n=5 per group. Scale bars, 200 μm. FIG. 4D graphically illustrates relative quantification of tube branch numbers in a Matrigel tube formation assay of endothelial progenitor outgrowth cells (EPOCs) of various genotypes cultured under different conditions in a Matrigel tube formation assay. Where indicated, a SLIT3-neutralizing antibody or a related isotype control was added. n=5 per group. FIG. 4E shows representative con focal images of CD31-immunostained sections of the bone marrow cavity from the femur of 2-week-old mice. n=4 per group. Scale bars, 300 μm (lower power) and 100 μm (higher power). FIG. 4F shows representative confocal images of endomucin-immunostained sections of the bone marrow cavity from the femur of 2-week-old mice. n=4 per group. Scale bars, 300 μm (lower power) and 100 μm (higher power). FIG. 4G-1 graphically illustrates quantification of relative CD31-positive vessel area in the bone marrow cavity from the femur of 2-week-old mice. n=4 per group. 4G-2 graphically illustrates quantification of relative endomucin-positive vessel area in the bone marrow cavity from the femur of 2-week-old mice. n=4 per group. Scale bars, 300 μm (lower power) and 100 μm (higher power). FIG. 4H-1 shows representative images of the L3 vertebrae in Slit3^{+/+} mice and Slit3^{-/-} mice. Scale bars, 500 μm. FIG. 4H-2 graphically illustrates quantitative analysis of bone volume/total volume in Slit3^{+/+} mice and Slit3^{-/-} mice at 6-weeks of age. n=7 per group. FIG. 4H-3 graphically illustrates quantitative analysis of cortical bone thickness in Slit3^{+/+} mice and Slit3^{-/-} mice at 6-weeks of age. n=7 per group. FIG. 4I-1 shows representative μCT images of trabecular bone in the distal femur metaphysis in Slit3^{+/+} mice (n=8) and Slit3^{-/-} mice. Scale bars, 1 mm. FIG. 4I-2 graphically illustrates quantitative analysis of bone volume/total volume in Slit3^{+/+} mice (n=8) and Slit3^{-/-} mice (n=6) at 12-weeks of age. FIG. 4H-3 graphically illustrates quantitative analysis of cortical bone thickness in Slit3^{+/+} mice (n=8) and Slit3^{-/-} mice (n=6) at 12-weeks of age. FIG. 4J shows representative confocal images of CD31 (top) immunostained, and CD31 and endomucin dual-immunostained, bone sections from the femurs of 2-week-old OSX-cre and Slit3^{osx} mice. n=4 per group. Scale bars, 300 μm (lower power) and 100 μm (higher power). FIG. 4K-1 shows representative μCT images of trabecular bone in the distal femur metaphysis of OSX-cre (n=6) and Slit3^{osx} mice (n=6) at 3-weeks of age. Scale bars, 1 mm FIG. 4K-2 graphically illustrates quantitative analysis of bone volume/total volume in OSX-cre (n=6) and Slit3^{osx} mice (n=6) at 3-weeks of age. FIG. 4L-1 shows representative μCT images of cortical bone in the distal femur metaphysis of OSX-cre (n=6) and Slit3^{osx} mice (n=6) at 3-weeks of age. Scale bars, 1 mm FIG. 4L-2 graphically illustrates quantitative analysis of cortical bone thickness in OSX-cre (n=6) and Slit3^{osx} mice (n=6) at 3-wceks of age. Values represent mean±s.e.m; *P<0.05, **P<0.01, ***P<0.001 and ****p<0.0001 by an unpaired two-tailed Student's t-test or one-way ANOVA followed by a Tukey's posttest in all panels.

FIG. 5A-5H illustrate that Slit3 contributes to the ability of Shn3 to control osteogenesis and CD31^{hi}EMCN^{hi} endothelium in vivo. FIG. 5A shows representative confocal images of CD31 and endomucin dual-immunostained bone sections. GP, growth plate. Scale bars, 100 μm. FIG. 5B-1 illustrates representative flow cytometry dot plots of CD31^{hi}Emcn^{hi} endothelial cells (b, right) of the femur in 2-week-old Shn3^{+/+} Slit3^{+/+} (n=5), Shn3^{+/+} Slit3^{-/-} (n=3), Shn3^{-/-} Slit3^{+/+} (n=5) and Shn3^{-/-} Slit3^{-/-} mice (n=5). FIG. 5B-2 illustrates relative frequency of CD31^{hi}Emcn^{hi} endothelial cells of the femur in 2-week-old Shn3^{+/+} Slit3^{+/+} (n=5), Shn3^{+/+} Slit3^{-/-} (n=3), Shn3^{-/-} Slit3^{+/+} (n=5) and Shn3^{-/-} Slit3^{-/-} mice (n=5). FIG. 5C-1 shows representative μCT images of the trabecular bone in the distal femur metaphysis of 6-week-old Shn3^{+/+} Slit3^{+/+} (n=7), Shn3^{+/+} Slit3^{-/-} (n=7), Shn3^{-/-} Slit3^{+/+} (n=7) and Shn3^{-/-} Slit3^{-/-} mice (n=6). Scale bar, 1 mm. FIG. 5C-2 graphically illustrates percent BV/TV (bone volume/total volume) of 6-week-old Shn3^{+/+} Slit3^{+/+} (n=7), Shn3^{+/+} Slit3^{-/-} (n=7). Shn3^{-/-} Slit3^{+/+} (n=7) and Shn3^{-/-} Slit3^{-/-} mice (n=6). FIG. 5D shows representative images of calcein labelling of 6-week-old Shn3^{+/+} Slit3^{+/+} (n=5), Shn3^{+/+} Slit3^{-/-} (n=6), Shn3^{-/-} Slit^{+/+} (n=4) and Shn3^{-/-} Slit3^{-/-} mice (n=5). Scale bars, 100 μm. FIG. 5E graphically illustrates histomorphometric analysis of mineral apposition rate (MAR, mm day⁻¹) in 6-week-old Shn3^{+/+} Slit3^{+/+} (n=5), Shn3^{+/+} Slit3^{-/-} (n=6), Shn3^{-/-} Slit3^{+/+} (n=4) and Shn3^{-/-} Slit3^{-/-} mice (n=5). FIG. 5F-1 graphically illustrates bone volume/total volume in 6-week-old Shn3^{+/+} Slit3^{+/+} (n=5). Shn3^{+/+} Slit3^{-/-} (n=6), Shn3^{-/-} Slit3^{+/+} (n=4) and Shn3^{-/-} Slit3^{-/-} mice (n=5) in 6-week-old Shn3^{+/+} Slit3^{+/+} (n=5), Shn3^{+/+} Slit3^{-/-} (n=6), Shn3^{+/+} Slit3^{+/+} (n=4) and Shn3^{-/-} Slit3^{-/-} mice (n=5). FIG. 5F-2 graphically illustrates bone formation rate/bone surface (BFR/BS, mm3 mm⁻² yr⁻¹) in 6-week-old Shn3^{+/+} Slit3^{+/+} (n=5), Shn3^{+/+} Slit3^{-/-} (n=6), Shn3^{-/-} Slit3^{+/+} (n=4) and Shn3^{-/-} Slit3^{-/-} mice (n=5). FIG. 5G-1 shows representative μCT images of the trabecular bone in the distal femur metaphysis in 12-week-old Robo1^{+/+} (n=8) and Robo1^{-/-} mice (n=6). Scale bars, 1 mm. FIG. 5G-2 graphically illustrates bone volume/total volume in 12-week-old Robo1^{+/+} (n=8) and Robo1^{-/-} mice (n=6). Scale bars, 1 mm. FIG. 5H shows representative confocal images of CD31-stained, endomucin-stained and DAPI immunostained femurs from 2-week-old Robo1^{-/-} mice (n=3) and littermate controls (n=3). Arrows highlight the region containing CD31^{hi}EMCN^{hi} endothelium near the growth plate (GP). Scale bars, 100 μm. Values represent mean±s.e.m.; *P<0.05, **P<0.01, ***P<0.001 and ****p<0.0001 by an unpaired two-tailed Student's t-test or by one-way ANOVA followed by a Tukey's posttest in all panels.

FIG. 6A-6N shows that administration of recombinant SLIT3 has therapeutic effects on bone fracture healing and ovariectomy-induced bone loss. FIG. 6A shows representative μCT images of mouse femurs 21 days after open femoral midshaft fracture in Shn3^{+/+} Slit3^{+/+} Shn3^{+/+} Slit3^{-/-}, Shn3^{-/-} Slit3^{+/+} and Shn3^{-/-} Slit3^{-/-} mice. Scale bar=1 mm in μCT. FIG. 6B shows representative μCT images of H&E stained and mouse femurs subjected to EMCN immunohistochemistry 21 days after open femoral midshaft fracture in Shn3^{+/+} Slit3^{+/+}, Shn3^{+/+} Slit3^{-/-}, Shn3^{-/-} Slit3^{+/+} and Shn3^{-/-} Slit3^{-/-} mice. Arrowheads highlight EMCN positive vessels located in the fracture callus. Scale bar=200 μm in H&E and 100 μm in EMCN immunohistochemistry (IHC). FIG. 6C graphically illustrates non-union frequency of the fractured femora 4 weeks after open femoral midshaft fracture Shn3^{+/+} Slit3^{+/+}, Shn3^{+/+} Slit3^{-/-}, Shn3^{-/-} Slit3^{+/+} and Shn3^{-/-} Slit3^{-/-} mice (n≥4, per group). FIG. 6D graphically illustrates μCT measurement of BV/TV (bone volume/total volume) in callus area of the fractured femora 4 weeks after open femoral midshaft fracture Shn3^{+/+} Slit3^{+/+}, Shn3^{+/+} Slit3^{+/+}, Shn3^{+/+} Slit3^{+/+} and Shn3^{-/-} Slit3^{-/-} mice (n≥4, per group). FIG. 6E-1 graphically illustrates EMCN positive vessel number in the fractured femora 4 weeks after open femoral midshaft fracture Shn3^{+/+} Slit3^{+/+}, Shn3^{+/+} Slit3^{-/-}, Shn3^{-/-} Slit3^{+/+} and Shn3^{-/-} Slit3^{-/-} mice (n≥4, per group). FIG. 6E-2 graphically illustrates results maximum load as assessed by bio-mechanical testing of the maximum compressive load of the fractured femora 4 weeks after open femoral midshaft fracture Slit3^{+/+} Slit^{+/+}, Shn3^{+/+} Slit3^{-/-}, Shn3^{-/-} Slit3^{+/+} and Shn3^{-/-} Slit3^{-/-} mice (n≥4, per group). FIG. 6F shows representative μCT images of mouse femurs 21 days post-fracture with IV injection of SLITS or PBS. Scale bar=1 mm for μCT. FIG. 6G shows representative H&E stained and EMCN (insert) immunohistochemically stained images of mouse femurs 21 days post-fracture with IV injection of SLITS or PBS. Scale bar=1 mm for μCT, 200 μm in H&E and 100 μm in EMCN IHC. FIG. 6H graphically illustrates μCT measurement of BV/TV in callus area of femurs after IV injection of SLITS or PBS 21 days after fracture (n≥4, per group). FIG. 6I-1 graphically illustrates EMCN positive vessel number in femurs after IV injection of SLITS or PBS 21 days after fracture (n≥4, per group). FIG. 6I-2 graphically illustrates EMCN positive vessel volume in femurs after IV injection of SLITS or PBS 21 days after fracture (n≥4, per group). FIG. 6J-1 graphically illustrates results of bio-mechanical test of maximum compressive load of femurs after IV injection of SLITS or PBS 21 days after fracture (n≥4, per group). FIG. 6J-2 graphically illustrates results of bio-mechanical test of stiffness of femurs after IV injection of SLITS or PBS 21 days after fracture (n≥4, per group). FIG. 6K-1 graphically illustrates results of measurement of fracture callus BV/TV of mouse femurs harvested 21 days post-fracture with insertion of a gelatin sponge soaked with SLITS or vehicle. A no sponge control is included. FIG. 6K-2 graphically illustrates results of biomechanical testing of maximum load of mouse femurs harvested 21 days post-fracture with insertion of a gelatin sponge soaked with SLIT3 or vehicle. A no sponge control is included. FIG. 6L shows representative images of CD31 and EMCN dual-immunostained callus sections of mouse femurs 21 days post-fracture with insertion of a gelatin sponge soaked with SLIT3 or vehicle at the time of fracture. Scale bars, 300 μm, n=3 per group. FIG. 6M shows representative μCT images of the trabecular bone in the distal femur of mice after sham-operation (Sham), or in ovariectomized (OVX) mice, where the OVX mice were treated with vehicle, SLIT3, or parathyroid hormone (PTH) injection. Scale bars, lima FIG. 6N graphically illustrates relative quantitative analysis of bone volume/total volume in trabecular bone in the distal femur of mice after sham-operation (Sham), or in ovariectomized (OVX) mice, where the OVX mice were treated with vehicle, SLIT3, or parathyroid hormone (PTH) injection. Values represent mean±s.e.m; *P<0.05, **P<0.01, ***p<0.001 and ****p<0.0001 by a Fisher's exact test (FIG. 6C) unpaired two-tailed Student's t-test or by one-way ANOVA followed by a Tukcy's posttest in all other panels.

FIG. 7A-7C illustrate that Shn3^{-/-} mice have increased skeletal vasculature and CD31^{hi}EMCN^{hi} endothelium. FIG. 7A shows representative confocal images of heart, brain, lung and kidney from 2-week-old mice stained for endomucin and 4′,6-diamidino-2-phenylindole (DAPI). Scale bars, 100 μm, n=3 per group. FIG. 7B-1 shows representative images of CD31-positive (upper) and endomucin-positive (lower) vessel area of neonatal mice, n=4 per group. FIG. 7B-2 graphically illustrates quantification of CD31-positive (upper) and endomucin-positive (lower) vessel area of neonatal mice, n=4 per group. FIG. 7C shows a schematic representation of the strategy used for flow cytometry for analysis of CD31^{hi}EMCN^{hi} endothelial cells. Values represent mean±s.e.m.; NS, *P<0.05 and **P<0.01 by an unpaired two-tailed Student's t-test in all panels.

FIG. 8A-8H illustrates that ablation of Shn3 in osteoblasts enhances osteogenesis and angiogenesis in vivo. FIG. 8A-1 graphically illustrates percent BV/TV of trabecular bone in Shn3^{f/f} and Shn3^{osx} mice at 8-weeks of age (male, n≥4). FIG. 8A-2 graphically illustrates cortical bone thickness in Shn3^{f/f} and Shn3^{osx} mice at 8-weeks of age (male, n≥4). FIG. 8B-1 shows representative confocal images of DAPI stained metatarsals from OSX-Cre; R26-mT/mG mice at 7-days of age (male, n=3). Endosteal surface (ES), Cortical bone (CB) and Periosteal surface (PS). Scale bars, 100 μm. FIG. 8B-2 shows representative confocal images of direct eGFP/tdTomato fluorescence in metatarsals from OSX-Cre; R26-mT/mG mice at 7-days of age (male, n=3). Red: membrane-targeted tdTomato; Green: membrane-targeted eGFP; Blue: DAPI. Endosteal surface (ES), Cortical bone (CB) and Periosteal surface (PS). Scale bars, 100 μm. FIG. 8C-1 shows representative μCT images of bone in Shn3^{f/f} and Shn3^{dmp1} mice at 8-weeks of age (male, n≥5). Scale bars, 500 μm. FIG. 8C-2 graphically illustrates cortical bone thickness in Shn3^{f/f} and Shn3^{dmp1} mice at 8-weeks of age (male, n≥5). Scale bars, 500 μm. FIG. 8D-1 graphically illustrates histomorphometric quantification of mineral apposition rate (MAR, mm day⁻¹) at the endosteal and periosteal bone surfaces of the L3 vertebrae in Shn3^{f/f} (n=5) and Shn3^{dmp1} mice (n=5) at 8-weeks of age. FIG. 8D-2 graphically illustrates histomorphometric quantification of bone formation rute/bone surface (BFR/BS, mm³ mm⁻² yr⁻¹) at the endosteal and periosteal bone surfaces of the L3 vertebrae in Shn3^{f/f} (n=5) and Shn3^{dmp1} mice (n=5) at 8-weeks of age. FIG. 8E-1 graphically illustrates histomorphometric quantification of osteoblast surface/bone surface (Ob. S/BS, %) at the endosteal bone surfaces of the U vertebrae in Shn3^{f/f} mice (n=5) and Shn3^{dmp1} mice (n=5) at 8-weeks of age. FIG. 8E-2 graphically illustrates osteoclast number/bone perimeter (No. Oc./B. Pm) at the endosteal bone surfaces of the L3 vertebrae in Shn3^{f/f} mice (n=5) and Shn3^{dmp1} mice (n=5) at 8-weeks of age. FIG. 8F shows representative confocal images of femurs from 2-week-old mice stained for CD31 (red) and DAPI (blue) (Top, low power; Bottom, high power). FIG. 8G representative confocal images of femurs from 2-week-old mice stained for Endomucin (red) and DAPI (blue) (Top, low power; Bottom, high power). GP, growth plate; CB, cortical bone. Scale bars, 300 μm (lower power); 100 μm (high power). FIG. 8H-1 graphically illustrates the percent of CD31-postivc vessels in the bone marrow cavity in Shn3^{f/f} and Shn3^{dmp1} mice (n=4). FIG. 8H-2 graphically illustrates the percent of endomucin-positive vessels in the bone marrow cavity in Shn3^{f/f} and Shn3^{dmp1} mice (n=4). Values represent mean±s.e.m.; ns, not significant, *P<0.05, **P<0.01, ***P<0.001 and ****p<0.0001 by an unpaired two-tailed Student's t-test in all panels.

FIG. 9A-9G illustrate that Shn3 in endothelial cells is dispensable for osteogenesis and angiogenesis in vivo. FIG. 9A graphically illustrates Shn3 expression in bone marrow derived endothelial cells sorted from Shn3^{f/f} and Shn3^{cdh5} mice as quantified by quantitative real-time PCR analysis of (n=4). FIG. 9B shows representative confocal images of femurs from 2-week-old mice stained for CD31 and DAPI (top, lower power; bottom, high power). GP, growth plate; CB, cortical bone. Scale bars, 300 μm (lower power); 100 μm (high power). FIG. 9C shows representative confocal images of femurs from 2-week-old mice stained for endomucin and DAPI (top, lower power; bottom, high power). GP, growth plate; CB, cortical bone. Scale bars, 300 μm (lower power); 100 μm (high power). FIG. 9D-1 graphically illustrates quantification of the staining performed as described for FIG. 9B. FIG. 9D-2 graphically illustrates quantification of the staining performed as described for FIG. 9C. FIG. 9E shows representative confocal images of CD31 and endomucin immunostained sections from the femurs of 2-week-old mice (n=4 per group). GP, growth plate. FIG. 9F shows representative μCT images of trabecular bone in the distal femur in Shn3^{f/f} and Shn3^{cdh5} mice at 8-weeks of age (male, n≥6). FIG. 9G graphically illustrates results of relative quantitative analysis of bone volume/total volume (g) in Shn3^{f/f} and Shn3^{cdh5} mice at 8-weeks of age (male, n≥6). Values represent mean±s.e.m; ns, not significant and ***p<0.001 by an unpaired two-tailed Student's t-test in all panels.

FIG. 10 illustrates inducible inhibition of Shn3 in osteoblasts increases cortical bone thickness. Quantitative μCT analysis of cortical bone thickness in Shn3^{f/f} (oil=7; tamoxifen=7) and Shn3^{ocn-ert2} mice (oil=6; tamoxifen=8) at 12-weeks after tamoxifen injection or corn oil injection at 4-weeks of age. Values represent mean±s.e.m.; n.s., not significant and **P<0.01 by one-way ANOVA followed by a Tukey's posttest.

FIG. 11A-11F show that Slit3 is expressed in osteoblasts. FIG. 11A graphically illustrates results of quantitative real-time PCR analysis of Slit3 expression in mouse brain, lung, kidney, heart, liver, spleen, bone marrow, bones (femur and tibia after bone marrow flushing), primary osteoblasts and osteoclasts (n=4). FIG. 11B graphically illustrates results of quantitative real-time PCR analysis of Slit3 expression during human CD14⁺ cell differentiation with M-CSF and RANKL. Human brain cDNA (Clontech) as a positive control. FIG. 11C-1 graphically illustrates quantification of Alpl in human mesenchymal stem cells differentiation (n=4). FIG. 11C-2 graphically illustrates quantification of Lbsp in human mesenchymal stem cells differentiation (n=4). FIG. 11C-3 graphically illustrates quantification of Slit3 in human mesenchymal stem cells differentiation (n=4). FIG. 11D-1 graphically illustrates quantification of mRNA (mRNA) levels of Slit3 in murine primary osteoblasts (POBs) transduced with a control vector or a construct expressing SHN3 and subsequently cultured under osteogenic conditions. n=4 per group. FIG. 11D-2 graphically illustrates quantification of protein levels of Slit3 in murine primary osteoblasts (POBs) transduced with a control vector or a construct expressing SHN3 and subsequently cultured under osteogenic conditions. n=4 per group. FIG. 11E graphically illustrates quantification of PDGF-BB in osteoblast CM and serum from Shn3^{-/-} mice by ELISA analyses using littermate controls. FIG. 11F graphically illustrates quantification of Slit3 expression as measured by quantitative real-time PCR analysis of in POBs treated with the indicated doses of trametinib (TTNB). n=6 per group. Values represent mean±s.e.m.; n.s., not significant, **P<0.01 and ****p<0.0001 by an unpaired two-tailed Student's t-test or one-way ANOVA followed by a Tukey's posttest in all panels.

FIG. 12 illustrates in vivo expression of Slit3 and Robo1. Representative confocal images of femurs from 2-week-old Slit3^{+/-} or Robo1^{+/-} mice with a lacZ knock-in and the control mice stained for beta-galactosidase, Endomucin and DAPI (top, lower power, bottom, high power). GP, growth plate. Scale bars, 100 μm (lower power); 40 μm (high power). n=3 per group.

FIG. 13A-13B show that SLIT3 has proangiogenic activity in vitro. FIG. 13A-1 shows representative images from a wound healing assay of bone marrow-derived endothelial progenitor outgrowth cells (EPOCs) with SLIT3 stimulation. n=3 per group. FIG. 13A-2 graphically illustrates relative quantification results from a wound healing assay of bone marrow-derived endothelial progenitor outgrowth cells (EPOCs) with SLIT3 stimulation. n=3 per group. FIG. 13B graphically illustrates results of an Alamar blue based proliferation assay in EPOCs with SLIT3 treatment. n=5 per group. Values represent mean±s.e.m.; *P<0.05, **P<0.01, ***P<0.001 and ****p<0.0001 by a two-way ANOVA followed by a Tukey's posttest in all panels.

FIG. 14A-14C show that SLIT1 and SLIT2 have proangiogenic activity. FIG. 14A-1 shows representative images of a Matrigel tube formation assay using EPOCs after SLIT1 treatment. n=5 per group. FIG. 14A-2 graphically illustrates relative quantification of tube branch numbers of a Matrigel tube formation assay in EPOCs after SLIT1 treatment. n=5 per group. FIG. 14B-1 shows representative images of a Matrigel tube formation assay using EPOCs after SLIT2 treatment. n=5 per group. FIG. 14B-2 graphically illustrates relative quantification of tube branch numbers of a Matrigel tube formation assay in EPOCs after SLIT2 treatment n=5 per group. FIG. 14C-I shows representative flow cytometry dot plots of CD31^{hi}Emcn^{hi} endothelial cells from femurs of 8-week-old B6 mice after twice weekly IV injection of SLIT2 (1 mg/kg) or vehicle initiated 21 days prior to sacrifice. FIG. 14C-2 graphically illustrates relative frequency of CD31^{hi}Emcn^{hi} endothelial cells (right) in femurs of 8-week-old B6 mice after twice weekly IV injection of SLIT2 (1 mg/kg) or vehicle initiated 21 days prior to sacrifice, Vehicle (n=5), SLIT2 (n=4). Values represent mean±s.e.m.; *P<0.05. **P<0.01, ***P<0.001 and ****p<0.0001 by an unpaired two-tailed Student's t-test or a two-way ANOVA followed by a Tukey's posttest in all panels.

FIG. 15 illustrates that SLIT3 induces activation of Hippo and ERK MAPK pathway signaling in bone marrow derived endothelial cells. Effects of Slit3 treatment on YAP, AKT and ERK phosphorylation in mouse EPOCs after serum starvation are illustrated.

FIG. 16A-16B show that SLIT3 induces production of skeletal CD31^{hi}EMCN^{hi} cells in vitro and in vivo. FIG. 16A shows representative flow cytometry plots (left) and relative frequency of CD31^{hi}Emcn^{hi} endothelial cells (right) in cultures of EPOCs after SLIT3 stimulation, FGF2 as a positive control. FIG. 16B shows representative flow cytometry plots (left) and relative frequency of CD31^{hi}Emcn^{hi} endothelial cells (right) in the Ter119⁻, CD45⁻ cell fraction isolated from the femurs of 8-week-old B6 mice after twice weekly IV injection of SLIT3 (1 mg/kg) or vehicle initiated 21 days prior to sacrifice. (Vehicle=5; SLIT3=4). Values represent mean±s.e.m.; *P<0.05 and **P<0.01 by an unpaired two-tailed Student's t-test or by one-way ANOVA followed by a Tukey's posttest in all panels.

FIG. 17A-17G show that SLIT3 does not directly regulate osteoblast differentiation or activity. FIG. 17A-1 graphically illustrates body weight of 1-month old Slit3^{-/-} mice and littermate controls (1M, n=4). FIG. 17A-2 graphically illustrates body weight of 3-month old Slit3^{-/-} mice and littermate controls (3M, n≥5). FIG. 17B-1 graphically illustrates the trabecular osteoclast number/bone perimeter (No. Oc./B. Pm) of the L3 vertebrae in Slit3^{-/-} mice (n=7) and littermate controls mice (n=7) at 6-weeks of age. FIG. 17B-2 graphically illustrates ELISA results for type I collagen carboxy-terminal telopeptide (CTX) levels in serum samples collected from Slit3^{-/-} mice (n=8) and littermate controls (n=6) at 6 weeks of age. FIG. 17C-1 graphically illustrates quantitative real-time PCR analysis of Sp7 expression levels in mouse primary osteoblasts derived from neonatal Slit3^{-/-} mice and littermate controls (n=4 per group) after 6 days of culture under osteoblast differentiation conditions. FIG. 17C-2 graphically illustrates quantitative real-time PCR analysis of Runx2 expression levels in mouse primary osteoblasts derived from neonatal Slit3^{-/-} mice and littermate controls (n=4 per group) after 6 days of culture under osteoblast differentiation conditions. FIG. 17C-3 graphically illustrates quantitative real-time PCR analysis of lbsp expression levels in mouse primary osteoblasts derived from neonatal Slit3^{-/-} mice and littermate controls (rt=4 per group) after 6 days of culture under osteoblast differentiation conditions. FIG. 17C-4 graphically illustrates quantitative real-time PCR analysis of Bglap expression levels in mouse primary osteoblasts derived from neonatal Slit3^{-/-} mice and littermate controls (n=4 per group) after 6 days of culture under osteoblast differentiation conditions. FIG. 17D graphically illustrates alkaline phosphatase (ALP) activity in mouse primary osteoblasts derived from neonatal Slit3^{-/-} and littermate control mice (n=5) after 6 days of culture under osteoblast differentiation conditions. FIG. 17E-1 shows a mineralization assay of mouse primary osteoblasts derived from neonatal Slit3^{-/-} and littermate control mice (n=5) after 6 days of culture under osteoblast differentiation conditions. FIG. 17E-2 graphically illustrates results of a mineralization assay of mouse primary osteoblasts derived from neonatal Slit3^{-/-} and littermate control mice (n=5) after 6 days of culture under osteoblast differentiation conditions. FIG. 17F graphically illustrates results of an ALP activity assay in human mesenchymal stromal cells treated with different doses of recombinant SLIT3 after 6 days of osteogenic culture. Values represent mean±s.e.m.; n.s., not significant and *P<0.05 by an unpaired two-tailed Student's t-test or by one-way ANOVA followed by a Tukey's posttest in all panels. FIG. 17G graphically illustrates results of an ALP activity assay in human mesenchymal stromal cells treated with different doses of recombinant Robo1-Fc after 6 days of osteogenic culture. Values represent mean±s.e.m.; n.s., not significant and *P<0.05 by an unpaired two-tailed Student's t-test or by one-way ANOVA followed by a Tukey's posttest in all panels.

FIG. 18A-18E illustrate that ablation of Slit3 in osteoblasts impairs osteogenesis and angiogenesis in vivo. FIG. 18A illustrates protein levels of SLIT3 in primary osteoblasts collected from Slit3^{f/f} mice subsequently transduced with Cre expressing or control lentivirus. FIG. 18B shows representative μCT images of the trabecular bone in the distal femur in Slit3^{f/f} (n=4) and Slit3^{osx} mice (n=6) at 8-weeks of age. Scale bars, 1 mm. FIG. 18C-1 graphically illustrates bone volume/total volume in Slit3^{f/f} (n=4) and Slit3^{osx} mice (n=6) at 8-weeks of age. FIG. 18C-2 graphically illustrates cortical bone thickness in Slit^{f/f} (n=4) and Slit3^{osx} mice (n=6) at 8-weeks of age. FIG. 18D-1 shows representative μCT images of the trabecular bone in the distal femur in Slit3^{f/f} (n=6) and Slit3^{dmp1} mice (n=6) at 8-weeks of age. Scale bars, 1 mm. FIG. 18D-2 graphically illustrates bone volume/total volume in Slit3^{f/f} (n=6) and Slit3^{dmp1} mice (n=6) at 8-weeks of age. FIG. 18E-1 shows representative flow cytometry plots within the Ter119⁻, CD45⁻ cell fraction isolated from the femur of 2-week-old Slit3^{f/f} and Slit3^{dmp1} mice (n=4 per group). Values represent mean±s.e.m.; *P<0.05 and **P<0.01 by an unpaired two-tailed Student's t-test in all panels. FIG. 18E-2 graphically illustrates the relative frequency of CD31^{hi}Emcn^{hi} endothelial cells within the Ter119⁻, CD45⁻ cell fraction isolated from the femur of 2-week-old Slit3^{f/f} and Slit3^{dmp1} mice (n=4 per group). Values represent mean±s.e.m.; *P<0.05 and **P<0.01 by an unpaired two-tailed Student's t-test in all panels.

FIG. 19A-19B show that skeletal functions of Slit3 do not map to endothelial cells or neurons. FIG. 19A-1 shows representative μCT images of the trabecular bone in the distal femur and quantitative analysis of bone volume/total volume in Slit3^{f/f} (n=6) and Slit3^{cdh5} mice (rt=5) at 8-weeks of age. Scale bars, 1 mm. FIG. 19A-2 graphically illustrates bone volume/total volume in Slit3^{f/f} (n=6) and Slit3_{cdh5} mice (n=5) at 8-weeks of age. FIG. 19B-1 shows representative μCT images of the trabecular bone in the distal femur of Slit3^{f/f} (n=5) and Slit3^{syn1} mice (n=6) at 8-weeks of age. Scale bars, 1 mm. Values represent mean±s.e.m.; n.s., not significant by an unpaired two-tailed Student's t-test in all panels. FIG. 19B-2 graphically illustrates bone volume/total volume in Slit3^{f/f} (n=5) and Slit3^{syn1} mice (n=6) at 8-weeks of age. Scale bars, 1 mm. Values represent mean±s.e.m.; n.s., not significant by an unpaired two-tailed Student's t-test in all panels.

FIG. 20A-20H illustrates that ROBO1 is a key receptor of SLIT3 signals regulating skeletal angiogenesis and bone mass accrual. FIG. 20A is a heatmap of SLIT/ROBO expression from three sets per genotype of independently derived CD31^{hi}EMCN^{hi} endothelial cells sorted from Shn3^{f/f} and Shn3^{dmp1} mice. FIG. 20B graphically illustrates results of quantitative real-time PCR analysis of Robo1-4 expression in murine osteoblasts using a murine total brain mRNA preparation as a positive control, (n=4). FIG. 20C graphically illustrates results of quantitative real-time PCR analysis of Robo1-4 expression in murine osteoclasts using a murine total brain mRNA preparation as a positive control, (n=4). FIG. 20D-1 shows representative μCT images of the trabecular bone in the distal femur metaphysis of trabecular bone mass (right) in Robo1^{-/-} and Robo3^{+/+} mice at 6-weeks of age (male, n≥6). FIG. 20D-2 graphically illustrates quantitative analysis of trabecular bone mass (right) in Robo1^{-/-} and Robo1^{+/+} mice at 6-weeks of age (male, n≥6). FIG. 20E-1 shows representative μCT images of the trabecular bone in the distal femur metaphysis of Robo4^{-/-} and Robo4^{+/+} mice at 6-weeks of age (male, n=6). FIG. 20E-2 graphically illustrates quantitative analysis of trabecular bone mass in Robo4^{-/-} and Robo4^{+/+} mice at 6-weeks of age (male, n=6). FIG. 20F shows representative flow cytometry plots of CD31^{hi}EMCN^{hi} endothelial cells and a heatmap of SLIT/ROBO expression from two sets per genotype of independently derived CD31^{hi}EMCN^{hi} endothelial cells sorted from Robo3^{+/+} and Robo1^{-/-} mice. FIG. 20G-1 graphically illustrates mRNA levels of Robo1 in mouse EPOCs expressing a control vector or Robo1 shRNA. Values represent mean±s.e.m.; n.s., not significant and *P<0.05 by an unpaired two-tailed Student's t-test in all panels. FIG. 20G-2 shows protein levels of Robo1 in mouse EPOCs expressing a control vector or Robo1 shRNA. Values represent mean±s.e.m.; n.s., not significant and *P<0.05 by an unpaired two-tailed Student's t-test in all panels. FIG. 20H-1 graphically illustrates mRNA levels of Robo1 in mouse EPOCs expressing a control vector or Robo1 shRNA. Brain is included as a positive control for Robo2 expression. Values represent mean±s.e.m; n.s., not significant and *P<0.05 by an unpaired two-tailed Student's t-test in all panels. FIG. 20H-2 illustrates protein levels of Robo2 in mouse EPOCs expressing a control vector or Robo1 shRNA. Brain is included as a positive control for Robo2 expression. Values represent mean±s.e.m.; n.s., not significant and *P<0.05 by an unpaired two-tailed Student's t-test in all panels.

FIG. 21A-21D shows that the hippo pathway is a key mediator of SLIT3/ROBO1-induced tube formation. FIG. 21A-1 shows representative images from EPOCs transduced with a Robo1 or control shRNA, treated with SLIT3 or FGF2, and subjected to a Matrigel tube formation assay. n=5 per group. FIG. 21A-2 graphically illustrates tube branch numbers for EPOCs transduced with a Robo1 or control shRNA, treated with SLIT3 or FGF2, and subjected to a Matrigel tube formation assay. n=5 per group. FIG. 21B-1 shows an immunoblot of EPOC proteins after EPOCs were transduced with a control shRNA, where immunoblotting was conducted with the indicated antibodies after SLIT3 treatment. FIG. 21B-2 shows an immunoblot of EPOC proteins after EPOCs were transduced with a Robo1 shRNA, where immunoblotting was conducted with the indicated antibodies after SLIT3 treatment. FIG. 21C illustrates protein levels of YAP as determined by immunoblotting of mouse EPOCs expressing a YAP or control shRNA. FIG. 21D-1 shows a Matrigel tube formation assay where EPOCs were first transduced with a Robo1 or control shRNA, then treated with SLIT3 or FGF2, and placed in a Matrigel tube formation assay; n=5 per group. Scale bars, 100 μm. Values represent mean±s.e.m.; n.s., not significant, *P<0.05, **P<0.01 and ***P<0.001 by a one-way ANOVA followed by a Tukey's posttest in all panels. FIG. 21D-2 graphically illustrates tube branch numbers formed in a Matrigel tube formation assay by EPOCs that were first transduced with a Robo1 or control shRNA, then treated with SLIT3 or FGF2, and placed in a Matrigel tube formation assay; n=5 per group. Scale bars, 100 μm. Values represent mean±s.e.m.; n.s., not significant, *P<0.05, **P<0.01 and ***p<0.001 by a one-way ANOVA followed by a Tukey's posttest in all panels.

FIG. 22A-22B illustrate that interaction between Slit3 and Shn3 null alleles is intrinsic to osteoblasts. FIG. 22A-1 shows representative μCT images of the trabecular bone in the distal femur metaphysis of 8-week-old OSX cre nice. Scale bar, 1 mm. FIG. 22A-2 shows representative μCT images of the trabecular bone in the distal femur metaphysis of 8-week-old OSX cre-Slit3^{f/+} mice. Scale bar, 1 mm. FIG. 22A-3 shows representative μCT images of the trabecular bone in the distal femur metaphysis of 8-week-old Osx cre-Shn3^{f/+} mice. Scale bar, 1 mm. FIG. 22A-4 shows representative μCT images of the trabecular bone in the distal femur metaphysis of 8-week-old OSX cre-Slit3^{f/+}Shn3^{f/+} nice. Scale bar, lima FIG. 22B graphically illustrates BV/TV analysis of 8-week-old OSX ere, OSX cre-Slit3^{f/+}, Osx cre-Shn3^{f/+} and OSX ere-Slit3^{f/+}Shn3^{f/+} mice.

FIG. 23A-23B SLIT3 is expressed in osteoblasts present in human fracture callus tissue. FIG. 23A shows an image of a human fracture callus that was immunohistochemically stained for SLIT3. Scale bars, 100 μm. FIG. 23B-1 shows an image of a human fracture callus that was H&E stained. Scale bars, 100 μm. FIG. 23B-2 shows an image of a human fracture callus that was immunohistochemically stained for CD31. The images shown for FIGS. 23B-1 and 23B-2 were from serial sections from human fracture callus tissue. Scale bars, 100 μm.

FIG. 24A-24C illustrates that systemic administration of SLIT3 enhances bone-fracture healing. FIG. 24A graphically illustrates the quantity of EMCN-positive vessel volume/total volume (Relative vessel volume (%)) in a callus area as determined by histomorphometry of femurs 21 days after fracture in male Shn3^{+/+} Slit3^{+/+}, Shn3^{+/+} Slit3^{-/-}, Shn3^{-/-} Slit3^{+/+} and Shn3^{-/-} Slit3^{-/-} mice. FIG. 24B graphically illustrates the stiffness determined by bio-mechanical testing of femurs 21 days after fracture in male Shn3^{+/+} Slit3^{+/+}, Shn3^{+/+} Slit3^{-/-}, Shn3^{-/-} Slit3^{+/+} and Shn3^{-/-} Slit3^{-/-} mice. FIG. 24C shows images of fracture callus tissue in mice treated with IV injection of SLIT3 or vehicle and at 21 days post-fracture callus tissue was harvested and immunostained for CD31 and EMCN. Scale bars, 100 μm, n=3 per group. Values represent mean±s.e.m.; **P<0.01 and ***p<0.001 by one-way ANOVA followed by a Tukey's posttest in all panels.

FIG. 25A-25D illustrates that systemic administration of SLIT3 does not cause abnormalities in the amount or morphology of non-skeletal vasculature. FIG. 25A shows representative confocal images of retinal flat-mounts of 8-week-old mice stained with ve-cadherin (green) from mice 21 days post-fracture with IV injection of SLIT3 or Vehicle. Scale bars, 500 μm, n=3 per group. FIG. 25B shows representative confocal images of heart, kidney and lung sections stained for endomucin (red) and 4′,6-diamidino-2-phenylindole (DAPI, blue) from mice 21 days post-fracture with IV injection of SLIT3 or Vehicle. Scale bars, 100 μm, n=3 per group. FIG. 25C shows representative images of the whole brain sections stained for neurofilament-M (NF-M, green) and Niss1 (red) from mice 21 days post-fracture with IV injection of SLIT3 or Vehicle. Scale bars, 500 μm, n=3 per group. FIG. 25D shows representative images of the whole brain sections stained for neurofilament-M (NF-M, green) and Niss1 (red) from mice 21 days post-fracture with IV injection of SLIT3 or Vehicle, where the boxed areas of the upper images are shown with higher magnification below. Scale bars, 500 μm, n=3 per group.

FIG. 26A-26F illustrates that local delivery of SLIT3 enhances bone-fracture healing. FIG. 26A shows representative μCT images of mouse femurs harvested 21 days post-fracture with insertion of a gelatin sponge soaked with SLIT3 or vehicle. A no sponge control is included. Scale bar=1 mm. FIG. 26B shows representative image of CD31 and EMCN (red) dual immunostained callus sections of mouse femurs 21 days post-fracture with insertion of a gelatin sponge soaked with SLIT3 or vehicle at the time of fracture. Scale bars, 300 μm, n=3 per group. FIG. 26C shows representative confocal images of retinal flat-mounts of 8-week-old mice stained with ve-cadherin (green). Retinas were harvested from mice 21 days post-fracture with implantation of a gelatin sponge soaked with SLIT3 or vehicle at the time of fracture. Scale bars, 500 μm, n=3 per group. FIG. 26D shows representative images of the whole brain sections stained for neurofilament-M (NF-M, green) and Niss1 (red). Brains were harvested from mice 21 days post-fracture after implantation of a gelatin sponge soaked with SLIT3 or vehicle. Scale bars, 500 μm, n=3 per group. FIG. 26E shows representative μCT images of mouse femurs. Mice were subjected to femoral fracture created by a midshaft osteotomy which was subsequently stabilized by an intramedullary rod. At the time of fracture, a hydrogel construct containing SLIT3 or a control gel construct only containing PBS vehicle were implanted at the periosteal surface near the site of fracture. FIG. 26F shows representative μCT images of mouse femurs. Mice were subjected to femoral fracture as described for FIG. 26E and a construct including a SLIT3-containing collagen sponge or a PBS vehicle containing control collagen sponge was similarly implanted at the fracture site. SLIT3-eluting collagen sponges were also supplemented with additional targeted injection of SLIT3 performed into collagen sponge 7 days after fracture (FIG. 26F, right)

FIG. 27A-27F illustrate that SLIT3 has preventive effects in the ovariectomized (OVX) mouse model. FIG. 27A shows representative images of the uterus from sham-operated (Sham) and ovariectomized (OVX) mice. Scale bar, 500 μm. FIG. 27B graphically illustrates quantitative analysis of uterus weight in Sham and OVX mice. FIG. 27C-1 graphically illustrates cortical bone thickness of distal femoral metaphyseal regions from mice after sham operation, OVX and OVX with SLIT3 injection. FIG. 27C-2 graphically illustrates trabecular number (Tb. N) of distal femoral metaphyseal regions from mice after sham operation, OVX and OVX with SLIT3 injection. FIG. 27C-3 graphically illustrates trabecular thickness (Tb. Th) of distal femoral metaphyseal regions from mice after sham operation, OVX and OVX with SLIT3 injection. FIG. 27C-4 graphically illustrates trabecular bone space (Tb. Sp) of distal femoral metaphyseal regions from mice after sham operation, OVX and OVX with SLIT3 injection. FIG. 27D shows representative confocal images of CD31 (green) and endomucin (red) dual-immunostained bone sections of the L3 vertebrae from mice after sham operation, OVX or OVX with SLIT3 injection. GP, growth plate. Scale bars, 300 μm. FIG. 27E-1 graphically illustrates periosteal bone formation rates/bone surface (BFR/BS, mm³ mm⁻² yr⁻¹) of the L3 vertebrae from mice after sham operation, OVX or OVX with SLIT3 injection. FIG. 27E-2 graphically illustrates endosteal bone formation rates/bone surface (BFR/BS, mm⁻³ mm⁻² yr⁻¹) of the L3 vertebrae from mice after sham operation, OVX or OVX with SLIT3 injection. FIG. 27F-1 graphically illustrates osteoclast number/bone perimeter (No. Oc./B. Pm) in the endosteum of the L3 vertebrae from mice after sham operation, OVX or OVX with SLIT3 injection. FIG. 27F-2 graphically illustrates osteoblast surface/bone surface (Ob.S/BS, %) in the endosteum of the L3 vertebrae from mice after sham operation, OVX or OVX with SLIT3 injection. Values represent mean±s.e.m.; *P<0.05 and **P<0.01 by one-way ANOVA followed by a Tukey's posttest in all panels.

FIG. 28A-28C illustrate that SLIT3 has therapeutic effects in the ovariectomized (OVX) mouse model. FIG. 28A shows representative μCT images of the trabecular bone in the distal femur of mice after sham operation, OVX, or OVX with SLIT3 injection. Scale bar, 1 mm. FIG. 28B-1 graphically illustrates bone volume/total volume in mice after sham operation, OVX, or OVX with SLIT3 injection. FIG. 28B-2 graphically illustrates cortical bone thickness in mice after sham operation, OVX, or OVX with SLIT3 injection. FIG. 28C shows representative flow cytometry dot plots and relative frequency of CD31^{hi}Emcn^{hi} endothelial cells of the femur in mice after sham operation, OVX and OVX with SLIT3 injection. Expanded views of such representative flow cytometry dot plots are shown in U.S. Provisional Patent Application Ser. No. 62/630,557 filed Feb. 14, 2018 (which is incorporated herein by reference in its entirety). Values represent mean±s.e.m.; *P<0.05, ***P<0.01, ***P<0.001 and ****p<0.0001 by one-way ANOVA followed by a Tukey's posttest in all panels.

FIG. 29A-29E illustrate SLIT2 expression and function in the skeletal system. FIG. 29A shows μCT images of bone present in a fracture callus of mice. Five-week-old mice underwent a midshaft osteotomy model of femoral fracture and were subsequently treated with recombinant SLIT2 delivered at 1 mg/kg for each of 3-weekly IV injections or vehicle injections for a total period of 3 weeks. At the end of this period, effects on bone healing were determined by μCT of the fracture site. Shown are representative 3-dimensional reconstructions of μCT images. FIG. 29B graphically illustrates quantitation of the relative amount of bone present in the fracture callus for the bones described in FIG. 29A. FIG. 29C shows representative 3-dimensional reconstructions of μCT images of trabecular bone (B, BV/TV, bone volume/total volume). FIG. 29D graphically illustrates quantitation of the relative amount of bone present in trabecular bone (B, BV/TV, bone volume/total volume). For FIGS. 29B and 29C, 12-week old male mice with a conditional deletion of Slit2 in osteoblast cells mediated by osxterix-cre (Slit2^{osx} mice) were imaged and underwent analysis of bone mass by μCT. FIG. 29E-1 shows representative images of Alizarin red staining of human bone stromal cells infected with lentivirus expressing Slit2 shRNA, Slit3 shRNA, Robo1 shRNA or a GFP-targeting control shRNA cultured under osteogenic conditions for 21 days. FIG. 29E-2 graphically illustrates quantitative relative mineralization capacity in human bone stromal cells infected with lentivirus expressing Slit2 shRNA, Slit3 shRNA, Robo1 shRNA or a GFP-targeting control shRNA cultured under osteogenic conditions for 21 days.

FIG. 30A-30B illustrates SLIT3 functions in osteoblasts to regulate bone mass. FIG. 30A-1 shows representative images of trabecular bone from three-week old Slit3^{f/f} and Slit3^{osx} mice. FIG. 30A-2 graphically illustrates trabecular bone mass (% BV/TV) of three-week old Slit3^{f/f} and Slit3^{osx} mice. FIG. 30B-1 shows representative images of trabecular bone from twelve-week old Slit3^{f/f} and Slit3^{osx} mice. FIG. 30B-2 graphically illustrates trabecular bone mass (% BV/TV) of twelve-week old Slit3^{f/f} and Slit3^{osx} mice. Mice of the indicated genotypes were sacrificed at 3 and 12 weeks of age for FIGS. 30A and 30B, respectively. Trabecular bone mass was determined by μCT of the femur.

FIG. 31 shows that systemic treatment with SLIT3 induces vascular leakage in retinal vasculature. Five-week old mice were treated with recombinant SLIT3 delivered at 1 mg/kg for each of 3 weekly IV injections or vehicle injections for a total period of 3 weeks. PE-tagged dextran (red) was infused prior to sacrifice to measure vascular permeability and retinal vessels were visualized by infusion of FITC tagged anti-CDH5 antibodies (green). Retinal vessel morphology (green) and permeability (red) were visualized by fluorescence microscopy of retinal mounts.

FIG. 32 schematically illustrates the strategy used for flow cytometry for analysis of osteoclast progenitor (OCPs).

FIG. 33A-33E illustrate that SLIT3 is dispensable for osteoclastogenesis in vitro. FIG. 33A graphically illustrates mRNA expression of Slit3 in BMMs treated with RANKL for up to three days, as measured by quantitative real-time PCR analysis, where brain tissue was used as a positive control. (n=3). FIG. 33B shows an immunoblot illustrating SLIT3 protein expression in BMMs treated with RANKL for two days, where the whole brain lysate from Slit3^{+/+} and Slit3^{-/-} mice as the positive and negative control. FIG. 33C graphically illustrates mRNA expression of Ntftac1, Itgb3, Dcstamp, Ctsk, Calcr and Trap in BMMs treated with RANKL for three days as measured by quantitative real-time PCR analysis. FIG. 33D-1 shows osteoclast differentiation using WT BMMs stimulated with RANKL and recombinant SLIT3 for three days. TRAP staining was performed and the area of TRAP-positive multinucleated cells (MNCs) (≥3 nuclei/cell) per well relative to the WT control was calculated. FIG. 33D-2 graphically illustrates numbers of osteoclasts per well. FIG. 33E-1 shows representative images of osteoclasts cultured in the presence of various concentrations of SLIT3. FIG. 33E-2 graphically illustrates the numbers of osteoclasts per well in cultures described for FIG. 33E-1.

FIG. 34A-34E illustrate that deletion of SLIT3 results in normal bone volume and normal osteoclast numbers in vim. FIG. 34A-1 illustrates flow cytometric analysis of in vivo osteoclast precursor populations of Slit3^{+/+} and Slit3^{-/-} mice. Representative flow cytometry plots for each experiment are shown. FIG. 34A-2 graphically illustrates the frequency of osteoclast precursors in Slit3^{+/+} and Slit3^{-/-} mice. FIG. 34B-1 illustrates the amount of osteoclast precursors in Slit3^{f/f} mice and in Slit3^{f/f} mice bred to cre-deleter strains targeting osteoblasts via OSX-cre (Slit3^{osx} mice). FIG. 34B-2 graphically illustrates the frequency of osteoclast precursors in Slit3^{f/f} and Slit3^{osx} mice. FIG. 34C-1 illustrates the amount of osteoclast precursors Slit3^{f/f} mice and in Slit3^{f/f} mice that were bred to a cre-deleter strain targeting mature osteoclasts, CTSK-cre (Slit3^{ctsk}). FIG. 34C-2 graphically illustrates the frequency of osteoclast precursors in Slit3^{f/f} and Slit3^{ctsk} mice. FIG. 34D-1 shows images of osteoclast differentiation utilizing osteoclast precursor cells isolated from Slit3^{ctsk} mice and Slit3^{f/f}. FIG. 34D-2 graphically illustrates the numbers of osteoclasts per well generated from cultures of cells isolated from Slit3^{ctsk} mice and Slit3^{f/f}. FIG. 34E-1 shows representative micro-CT images of the distal femurs isolated from 5-week-old male Slit3^{ctsk} mice and Slit3^{f/f} mice. FIG. 34E-2 graphically illustrates femoral bone volume over total volume (BV/TV) of trabecular bone of the distal femurs isolated from 5-week-old littermate male Slit3^{ctsk} mice and Slit3^{f/f} mice. FIG. 34E-3 graphically illustrates cortical bone thickness of the distal femurs isolated from 5-week-old littermate male Slit3^{ctsk} mice and Slit3^{f/f} mice. FIG. 34E-4 graphically illustrates bone morphometric analysis (trabecular number (Tb. N)) of trabecular bone of the distal femurs isolated from 5-week-old littermate male Slit3^{ctsk} mice and Slit3^{f/f} mice. FIG. 34E-5 graphically illustrates bone morphometric analysis (trabecular thickness (Tb. Th)) of trabecular bone of the distal femurs isolated from 5-week-old littermate male Slit3^{ctsk} mice and Slit3^{f/f} mice. FIG. 34E-6 graphically illustrates bone morphometric analysis (trabecular space (Tb. Sp)) of trabecular bone of the distal femurs isolated from 5-week-old littermate male Slit3^{ctsk} mice and Slit3^{f/f} mice.

FIG. 35A-35D show that chimeric mice lacking SLIT3 in BM cells exhibit normal bone phenotype and skeletal vasculature. FIG. 35A is a schematic diagram illustrating the protocol for a BM transplantation experiment. FIG. 35B shows representative micro-CT images of trabecular bone from the distal femurs isolated from WT mice with bone marrow transplantation derived from Slit3^{+/+} and Slit3^{-/-} mice. FIG. 35C-1 graphically illustrates BV/TV of trabecular bone of the distal femurs isolated from Slit3^{+/+} and Slit3^{-/-} mice. FIG. 35C-2 graphically illustrates conical bone thickness of trabecular bone of the distal femurs isolated from Slit3^{+/+} and Slit3^{-/-} mice. FIG. 35C-3 graphically illustrates bone morphometric analysis (trabecular number (Tb. N)) of trabecular bone of the distal femurs isolated from Slit3^{+/+} and Slit3^{-/-} mice. FIG. 35C-4 graphically illustrates bone morphometric analysis (trabecular thickness (Tb. Th)) of trabecular bone of the distal femurs isolated from Slit3^{+/+} and Slit33^{-/-} mice. FIG. 35C-5 graphically illustrates bone morphometric analysis (trabecular space (Tb. Sp)) of trabecular bone of the distal femurs isolated from Slit33^{+/+} and Slit3^{-/-} mice. FIG. 35D shows histomorphometry and immunofluorescence staining of SLIT3 from osteoclast lineage cells illustrating that neither osteoclast numbers nor bone vasculature were altered by bone marrow transplantation and the absence of SLIT3 from osteoclast lineage cells.

### DETAILED DESCRIPTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that logical changes may be made without departing from the scope of the present invention. The following description of example embodiments is, therefore, not to be taken in a limited sense, and the scope of the present invention is defined by the appended claims.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

Recent studies have identified a specialized subset of CD31^{hi}EMCN^{hi} vascular endothelium that regulates bone formation. However, it remains unclear how CD31^{hi}EMCN^{hi} endothelium levels are coupled to anabolic bone formation. Analysis of a strain of mutant mice with elevated bone formation, Shn3^{-/-} mice, demonstrated an increase in CD31^{hi}EMCN^{hi} endothelium that cell-specific genetic models mapped to osteoblasts.

Transcriptomic analysis identified SLIT3 as an osteoblast derived, SHN3-regulated proangiogenic factor, and absence of Slit3 reduced skeletal CD31^{hi}EMCN^{hi} endothelium, resulted in low bone mass due to impaired bone formation, and partially reversed the high bone mass phenotype of Shn3^{-/-} mice. This coupling between osteoblasts and CD31^{hi}EMCN^{hi} endothelium is essential for bone healing, as shown by defective fracture repair in SLIT3-mutant mice and enhanced fracture repair in SHN3-mutant mice. Drugs that target the SLIT3 pathway are a novel class of vascular-targeted osteoanabolic therapy as administration of recombinant SLIT3 both enhanced bone-fracture healing and counteracted bone loss in a mouse model of postmenopausal osteoporosis.

SLIT agonists are an entirely novel class of pro-anabolic agents distinct from all existing drugs, as SLITs do not act directly on osteoblasts but rather indirectly promote bone formation by enhancing the formation of type H endothelium in bone, a specialized subtype of blood vessels that promote bone formation. This means that SLITs will likely have a distinct set of advantages versus all existing agents. Due to the known importance of blood vessels in fracture healing, we propose that this indication and other related orthopedic bone repair applications potentially represent a unique application for SLITs. In this respect, they may also function as growth factors applied locally to a site where enhanced bone repair is desired in a manner similar to recombinant BMPs.

Impaired fracture healing is observed in elderly patients, patients with systemic vascular diseases such as diabetes, patients with inflammatory disorders or chronic infection, or in patients with large traumatic bone defects (Buza and Einhorn 2016). For these classes of patients, a single bone fracture often results in many years of pain, severely impaired mobility and numerous attempts at surgical management of their fracture. From this perspective, developing a means for medical therapy to promote fracture healing is urgently needed. Interestingly, the phenotypes observed with disruption of the SHN3/SLIT3 axis may extend beyond simply promoting more bone formation, as the fracture callus observed in SHN3-deficient mice was markedly more mature, including displaying overall more mature lamellar bone in addition to enhanced recruitment of hematopoietic elements to the callus. Conversely, SLIT3-deficient mice displayed an arrest at early stages of fracture callus maturation, displaying a lack of propagation of the mineralization sites on either side of the callus.

We have demonstrated that exogenous SLIT3 promotes bone fracture healing and prevents bone loss in a model of postmenopausal osteoporosis. Notably, these findings contrast with a prior in vitro study indicating that SLIT2 suppresses osteoblast differentiation in vitro (Sun et al. 2009). These results show that agents targeting bone vasculature represent a novel class of bone anabolics and that vascular-targeted anabolics may have a synergistic or complimentary effect when used in combination with an osteoblast targeted anabolic such as a PTH analogue or an anti-SOST antibody.

Development of new categories of bone anabolic agents is especially important given the current limitations on the maximum duration of therapy with PTH-based anabolic agents. Likewise, in light of increasing evidence establishing that osteoporosis drugs can be used in a sequential or combination manner to obtain superior clinical outcomes, having therapeutic access to a larger diversity of anabolic pathways is highly desirable (Leder et al. 2015). Further enhancement of the magnitude of SLIT3 effect is possible by optimization of dosing and delivery strategies. For instance, bone targeting strategies such as a bisphosphonate conjugation or (AspSerSer)₆-liposomes and Aptamer-functionalized lipid nanoparticles may enhance the potency or anabolic effects of SLIT3 by increasing the fractional distribution to bone (Zhang, Guo, et al. 2012, Liang et al. 2015, Guan et al. 2012, Yao et al. 2013).

### Subjects

The subject may be any animal, including a human or non-human animal. Non-human animals include all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Preferred subjects include human subjects, for example, those in need of preventing bone loss or of promoting bone growth, strengthening, or healing, or in need of preventing bone growth. The subject can be diagnosed with such a condition by skilled artisans, such as a medical practitioner.

The methods described herein can be employed for subjects of any species, gender, age, ethnic population, or genotype. Accordingly, the term subject includes males and females, and it includes elderly, elderly-to-adult transition age adult subjects, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods described herein may be more appropriate for some ethnic populations such as Caucasians, especially northern European populations, as well as Asian populations.

The term subject also includes subjects of any genotype or phenotype. For example, any subject in need of treatment as described herein can be provided with such treatment. In addition, the subject can have the genotype or phenotype for any hair color, eye color, skin color or any combination thereof.

The term subject includes a subject of any body height, body weight, or any organ or body part size or shape.

A subject in need of prevention of bone loss or of promoting bone growth, bone strengthening, or bone healing includes a subject who has experienced a bone defect, fracture or break, a tooth replacement, either replacement of a subjects' own tooth or a prosthetic tooth, or ameliorate symptoms of an ongoing condition, such as for example, bone loss associated with, for example peri-menopause or menopause. The fracture or defect be congenital, a result of aging, of an accident, or a surgical procedure. It may for example the result of a bone or spine fusion procedure, or a tumor resection, or any procedure where bone may be resected or drilled, including the placements of implants or other medical hardware in or on any bone, including but not limited to spine, knee, and hips. The subject may have received a bone graft. The subject may have a critical size defect, or a persistent non- or partial-union following a fracture. The subject may have a disease or condition that causes bone loss or degradation, such as osteoarthritis or rheumatoid arthritis or osteoporosis, including idiopathic osteoporosis, secondary osteoporosis, transient osteoporosis of the hip, osteomalacia, skeletal changes of hyperparathyroidism, chronic renal failure (renal osteodystrophy), osteitis deformans (Paget's disease of bone), osteolytic metastases, and osteopenia in which there is progressive loss of bone density and thinning of bone tissue. Osteoporosis and osteopenia can result not only from aging and reproductive status but can also be secondary to numerous diseases and disorders, as well as due to prolonged use of numerous medications, e.g., anticonvulsants, corticosteroids, and/or immunosuppressive agents. Other diseases in which osteoporosis may be secondary include, but are not limited to, juvenile rheumatoid arthritis, diabetes, osteogenesis imperfecta, hyperthyroidism, hyperparathyroidism, Cushing's syndrome, malabsorption syndromes, anorexia nervosa and/or kidney disease. In addition, numerous behaviors have been associated with osteoporosis, such as, prolonged inactivity or immobility, inadequate nutrition (especially calcium, vitamin D), excessive exercise leading to amenorrhea (absence of periods), smoking, and/or alcohol abuse. The subject may have any disease or condition known in the art wherein bone growth, bone strengthening, or prevention of bone loss would be beneficial. This can also include applications to enhance the integration of orthopedic hardware, such as used in joint arthoplasty, spinal fusion, or internal or external fixation of bone with the surrounding bone to prevent hardware loosening. Similar applications include counteracting the osteolysis observed at the site of orthopedic implants due to implant wear particles, infection, inflammation or other causes.

A subject in need of preventing bone growth includes subjects with conditions in which there is premature fusing of two or more bones, or bone density is too high, such as for example, craniosynostosis (synostosis), osteopetrosis (including malignant infantile form, intermediate form, and adult form), heterotopic ossification secondary to burn, traumatic injury or other condition, fibrodysplasia ossificans progressiva, osteitis deformans (Paget's disease of bone), primary extra-skeletal bone formation, e.g., multiple miliary osteoma cutis of the face, and osteitis condensans, or other diseases or conditions known in the art.

### SLIT3 or SLIT2 Agents

For the purposes of this application, a SLIT3 or SLIT2 agent is a nucleic acid or protein construct, wherein the SLIT 3 or SLIT2 nucleic acid expresses a SLIT3 or SLIT3 protein construct or polypeptide.

One example of a Homo sapiens SLIT3 protein sequence (e.g., with NCBI accession number AAQ89243) is shown below as SEQ ID NO:1.

| | |
|---|---|
| 1 | MAPGWAGVGA AVRARLALAL ALASVLSGPP AVACPTKCTC |
| 41 | SAASVDCHGL GLRAVPRGIP RNAERLDLDR NNITRITKMD |
| 81 | FAGLKNLRVL HLEDNQVSVI ERGAFQDLKQ LERLRLNKNK |
| 121 | LQVLPELLFQ STPKLTRLDL SENQIQGIPR KAFRGITDVK |
| 161 | NLQLDNNHIS CIEDGAFRAL RDLEILTLNN NNISRILVTS |
| 181 | FNHMPKIRTL RLHSNHLYCD CHLAWLSDWL RQRRTVGQFT |
| 241 | LCMAPVHLRG FNVADVQKKE YVCPAPHSEP PSCNANSISC |
| 281 | PSPCTCSNNI VDCRGKGLME IPANLPEGIV EIRLEQNSIK |
| 321 | AIPAGAFTQY KKLKRIDISK NQISDIAPDA FQGLKSLTSL |
| 361 | VLYGNKITEI AKGLFDGLVS LQLLLINANK INCLRVNTFQ |
| 401 | DLQNLNLLSL YDNKLQTISK GLFAPLQSIQ TLHLAQNPFV |
| 441 | CDCHLKWLAD YLQDNPIETS GARCSSPRRL ANKRISQIKS |
| 481 | KKFRCSGSED YRSRFSSECF MDLVCPEKCR CEGTIVDCSN |
| 521 | QKLVRIPSHL PEYVTDLRLN DNEVSVLEAT GIFKKLPNLR |
| 561 | KINLSNNKIK EVREGAFDGA ASVQELMLTG NQLETVHGRV |
| 601 | FRGLSGLKTL MLRSNLISCV SNDTFAGLSS VRLLSLYDNR |
| 641 | ITTITPGAFT TLVSLSTINL LSNPFNCNCH LAWLGKWLRK |
| 681 | RRIVSGNPRC QKPFFLKEIP IQDVAIQDFT CDGNEESSCQ |
| 721 | LSPRCPEQCT CMETVVRCSN KGLRALPRGM PKDVTELYLE |
| 761 | GNHLTAVPRE LSALRHLTLI DLSNNSISML TNYTFSNMSH |
| 801 | LSTLILSYNR LRCIPVHAFN GLRSLRVLTL HGNDISSVPE |
| 841 | GSFNDLTSLS HLALGTNPLH CDCSLRWLSE WVKAGYKEPG |
| 881 | IARCSSPEPM ADRLLLTTPT HRFQCKGPVD INIVAKCNAC |
| 921 | LSSPCKNNGT CTQDPVELYR CACPYSYKGK DCTVPINTCI |
| 961 | QNPCQHGGTC HLSDSHKDGF SCSCPLGYEG QRCEINPDDC |
| 1001 | EDNDCENNAT CVDGINNYVC ICPPNYTGEL CDEVIDHCVP |
| 1041 | ELNLCQHEAK CIPLDKGFSC ECVPGYSGKL CETDNDDCVA |
| 1081 | HKCRHGAQCV DTINGYTCTC PQGFSGPFCE HPPPMVLLQT |
| 1121 | SPCDQYECQN GAQCIVVQQE PTCRCPPGFA GPRCEKLITV |
| 1161 | NFVGKDSYVE LASAKVRPQA NISLQVATDK DNGILLYKGD |
| 1201 | NDPLALELYQ GHVRLVYDSL SSPPTTVYSV ETVNDGQFHS |
| 1241 | VELVTLNQTL NLVVDKGTPK SLGKLQKQPA VGINSPLYLG |
| 1281 | GIPTSTGLSA LRQGTDRPLG GFHGCIHEVR INNELQDFKA |
| 1321 | LPPQSLGVSP GCKSCTVCKH GLCRSVEKDS VVCECRPGWT |
| 1361 | GPLCDQEARD PCLGHRCHHG KCVATGTSYM CKCAEGYGGD |
| 1401 | LCDNKNDSAN ACSAFKCHHG QCHISDQGEP YCLCQPGFSG |
| 1441 | EHCQQENPCL GQVVREVIRR QKGYASCATA SKVPIMECRG |
| 1481 | GCGPQCCQPT RSKRRKYVFQ CTDGSSFVEE VERHLECGCL |
| 1521 | ACS |

Amino acids at plus or minus one (±1) position of amino add positions at any of 565, 566, 662, 761, 784, 832, 853, 855, or 869 (highlighted above) within SLIT3 proteins (e.g. having SEQ ID NO: 1) can be involved in substrate binding. Amino adds at plus or minus one (±1) position of amino add positions at any of 956-994, 998-1031, 1035-1072, 1074-1110, within SLIT3 proteins (e.g. having SEQ ID NO: 1) can be at least part of a calcium-binding EGF-like domain. For example, amino acids at plus or minus one (±1) position of amino acid positions at any of 1074, 1077, or 1091 within SLIT3 proteins (e.g. having SEQ ID NO: 1) can be at least part of a calcium-binding site (ion binding). Amino acids at plus or minus one (±1) position of amino acid positions at any of 1123-1153 or 1466-1521 within SLIT3 proteins (e.g. having SEQ ID NO: 1) can be at least part of an EGF-like domain. Amino acids at plus or minus one (±1) position of amino acid positions at any of 1188-1314 within SLIT3 proteins (e.g. having SEQ ID NO: 1) can be at least part of a Laminin G domain.

An example of a nucleic acid that encodes the SEQ ID NO:1 SLIT3 protein is shown (NCBI accession number AY358884; SEQ ID NO:2).

| | |
|---|---|
| 1 | CGCGCTCCCC GCGCGCCTCC TCGGGCTCCA CGCGTCTTGC |
| 41 | CCCGCAGAGG CAGGCTCCTC CAGGAGCGGG GCCCTGCACA |
| 81 | CCATGGCCCC CGGGTGGGCA GGGGTCGGCG CCGCCGTGCG |
| 121 | CGCCCGCCTG GCGCTGGCCT TGGCGCTGGC GAGCGTCCTG |
| 161 | AGTGGGCCTC CAGCCGTCGC CTGCCCCACC AAGTGTACCT |
| 201 | GCTCCGCTGC CAGCGTGGAC TGCCACGGGC TGGGCCTCCG |
| 241 | CGCGGTTCCT CGGGGCATCC CCCGCAACGC TGAGCGCCTT |
| 281 | GACCTGGACA GAAATAATAT CACCAGGATC ACCAAGATGG |
| 321 | ACTTCGCTGG GCTCAAGAAC CTCCGAGTCT TGCATCTGGA |
| 361 | AGACAACCAG GTCAGCGTCA TCGAGAGAGG CGCCTTCCAG |
| 401 | GACCTGAAGC AGCTAGAGCG ACTGCGCCTG AACAAGAATA |
| 441 | AGCTGCAAGT CCTTCCAGAA TTGCTTTTCC AGAGCACGCC |
| 481 | GAAGCTCACC AGACTAGATT TGAGTGAAAA CCAGATCCAG |
| 521 | GGGATCCCGA GGAAGGCGTT CCGCGGCATC ACCGATGTGA |
| 561 | AGAACCTGCA ACTGGACAAC AACCACATCA GCTGCATTGA |
| 601 | AGATGGAGCC TTCCGAGCGC TGCGCGATTT GGAGATCCTT |
| 641 | ACCCTCAACA ACAACAACAT CAGTCGCATC CTGGTCACCA |
| 681 | GCTTCAACCA CATGCCGAAG ATCCGAACTC TGCGCCTCCA |
| 721 | CTCCAACCAC CTCTACTGCG ACTGCCACCT GGCCTGGCTC |
| 761 | TCGGATTGGC TGCGACAGCG ACGGACAGTT GGCCAGTTCA |
| 801 | CACTCTGCAT GGCTCCTGTG CATTTGAGGG GCTTCAACGT |
| 841 | GGCGGATGTG CAGAAGAAGG AGTACGTGTG CCCAGCCCCC |
| 881 | CACTCGGAGC CCCCATCCTG CAATGCCAAC TCCATCTCCT |
| 921 | GCCCTTCGCC CTGCACGTGC AGCAATAACA TCGTGGACTG |
| 961 | TCGAGGAAAG GGCTTGATGG AGATTCCTGC CAACTTGCCG |
| 1001 | GAGGGCATCG TCGAAATACG CCTAGAACAG AACTCCATCA |
| 1041 | AAGCCATCCC TGCAGGAGCC TTCACCCAGT ACAAGAAACT |
| 1081 | GAAGCGAATA GACATCAGCA AGAATCAGAT ATCGGATATT |
| 1121 | GCTCCAGATG CCTTCCAGGG CCTGAAATCA CTCACATCGC |
| 1161 | TGGTCCTGTA TGGGAACAAG ATCACCGAGA TTGCCAAGGG |
| 1201 | ACTGTTTGAT GGGGTGGTGT CCCTACAGCT GCTCCTCCTC |
| 1241 | AATGCCAACA AGATCAACTG CCTGCGGGTG AACACGTTTC |
| 1281 | AGGACCTGCA GAACCTCAAG TTGCTCTCCC TGTATGACAA |
| 1321 | CAAGCTGCAG ACCATCAGCA AGGGGCTCTT CGCCCCTCTG |
| 1361 | CAGTCCATCC AGACACTCCA CTTAGCCCAA AACCCATTTG |
| 1401 | TGTGCGACTG CCACTTGAAG TGGCTGGCCG ACTACCTCCA |
| 1441 | GGACAACCCC ATCGAGACAA GCGGGGCCCG CTGCAGCAGC |
| 1481 | CCGCGCCGAC TCGCCAACAA GCGCATCAGC CAGATCAAGA |
| 1521 | GCAAGAAGTT CCGCTGCTCA GGCTCCGAGG ATTAGCGCAG |
| 1561 | CAGGTTCAGC AGCGAGTGCT TCATGGACCT CGTGTGCCCC |
| 1601 | GAGAAGTGTC GCTGTGAGGG CACGATTGTG GACTGCTCCA |
| 1641 | ACCAGAAGCT GGTCCGCATC CCAAGCCACC TCCCTGAATA |
| 1681 | TGTCACCGAC CTGCGACTGA ATGACAATGA GGTATCTGTT |
| 1721 | CTGGAGGCCA CTGGCATCTT CAAGAAGTTG CCCAACCTGC |
| 1761 | GGAAAATAAA TCTGAGTAAC AATAAGATCA AGGAGGTGCG |
| 1801 | AGAGGGAGCT TTCGATGGAG CAGCCAGCGT GCAGGAGCTG |
| 1841 | ATGCTGACAG GGAACCAGCT GGAGACCGTG CACGGGCGCG |
| 1881 | TGTTCCGTGG CCTCAGTGGC CTCAAAACCT TGATGCTGAG |
| 1921 | GAGTAACTTG ATCAGCTGTG TGAGTAATGA CACCTTTGCC |
| 1961 | GGCCTGAGTT CGGTGAGACT GCTGTCCCTC TATGACAATC |
| 2001 | GGATCACCAC CATCACCCCT GGGGCCTTCA CCACGCTTGT |
| 2041 | CTCCCTGTCC ACCATAAACC TCCTGTCCAA CCCCTTCAAC |
| 2081 | TGCAACTGCC ACCTGGCCTG GCTCGGCAAG TGGTTGAGGA |
| 2121 | AGAGGCGGAT CGTCAGTGGG AACCCTAGGT GCCAGAAGCC |
| 2161 | ATTTTTCCTC AAGGAGATTC CCATCCAGGA TGTGGCCATC |
| 2201 | CAGGACTTCA CCTGTGATGG CAACGAGGAG AGTAGCTGCC |
| 2241 | AGCTGAGCCC GCGCTGCCCG GAGCAGTGCA CCTGTATGGA |
| 2281 | GACAGTGGTG CGATGCAGCA ACAAGGGGCT CCGCGCCCTC |
| 2321 | CCCAGAGGCA TGCCCAAGGA TGTGACCGAG CTGTACCTGG |
| 2361 | AAGGAAACCA CCTAACAGCC GTGCCCAGAG AGCTGTCCGC |
| 2401 | CCTCCGACAC CTGACGCTTA TTGACCTGAG CAACAACAGC |
| 2441 | ATCAGCATGC TGACCAATTA CACCTTCAGT AACATGTCTC |
| 2481 | ACCTCTCCAC TCTGATCCTG AGCTACAACC GGCTGAGGTG |
| 2521 | CATCCCCGTC CACGCCTTCA ACGGGCTGCG GTCCCTGCGA |
| 2561 | GTGCTAACCC TCCATGGCAA TGACATTTCC AGCGTTCCTG |
| 2601 | AAGGCTCCTT CAACGACCTC ACATCTCTTT CCCATCTGGC |
| 2641 | GCTGGGAACC AACCCACTCC ACTGTGACTG CAGTCTTCGG |
| 2681 | TGGCTGTCGG AGTGGGTGAA GGCGGGGTAC AAGGAGCCTG |
| 2721 | GCATCGCCCG CTGCAGTAGC CCTGAGCCCA TGGCTGACAG |
| 2761 | GCTCCTGCTC ACCACCCCAA CCCACCGCTT CCAGTGCAAA |
| 2801 | GGGCCAGTGG ACATCAACAT TGTGGCCAAA TGCAATGCCT |
| 2841 | GCCTCTCCAG CCCGTGCAAG AATAACGGGA CATGCACCCA |
| 2881 | GGACCCTGTG GAGCTGTACC GCTGTGCCTG CCCCTACAGC |
| 2921 | TACAAGGGCA AGGACTGCAC TGTGCCCATC AACACCTGCA |
| 2961 | TCCAGAACCC CTGTCAGCAT GGAGGCACCT GCCACCTGAG |
| 3001 | TGACAGCCAC AAGGATGGGT TCAGCTGCTC CTGCCCTCTG |
| 3041 | GGCTTTGAGG GGCAGCGGTG TGAGATCAAC CCAGATGACT |
| 3081 | GTGAGGACAA CGACTGCGAA AACAATGCCA CCTGCGTGGA |
| 3121 | CGGGATCAAC AACTACGTGT GTATCTGTCC GCCTAACTAC |
| 3261 | ACAGGTGAGC TATGCGACGA GGTGATTGAC CACTGTGTGC |
| 3201 | CTGAGCTGAA CCTCTGTCAG CATGAGGCCA AGTGCATCCC |
| 3241 | CCTGGACAAA GGATTCAGCT GCGAGTGTGT CCCTGGCTAC |
| 3281 | AGCGGGAAGC TCTGTGAGAC AGACAATGAT GACTGTGTGG |
| 3321 | CCCACAAGTG CCGCCACGGG GCCCAGTGCG TGGACACAAT |
| 3361 | CAATGGCTAC ACATGCACCT GCCCCCAGGG CTTCAGTGGA |
| 3401 | CCCTTCTGTG AACACCCCCC ACCCATGGTC CTACTGCAGA |
| 3441 | CCAGCCCATG CGACCAGTAC GAGTGCCAGA ACGGGGCCCA |
| 3481 | GTGCATCGTG GTGCAGCAGG AGCCCACCTG CCGCTGCCCA |
| 3521 | CCAGGCTTCG CCGGCCCCAG ATGCGAGAAG CTCATCACTG |
| 3361 | TCAACTTCGT GGGCAAAGAC TCCTACGTGG AACTGGCCTC |
| 3601 | CGCCAAGGTC CGACCCCAGG CCAACATCTC CCTGCAGGTG |
| 3641 | GCCACTGACA AGGACAACGG CATCCTTCTC TACAAAGGAG |
| 3681 | ACAATGACCC CCTGGCACTG GAGCTGTACC AGGGCCACGT |
| 3721 | GCGGCTGGTC TATGACAGCC TGAGTTCCCC TCCAACCACA |
| 3761 | GTGTACAGTG TGGAGACAGT GAATGATGGG CAGTTTCACA |
| 3801 | GTGTGGAGCT GGTGACGCTA AACCAGACCC TGAAGCTAGT |
| 3841 | AGTGGACAAA GGAACTCCAA AGAGCCTGGG GAAGCTCCAG |
| 3881 | AAGCAGCCAG CAGTGGGCAT CAACAGCCCC CTCTACCTTG |
| 3921 | GAGGCATCCC CACCTCCACC GGCCTCTCCG CCTTGCGCCA |
| 3961 | GGGCACGGAC CGGCCTCTAG GCGGCTTCCA CGGATGCATC |
| 4001 | CATGAGGTGC GCATCAACAA CGAGCTGCAG GACTTCAAGG |
| 4041 | CCCTCCCACC ACAGTCCCTG GGGGTGTCAC CAGGCTGCAA |
| 4081 | GTCCTGGACC GTGTGCAAGC ACGGCCTGTG CCGCTCCGTG |
| 4121 | GAGAAGGACA GCGTGGTGTG CGAGTGCCGC CCAGGCTGGA |
| 4161 | CCGGCCCACT CTGCGACCAG GAGGCCCGGG ACCCCTGCCT |
| 4201 | CGGCCACAGA TGCCACCATG GAAAATGTGT GGCAACTGGG |
| 4241 | ACCTCATACA TGTGCAAGTG TGCCGAGGGC TATGGAGGGG |
| 4281 | ACTTGTGTGA CAACAAGAAT GAfTCTGCCA ATGCCTGCTC |
| 4321 | AGCCTTCAAG TGTCACCATG GGCAGTGCCA CATCTCAGAC |
| 4361 | CAAGGGGAGC CCTACTGCCT GTGCCAGCCC GGCTTTAGCG |
| 4441 | GCGAGCACTG CCAACAAGAG AATCCGTGCC TGGGACAAGT |
| 4441 | AGTCCGAGAG GTGATCCGCC GCCAGAAAGG TTATGCATCA |
| 4481 | TGTGCCACAG CCTCCAAGGT GCCCATCATG GAATGTCGTG |
| 4521 | GGGGCTGTGG GCCCCAGTGC TGCCAGCCCA CCCGCAGCAA |
| 4561 | GCGGCGGAAA TACGTCTTCC AGTGCACGGA CGGCTCCTCG |
| 4601 | TTTGTAGAAG AGGTGGAGAG ACACTTAGAG TGCGGCTGCC |
| 4641 | TCGCGTGTTC CTAAGCCCCT GCCCGCCTGC CTGCCACCTC |
| 4681 | TCGGACTCCA GCTTGATGGA GTTGGGACAG CCATGTGGGA |
| 4721 | CCCCCTGGTG ATTCAGCATG AAGGAAATGA AGCTGGAGAG |
| 4761 | GAAGGTAAAG AAGAAGAGAA TATTAAGTAT ATTGTAAAAT |
| 4801 | AAACAAAAAA TAGAACTTAA AAAAAAAAAA AAAAAAAAAA |
| 4841 | AA |

Another example of a Homo sapiens SLIT3 protein sequence (e.g., with NCBI accession number NP_003053) is shown below as SEQ ID NO:3.

| | |
|---|---|
| 1 | MAPGWAGVGA AVRARLALAL ALASVLSGPP AVACPTKCTC |
| 41 | SAASVDCHGL GLRAVPRGIP RNAERLDLDR NNITRITKMD |
| 81 | FAGLKNLRVL HLEDNQVSVI ERGAFQDLKQ LERLRLNKNK |
| 121 | LQVLPELLFQ STPKLTRLDL SENQIQGIPR KAFRGITDVK |
| 161 | NLQLDNNHIS CIEDGAFRAL RDLEILTLNN NNISRILVTS |
| 201 | FNHMPKIRTL RLHSNHLYCD CHLAWLSDWL RQRRTVGQFT |
| 241 | LCMAPVHLRG ENVADVQKKE YVCPAPHSEP PSCNANSISC |
| 281 | PSPCTCSNNI VDCRGKGLME IPANLPEGIV EIRLEQNSIK |
| 321 | AIPAGAFTQY KKLKRIDISK NQISDIAPDA FQGLKSLTSL |
| 361 | VLYGNKITEI AKGLFDGLVS LQLLLLNANK INCLRVNTFQ |
| 401 | DLQNLNLLSL YDNKLQTISK GLFAPLQSIQ TLHLAQNPFV |
| 441 | CDCHLKWLAD YLQDNPIETS GARCSSPRRL ANKRISQIKS |
| 481 | KKFRCSGSED YRSRFSSECF MDLVCPEKCR CEGTIVDCSN |
| 521 | QKLVRIPSHL PEYVTDLRLN DNEVSVLEAT GIFKKLPNLR |
| 561 | KINLSNNKIK EVREGAFDGA ASVQELMLTG NQLETVHGRV |
| 601 | FRGLSGLKTL MLRSNLIGCV SNDTFAGLSS VRLLSLYDNR |
| 641 | ITTITPGAFT TLVSLSTINL LSNPFNCNCH LAWLGKWLRK |
| 681 | RRIVSGNPRC QKPFFIKEIP IQDVAIQDFT CDGNEESSCQ |
| 721 | LSPRCPEQCT CMETVVRCSN KGLRALPRGM PKDVTELYLE |
| 761 | GNHLTAVPRE LSALRHLTLI DLSNNSISML TNYTFSNMSH |
| 801 | LSTLILSYNR LRCIPVHAFN GLRSLRVLTL HGNDISSVPE |
| 841 | GSFNDLTSLS HLALGTNPLH CDCSLRWLSE WVKAGYKEPG |
| 881 | IARCSSPEPM ADRLLLTTPT HRFQCKGPVD INIVAKCNAC |
| 921 | LSSPCKNNGT CTQDPVELYR CACPYSYKGK DCTVPINTCI |
| 961 | QNPCQHGGTC HLSDSHKDGF SCSCPLGFEG QRCEINPDDC |
| 1001 | EDNDCENNAT CVDGINNYVC ICPPNYTGEL CDEVIDHCVP |
| 1041 | ELNLCQHEAK CIPLDKGFSC ECVPGYSGKL CETDNDDCVA |
| 1081 | HKCRHGAQCV DTINGYTCTC PQGFSGPFCE HPPPMVLLQT |
| 1121 | SPCDQYECQN GAQCIVVQQE PTCRCPPGFA GPRCEKLITV |
| 1161 | NFVGKDSYVE LASAKVRPQA NISLQVATDK DNGILLYKGD |
| 1201 | NDPLALELYQ GHVRLVYDSL SSPPTTVYSV ETVNDGQFHS |
| 1241 | VELVTLNQTL NLVVDKGTPK SLGKLQKQPA VGINSPLYLG |
| 1281 | GIPTSTGLSA LRQGTDRPLG GFHGCIHEVR INNELQDFKA |
| 1321 | LPPQSLGVSP GCKSCTVCKH GLCRSVEKDS VVCECRPGWT |
| 1361 | GPLCDQEARD PCLGERCHHG KCVATGTSYM CKCAEGYGGD |
| 1401 | LCDNKNDSAN ACSAFKCHHG QCHISDQGEP YCLCQPGFSG |
| 1441 | EHCQQENPCL GQVVREVIRR QKGYASCATA SKVPTMECRG |
| 1481 | GCGPQCCQPT RSKRRKYVFQ CTDGSSFVEE VERHLECGCL |
| 1521 | ACS |

Another example of a Homo sapiens SLIT3 protein sequence (e.g., with NCBI accession number XP_016865268) is shown below as SEQ ID NO:4.

| | |
|---|---|
| 1 | MMGDLDRNNI TRITKMDFAG LKNLRVLHLE DNQVSVIERG |
| 41 | AFQDLKQLER LRLNKNKLQV LPELLFQSTP KLTRLDLSEN |
| 81 | QIQGIPRKAF RGITDVKNLQ LDNNHISCIE DGAFRALRDL |
| 121 | EILTLNNNNI SRILVTSFNH MPKIRTLRLH SNHLYCDCHL |
| 161 | AWLSDWLRQR RTVGQFTLCM APVHLRGFNV ADVQKKEYVC |
| 201 | PAPHSEPPSC NANSISCPSP CTCSNNIVDC RGKGLMEIPA |
| 241 | NLPEGIVEIR LEQNSIKAIP AGAFTQYKKL KRIDISKNQI |
| 281 | SDIAPDAFQG LKSLTSLVLY GNKITEIVKG LEDGLVSLQL |
| 321 | LLLNANKINC LRVNTFQDLQ NLNLLSLYDN KLQTISKGLF |
| 361 | APLCSIQTLH LAQNPFVCDC HLKWLADYLQ DNPIETSGAR |
| 401 | CSSPRRLANK RISQIKSKFF RCSGSEDYRS RFSSECFMDL |
| 441 | VCPEKCRCEG TIVDCSNQKL VRIPSHLPEY VTDLRLNDNE |
| 481 | VSVLEATGIF KKLPNLRKIN LSNNKTKEVR EGAFDGAASV |
| 521 | QELMLTGNQL ETVHGRVFRG LSGLKTLMLR SNLIGCVSND |
| 561 | TFAGLSSVRL LSLYDNRITT ITPGAFTTLV SLSTINLLSN |
| 601 | PFNCNCHLAW LGKWLRKRRI VSGNPRCQKP FFLKEIPIQD |
| 641 | VAIQDFTCDG NEESSCQLSP RCPEQCTCME TVVRCSNKGL |
| 681 | RALPRGMPKD VTELYLEGNH LTAVPRELSA LRHLTLIDLS |
| 721 | NNSISMLTNY TFSNMSHLST LILSYNRLRC IPVHAFNGLR |
| 761 | SLRVLTLHGN DISSVPEGSF NDLTSLSHLA LGTNPLHCDC |
| 801 | SLRWLSEWVK AGYKEPGIAR CSSPEPMADR LLLTTPTHRF |
| 841 | QCKGPVDINI VAKCNACLSS PCKNNGTCTQ DPVELYRCAC |
| 881 | PYSYKGKDCT VPINTCIQNP CQHGGTCHLS DSHKDGFSCS |
| 921 | CPLGFEGQRC EINPDDCEDN DCENNATCVD GINNYVCICP |
| 961 | PNYTGELCDE VIDHCVPELN LCQHEAKCIP LDKGFSCECV |
| 1001 | PGYSGKLCET DNDDCVAHKC RHGAQCVDTI NGYTCTCPQG |
| 1041 | FSGPFCEHPP PMVLLQTSPC DQYECQNGAQ CIVVQQEPTC |
| 1081 | RCPPGFAGPR CEKLITVNFV GKDSYVELAS AKVRPQANIS |
| 1121 | LQVATDKDNG ILLYKGDNDP LALELYQGHV RLVYDSLSSP |
| 1161 | PTTVYSVETV NDGOFHSVEL VTLNQTLNLV VDKGTPKSLG |
| 1201 | KLQKQPAVGI NSPLYLGGIP TSTGLSALRQ GTDRPLGGFH |
| 1241 | GCIHEVRINN ELQDFKALPP QSLGVSPGCK SCTVCKHGLC |
| 1281 | RSVEKDSVVC ECRPGWTGPL CDQEARDPCL GHRCHHGKCV |
| 1321 | ATGTSYMCKC AEGYGGDLCD NKNDSANACS AFKCHHGQCH |
| 1361 | ISDQGEPYCL CQPGFSGEHC QQENPCLGQV VREVIRRQKG |
| 1401 | YASGATASKV PIMECRGGCG PQCGQPTRSK RRKYVFQCTD |

1441 GSSFVEEVER HLECGCLACS

A comparison of the SEQ ID NO: 1 and SEQ ID NO:3 SLIT3 amino add sequences is shown bellow.

99.9% identity in 1523 residues overlap; Score: 8255. Gap
frequency: 0.0%
Seq1 1
MAPGWAGVGAAVRARLALALALASVLSGPPAVACPTKCTCSAASVDCHGLGLRAVPRGIP
Seq3 1
MAPGWAGVGAAVRARLALALALASVLSGPPAVACPTKCTCSAASVDCHGLGLRAVPRGIP
************************************************************
Seq1 61
RNAERLDLDRNNITRITKMDFAGLKNLRVLHLEDNQVSVIERGAFQDLKQLERLRLNKNK
Seq3 61
RNAERLDLDRNNITRITKMDFAGLKNLRVLHLEDNQVSVIERGAFQDLKQLERLRLNKNK
************************************************************
Seq1 121
LQVLPELLFQSTPKLTRLDLSENQIQGIPRKAFRGITDVKNLQLDNNHISCIEDGAFRAL
Seq3 121
LQVLPELLFQSTPKLTRLDLSENQIQGIPRKAFRGITDVKNLQLDNNHISCIEDGAFRAL
************************************************************
Seq1 181
RDLEILTINNNNISRILVTSFNHMPKIRTLRLHSNHLYCDCHLAWLSDWLRQRRTVGQFT
Seq3 181
RDLEILTLNNNNISRILVTSFNHMPKIRTLRLHSNHLYCDCHLAWLSDWLRQRRTVGQFT
************************************************************
Seq1 241
LCMAPVHLRGFNVADVQKKEYVCPAPHSEPPSCNANSISCPSPCTCSNNIVDCRGKGLME
Seq3 241
LCMAPVHLRGFNVADVQKKEYVCPAPHSEPPSCNANSISCPSPCTCSNNIVDCRGKGLME
************************************************************
Seq1 301
IPANLPEGIVEIRLEQNSIKAIPAGAFTQYKKLKRIDISKNQISDIAPDAFQGLKSLTSL
Seq3 301
IPANLPEGIVEIRLEQNSIKAIPAGAFTQYKKLKRIDISKNQISDIAPDAFQGLKSLTSL
************************************************************
Seq1 361
VLYGNKITEIAKGLFDGLVSLQLLLLNANKINCLRVNTFQDLQNLNLLSLYDNKLQTISK
Seq3 361
VLYGNKITEIAKGLFDGLVSLQLLLLNANKINCLRVNTFQDLQNLNLLSLYDNKLQTISK
************************************************************
Seq1 421
GLFAPLQSIQTLHLAQNPFVCDCHLKWLADYLQDNPIETSGARCSSPRRLANKRISQIKS
Seq3 421
GLFAPLQSIQTLHLAQNPFVCDCHLKWLADYLQDNPIETSGARCSSPRRLANKRISQIKS
************************************************************
Seq1 481
KKFRCSGSEDYRSRFSSECFMDLVCPEKCRCEGTIVDCSNQKLVRIPSHLPEYVIDLRLN
Seq3 481
KKFRCSGSEDYRSRFSSECFMDLVCPEKCRCEGTIVDCSNQKLVRIPSHLPEYVTDLRLN
************************************************************
Seq1 541
DNEVSVLEATGIFKKLPNLRKINLSNNKIKEVREGAFDGAASVQELMLTGNQLETVHGRV
Seq3 541
DNEVSVLEATGIFKKLPNLRKINLSNNKIKEVREGAFDGAASVQELMLTGNQLETVHGRV
************************************************************
Seq1 601
FRGLSGLKTLMLRSNLISCVSNDTFAGLSSVRLLSLYDNRITTITPGAFTTLVSLSTINL
Seq3 601
FRGLSGLKTLMLRSNLIGCVSNDTFAGLSSVRLLSLYDNRITTITPGAFTTLVSLSTINL
***************** ******************************************
Seq1 661
LSNPFNCNCHLAWLGKWLRKRRIVSGNPRCQKPFFLKEIPIQDVAIQDFTCDGNEESSCQ
Seq3 661
LSNPFNCNCHLAWLGKWLRKRRIVSGNPRCQKPFFLKEIPIQDVAIQDFTCDGNEESSCQ
************************************************************
Seq1 721
LSPRCPEQCTCMETVVRCSNKGLRALPRGMPKDVTELYLEGNHLTAVPRELSALRHLTLI
Seq3 721
LSPRCPEQCTCMETVVRCSNKGLRALPRGMPKDVTELYLEGNHLTAVPRELSALRHLTLI
************************************************************
Seq1 781
DLSNNSISMLTNYTFSNMSHLSTLILSYNRLRCIPVHAFNGLRSLRVLTLHGNDISSVPE
Seq3 781
DLSNNSISMLTVYTFSNMSHLSTLILSYNRLRCIPVHAFNGLRSLRVLTLHGNDISSVPE
************************************************************
Seq1 841
GSFNDLTSLSHLALGTNPLHCDCSLRWLSEWVKAGYKEPGIARCSSPEPMADRLLLTTPT
Seq3 841
GSFNDLTSLSHLALGTNPLHCDCSLRWLSEWVKAGYKEPGIARCSSPEPMADRLLLTTPT
************************************************************
Seq1 901
HRFQCKGPVDINIVAKCNACLSSPCKNNGTCTQDPVELYRCACPYSYKGKDCTVPINICI
Seq3 901
HRFQCKGPVDINIVAKCNACLSSPCKNNGTCTQDPVELYRCACPYSYKGKDCTVPINICI
************************************************************
Seq1 961
QNPCQHGGTCHLSDSHKDGFSCSCPLGFEGQRCEINPDDCEDNDCENNATCVDGINNYVC
Seq3 961
QNPCQHGGTCHLSDSHKDGFSCSCPLGFEGQRCEINPDDCEDNDCENNATCVDGINNYVC
************************************************************
Seq1 1021
ICPPNYTGELCDEVIDHCVPELNLCQHEAKCIPLDKGFSCECVPGYSGKLCETDNDDCVA
Seq3 1021
ICPPNYTGELCDEVIDHCVPELNLCQHEAKCIPLDKGFSCECVPGYSGKLCETDNDDCVA
************************************************************
Seq1 1081
HKCRHGAQCVDTINGYTCTCPQGFSGPFCEHPPPMVLLQTSPCDQYECQNGAQCIVVQQE
Seq3 1081
HKCRHGAQCVDTINGYTCTCPQGFSGPFCEHPPPMVLLQTSPCDQYECQNGAQCIVVQQE
************************************************************
Seq1 1141
PTCRCPPGFAGPRCEKLITVNFVGKDSYVELASAKVRPQANISLQVATDKDNGILLYKGD
Seq3 1141
PTCRCPPGFAGPRCEKLITVNFVGKDSYVELASAKVRPQANISLQVATDKDNGILLYKGD
************************************************************
Seq1 1201
NDPLALELYQGHVRLVYDSLSSPPTTVYSVETVNDGQFHSVELVTLNQTLNLVVDKGTPK
Seq3 1201
NDPLALELYQGHVRLVYDSLSSPPTTVYSVETVNDGQFHSVELVTLNQTLNLVVDKGTPK
************************************************************
Seq1 1261
SLGKLQKQPAVGINSPLYLGGIPTSTGLSALRQGTDRPLGGFHGCIHEVRINNELQDFKA
Seq3 1261
SLGKLQKQPAVGINSPLYLGGIPTSTGLSALRQGTDRPLGGFHGCIHEVRINNELQDFKA
************************************************************
Seq1 1321
LPPQSLGVSPGCKSCTVCKHGLCRSVEKDSVVCECRPGWTGPLCDQEARDPCLGHRCHHG
Seq3 1321
LPPOSLGVSPGCKSCTVCKHGLCRSVEKDSVVCECRPGWTGPLCDQEARDPCLGHRCHHG
************************************************************
Seq1 1381
KCVATGTSYMCKCAEGYGGDLCDNKNDSANACSAFKCHHGQCHISDQGEPYCLCQPGFSG
Seq3 1381
KCVATGTSYMCKCAEGYGGDLCDNKNDSANACSAFKCHHGQCHISDQGEPYCLCQPGFSG
************************************************************
Seq1 1441
EHCQQENPCLGQVVREVIRRQKGYASCATASKVPIMECRGGCGPQCCQPTRSKRRKYVFQ
Seq3 1441
EHCQQENPCLGQVVREVIRRQKGYASCATASKVPIMECRGGCGPQCCQPTRSKRRKYVFQ
************************************************************
Seq1 1501 CTDGSSFVEEVERHLECGCLACS
Seq3 1501 CTDGSSFVEEVERHLECGCLACS
***********************

A comparison of the SEQ ID NO: 1 and SEQ ID NO:4 SLIT3 amino add sequences is shown below.

99.9% identity in 1457 residues overlap; Score: 7909.0; Gap
frequency: 0.0%
Seq1 67
DLDRNNITRITKMDFAGLKNLRVLHLEDNQVSVIERGAFQDLKQLERLRLNKNKLQVLPE
Seq4 4
DLDRNNITRITHMDFAGLKNLRVLHLEDNQVSVIERGAFQDLKQLERLRLNKNKLQVLPE
************************************************************
Seq1 127
LLFQSTPKLTRLDLSENQIQGIPRKAFRGITDVKNLQLDNNHISCIEDGAFRALRDLEIL
Seq4 64
LLFQSTPKLTRLDLSENQIQGIPRKAFRGITDVKNLQLDNNHISCIEDGAFRALRDLEIL
************************************************************
Seq1 187
TLNNNNISRILVTSFNHMPKIRTLRLHSNHLYCDCHLAWLSDWLRQRRTVGQFTLCMAPV
Seq4 124
TLNNNNISRILVTSFNHMPKIRTLRLHSNHLYCDCHLAWLSDWLRQRRTVGQFTLCMAPV
************************************************************
Seq1 247
HLRGFNVADVQKKEYVCPAPHSEPPSCNANSISCPSPCTCSNNIVDCRGKGLMEIPANLP
Seq4 184
HLRGFNVADVQKKEYVCPAPHSEPPSCNANSISCPSPCTCSNNIVDCRGKGLMEIPANLP
************************************************************
Seq1 307
EGIVEIRLEQNSIKAIPAGAFTQYKKLKRIDISKNQISDIAPDAFQGLKSLTSLVLYGNK
Seq4 244
EGIVEIRLEQNSIKAIPAGAFTQYKKLKRIDISKNQISDIAPDAFQGLKSLTSLVLYGNK
************************************************************
Seq1 367
ITEIAKGLFDGLVSLQLLLLNANKINCLRVNTFQDLQNLNLLSLYDNKLQTISKGLFAPL
Seq4 304
ITEIVKGLFDGLVSLQLLLLNANKINCLRVNTFQDLQNLNLLSLYDNKLQTISKGLFAPL
**** *******************************************************
Seq1 427
QSIQTLHLAQNPFVCDCHLKWLADYLQDNPIETSGARCSSPRRLANKRISQIKSKKFRCS
Seq4 364
QSIQTLHLAQNPFVCDCHLKWLADYLQDNPIETSGARCSSPRRLANKRISQIKSKKFRCS
************************************************************
Seq1 487
GSEDYRSRFSSECFMDLVCPEKCRCEGTIVDCSNQKLVRIPSHLPEYVIDLRLNDNEVSV
Seq4 424
GSEDYRSRFSSECFMDLVCPEKCRCEGTIVDCSNQKLVRIPSHLPEYVTDLRLNDNEVSV
************************************************************
Seq1 547
LEATGIFKKLPNLRKINLSNNKIKEVREGAFDGAASVQELMLTGNQLETVHGRVFRGLSG
Seq4 484
LEATGIFKKLPNLRKINLSNNKIKEVREGAFDGAASVQELMLTGNQLETVHGRVFRGLSG
************************************************************
Seq1 607
LKTLMLRSNLISCVSNDTFAGLSSVRLLSLYDNRITTITPGAFTTLVSLSTINLLSNPFN
Seq4 544
LKTLMLRSNLIGCVSNDTFAGLSSVRLLSLYDNRITTITPGAFTTLVSLSTINLLSNPFN
*********** ************************************************
Seq1 667
CNCHLAWLGKWLRKRRIVSGNPRCQKPFFLKEIPIQDVAIQDFTCDGNEESSCQLSPRCP
Seq4 604
CNCHLAWLGKWLRKRRIVSGNPRCQKPFFLKEIPIQDVAIQDFTCDGNEESSCQLSPRCP
************************************************************
Seq1 727
EQCTCMETVVRCSNKGLRALPRGMPKDVTELYLEGNHLTAVPRELSALRHLTLTDLSNNS
Seq4 664
EQCTCMETVVRCSNKGLRALPRGMPKDVTELYLEGNHLTAVPRELSALRHLTLIDLSNNS
************************************************************
Seq1 787
ISMLTNYTFSNMSHLSTLILSYNRLRCIPVHAFNGLRSLRVLTLHGNDISSVPEGSFNDL
Seq4 724
ISMLTNYTFSNMSHLSTLILSYNRLRCIPVHAFNGLRSLRVLTLHGNDISSVPEGSFNDL
************************************************************
Seq1 847
TSLSHLALGTNPLHCDCSLRWLSEWVKAGYKEPGIARCSSPEPMADRLLLTTPTHRFQCK
Seq4 784
TSLSHLALGTNPLHCDCSLRWLSEWVKAGYKEPGIARCSSPEPMADRLLLTTPTHRFQCK
************************************************************
Seq1 907
GPVDINIVAKCNACLSSPCKNNGTCTQDPVELYRCACPYSYKGKDCTVPINTCIQNPCQH
Seq4 844
GPVDINIVAKCNACLSSPCKNNGTCTQDPVELYRCACPYSYKGKDCTVPINTCIQNPCQH
************************************************************
Seq1 967
GGTCHLSDSHKDGFSCSCPLGFEGQRCEINPDDCEDNDCENNATCVDGINNYVCICPPNY
Seq4 904
GGTCHLSDSHKDGFSCSCPLGFEGQRCEINPDDCEDNDCENNATCVDGINNYVCICPPNY
************************************************************
Seq1 1027
TGELCDEVIDHCVPELNLCQHEAKCIPLDKGFSCECVPGYSGKLCETDNDDCVAHKCRHG
Seq4 964
TGELCDEVIDHCVPELNLCQHEAKCIPLDKGFSCECVPGYSGKLCETDNDDCVAHKCRHG
************************************************************
Seq1 1087
AQCVDTINGYTCTCPQGFSGPFCEHPPPMVLLQTSPCDQYECQNGAQCIVVQQEPTCRCP
Seq4 1024
AQCVDTINGYTCTCPQGFSGPFCEHPPPMVLLQTSPCDQYECQNGAQCIVVQQEPTCRCP
************************************************************
Seq1 1147
PGFAGPRCEKLITVNFVGKDSYVELASAKVRPQANISLQVAIDKDNGILLYKGDNDPLAL
Seq4 1084
PGFAGPRCEKLITVNFVGKDSYVELASAKVRPQANISLQVATDKDNGILLYKGDNDPLAL
************************************************************
Seq1 1207
ELYQGHVRLVYDSLSSPPTTVYSVETVNDGQFHSVELVTLNQTLNLVVDKGTPKSLGKLQ
Seq4 1144
ELYQGHVRLVYDSLSSPPTTVYSVETVNDGQFHSVELVTLNQTLNLVVDKGTPKSLGKLQ
************************************************************
Seq1 1267
KQPAVGINSPLYLGGIPTSTGLSALRQGTDRPLGGFHGCIHEVRINNELQDFKALPPQSL
Seq4 1204
KQPAVGINSPLYLGGIPTSTGLSALRQGTDRPLGGFHGCIHEVRINNELQDFKALPPQSL
************************************************************
Seq1 1327
GVSPGCKSCTVCKHGLCRSVEKDSVVCECRPGWTGPLCDQEARDPCLGHRCHHGKCVATG
Seq4 1264
GVSPGCKSCTVCKHGLCRSVEKDSVVCECRPGWTGPLCDQEARDPCLGHRCHHGKCVATG
************************************************************
Seq1 1387
TSYMCKCAEGYGGDLCDNKNDSANACSAFKCHHGQCHISDQGEPYCLCQPGFSGEHCQQE
Seq4 1324
TSYMCKCAEGYGGDLCDNKNDSANACSAFKCHHGQCHISDQGEPYCLCQPGFSGEHCQQE
************************************************************
Seq1 1447
NPCLGQVVREVIRRQKGYASCATASKVPIMECRGGCGPQCCQPTRSKRRKYVFQCTDGSS
Seq4 1384
NPCLGQVVREVIRRQKGYASCATASKVPIMECRGGCGPQCCQPTRSKRRKYVFQCIDGSS
************************************************************
Seq1 1507 FVEEVERHLECGCLACS
Seq4 1444 FVEEVERHLECGCLACS
*****************

Such sequence comparisons illustrate portions of the SLIT3 protein that tend to be conserved, and portions of the SLITS protein that are not so conserved.

An example of a SLIT2 protein sequence is shown below (NCBI accession number AAD25539; SEQ ID NO:5).

| | |
|---|---|
| 1 | MRGVGWQMLS LSLGLVLAIL NKVAPQACPA QCSCSGSTVD |
| 41 | CHGLALRSVP RNIPRNTERL DLNGNNITRI TKTDFAGLRH |
| 81 | LRVLQLMENK ISTIERGAFQ DLKELERLRL NRNHLQLFPE |
| 121 | LLFLGTAKLY RLDLSENQIQ AIPRKAFRGA VDIKNLQLDY |
| 161 | NQISCIEDGA FRALRDLEVL TLNNNNITRL SVASFNHMPK |
| 201 | LRTFRLHSNN LYCDCHLAWL SDWLRKRPRV GLYTQCMGPS |
| 241 | HLRGHNVAEV QKREFVCSDE EEGHQSFMAP SCSVLHCPAA |
| 281 | CTCSNNIVDC RGKGLTEIPT NLPETITEIR LEQNTIKVIP |
| 321 | PGAFSPYKKL RRIDLSNNQI SELAPDAFQG LRSLNSLVLY |
| 361 | GNKITELPKS LFEGLFSLQL LLLNANKINC LRVDAFQDLH |
| 401 | NLNLLSLYDN KLQTIAKGTF SPLRAIQTMH LAQNPFICDC |
| 441 | HLKWLADYLH TNPIETSGAR CTSPRRLANK RIGQIKSKKF |
| 481 | RCSGTEDYRS KLSGDCFADL ACPEKCRCEG TTVDCSNQKL |
| 521 | NKIPEHIPQY TAELRLNNNE FTVLEATGIF KKLPQIRKIN |
| 561 | FSNNKITDIE EGAFEGASGV NEILLTSNRL ENVQHKMFKG |
| 601 | LESLKTLMLR SNRITCVGND SFIGLESVRL LSLYDNQITT |
| 641 | VAPGAFDTLH SLSTLNLLAN PENCNCYLAW LGEWLRKKRI |
| 681 | VTGNPRCQKP YFLKEIPIQD VAIQDFTCDD GNDDNSCSPL |
| 721 | SRCPTECTCL DTVVRCSNKG LKVLPKGIPR DVTELYLDGN |
| 761 | QFTLVPKELS NYKELTLIDL SNNRISTLSN QSFSNMTQLL |
| 801 | TLILSYNRLR CIPPRTFDGL KSLRLLSLHG NDISVVPEGA |
| 841 | FNDLSALSHL AIGANPLYCD CNMQWLSDWV KSEYKEPGIA |
| 881 | RCAGPGEMAD KLLLTTPSKK FTCQGPVDVN ILAKCNPCLS |
| 921 | NPCKNDGTCN SDPVDFYRCT CPYGFKGQDC DVPIHACISN |
| 961 | PCKHGGTCHL KEGEEDGFWC ICADGFEGEN CEVNVDDCED |
| 1001 | NDCENNSTCV DGINNYTCLC PPEYTGELCE EKLDFCAQDL |
| 1041 | NPCQEDSKCI LTPKGFKCDC TPGYVGEHCD IDFDDCQDNK |
| 1081 | CKNGAECTDA VNGYTCICPE GYSGLFCEFS PPMVLPRTSP |
| 1121 | CDNFDCQNGA QCIVRINEPI CQCLPGYQGE KCEKLVSVNF |
| 1161 | INKESYLQIP SAKVRPQTNI TLQIATDEDS GILLYKGDKD |
| 1201 | HIAVELYRGR VRASYDTGSH PASAIYSVET INDGNFHIVE |
| 1241 | LLALDQSLSL SVDGGNPKII TNLSKQSTLN FDSPLYVGGM |
| 1281 | PGKSNVASLR QAPGQNGTSF HGCIRNLYIN SELQDFQKVP |
| 1321 | MQTGILPGCE PCHKKVCAHG TCWQSSQAGF TCECQEGWMG |
| 1361 | PLCDQRTNDP CLGNKCVHGT CLPINAFSYS CKCLEGHGGV |
| 1401 | LCDEEEDLFN PCQAIKCKHG KCRLSGLGQP YCECSSGYTG |
| 1441 | DSCDREISCR GERIRDYYQK QQGYAACQTT KKVSRLECRG |
| 1481 | GCAGGQCCGP LRSKRRKYSF ECTDGSSFVD EVEKVVKCGC |
| 1521 | TRCVS |

An example of a nucleic acid that encodes the SEQ ID NO:5 SLIT2 protein is shown (NCBI accession number AF133270; SEQ ID NO:6).

| | |
|---|---|
| 1 | ATGCGCGGCG TTGGCTGGCA GATGCTGTCC CTGTCGCTGG |
| 41 | GGTTAGTGCT GGCGATCCTG AACAAGGTGG CACCGCAGGC |
| 81 | GTGCCCGGCG CAGTGCTCTT GCTCGGGCAG CACAGTGGAC |
| 121 | TGTCACGGGC TGGCGCTGCG CAGCGTGCCC AGGAATATCC |
| 161 | CCCGCAACAC CGAGAGACTG GATTTAAATG GAAATAACAT |
| 201 | CACAAGAATT ACGAAGACAG ATTTTGCTGG TCTTAGACAT |
| 241 | CTAAGAGTTC TTCAGCTTAT GGAGAATAAG ATTAGCACCA |
| 281 | TTGAAAGAGG AGCATTCCAG GATCTTAAAG AACTAGAGAG |
| 321 | ACTGCGTTTA AACAGAAATC ACCTTCAGCT GTTTCCTGAG |
| 361 | TTGCTGTTTC TTGGGACTGC GAAGCTATAC AGGCTTGATC |
| 401 | TCAGTGAAAA CCAAATTCAG GCAATCCCAA GGAAAGCTTT |
| 441 | CCGTGGGGCA GTTGACATAA AAAATTTGCA ACTGGATTAC |
| 481 | AACCAGATCA GCTGTATTGA AGATGGGGCA TTCAGGGCTC |
| 521 | TCCGGGACCT GGAAGTGCTC ACTCTCAACA ATAACAACAT |
| 561 | TACTAGACTT TCTGTGGCAA GTTTCAACCA TATGCCTAAA |
| 601 | CTTAGGACTT TTCGACTGCA TTCAAACAAC CTGTATTGTG |
| 641 | ACTGCCACCT GGCCTGGCTC TCCGACTGGC TTCGCAAAAG |
| 681 | GCCTCGGGTT GGTCTGTACA CTCAGTGTAT GGGCCCCTCC |
| 721 | CACCTGAGAG GCCATAATGT AGCCGAGGTT CAAAAACGAG |
| 761 | AATTTGTCTG CAGTGATGAG GAAGAAGGTC ACCAGTCATT |
| 801 | TATGGCTCCT TCTTGTAGTG TTTTGCACTG CCCTGCCGCC |
| 841 | TGTACCTGTA GCAACAATAT CGTAGACTGT CGTGGGAAAG |
| 881 | GTCTCACTGA GATCCCCACA AATCTTCCAG AGACCATCAC |
| 921 | AGAAATACGT TTGGAACAGA ACACAATCAA AGTCATCCCT |
| 961 | CCTGGAGCTT TCTCACCATA TAAAAAGCTT AGACGAATTG |
| 1001 | ACCTGAGCAA TAATCAGATC TCTGAACTTG CACCAGATGC |
| 1041 | TTTCCAAGGA CTACGCTCTC TGAATTCACT TGTCCTCTAT |
| 1081 | GGAAATAAAA TCACAGAACT CCCCAAAAGT TTATTTGAAG |
| 1121 | GACTGTTTTC CTTACAGCTC CTATTATTGA ATGCCAAGAA |
| 1161 | GATAAAGTGC CTTCGGGTAG ATGCTTTTCA GGATCTCCAC |
| 1201 | AACTTGAACC TTCTCTCCCT ATATGACAAC AAGCTTCAGA |
| 1241 | CCATCGCCAA GGGGACCTTT TCACCTCTTC GGGCCATTCA |
| 1281 | AACTATGCAT TTGGCCCAGA ACCCCTTTAT TTGTGACTGC |
| 1321 | CATCTCAAGT GGCTAGCGGA TTATCTCCAT ACCAACCCGA |
| 1361 | TTGAGACCAG TGGTGCCCGT TGCACCAGCC CCCGCCGCCT |
| 1401 | GGCAAACAAA AGAATTGGAC AGATCAAAAG CAAGAAATTC |
| 1441 | CGTTGTTCAG GTACAGAAGA TTATCGATCA AAATTAAGTG |
| 1481 | GAGACTGCTT TGCGGATCTG GCTTGCCCTG AAAAGTGTCG |
| 1521 | CTGTGAAGGA ACCACAGTAG ATTGCTCTAA TCAAAAGCTC |
| 1561 | AACAAAATCC CGGAGCACAT TCCCCAGTAC ACTGCAGAGT |
| 1601 | TGCGTCTCAA TAATAATGAA TTTAGCGTGT TGGAAGCCAC |
| 1641 | AGGAATCTTT AAGAAACTTC CTCAATTACG TAAAATAAAC |
| 1681 | TTTAGCAACA ATAAGATCAC AGATATTGAG GAGGGAGCAT |
| 1721 | TTGAAGGAGC ATCTGGTGTA AATGAAATAC TTCTTACGAG |
| 1761 | TAATCGTTTG GAAAATGTGC AGCATAAGAT GTTCAAGGGA |
| 1801 | TTGGAAAGCC TCAAAACTTT GATGTTGAGA AGCAATCGAA |
| 1841 | TAACCTGTGT GGGGAATGAC AGTTTCATAG GACTCAGTTC |
| 1881 | TGTGCGTTTG CTTTCTTTGT ATGATAATCA AATTACTACA |
| 1921 | GTTGCACCAG GGGCATTTGA TACTCTCCAT TCTTTATCTA |
| 1961 | CTCTAAACCT CTTGGCCAAT CCTTTTAACT GTAACTGCTA |
| 2001 | CCTGGCTTGG TTGGGAGAGT GGCTGAGAAA GAAGAGAATT |
| 2041 | GTCACGGGAA ATCCTAGATG TCAAAAACCA TACTTCCTGA |
| 2081 | AAGAAATACC CATCCAGGAT GTGGCCATTC AGGAGTTCAC |
| 2121 | TTGTGATGAC GGAAATGATG ACAATAGTTG CTCCCCACTT |
| 2161 | TCTCGCTGTC CTACTGAATG TACTTGCTTG GATACAGTCG |
| 2201 | TCCGATGTAG CAACAAGGGT TTGAAGGTCT TGCCGAAAGG |
| 2241 | TATTCCAAGA GATGTCACAG AGTTGTATCT GGATGGAAAC |
| 2281 | CAATTTACAC TGGTTCCCAA GGAAGTCTCC AACTACAAAC |
| 2321 | ATTTAACACT TATAGACTTA AGTAACAACA GAATAAGCAC |
| 2361 | GCTTTCTAAT CAGAGCTTCA GCAACATGAC CCAGCTCCTC |
| 2401 | ACCTTAATTC TTAGTTACAA CCGTCTGAGA TGTATTCCTC |
| 2441 | CTCGCACCTT TGATGGATTA AAGTCTCTTC GATTACTTTC |
| 2481 | TCTACATGGA AATGACATTT CTGTTGTGCC TGAAGGTGCT |
| 2521 | TTCAATGATC TTTCTGCATT ATCACATCTA GCAATTGGAG |
| 2561 | CCAACCCTCT TTACTGTGAT TGTAACATGC AGTGGTTATC |
| 2601 | CGACTGGGTG AAGTCGGAAT ATAAGGAGCC TGGAATTGCT |
| 2641 | CGTTGTGCTG GTCCTGGAGA AATGGCAGAT AAACTTTTAC |
| 2681 | TCACAACTCC CTCCAAAAAA TTTACCTGTC AAGGTCCTGT |
| 2721 | GGATGTCAAT ATTCTAGCTA AGTGTAACCC CTGCCTATCA |
| 2761 | AATCCGTGTA AAAATGATGG CACATGTAAT AGTGATCCAG |
| 2801 | TTGACTTTTA CCGATGCACC TGTCCATATG GTTTCAAGGG |
| 2841 | GCAGGAGTGT GATGTCCCAA TTCATGCCTG CATCAGTAAC |
| 2881 | CCATGTAAAC ATGGAGGAAG TTGCCACTTA AAGGAAGGAG |
| 2921 | AAGAAGATGG ATTCTGGTGT ATTTGTGCTG ATGGATTTGA |
| 2961 | AGGAGAAAAT TGTGAAGTCA ACGTTGATGA TTGTGAAGAT |
| 3001 | AATGACTGTG AAAATAATTC TACATGTGTC GATGGCATTA |
| 3041 | ATAACTACAG ATGCCTTTGC CCACCTGAGT ATACAGGTGA |
| 3081 | GTTGTGTGAG GAGAAGGTGG ACTTCTGTGC CCAGGACCTG |
| 3121 | AACCCCTGCC AGCACGATTC AAAGTGCATC CTAACTCCAA |
| 3161 | AGGGATTCAA ATGTGACTGC ACACCAGGGT ACGTAGGTGA |
| 3201 | ACACTGCGAC ATCGATTTTG ACGACTGCCA AGACAACAAG |
| 3241 | TGTAAAAACG GAGCCCACTG CACAGATGCA GTGAACGGCT |
| 3281 | ATACGTGCAT ATGCCCCGAA GGTTACAGTG GCTTGTTCTG |
| 3321 | TGAGTTTTCT CCACCCATGG TCCTCCCTCG TACCAGCCCC |
| 3361 | TGTGATAATT TTGATTGTCA GAATGGAGCT CAGTGTATCG |
| 3401 | TCAGAATAAA TGAGCCAATA TGTCAGTGTT TGCCTGGCTA |
| 3441 | TCAGGGAGAA AAGTGTGAAA AATTGGTTAG TGTGAATTTT |
| 3481 | ATAAACAAAG AGTCTTATCT TCAGATTCCT TCAGCCAAGG |
| 3521 | TTCGGCCTCA GACGAACATA ACACTTCAGA TTGCCACAGA |
| 3561 | TGAAGACAGC GGAATCCTCC TGTATAAGGG TGACAAAGAC |
| 3601 | CATATCGCGG TAGAACTCTA TCGGGGGCGT GTTCGTGCCA |
| 3641 | GCTATGACAC CGGCTCTCAT CCAGCTTCTG CCATTTACAG |
| 3681 | TGTGGAGACA ATCAATGATG GAAACTTCCA CATTGTGGAA |
| 3721 | CTACTTGCCT TGGATCAGAG TCTCTCTTTG TCCGTGGATG |
| 3761 | GTGGGAACCC CAAAATCATC ACTAACTTGT CAAAGCAGTC |
| 3801 | CACTCTGAAT TTTGACTCTC CACTCTATGT AGGAGGCATG |
| 3841 | CCAGGGAAGA GTAACGTGGC ATCTCTGCGC CAGGCCCCTG |
| 3881 | GGCAGAACGG AACCAGCTTC CACGGCTGCA TCCGGAACCT |
| 3921 | TTACATCAAC AGTGAGCTGC AGGACTTCCA GAAGGTGCCG |
| 3961 | ATGCAAACAG GCATTTTGCC TGGCTGTGAG CCATGCCACA |
| 4001 | AGAAGGTGTG TGCCCATGGG ACATGCCAGC CCAGCAGCCA |
| 4041 | GGCAGGCTTC ACCTGCGAGT GCCAGGAAGG ATGGATGGGG |
| 4081 | CCCCTCTGTG ACCAACGGAC CAATGACCCT TGCCTTGGAA |
| 4121 | ATAAATGCGT ACATGGCACC TGCTTGCCCA TCAATGCGTT |
| 4161 | CTCCTACAGC TGTAAGTGCT TGGAGGGCCA TGGAGGTGTC |
| 4201 | CTCTGTGATG AAGAGGAGGA TCTGTTTAAC CCATGCCAGG |
| 4241 | CGATCAAGTG CAAGCATGGG AAGTGCAGGC TTTCAGGTCT |
| 4281 | GGGGCAGCCC TACTGTGAAT GCAGCAGTGG ATACACGGGG |
| 4321 | GACAGCTGTG ATCGAGAAAT CTCTTGTCGA GGGGAAAGGA |
| 4361 | TAAGAGATTA TTACCAAAAG CAGCAGGGCT ATGCTGCTTG |
| 4441 | CCAAACAACC AAGAAGGTGT CCCGATTAGA GTGCAGAGGT |
| 4441 | GGGTGTGCAG GAGGGCAGTG CTGTGGACCG CTGAGGAGCA |
| 4481 | AGCGGCGGAA ATACTCTTTC GAATGCACTG ACGGCTCCTC |
| 4521 | CTTTGTGGAC GAGGTTGAGA AAGTGGTGAA GTGCGGCTGT |
| 4561 | ACGAGGTGTG TGTCCTAAAC ACACTCCCGG CAGCTCTGTC |
| 4601 | TTTGGAAAAG GTTGTATACT TCTTGACCAT GTGGGACTAA |
| 4641 | TGAATGCTTC ATAGTGGAAA TATTTGAAAT ATATTGTAAA |
| 4681 | ATACAGAACA GACTTATTTT TATTATGAGA ATAAAGACTT |
| 4721 | TTTTTCTGCA TTTGGAAAAA AAAAAAAAAA AAACTCGA |

A comparison of the SLITS with SEQ ID NO:1, and the SLIT2 with SEQ ID NO:5 amino acid sequences is shown below.

```
67.0% identity in 1515 residues overlap; Score: 5664.0; Gap
frequency: 0.7%
Seq1 16
LALALALASVLSGPPAVACPTKCTCSAASVDCHGLGLRAVPRGIPRNAERLDLDRNNITR
Seq5 11
LSLGLVLA-ILNKVAPQACPAQCSCSGSTVDCHGLALRSVPRNIPRNTERLDLNGNNITR
* * * ** * *** * ** ****** ** *** **** ***** *****
Seq1 76
ITKMDFAGLKNLRVLHLEDNQVSVIERGAFQDLKQLERLRLNKNKLQVLPELLFQSTPKL
Seq5 70
ITKTDFAGLRHLRVLQLMENKISTIERGAFQDLKELERLRLNRNHLQLFPELLFLGTAKL
*** ***** **** * * * ********** ******* * ** ***** * **
Seq1 136
TRLDLSENQIQGIPRKAFRGITDVKNLQLDNNHISCIEDGAFRALRDLEILTLNNNNISR
Seq5 130
YRLDLSENQIQAIPRKAFRGAVDIKNLQLDYNQISCIEDGAFRALRDLEVLTLNNNNITR
  ********** ******** * ****** * *************** ******* *
Seq1 196
ILVTSFNHMPKIRTLRLHSNHLYCDCHLAWLSDWLRQRRTVGQFTLCMAPVHLRGFNVAD
Seq5 190
LSVASFNHMPKLRTFRLHSNNLYCDCHLAWLSDWLRKRPRVGLYTQCMGPSHLRGHNVAE
    * ******* ** ***** *************** * ** * ** * **** ***
Seq1 256
VQKKEYVCPAPHSEPPSCNANSIS---CPSPCTCSNNIVDCRGKGLMEIPANLPEGIVEI
Seq5 250
VQKREFVCSDEEEGHQSFMAPSCSVLHCPAACTCSNNIVDCRGKGLTEIPTNLPETITEI
*** * ** * * * * ** *************** *** **** * **
Seq1 313
RLEQNSIKAIPAGAFTQYKKLKRIDISKNQISDIAPDAFQGLKSLTSLVLYGNKITEIAK
Seq5 310
RLEQNTIKVIPPGAFSPYKKLRRIDLSNNQISELAPDAFQGLRSLNSLVLYGNKITELPK
***** ** ** *** **** *** * **** ******** ** *********** *
Seq1 373
GLFDGLVSLQLLLLNANKINCLRVNTFQDLQNLNLLSLYDNKLQTISKGLFAPLQSIQTL
Sec5 370
SLFEGLFSLQLLLLNANKINCLRVDAFQDLHNLNLLSLYDNKLQTIAKGTFSPLRAIQTM
  ** ** ***************** **** *************** ** * ** ***
Seq1 433
HLAQNPFVCDCHLKWLADYLQDNPIETSGARCSSPRRLANKRISQIKSKKFRCSGSEDYR
Seq5 430
HLAQNPFICDCHLKWLADYLHTNPIETSGARCTSPRRLANKRIGQIKSKKFRCSGTEDYR
******* ************ ********** ********** *********** ****
Seq1 493
SRFSSECFMDLVCPEKCRCEGTIVDCSNQKLVRIPSHLPEYVTDLRLNDNEVSVLEATGI
Seq5 490
SKLSGDCFADLACPEKCRCEGTTVDCSNQKLNKIPEHIPQYTAELRLNNNEFTVLEATGI
* * ** ** ********** ******** ** * * * **** ** *******
Seq1 553
FKKLPNLRKINLSNNKIKEVREGAFDGAASVQELMLTGNQLETVHGRVFRGLSGLKTLML
Seq5 550
FKKLPQLRKINFSNNKITDIEEGAFEGASGVNEILLTSNRLENVQHKMFKGLESLKTLML
***** ***** ***** **** ** * * ** * ** * * ** ******
Seq1 613
RSNLISCVSNDTFAGLSSVRLLSLYDNRITTITPGAFTTLVSLSTINLLSNPFNCNCHLA
Seq5 610
RSNRITCVGNDSFIGLSSVRLLSLYDNQITTVAPGAFDTLHSLSTLNLLANPFNCNCYLA
*** * ** ** * ************* *** **** ** **** *** ******* **
Seq1 673
WLGKWLRKRRIVSGNPRCQKPFFLKEIPIQDVAIQDFTCD-GNEESSCQLSPRCPEQCTC
Seq5 670
WLGEWLRKKRIVIGNPRCQNPYFLKEIPIQDVAIQDFTCDDGNDDNSCSPLSRCPTECTC
*** **** *** ******** ****************** ** ** *** ***
Seq1 732
METVVRCSNKGLRALPRGMPKDVTELYLEGNHLTAVPRELSALRHLTLIDLSNNSISMLT
Seq5 730
LDTVVRCSNKGLKVLPKGIPRDVTELYLDGNQFTLVPKELSNYKHLTLIDLSNNRISTLS
    ********** ** * * ******* ** * ** *** ********** ** *
Seq1 792
NYTFSNMSHLSTLILSYNRLRCIPVHAFNGLRSLRVLTLHGNDISSVPEGSFNDLTSLSH
Seq5 790
NQSFSNMTQLLTLILSYNRLRCIPPRTFDGLKSLRLLSLHGNDISVVPEGAFNDLSALSH
* **** * ************* * ** *** * ******* **** **** ***
Seq1 852
LALGTNPLHCDCSLRWLSEWVKAGYKEPGIARCSSPEPMADRLLLTTPTHRFQCKGPVDI
Seq5 850
LAIGANPLYCDCNMQWLSDWVKSEYKEPGIARCAGPGEMADKLLLTTPSKKFTCQGPVDV
** * *** *** *** *** ********* * *** ****** * * ****
Seq1 912
NIVAKCNACLSSPCKNNGTCTQDPVELYRCACPYSYKGKDCTVPINTCIQNPCQHGGTCH
Seq5 910
NILAKCNPCLSNPCKNDGTCNSDPVDFYRCTCPYGFKGQDCDVPIHACISNPCKHGGTCH
** **** *** **** *** *** *** *** ** ** *** ** *** ******
Seq1 972
LSDSHKDGFSCSCPLGFEGQRCEINPDDCEDNDCENNATCVDGINNYVCICPPNYTGELC
Seq5 970
LKEGEEDGFWCICADGFEGENCEVNVDDCEDNDCENNSTCVDGINNYTCLCPPEYTGELC
* *** * * **** ** * *********** ********* * *** ******
Seq1 1032
DEVIDHCVPELNLCQHEAKCIPLDKGFSCECVPGYSGKLCETDNDDCVAHKCRHGAQCVD
Seq5 1030
EEKLDFCAQDLNPCQHDSKCILTPKGFKCDCTPGYVGEHCDIDFDDCQDNKCKNGAHCTD
  * * * ** *** *** *** * * *** * * * *** ** ** * *
Seq1 1092
TINGYTCTCPQGFSGPFCEHPPPMVLLQTSPCDQYECQNGAQCIVVQQEPTCRCPPGFAG
Seq5 1090
AVNGYTCICPEGYSGLFCEFSPPMVLPRTSPCDNFDCQNGAQCIVRINEPICQCLPGYQG
    ***** ** * ** *** ***** ***** ********* ** * * ** *
Seq1 1152
PRCEKLITVNFVGKDSYVELASAKVRPQANISLQVATDKDNGILLYKGDNDPLALELYQG
Seq5 1150
EKCEKLVSVNFINKESYLQIPSAKVRPQTNITLQIATDEDSGILLYKGDKDHIAVELYRG
    **** *** * ** ******* ** ** *** * ******** * * *** *
Seq1 1212
HVRLVYDSLSSPPTTVYSVETVNDGQFHSVELVTLNQTLNLVVDKGTPKSLGKLQKQPAV
Seq5 1210
RVRASYDTGSHPASAIYSVETINDGNFHIVELLALDQSLSLSVDGGNPKIITNLSKQSTL
  ** ** * * ***** *** ** *** * * * * ** * ** * **
Seq1 1272
GINSPLYLGGIPTSTGLSALRQGTDRPLGGFHGCIKEVRINNELQDFKALPPQSLGVSPG
Seq5 1270
NFDSPLYVGGMPGKSKVASLRQAPGQNGTSFHGCIRNLYINSELQDFQKVPMQT-GILPG
      **** ** * *** ***** ** ***** * * * **
Seq1 1332
CKSC--TVCKHGLCRSVEKDSVVCECRPGWTGPLCDQEARDPCLGHRCHHGKCVATGT-S
Seq1 1329
CEPCHKKVCAHGTCQPSSQAGFTCECQEGWMGPLCDQRTNDPCLGNKCVHGTCLPINAFS
* * ** ** * *** ** ****** ***** * ** * *
Seq1 1389
YMCKCAEGYGGDLCDNKNDSANACSAFKCHHGQCHISDQGEPYCLCQPGFSGEHCQQENP
Seq5 1389
YSCKCLEGHGGVLCDEEEDLFNPCQAIKCKHGKCRLSGLGQPYCECSSGYTGDSCDREIS
* *** ** ** *** * * * * ** ** * * * *** * * * * *
Seq1 1449
CLGQVVREVIRRQKGYASCATASKVPIMECRGGC-GPQCCQPTRSKRRKYVFQCTDGSSF
Seq5 1449
CRGERIRDYYQKQQGYAACQTTKKVSRLECRGGCAGGQCCGPLRSKRRKYSFECTDGSSF
* * * * *** * * ** ****** * *** * ******* * *******
Seq1 1508 VEEVERELECGCLAC
Seq3 1509 VDEVEKVVKCGCTKC
                    * *** *** *
```

As illustrated, the SLIT # and SLIT2 proteins with SEQ ID NOs:1 and 5, respectively, share about 67% sequence identity but as described herein SLIT2 and SLIT3 proteins are both useful in the described methods. Hence, SLIT proteins can have sequence variations without loss of function.

Another example of a SLIT2 protein sequence is shown below (NCBI accession number AAI43979; SEQ ID NO:7).

| | |
|---|---|
| 1 | MRGVGWQMLS LSLGLVLAIL NKVAPQACPA QCSCSGSTVD |
| 41 | CHGLALRSVP RNIPRNTERL DLNGNNITRI TKTDFAGLRH |
| 81 | LRVLQLMENK ISTIERGAFQ DLKELERLRL NRNHLQLFPE |
| 121 | LLFLGTAKLY RLDLSENQIQ AIPRKAFRGA VDIKNLQLDY |
| 161 | NQISCIEDGA FRALRDLEVL TLNNNNITRL SVASFNHMPK |
| 201 | LRTFRLHSNN LYCDCHLAWL SDWLRQRPRV GLYTQCMGPS |
| 241 | HLRGHNVAEV QKREFVCSGH QSFMAPSCSV LHCPAACTCS |
| 281 | NNIVDCRGKG LTEIPTNLPE TITEIRLEQN TIKVIPPGAF |
| 321 | SPYKKLRRID LSNNQISELA PDAFQGLRSL NSLVLYGNKI |
| 361 | TELPKSLFEG LFSLQLLLLN ANKINCLRVD AFQDLHNLNL |
| 401 | LSLYDNKLQT IAKGTFSPLR AIQTMHLAQN PFICDCHLKW |
| 441 | LADYLHTNPI ETSGARCTSP RRLANKRIGQ IKSKKFRCSG |
| 481 | TEDYRSKLSG DCFADLACPE KCRCEGTTVD CSNQKLNKIP |
| 521 | EHIPQYTAEL RLNNNEFTVL EATGIFKKLP QLRKINFSNN |
| 561 | KITDIEEGAF EGASGVNEIL LTSNRLENVQ HKMFKGLESL |
| 601 | KTLMLRSNRI TCVGNDSFIG LSSVRLLSLY DNQITTVAPG |
| 641 | AFDTLHSLST LNLLANPFNC NCYLAWLGEW LRKKRIVTGN |
| 681 | PRCQKPYFLK EIPIQDVAIQ DFTCDDGNDD NSCSPLSRCP |
| 721 | TECTCLDTVV RCSNKGLKVL PKGIPRDVTE LYLDGNQFTL |
| 761 | VPKELSNYKH LTLIDLSNNR ISTLSNQSFS NMTQLLTLIL |
| 801 | SYNRLRCIPP RTFDGLKSLR LLSLHGNDIS VVPEGAFNDL |
| 841 | SALSHLAIGA NPLYCDCNMQ WLSDWVKSEY KEPGIARCAG |
| 881 | PGEMADKLLL TTPSKKFTCQ GPVDVNILAK CNPCLSNPCK |
| 921 | NDGTCNSDPV DFYRCTCPYG FKGQDCDVPI HACISNPCKH |
| 961 | GGTCHLKEGE EDGFWCICAD GFEGENCEVN VDDCEDNDCE |
| 1001 | NNSTCVDGIN NYTCLCPPEY TGELCEEKLD FCAQDLNPCQ |
| 1041 | HDSKCILTPK GFKCDCTPGY VGEHCDIDFD DCQDNKCKNG |
| 1081 | AHCTDAVNGY TCICPEGYSG LFCEFSPPMV LPRTSPCDNF |
| 1121 | DCQNGAQCIV RINEPICQCL PGYQGEKCEK LVSVNFINKE |
| 1161 | SYLQIPSAKV RPQTNITLQI ATDEDSGILL YKGDKDHIAV |
| 1201 | ELYRGRVRAS YDTGSHPASA IYSVETINDG NFHIVELLAL |
| 1241 | DQSLSLSVDG GNPKIITNLS KQSTLNFDSP LYVGGMPGKS |
| 1281 | NVASLRQAPG QNGTSFHGCI RNLYINSELQ DFQKVPMQTG |
| 1321 | ILPGCEPCHK KVCAHGTCQP SSQAGFTCEC QEGWMGPLCD |
| 1361 | QRTNDPCLGN KCVHGTCLPI NAFSYSCKCL EGHGGVLCDE |
| 1401 | EEDLFNPCQA IKCKHGKCRL SGLGQPYCEC SSGYTGDSCD |
| 1441 | REISCRGERI RDYYQKQQGY AACQTTKKVS RLECRGGCAG |
| 1481 | GQCCGPLRSK RRKYSFECTD GSSFVDEVEK VVKCGCTRCV |
| 1521 | S |

Another example of a SLIT2 protein sequence is shown below (NCBI accession number NP_001276065 XP_005248270; SEQ ID NO:8).

| | |
|---|---|
| 1 | MRGVGWQMLS LSLGLVLAIL NKVAPQACPA QCSCSGSTVD |
| 41 | CHGLALRSVP RNIPRNTERL DLNGNNITRI TKTDFAGLRH |
| 81 | LRVLQLMENK ISTIERGAFQ DLKELERLRL NRNHLQLFPE |
| 121 | LLFLGTAKLY RLDLSENQIQ AIPRKAFRGA VDIKNLQLDY |
| 161 | NQISCIEDGA FRALRDLEVL TLNNNNITRL SVASFNHMPK |
| 201 | LRTFRLHSNN LYCDCHLAWL SDWLRQRPRV GLYTQCMGPS |
| 241 | HLRGHNVAEV QKREFVCSGH QSFMAPSCSV LHCPAACTCS |
| 281 | NNIVDCRGKG LTEIPTNLPE TITEIRLEQN TIKVIPPGAF |
| 321 | SPYKKLRRID LSNNQISELA PDAFQGLRSL NSLVLYGNKI |
| 361 | TELPKSLFEG LFSLQLLLLN ANKINCLRVD AFQDLHNLNL |
| 401 | LSLYDNKLQT IAKGTFSPLR AIQTMHLAQN PFICDCHLKW |
| 441 | LADYLHTNPI ETSGARCTSP RRLANKRIGQ IKSKKFRCSG |
| 481 | TEDYRSKLSG DCFADLACPE KCRCEGTTVD CSNQKLNKIP |
| 521 | EHIPQYTAEL RLNNNEFTVL EATGIFKKLP QLRKINFSNN |
| 561 | KITDIEEGAF EGASGVNEIL LTSNRLENVQ HKMFKGLESL |
| 601 | KTLMLRSNRI TCVGNDSFIG LSSVRLLSLY DNQITTVAPG |
| 641 | AFDTLHSLST LNLLANPFNC NCYLAWLGEW LRKKRIVTGN |
| 681 | PRCQKPYFLK EIPIQDVAIQ DFTCDDGNDD NSCSPLSRCP |
| 721 | TECTCLDTVV RCSNKGLKVL PKGIPRDVTE LYLDGNQFTL |
| 761 | VPKELSNYKH LTLIDLSNNR ISTLSNQSFS NMTQLLTLIL |
| 801 | SYNRLRCIPP RTFDGLKSLR LLSLHGNDIS VVPEGAFNDL |
| 841 | SALSHLAIGA NPLYCDCNMQ WLSDWVKSEY KEPGIARCAG |
| 881 | PGEMLDKLLL TTPSKKFTCQ GPVDVNILAK CNPCLSNPCK |
| 921 | NDGTCNSDPV DFYRCTCPYG FKGQDCDVPI HACISNPCKH |
| 961 | GGTCHLKEGE EDGFWCICAD GFEGENCEVN VDDCEDNDCE |
| 1001 | NNSTCVDGIN NYTCLCPPEY TGELCEEKLD FCAQDLNPCQ |
| 1041 | HDSKCILTPK GFKCDCTPGY VGEHCDTDFD DCQDNKCKNG |
| 1081 | AHCTDAVNGY TCICPEGYSG LFCEFSPPMV LPRTSPCDNF |
| 1121 | DCQNGAQCIV RINEPICQCL PGYQGEKCEK LVSVNFINKE |
| 1161 | SYLQIPSAKV RPQTNITLQI ATDEDSGILL YKGDKDHIAV |
| 1201 | ELYRGRVRAS YDTGSHPASA IYSVETINDG NFHIVELLAL |
| 1241 | DQSLSLSVDG GNPKIITNLS KQSTLNFDSP LYVGGMPGKS |
| 1281 | NVASLRQAPG QNGTSFHGCI RNLYINSELQ DFQKVPMQTG |
| 1321 | ILPGCEPCHK KVCAHGTCQP SSQAGFTCEC QEGWMGPLCD |
| 1361 | ORTNDPCLGN KCVHGYCLPI NAFSYSCKCL EGHGGVLCDE |
| 1401 | EEDLFNPCQA IKCKHGKCRL SGLGQPYCEC SSGYTGDSCD |
| 1441 | REISCRGERI RDYYQKQQGY AACQTTKKVS RLECRGGCAG |
| 1481 | GQCCGPLRSK RRKYSFECTD GSSFVDEVEK VVKCGCTRCV |
| 1521 | S |

SLIT3 or SLIT2 is the designation for a human gene and its associated protein, referred to in databases for example, as Entrez Gene: 6586. Ensembl: ENSG00000184347, OMIM: 60374, and 5 UniProtKB: O75094. For example, the sequence of the UniProtKB: 075094 SLIT3 protein is shown below as SEQ ID NO:9.

```
                10 20 30 40
MAPGWAGVGA AVRARLALAL ALASVLSGPP AVACPTKCTC
                50 60 70 80
SAASVDCHGL GLRAVPRGIP RNAERLDLDR NNITRITKMD
                90 100 110 120
FAGLKNLRVL HLEDNQVSVI ERGAFQDLKQ LERLRLNKNK
              130 140 150 160
LQVLPELLFQ STPKLTRLDL SENQIQGIPR KAFRGITDVK
              170 180 190 200
NLQLDNNHIS CIEDGAFRAL RDLEILTLNN NNISRILVTS
              210 220 230 240
FNHMPKIRTL RLHSNHLYCD CHLAWLSDWL RQRRTVGQFT
              250 260 270 280
LCMAPVHLRG FNVADVQKKE YVCPAPHSEP PSCNANSISC
              290 300 310 320
PSPCTCSNNI VDCRGKGLME IPANLPEGIV EIRLEQNSIK
              330 340 350 360
AIPAGAFTQY KKLKRIDISK NQISDIAPDA FQGLKSLTSL
              370 380 390 400
VLYGNKITEI VKGLFDGLVS LQLLLLNANK INCLRVNTFQ
              410 420 430 440
DLQNLNLLSL YDNKLQTISK GLFAPLQSIQ TLHLAQNPFV
              450 460 470 480
CDCHLKWLAD YLQDNPIETS GARCSSPRRL ANKRISQIKS
              490 500 510 520
KKFRCSGSED YRSRFSSECF MDLVCPEKCR CEGTIVDCSN
              530 540 550 560
QKLVRIPSHL PEYVTDLRLN DNEVSVLEAT GIFKKLPNLR
              570 580 590 600
KINLSNNKIK EVREGAFDGA ASVQELMLTG NQLETVHGRV
              610 620 630 640
FRGLSGLKTL MLRSNLIGCV SNDTFAGLSS VRLLSLYDNR
              650 660 670 680
ITTITPGAFT TLVSLSTINL LSNPFNCNCH LAWLGKWLRK
              690 700 710 720
RRIVSGNPRC QKPFFLKEIP IQDVAIQDFT CDGNEESSCQ
              730 740 750 760
LSPRCPEQCT CMETVVRCSN KGLRALPRGM PKDVIELYLE
              770 780 790 800
GNHLTAVPRE LSALRHLTLI DLSNNSISML TNYTFSNMSH
              810 820 830 840
LSTLILSYNR LRCIPVHAFN GLRSLRVLTL HGNDISSVPE
              850 660 870 880
GSFNDLTSLS HLALGTNPLH CDCSLRWLSE WVKAGYKEPG
              890 900 910 920
IARCSSPEPM ADRLLLTTPT HRFQCKGPVD INIVAKCNAC
              930 940 950 960
LSSPCKNNGT CTQDPVELYR CACPYSYKGK DCTVPINTCI
              970 980 990 1000
QNPCQHGGTC HLSDSHKDGF SCSCPLGFEG QRCEINPDDC
            1010 1020 1030 1040
EDNDCENNAT CVDGINNYVC ICPPNYTGEL CDEVIDHCVP
            1050 1060 1070 1080
ELNLCQHEAK CIPLDKGFSC ECVPGYSGKL CETDNDDCVA
            1090 1100 1110 1120
HKCRHGAQCV DTINGYTCTC PQGFSGPFCE HPPPMVLLQT
            1130 1140 1150 1160
SPCDQYECQN GAQCIVVQQE PTCRCPPGFA GPRCEKLITV
            1170 1180 1190 1200
NFVGKDSYVE LASAKVRPQA NISLQVATDK DNGILLYKGD
            1210 1220 1230 1240
NDPLALELYQ GHVRLVYDSL SSPPTTVYSV ETVNDGQFHS
            1250 1260 1270 1280
VELVTLNQTL NLVVDKGTPK SLGKLQKQPA VGINSPLYLG
            1290 1300 1310 1320
GIPTSTGLSA LRQGTDRPLG GFHGCIHEVR INNELQDFKA
            1330 1340 1350 1360
LPPQSLGVSP GCKSCTVCKH GLCRSVEKDS VVCECRPGWT
            1370 1380 1390 1400
GPLCDQEARD PCLGHRCHHG KCVATGTSYM CKCAEGYGGD
            1410 1420 1430 1440
LCDNKNDSAN ACSAFKCHHG QCHISDQGEP YCLCQPGFSG
            1450 1460 1470 1480
EHCQQENPCL GQVVREVIRR QKGYASCATA SKVPIMECRG
            1490 1500 1510 1520
GCGPQCCQPT RSKRRKYVFQ CTDGSSFVEE VERHLECGCL ACS
```

SLIT2 and SLIT3 are conserved genes, with orthologs in around 160 organisms other than humans, including chimpanzee, Rhesus monkey, dog, cow, mouse, rat, chicken, zebrafish, and frog. Other sequences of the SLIT2 and SLIT3 genes, cDNAs, mRNAs, and proteins are available.

As a composition, the SLIT3 or SLIT2 agent may be delivered as cDNA in a vector, for example a viral vector, as an mRNA construct, or as a protein. The DNA, RNA, or amino acid may be the same as the human reference sequences or may have a sequence in one or more monomers (e.g., nucleotides or amino acids) has been substituted with other, chemically, sterically and/or electronically similar one, without substantially altering the biological activity.

As employed herein, the term “substantially the same sequence” refers to sequences having at least about 60% sequence homology or identity with respect to any of the sequences described herein (“reference sequences”) and retaining comparable functional and biological activity characteristic of the protein, DNA, or mRNA defined by the reference sequences described. In some cases, molecules having “substantially the same sequence” will have at least about 80%, or about 90% identity with respect to a reference sequence; or with greater than about 95%, or 96%, or 97%, or 98%, or 99% sequence identity. It is recognized, however, that a sequence containing less than the described levels of sequence identity arising as splice variants or that are modified by conservative substitutions are also encompassed within the scope of the present invention. The degree of sequence homology is determined by conducting an amino acid sequence similarity search of a protein data base, such as the database of the National Center for Biotechnology Information (NCBI; see website at ncbi.nlm.nih.gov/BLAST/), using a computerized algorithm, such as PowerBLAST, QBLAST, PSI-BLAST, PHI-BLAST, gapped or ungapped BLAST.

Also encompassed within “SLIT3 or SLIT2 agent” are biologically functional or active analogs of SLIT3 or SLIT2. The term “analog” includes any polypeptide or polynucleotide having a sequence substantially identical to a sequence specifically referenced herein in which one or more residues have been conservatively substituted with a functionally similar residue and which displays the ability to mimic the biological activity of SLID or SLID. Examples of conservative substitutions for amino acids include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic add or glutamic acid for another.

The phrase “conservative substitution” also includes the use of a chemically derivatized residue in place of a non-derivatized residue, provided that the polypeptide or expressed polypeptide displays the requisite biological activity.

“Chemical derivative” refers to a subject polypeptide or polynucleotide having one or more residues chemically derivatized by reaction of a functional side group.

Such derivatized molecules include with respect to polypeptides, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine. The useful polypeptide can also include any polypeptide having one or more additions and/or deletions of residues, relative to the sequence of a polypeptide whose sequence is shown herein. For example, useful polypeptides can include one or more amino acid substitution, deletion, insertion, or other modification so long as the at least some SLIT3 or SLIT2 biological activity is maintained.

For example, useful SLIT2 and/or SLIT3 variant polypeptides with sequence variations compared to the sequences described herein can have at least 20%, or at least 30%, or at least 40%, or at least 50%, or at least 60%, or at least 70%, or at least 75%, or at least 80%, or at least 85%, or at least 90%, or at least 95% of the activity of an unmodified SLIT2 and/or SLIT3 protein (e.g., a wild type protein or a SLIT2 or SLIT3 protein with a sequence described herein).

### SLIT3, SLIT2 or SHN3-Interfering Agents

Described herein are also SLIT3, SLIT2 or SHN3-interfering agents, which include antibodies and small interfering RNA (siRNA) molecules against SLIT3 or SLIT2, and methods of using same to prevent bone growth. Anti-SLIT3, SLIT2 or SHN3antibodies and siRNA molecules targeted to the SLIT3, or SLIT2 gene (“SLIT3, or SLIT2 siRNA”) have been found to prevent bone growth. However, reduction of SHN3 expression can enhance bone growth.

### Small Interfering RNA as SLIT3, SLIT2 or SHN3-Interfering Agents

The SLIT3, SLIT2 or SHN3 siRNA are targeted to a mammalian SLIT3, SLIT2 or SHN3 genes and can inhibit the expression of such target genes, respectively. The SLIT3, SLIT2 or SHN3 siRNA include a specific antisense sequence that is complementary to a portion of the mRNA transcribed from the target gene (i.e. the target mRNA) and can be double stranded (i.e. composed of an antisense strand, comprising the specific antisense sequence, and a complementary sense strand) or single-stranded (i.e. composed of an antisense strand, comprising the specific antisense sequence, only) as described in more detail below. Short-hairpin siRNA (shRNA) against SLIT3, SLIT2 or SHN3 are also described herein.

As Ls known in the art, the specificity of siRNA molecules is determined by the binding of the antisense strand of the molecule to its target mRNA. Effective siRNA molecules are generally less than 30 to 35 base pairs in length to prevent them triggering non-specific RNA interference pathways in the cell via the interferon response, although longer siRNA can also be effective

Design and construction of siRNA molecules is known in the art [see, for example. Elbashir, et al., Nature, 411:494-498 (2001); Bitko and Barik, BMC Microbiol., 1:34 (2001)]; Elbashir, S. M., et al. (2001) EMBO J. 20, 6877-6888; and Zamore, P. D., et al. (2000) Cell 101, 25-33). Use of SLIT3, SLIT2, or SHN3 siRNA is also disclosed in the Examples herein.

In one embodiment, the target mRNA sequence for the SLIT3, SLIT2, or SHN3 siRNA is selected from the entire SLIT3, SLIT2, or SHN3 mRNA sequence. In another embodiment, the target mRNA sequence for the SLIT3, SLIT2, or SHN3 siRNA is selected from the 5′ untranslated region of the SLIT3, SLIT2, or SHN3 mRNA. In still another embodiment, the target mRNA sequence for the SLIT3, SLIT2, or SHN3 siRNA is selected from the 3′ untranslated region of the SLIT3, SLIT2, or SHN3 mRNA. In one embodiment, the mRNA target sequence for the SLIT3, SLIT2, or SHN3 siRNA is within the coding region of the target mRNA. In another embodiment, the target sequence for the SLIT3, SLIT2, or SHN3 siRNA is selected from the region of the target mRNA beginning 50 to 100 nucleotides downstream of the start codon and ending at the stop codon. In an additional embodiment, the target sequence for the SLIT3, SLIT2, or SHN3 siRNA is selected from the 3′ end of the coding region, for example, the region of the target mRNA beginning 500 to 600 nucleotides downstream of the start codon and ending at the stop codon. In a further embodiment, the target mRNA sequence for the SLIT3, SLIT2, or SHN3 siRNA is within an individual exon. In another embodiment, the target mRNA sequence for the SLIT3, SLIT2, or SHN3 siRNA is selected from a region of the target mRNA which spans an exon-exon junction.

In another embodiment, a target mRNA sequence is selected that comprises the sequence 5′-AA(Nₓ)-3′ or 5′-NA(Nₓ)-3′, where N is any nucleotide and “x” is an integer between 10 and 50. In another embodiment, “x” is between 15 and 30. In yet another embodiment, “x” is between 19 and 23. In a further embodiment, “x” is 19 or 20.

In another embodiment, a target mRNA sequence is selected that comprises between about 30% and about 70% G/C content. In another embodiment, a target sequence is selected that comprises between about 30% and about 60% G/C content. In another embodiment a target sequence is selected that comprises between about 35% and about 55% G/C content.

Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi.

Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). The spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). The spacer sequence is typically an unrelated nucleotide sequence that is situated between two complementary nucleotide sequence regions which, when annealed into a double-stranded nucleic acid, comprise a shRNA (see, for example, Brummelkamp et al., 2002 Science 296:550; Paddison et al., 2002 Genes Develop. 16:948; Paul et al., Nat. Biotechnol. 20:505-508 (2002); Grabarek et al., BioTechniques 34:734-44 (2003)). The spacer sequence generally comprises between about 3 and about 100 nucleotides.

In one embodiment, the siRNA molecule is a double- or single-stranded siRNA between about 15 and about 40 nucleotides in length. In another embodiment, the siRNA molecule is a double- or single-stranded siRNA between about 15 and about 35 nucleotides in length. In another embodiment, the siRNA molecule is a double- or single-stranded siRNA between about 17 and about 30 nucleotides in length. In another embodiment, the siRNA molecule is a double- or single-stranded siRNA between about 19 and about 25 nucleotides in length. In another embodiment, the siRNA molecule is a double- or single-stranded siRNA between about 21 to about 23 nucleotides in length.

In an alternative embodiment, the siRNA molecule is a shRNA molecule or circular siRNA molecule between about 50 and about 100 nucleotides in length. In a further embodiment, the siRNA molecule is a shRNA molecule between about 50 to about 60 nucleotides in length.

The specific antisense sequence comprised by the siRNA molecule can be identical or substantially identical to the complement of the target sequence. In one embodiment, the specific antisense sequence comprised by the siRNA molecule is at least about 75% identical to the complement of the target mRNA sequence. In another embodiment, the specific antisense sequence comprised by the siRNA molecule is at least about 90% identical to the complement of the target mRNA sequence. In a further embodiment, the specific antisense sequence comprised by the siRNA molecule is at least about 95% identical to the complement of the target mRNA sequence. In another embodiment, the specific antisense sequence comprised by the siRNA molecule is at least about 97% or 98% identical to the complement of the target mRNA sequence. Methods of determining sequence identity are known in the art and can be determined, for example, by using the BLASTN program of the University of Wisconsin Computer Group (GCG) software or provided on the NCBI website.

The specific antisense sequence of the siRNA molecules described herein may exhibit variability by differing (e.g. by nucleotide substitution, including transition or transversion) at one, two, three, four or more nucleotides from the sequence of the target mRNA. When such nucleotide substitutions are present in the antisense strand of a dsRNA molecule, the complementary nucleotide in the sense strand with which the substitute nucleotide would typically form hydrogen bond base-pairing may or may not be correspondingly substituted dsRNA molecules in which one or more nucleotide substitution occurs in the sense sequence, but not in the antisense strand, are also contemplated. When the antisense sequence of a siRNA molecule comprises one or more mismatches between the nucleotide sequence of the siRNA and the target nucleotide sequence, as described above, the mismatches may be found at the 3′ terminus, the 5′ terminus or in the central portion of the antisense sequence.

A modified siRNA molecule can comprise one or more modified nucleotides, for example, a siRNA molecule comprising modified ribonucleotide(s) can comprise about 5% to about 100% modified nucleotides (for example, 5%, 10%, 15%, 20%, 25%, 30%, 35%, 40%, 45%, 50%, 55%, 60%, 65%, 70%, 75%, 80%, 85%, 90%, 95% or 100% modified nucleotides). The actual percentage of modified nucleotides present in a given siRNA molecule will depend on the total number of nucleotides present in the siRNA. If the siRNA molecule is a single-stranded RNA (ssRNA) molecule, the percent modification will be based upon the total number of nucleotides present in the ssRNA molecule. When the siRNA molecule is a dsRNA molecule, the percent modification can be based upon the total number of nucleotides present in the sense strand, antisense strand, or both the sense and antisense strands of the molecule. In accordance with the present invention, a siRNA molecule that comprises one or more modified nucleotides or linkages maintains the ability to inhibit expression of the target gene.

A nucleoside is a base-sugar combination and a nucleotide is a nucleoside that further includes a phosphate group covalently linked to the sugar portion of the nucleoside. In forming RNA molecules, the phosphate groups covalently link adjacent nucleosides to one another to form a linear polymeric compound, with the normal linkage or backbone of RNA being a 3′ to 5′ phosphodiester linkage. Specific examples of siRNA molecules useful in this invention include siRNA molecules containing modified backbones or non-natural internucleoside linkages. siRNA molecules having modified backbones include both those that retain a phosphorus atom in the backbone and those that lack a phosphorus atom in the backbone.

The siRNA molecules can comprise one or more 5′ and/or 3′-cap structures. The siRNA molecule can comprise a cap structure at the 3′-end of the sense strand, the antisense strand, or both the sense and antisense strands; or at the 5′-end of the sense strand, the antisense strand, or both the sense and antisense strands of the siRNA molecule. Alternatively, the siRNA molecule can comprise a cap structure at both the 3′-end and 5′-end of the siRNA molecule. The term “cap structure” refers to a chemical modification incorporated at either terminus of an oligonucleotide, which protects the molecule from exonuclease degradation, and may also facilitate delivery and/or localization within a cell.

The present invention also contemplates siRNA comprising ribonucleotide mimetics in which both the sugar and the internucleoside linkage of the nucleotide units are replaced with novel groups. Modified siRNA molecules may also contain one or more substituted sugar moieties and may also include modifications or substitutions to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases known in the art.

Another modification applicable to the siRNA molecules is the chemical linkage to the siRNA molecule of one or more moieties or conjugates which enhance the activity, cellular distribution, cellular uptake, bioavailability, pharmacokinetic properties and/or stability of the siRNA molecule. The conjugate molecule can be linked to the siRNA molecule by way of a linker, for example, via a biodegradable linker. The conjugate molecule can be attached at the 3′-end of the sense strand, the antisense strand, or both the sense and antisense strands of the siRNA molecule. Alternatively, the conjugate molecule can be attached at the 5′-end of the sense strand, the antisense strand, or both the sense and antisense strands of the siRNA molecule. It is also contemplated that a conjugate molecule can be attached at both the 3′-end and 5′-end of the siRNA molecule. When more than one conjugate molecule is attached to the siRNA molecule, the conjugate molecules can be the same or different.

One skilled in the art will recognize that it is not necessary for all positions in a given siRNA molecule to be uniformly modified. The present invention, therefore, contemplates the incorporation of more than one of the aforementioned modifications into single siRNA molecules.

### Antibodies as SLIT3, SLIT2, or SHN3-Interfering Agents

In one aspect, the present disclosure relates to use of isolated antibodies, which may monoclonal antibodies, which may be humanized or fully human monoclonal antibodies that bind specifically to SLIT3, SLIT2 or SHN3. In certain embodiments, the antibodies of the invention exhibit one or more desirable functional properties, such as high affinity binding to SLIT3, SLIT2, or SHN3, or the ability to inhibit binding of SLIT2 or SLIT3 to a ROBO receptor.

In another aspect, the disclosure pertains to methods of preventing bone growth in a subject using anti-SLIT2 or SLIT3 antibodies. In another aspect, the disclosure pertains to methods of increasing bone growth in a subject using anti-SHN3 antibodies.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g. a ROBO-binding domain of SLIT2 or SLIT3). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Wand et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sri. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds SLIT3, SLIT2 or SHN3 is substantially free of antibodies that specifically bind antigens other than SLIT3, SLIT2 or SHN3). An isolated antibody that specifically binds SLIT3, SLIT2, or SHN3 may, however, have cross-reactivity to other antigens, such as SLIT1 or SLIT-family molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human SLIT3, SLIT2, or SHN3” is intended to refer to an antibody that binds to human SLIT2 or SLIT2 with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}.” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human SLIT3, SLIT2, or SHN3. Preferably, an antibody of the invention binds to SLIT3, SLIT2, or SHN3 with high affinity, for example with a K_{D} of 1×10⁻⁷ M or less. The anti-SLIT2, anti-SLIT3, or anti-SHN3 antibodies preferably exhibit one or more of the following characteristics:

(a) binds to human SLIT3, SLIT2, or SHN3 with a K_{D} of 1×10⁻⁷ M or less;

(b) anti-SLIT2 or anti-SLIT3 prevents bone growth; or

(c) anti-SHN3 increases bone growth.

Preferably, the antibody binds to human SLIT3, SLIT2, or SHN3 with a K_{D} of 5×10⁻⁸ M or less, binds to human SLIT3, SLIT2, or SHN3 with a K_{D} of 1×10⁻⁸ M or less, binds to human SLIT3, SLIT2, or SHN3 with a K_{D} of 5×10⁻⁹ M or less, binds to human SLIT3, SLIT2, or SHN3 with a K_{D} of 4×10⁻⁹ M or less, binds to human SLIT3, SLIT2, or SHN3 with a K_{D} of 2×10⁻⁹ M or less, or binds to human SLIT3, SLIT2, or SHN3 with a K_{D} of between 1×10⁻⁹ M and 1×10⁻¹⁰ M or less.

Standard assays to evaluate the binding ability of the antibodies toward SLIT3. SLIT2, or SHN3 are available, including for example, ELISAs, Western blots and RIAs. Suitable assays are described in detail in the Examples. The binding kinetics (e.g., binding affinity) of the antibodies also can be assessed by standard assays known in the art, such as by Biacore™ analysis.

Given that each of the subject antibodies can bind to SLIT3, SLIT2, or SHN3, the V_{L} and V_{H} sequences can be “mixed and matched” to create other anti-SHN3, anti-SLIT2 or anti-SLIT3 binding molecules of the invention. SLIT3, SLIT2, or SHN3 binding of such “mixed and matched” antibodies can be tested using the binding assays described above and in the examples (e.g., ELISAs). Preferably, when V_{L} and V_{H} chains are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:

(a) a heavy chain variable region comprising an amino acid sequence; and

(b) a light chain variable region comprising an amino acid sequence;

wherein the antibody specifically binds SLIT3, SLIT2, or SHN3, preferably human SLIT3, SLIT2, or SHN3.

Given that each of these antibodies can bind to SLIT3. SLIT2, or SHN3 and that antigen-binding specificity is provided primarily by the CDR1, CDR2, and CDR3 regions, the V_{H} CDR1, CDR2, and CDR3 sequences and Vₖ CDR1, CDR2, and CDR3 sequences can be “mixed and matched” (i.e., CDRs from different antibodies can be mixed and match, although each antibody must contain a V_{H} CDR1, CDR1, and CDR3 and a Vₖ CDR1, CDR2, and CDR3) to create other anti-SLIT2 or anti-SLIT3 binding molecules of the invention. SLIT3, SLIT2, or SHN3 binding of such “mixed and matched” antibodies can be tested using the binding assays described above and in the Examples (e.g., ELISAs, Biacore analysis). Preferably, when V_{H} CDR sequences are mixed and matched, the CDR1, CDR2 and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence preferably is replaced with a structurally similar CDR sequence(s). Novel V_{H} and V_{L} sequences can be created by substituting one or more V_{H} and/or V_{L} CDR region sequences with structurally similar sequences from the CDR sequences of the antibodies disclosed herein.

It is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example. Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin .alpha..sub.v.beta..sub.3 antibodies using a heavy and light chain variable CDR3 domain

### Bispecific Molecules

In another aspect, the present invention features bispecific molecules comprising an anti-SLIT3, SLIT2 or SHN3 antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for SLIT3, SLIT2, or SHN3 and a second binding specificity for a second target epitope. In some cases, the second target epitope is an Fc receptor, e.g., human FcγRI (CD64) or a human

### Gene Transfer Vectors

SLIT3 or SLIT2 nucleic acid constructs, be they intended to generate SLIT3 or SLIT2, or to interfere with SLIT3 or SLIT2 function, may be delivered via gene transfer vectors. Similarly, SHN2 interfering nucleic acids (that can reduce SHN3 expression) may be delivered via gene transfer vectors.

The invention also provides a gene transfer vector comprising a nucleic acid sequence which encodes a SLIT3 or SLIT2 polypeptide or an interfering RNA molecule against SLIT3, SHN3, or SLIT2. The invention further provides a method of promoting or preventing bone growth, which method comprises administering to the mammal the above-described gene transfer vector. Various aspects of the inventive gene transfer vector and method are discussed below. Although each parameter is discussed separately, the inventive gene transfer vector and method comprise combinations of the parameters set forth below to treat a subject in need thereof. Accordingly, any combination of parameters can be used according to the inventive gene transfer vector and the inventive method.

For example, administration of a SLIT3 or SLIT2 polypeptide, or a gene transfer vector comprising a nucleic acid sequence which encodes a SLIT3 or SLIT2 polypeptide, can increase bone volume or the maximal load tolerated by a bone by at least 5%, or at least 10%, or at least 20%, or at least 25%, %, or at least 30%, or at least 40%, or at least 50%, or at least 60%, or at least 70%, or at least 75%, or at least 80%, or at least 90%, or at least 95%, compared to administration of a placebo (or no administration of SLIT3 or SLIT2 polypeptide or vector). In some cases, administration of a SLIT3 or SLIT2 polypeptide, or a gene transfer vector comprising a nucleic acid sequence which encodes a SLIT3 or SLIT2 polypeptide, can increase bone volume or the maximal load tolerated by a bone by at least 1.5-fold, or by at least 2-fold.

A “gene transfer vector” is any molecule or composition that can carry a heterologous nucleic acid sequence into a suitable host cell where synthesis of the encoded protein takes place. Typically, a gene transfer vector is a nucleic acid molecule that has been engineered, using recombinant DNA techniques that available to those of ordinary skill in the art, to incorporate the heterologous nucleic acid sequence. Desirably, the gene transfer vector is comprised of DNA. Examples of suitable DNA-based gene transfer vectors include plasmids and viral vectors. However, gene transfer vectors that are not based on nucleic acids, such as liposomes, are also known and used in the art. The inventive gene transfer vector can be based on a single type of nucleic add (e.g., a plasmid) or non-nucleic add molecule (e.g., a lipid or a polymer). The gene transfer vector can be integrated into the host cell genome or can be present in the host cell in the form of an episome.

In one embodiment, the gene transfer vector is a viral vector. Suitable viral vectors include, for example, lentiviral vectors, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition. Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

In an embodiment, the invention provides an adeno-associated virus (AAV) vector which comprises, consists essentially of, or consists of a nucleic acid sequence encoding a SLIT3 or SLIT2 polypeptide or an interfering RNA molecule against SLIT3 or SLIT2 or SHN3. When the inventive AAV vector consists essentially of a nucleic acid sequence encoding a SLITS or SLIT2 polypeptide or an interfering RNA molecule against SLITS or SLIT2 or SHN3, additional components can be included that do not materially affect the AAV vector (e.g., genetic elements such as poly(A) sequences or restriction enzyme sites that facilitate manipulation of the vector in vitro).

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (sec, e.g., U.S. Pat. Nos. 6,342,390 and 6,821,511). Host cells comprising an integrated AAV genome show no change in cell growth or morphology (sec, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell. 61:447 (1990)). These proteins also regulate transcription from endogenous AAV promoters and promoters within helper viruses (see, e.g., Pereira et al., J. Virol., 71:1079 (1997)). The other Rep proteins modify the function of Rep78 and Rep68. The cap genes encode the capsid proteins VP1, VP2, and VP3. The cap genes are transcribed from the p40 promoter.

The AAV vector may be generated using any AAV serotype known in the art. Several AAV serotypes and over 100 AAV variants have been isolated from adenovirus stocks or from human or nonhuman primate tissues (reviewed in, e.g., Wu et al., Molecular Therapy. 14(3): 316 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-6 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, Hum. Gene Ther., 16:541 (2005); and Wu et al., supra). Genome sequences of various AAV serotypes and comparisons thereof are disclosed in, for example, GenBank Accession numbers U89790, J01901, AF043303, and AF085716; Chiorini et al., J. Virol., 71:6823 (1997); Srivastava et al., J. Virol., 45:555 (1983); Chiorini et al., J. Virol., 72:1309 (1999); Rutledge et al., J. Virol., 72:309 (1998); and Wu et al., J. Virol., 74:8635 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (sec Bantel-Schaal et al., J. Virol., 73(2):939 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (e.g., functionally substitute) corresponding sequences from other serotypes during production of AAV particles in mammalian cells.

Generally, the cap proteins, which determine the cellular tropicity of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., 13(1): 1 (2006); Gao et al., J. Virol., 28:6381 (2004); Gao et al., Proc. Natl. Acad. Sci. USA. 99:11854 (2002); De et al., Mol. Ther., 13:67 (2006); and Gao et al., Mol. Ther., 12:77 (2006).

In one embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). In one embodiment, the AAV vector is generated using an AAV that infects a non-human primate pseudotyped with an AAV that infects humans. Examples of such pseudotyped AAV vectors are disclosed in, e.g., Cearley et al., Molecular Therapy, 13:528 (2006). In one embodiment, an AAV vector can be generated which comprises a capsid protein from an AAV that infects rhesus macaques pseudotyped with AAV2 inverted terminal repeats (ITRs). In some cases, the inventive AAV vector comprises a capsid protein from AAV 10 (also referred to as “AAVrh.10”), which infects rhesus macaques pseudotyped with AAV2 ITRs (see, e.g., Watanabe et al., Gene Ther., 17(8): 1042 (2010); and Mao et al., Hum. Gene Therapy. 22:1525 (2011)).

In addition to the nucleic acid sequence encoding a SLIT3 or SLIT2 polypeptide or an interfering RNA molecule against SLIT3 or SLIT2, the AAV vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

A variety of promoters, including constitutive, inducible, and repressible promoters, from a variety of different sources can be used in the vectors. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93:3346 (19%)), the T-REX™ system (Invitrogen, Carlsbad, Calif.), LACSWITCH™ System (Stratagene, San Diego, Calif.), and the Cre-ERT tamoxifen inducible recombinase system (Indra et al., Nuc. Acid. Res., 27:4324 (1999); Nuc. Acid. Res., 28:e99 (2000); U.S. Pat. No. 7,112,715; and Kramer & Fussenegger, Methods Mol. Biol., 308:123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A variety of enhancers from a variety of different sources are available or can be obtained within cloned polynucleotides (from, e.g., depositories such as the ATCC as well as other commercial or individual sources). A number of polynucleotides comprising promoters (such as the CMV promoter) also comprise enhancer sequences. Enhancers can be located upstream, within, or downstream of coding sequences. In some cases, the nucleic acid sequence encoding a SLIT3 or SLIT2 polypeptide or an interfering RNA molecule against SLIT3 or SLIT2, is operably linked to a CMV enhancer/chicken beta-actin promoter (also referred to as a “CAG promoter”) (see. e.g., Niwa et al., Gene. 108:193 (1991); Daly et al., Proc. Natl. Acad. Sci. U.S.A., 96:2296 (1999); and Sondhi et al., Mol. Ther., 15:481 (2007)).

Typically, AAV vectors are produced using well characterized plasmids. For example, human embryonic kidney 293T cells are transfected with one of the transgene specific plasmids and another plasmid containing the adenovirus helper and AAV rep and cap genes (specific to AAVrh.10, 8 or 9 as required). After 72 hours, the cells are harvested, and the vector is released from the cells by five freeze/thaw cycles. Subsequent centrifugation and benzonase treatment removes cellular debris and unencapsidated DNA. Iodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1× phosphate buffered saline. The viral titers may be measured by TaqMan® real-time PCR and the viral purity may be assessed by SDS-PAGE.

### Formulations and Delivery

The present invention also provides a method to prevent bone loss, or promote bone growth, strengthening, or healing in a subject in need thereof, by administering to said subject a SLIT3 or SLIT2 agent and it provides a method to prevent bone growth in a subject in need thereof, by administering to said subject a SLIT3 or SLIT2-interfering agent, in each case with a pharmaceutically acceptable excipient. In cases wherein the SLIT3 or SLIT2 agent or SLIT3 or SLIT2-interfcring agent is a nucleic acid, including interfering RNA, it may be carried in a vector.

In another aspect, the present invention provides pharmaceutically acceptable compositions which comprise a therapeutically-effective amount of one or more of a SLIT3 or SLIT2 agent or a SLIT3 or SLIT2-interfering agent, including vectors carrying them, as described above, formulated together with one or more pharmaceutically acceptable excipients. In another aspect, the present invention provides pharmaceutically acceptable compositions which comprise a therapeutically-effective amount of one or more of a SLIT3 or SLIT2 agent or a SLIT3 or SLIT2-interfering agent, as described above, formulated together with one or more pharmaceutically acceptable excipients and other therapeutically effective medications known in the art allowing for but not limited to combination therapies to improve overall efficacy of each individual therapeutic or to limit the concentration of either therapeutic to avoid side effects and maintain efficacy. The active ingredient and excipient(s) may be formulated into compositions and dosage forms. The active ingredient or compositions thereof can be administered with an orthopedic implant. For example, the active ingredient or compositions thereof can coat or be bound (covalently or non-covalently) to an orthopedic implant.

As described in detail below, the pharmaceutical compositions of the present invention may be specially formulated for administration in solid or liquid form, including those adapted for the following: (1) oral administration, for example, tablets, capsules, powders, granules, pastes for application to the tongue, aqueous or non-aqueous solutions or suspensions, drenches, or syrups; (2) parenteral administration, for example, by subcutaneous, intramuscular or intravenous injection as, for example, a sterile solution or suspension; (3) topical application, for example, as a cream, ointment or spray applied to bone, or provided in a depot formulation, for example embedded or coating a medical device, (4) intravaginally or intrarectally, for example, as a pessary, cream or foam; (5) sublingually or buccally, (6) ocularly; (7) transdermally; (8) nasally; or (9) locally. In some cases, administration is locally, for example, to a bone or site close to a bone.

A therapeutically effective amount of the pharmaceutical composition of the present invention is sufficient to treat or prevent a disease characterized by symptoms comprising loss of bone, weakened, damaged, degraded, or broken bones, or by excessive bone growth, or the risk of these things. The dosage of active ingredients) may vary, depending on the reason for use and the individual subject. The dosage may be adjusted based on the subject's weight, the age and health of the subject, and tolerance for the compound or composition.

For example, the proteins, nucleic acids encoding such proteins, inhibitors, nucleic adds encoding such peptide inhibitors, and combinations thereof, may be administered as single or divided dosages. For example, proteins, nucleic adds encoding such proteins, or inhibitors, nucleic acids encoding such peptide inhibitors, can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg, at least about 1 mg/kg to about 100 mg/kg, or at least about 0.1 mg/kg to about 50 to 100 mg/kg of body weight, although other dosages may provide beneficial results. The amount administered will vary depending on various factors including, but not limited to, the proteins, nucleic acids encoding such proteins, inhibitors, or nucleic acids encoding such peptide inhibitors chosen for administration, the disease, the weight the physical condition, the health, and the age of the mammal. Such factors can be determined using the information provided herein.

The phrase “pharmaceutically acceptable” is employed herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

Excipients are added to the composition for a variety of purposes. Diluents increase the bulk of a solid pharmaceutical composition and may make a pharmaceutical dosage form containing the composition easier for the patient and caregiver to handle. Diluents for solid compositions include, for example, microcrystalline cellulose (e.g. Avicel®), micro fine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methyl cellulose, polymethacrylates, povidone (e.g. Kollidon®, Plasdone®), pregelatinized starch, sodium alginate and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

In liquid pharmaceutical compositions of the present invention, the SLIT3 or SLIT2 agent, or SLIT3 or SLIT2-interfering agent, as described above, and any other solid excipients are dissolved or suspended in a liquid carrier such as water, water-for-injection, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the inventive gene transfer vector is administered in a composition formulated to protect the gene transfer vector from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the inventive method. Formulations for gene transfer vector —containing compositions are further described in, far example, Wright et al., Curr. Opin. Drug Discov. Devel., 6(2): 174-178 (2003) and Wright et al., Molecular Therapy, 12: 171-178 (2005)).

Solid and liquid compositions may also be dyed using any pharmaceutically acceptable colorant to improve their appearance and/or facilitate patient identification of the product and unit dosage level.

The dosage form of the present invention may be a capsule containing the composition, for example, a powdered or granulated solid composition of the invention, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

Suitable formulations for the composition include solid and semi-solid compositions. For example, vectors and/or proteins can be mixed with or complexed to a carrier to render it resistant to dispersion from the site of delivery, to inhibit acidic and enzymatic hydrolysis, or a combination thereof. In some cases, the vectors and/or proteins can be packaged in a carrier that includes one or more types of crystalline materials, amorphous materials, liposomes, lipids, charged lipids (e.g., cytofectins), DNA-protein complexes, and biopolymers. The compositions can be encapsulated, e.g., in liposomes or bioploymerws, or in a formulation that provides for slow or reduced release of the active ingredient. Liposomes can be used for encapsulation and transfection of nucleic acids and proteins in vitro. Synthetic cationic lipids can limit problems encountered with liposome-mediated transfection and can be used to prepare liposomes for in vivo transfection of a nucleic acids (Feigner et al, Proc. Natl. Acad. Sri. USA. 84:1413 (1987); Mackey et al, Proc. Natl Acad Sri. USA £5:8027 (1988); and Ulmer et al. Science 259:1145 (1993)). The use of cationic lipids may promote encapsulation of negatively charged nucleic acids and can promote fusion with negatively charged cell membranes (Feigner et al., Science 337:387 (1989)). Useful lipid compounds and compositions for transfer of nucleic acids are described in WO95/18863, WO96/17823 and U.S. Pat. No. 5,459,127. Lipofection can be used to introduce exogenous genes into the specific sites in vivo. Lipids may be chemically coupled to other molecules for targeting the complex to specific sites (e.g., to bone-related sites). Targeted peptides, e.g., hormones or neurotransmitters, and proteins such as antibodies, or non-peptide molecules (e.g., ligands) can be coupled to liposomes or to other carriers.

In some cases, the effect of a drug or active agent can be prolonged, for example, by slowing the absorption of the drug from local administration, subcutaneous injection, or intramuscular injection. This may be accomplished using a liquid suspension of crystalline or amorphous material having poor water solubility. The rate of absorption of the drug then depends upon its rate of dissolution which, in turn, may depend upon crystal size and crystalline form. Alternatively, delayed absorption of a locally-administered drug form is accomplished by dissolving or suspending the drug in an oil vehicle. Injectable depot forms can be made by forming microencapsulated matrices of the subject compounds in biodegradable polymers such as collagen, gelatin, or polylactide-polyglycolide. Depending on the ratio of active ingredient to polymer, and the nature of the particular polymer employed, the rate of active ingredient release can be controlled. Examples of other biodegradable polymers include poly(orthoesters) and poly(anhydrides). Depot injectable formulations are also prepared by entrapping the drug in liposomes or microemulsions which are compatible with body tissue.

With respect to delivering SLIT3 or SLIT2 agents, or SLIT3 or SLIT2 interfering agents to bone, methods can include delivery the active ingredient with bone morphogenic protein, platelet-derived growth factor, or a combination thereof to bone in the contexts of oral, periodontic, ortheopedic, spine, and other surgeries and procedures. See each of Shah et al. (2012) and Friedlaender, G E et al. (2013), each of which is incorporated herein by reference in its entirety. This includes versions of SLIT3 or SLIT2 protein or antibodies against SLIT3 or SLIT2, with additions of anionic amino acids such as glutamic and aspartic acid, intended to provide electrostatic targeting of SLIT3 or SLIT2 to bone surface.

### Liposomes and Nanoparticles

In an embodiment of the present invention, the pharmaceutical composition or formulation containing a SLIT3 or SLIT2 agent, or a SLIT3 or SLIT2 interfering agent can be encapsulated in a lipid formulation, for example as described in Semple et al., “Rational Design of Cationic Lipids for siRNA Delivery,” Nature Biotech. 28:172-176 (2010), WO2011/034798 to Bumcrot et al., WO2009/111658 to Bumcrot et al., and WO2010/105209 to Bumcrot et al., which are hereby incorporated by reference in their entirety.

In another embodiment of the present invention, the delivery vehicle is a nanoparticle. A variety of nanoparticle delivery vehicles are known in the art and are suitable for delivery of a SLIT3 or SLIT2 agent, or SLIT3 or SLIT2 interfering agent, of the invention (see e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy,” Adv. Exp. Med. Biol. 622:209-219 (2008), which is hereby incorporated by reference in its entirety), polyethylenimine-alt-poly(ethylene glycol) copolymers (Park et al., “Degradable Polyethylenimine-alt-Poly(ethylene glycol) Copolymers As Novel Gene Carriers,” J. Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnology 10(5):3280-3 (2010), which are hereby incorporated by reference in their entirety), and liposome-entrapped siRNA nanoparticles (Kenny et al., “Novel Multifunctional Nanoparticle Mediates siRNA Tumor Delivery, Visualization and Therapeutic Tumor Reduction In Vivo,” J. Control Release 149(2): 111-116 (2011), which is hereby incorporated by reference in its entirety). Other nanoparticle delivery vehicles suitable for use in the present invention include microcapsule nanotube devices disclosed in U.S. Patent Publication No. 2010/0215724 to Prakash et al., which is hereby incorporated by reference in its entirety.

SLIT3 or SLIT2 agents, or SLIT3 or SLIT2 interfering agents, including liposomes or nanoparticles carrying them or into which they are incorporated, may be targeted to bone using strategies such as a bisphosphonate conjugation or (AspSerSer)6-liposomes and Aptamer-functionalized lipid nanoparticles to increase the fractional distribution to bone (Zhang, Guo, et al. 2012, Liang et al. 2015, Guan et al. 2012, Yao et al. 2013).

### Polymers

Hydrophilic polymers suitable for use in the present invention can be those which are readily water-soluble. Hydrophilic polymers can also be covalently attached to a vesicle-forming lipid. The polymers employed can include those which are tolerated in vivo without toxic effects (i.e., are biocompatible). Suitable polymers include collagen, polyethylene glycol (PEG), polylactic (also termed polylactide), polyglycolic acid (also termed polyglycolide), a polylactic-polyglycolic acid copolymer, and polyvinyl alcohol. Polymers include those having a molecular weight of from about 100 or 120 daltons up to about 5,000 or 10,000 daltons, and for example those having a molecular weight of from about 300 daltons to about 5,000 daltons. In some cases, the polymer can be polyethylene glycol having a molecular weight of from about 100 to about 5,000 daltons or having a molecular weight of from about 300 to about 5,000 daltons. In some cases, the polymer is polyethylene glycol of 750 daltons (PEG(750)). Polymers may also be defined by the number of monomers therein; in some cases, polymers of at least about three monomers, such PEG polymers consisting of three monomers (approximately 150 daltons) can be used.

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

### Cyclodextrins

Cyclodextrins are cyclic oligosaccharides, consisting of 6, 7 or 8 glucose units, designated by the Greek letter alpha, beta, or gamma, respectively. Cyclodextrins with fewer than six glucose units are not known to exist. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17-beta-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility. Numerous cyclodextrins and methods for their preparation have been described.

### EXAMPLES

The present description is further illustrated by the following examples, which should not be construed as limiting in any way. The contents of all cited references (including literature references, issued patents, published patent applications as cited throughout this application) are hereby expressly incorporated by reference.

### Example 1: SHN3/SLIT3 are Important for Bone Regeneration Methods

### Genetically Modified Mice

Shn3^{-/-} (B ALB/c), Shn3^{KI/KI} (C57BL/6J), Shn3 floxed allele (C57BL/6J), Slit3^{-/-} (BALB/c), Robo1^{-/-} (ICR) and Robo4^{-/-} (C57BL/6J) mice were all previously reported, with Robo1^{-/-} mice being a generous gift from Dr. Marc Tessier-Lavigne (Jaworski and Tessier-Lavigne 2012, Shim et al. 2013, Wein et al. 2012, Zhang et al. 2009, Jones et al. 2008, Yuan et al. 2003). To generate Slit3 floxed mice, the SLIT3-F08 mouse embryonic stem (ES) cell line in which exon 8 is flanked by loxP sites was obtained from International Mouse Phenotyping Consortium (IMPC). After validation. F08 EC cells were injected into C57BL/6J blastocysts, and the derived chimeras displaying germline transmissions were selected for further breeding. The LacZ and neo cassettes were removed by intercrossing with transgenic mice expressing Flp recombinase, Slit3 floxed mice were backcrossed with C57BL/6J mice for 8 generations.

Transgenic mice expressing Cre recombinase under control of the cdh5 promoter (cdh5-Cre) (Chen et al. 2009), osterix promoter (osx-Cre) (Rodda and McMahon 2006), dmp1 promoter (dmp1-Cre) (Lu et al. 2007) and Osteocalcin-CreERT mice (Park et al. 2012) were mated with Shn3 floxed mice or Slit3 floxed mice to obtain various Shn3 or Slit3 conditional KO mouse. For postnatal activation of CreERT, 100 mg/kg tamoxifen (Sigma) in corn oil (Sigma) was intraperitoneally injected to one-month-old mice once a day for five consecutive days. Littermate controls were utilized for all experiments.

All animals were maintained in accordance with the National Institutes of Health Guide for the Care and Use of Laboratory Animals and were handled according to protocols approved by the Weill Cornell Medical College subcommittee on animal care (IACUC).

### μCT Analysis

Micro-CT (μCT) analysis was conducted on a Scanco Medical μCT 35 system at the Citigroup Biomedical Imaging Core using the previously described parameters. (Shim et al. 2013). μCT analysis was performed by an investigator blinded to the genotypes of the animals under analysis.

### Immunofluorescence, Histology and Histomorphometry

For immunofluorescence, fresh bone dissected and soft tissues from wild-type mice and mutant mice were collected and immediately fixed in ice-cold 4% paraformaldehyde solution for overnight. Decalcification was specially carried out with 0.5 M EDTA at 4° C. with constant shaking for bone samples from mice (age≥1 W). All samples were embedded in OCT compound (Sakura) and cut into 25-um-thick sagittal sections using a cryostat (Leica). Immunofluorescence staining, and analysis was performed as described by Fukuda et al. (2013) and Xu et al. (2017a, b). Briefly, after treatment with 0.2% Triton X-100 for 10 min, sections were blocked with 5% donkey serum at room temperature for 30 min and incubated overnight at 4° C. with antibodies: CD31 (553370, BD Pharmigen, 1 MOO), CD31 conjugated to Alexa Fluor 488 (FAB3628G, R&D Systems, 1:50), Endomucin (sc-65495, Santa Cruz. 1:100) or Beta Galactosidase antibody (GTX77365, Gene Tex, 1:100). Primary antibodies were visualized with species appropriate Alexa Fluor-coupled secondary antibodies (1:400, Molecular Probes). Nuclei were counterstained with DAPI. An Olympus IX81 confocal microscope or Zeiss LSM-880 confocal microscope was used to image samples. Quantification of skeletal vasculature was performed as previously described (Fukuda et al. 2013). Briefly, the CD31-positive or Endomucin-positive (red) area relative to the total bone marrow area (visualized in blue) was calculated using Image J software (see website at rsbweb,nih,gov/ij/).

For whole-mount immunostaining, retinas were collected from adult mice after systemic or local SLIT3 administration in bone fracture models.

For histological analysis, hindlimbs were dissected from the mice or human callus, fixed in 10% neutral buffered formalin for 24-48 hours, and decalcified by daily changes of 15% tetrasodium EDTA for 2 weeks. Tissues were dehydrated by passage through an ethanol series, cleared twice in xylene, embedded in paraffin, and sectioned at 7 μm thickness. Decalcified sections were stained with hematoxylin and eosin (H&E). We incubated the slides with primary antibody to mouse EMCN (sc-65495, Santa Cruz, 1:200) and human Slit3 (ab198726, Abcam 1:50) at 37° C. for 2 h and subsequently used a horseradish peroxidase-streptavidin detection system (Dako) to detect the immunoreactivity. The number and volume of positively stained vessels was measured in four random visual fields of callus in 3 sequential sections per mouse in each group.

For histomorphometry, mice were injected with calcein (25 mg/kg, Sigma), and undecalcified sections of the lumbar vertebrae were stained using von Kossa and TRAP as described by Fukuda et al. (2013). Static and dynamic histomorphometric analysis was performed with using the Osteomeasure Analysis System (Osteometrics) following standard nomenclature as described (Dempster et al. 2013). Bone volume/total volume (BV/TV), bone formation rate/bone surface (BFR/BS, μm³ μm⁻¹ yr⁻¹), mineral apposition rate (MM, um Day⁻¹), osteoblast surface/bone surface (Ob.S/BS, %) and osteoclast number/bone perimeter (No. OC/Bpm) were analyzed.

### Flow Cytometry and Cell Sorting

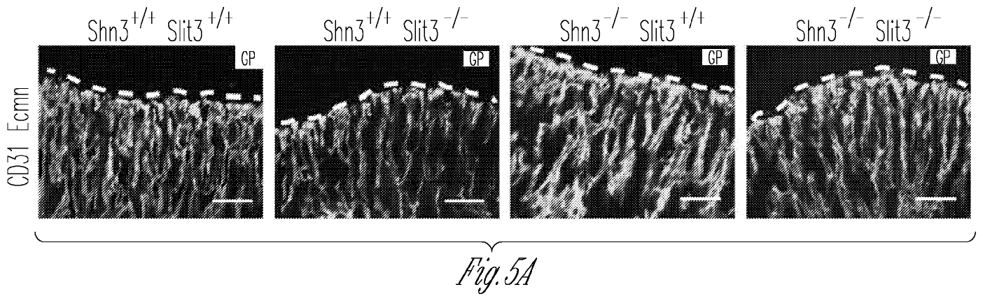

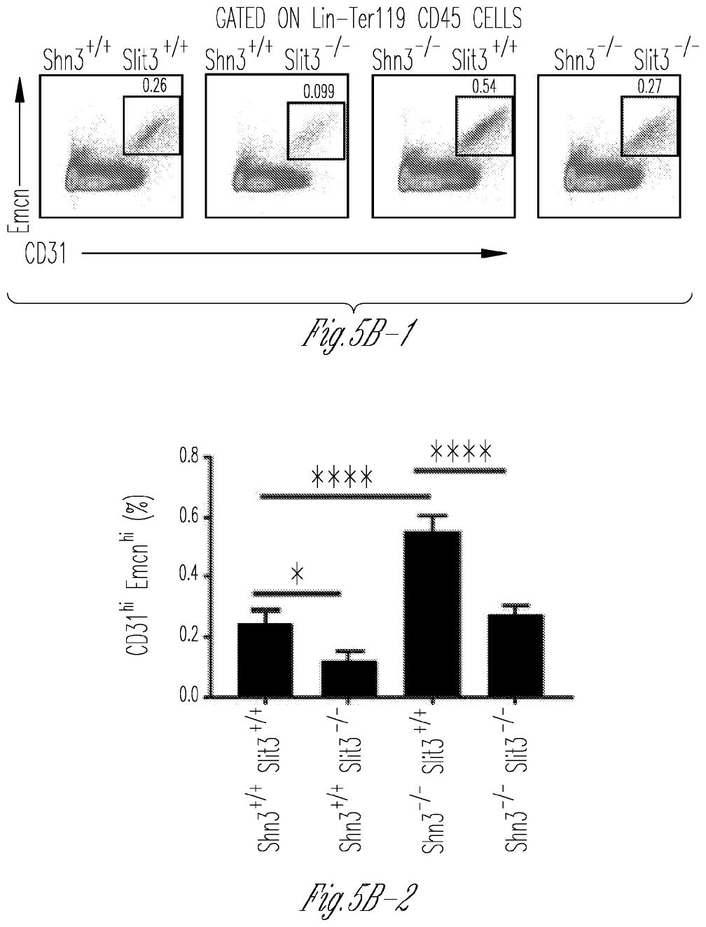

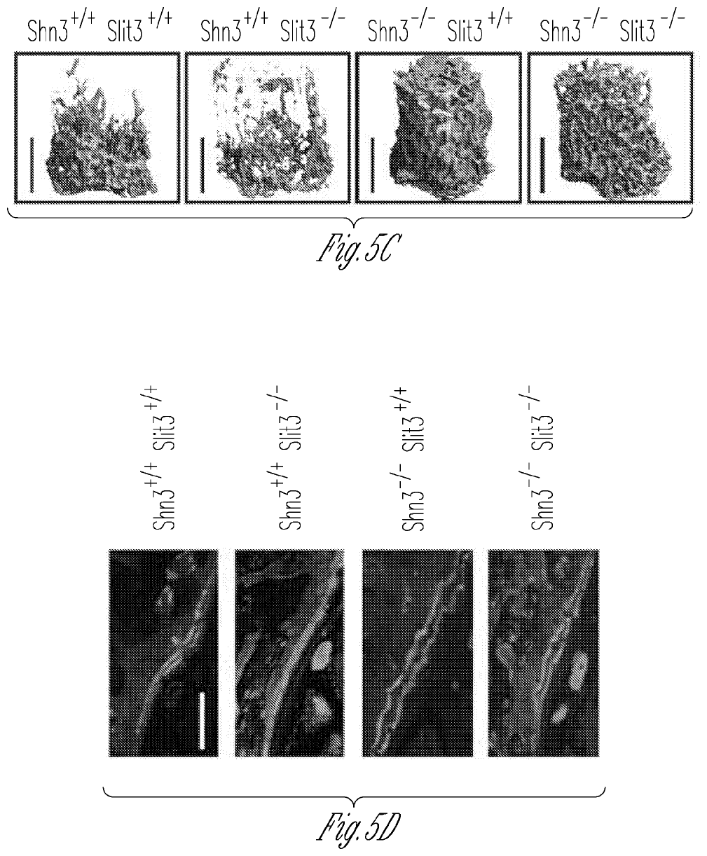

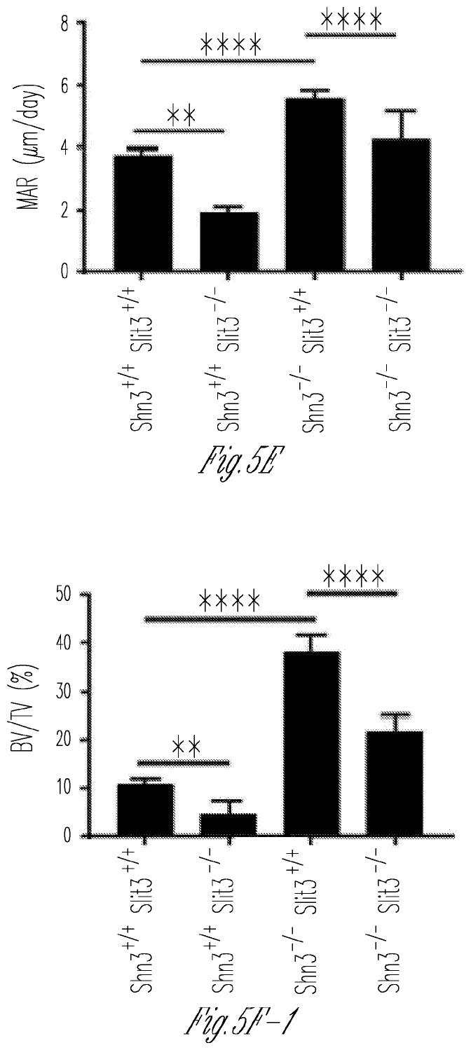

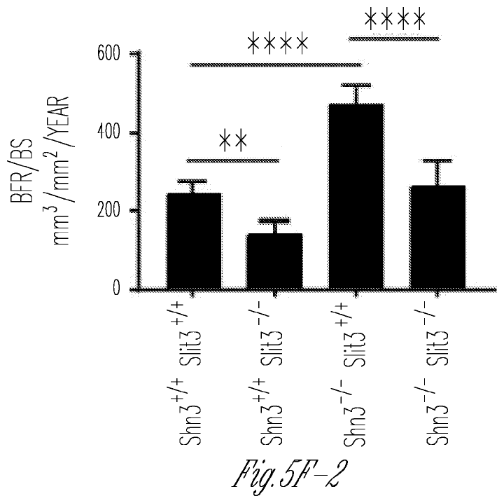

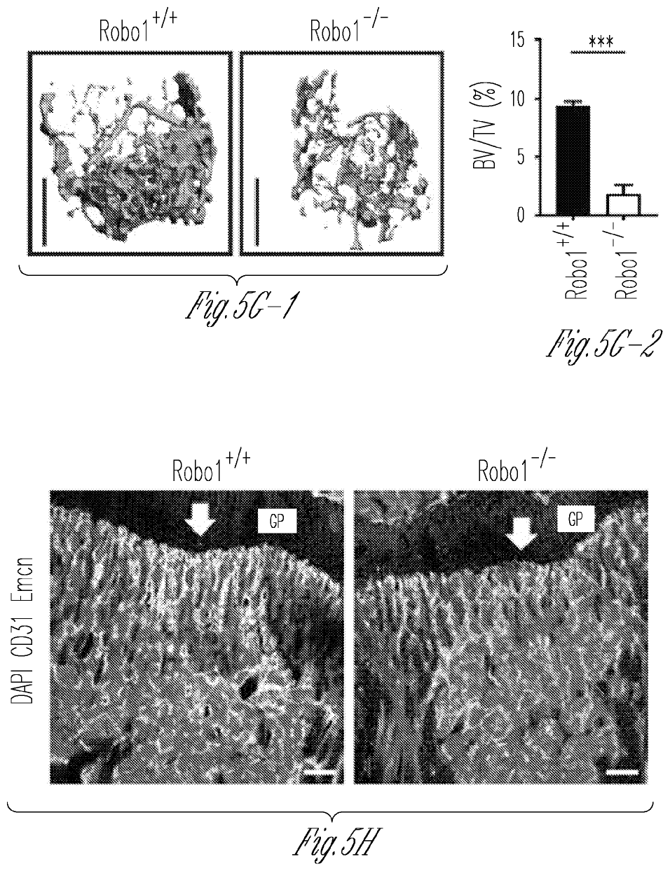

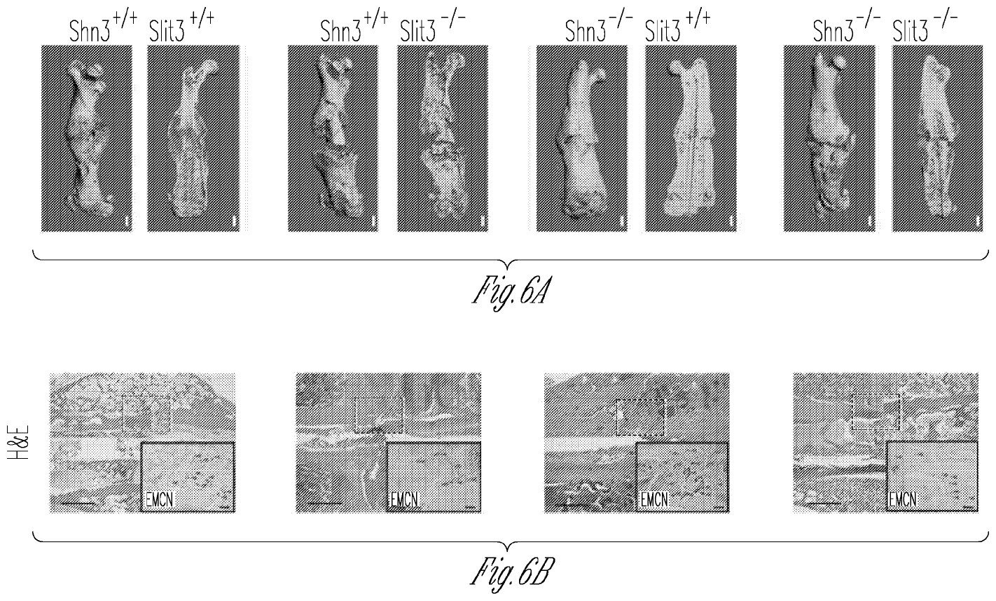

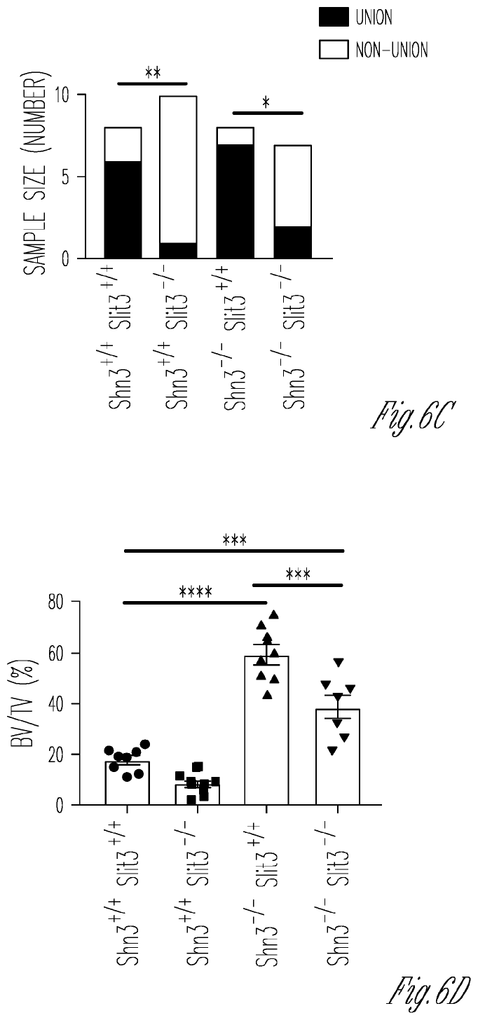

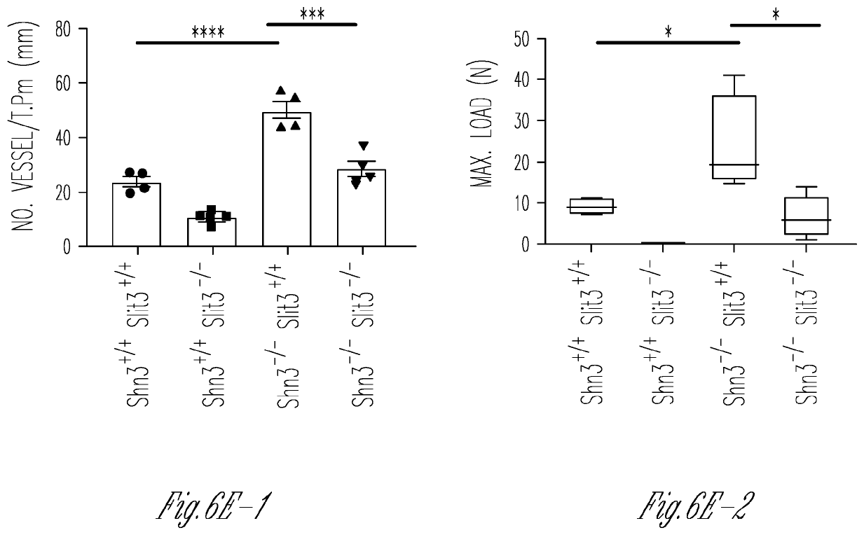

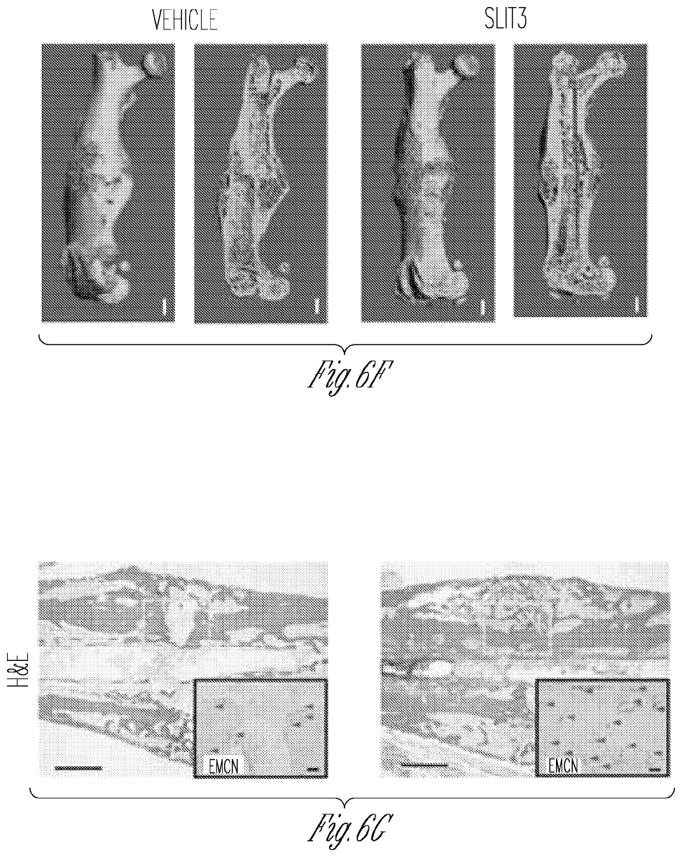

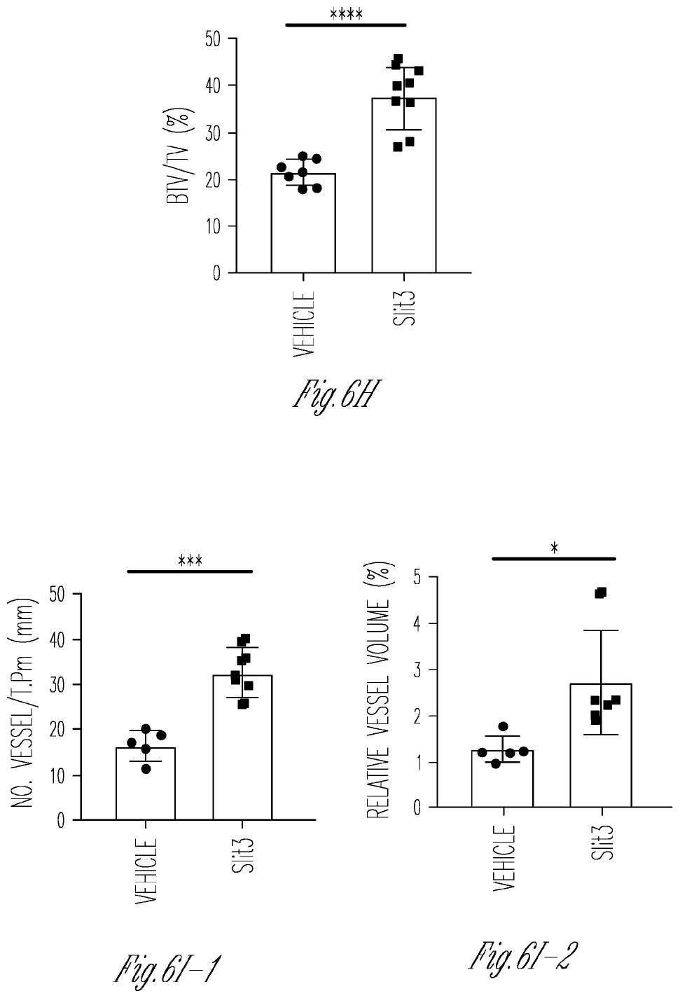

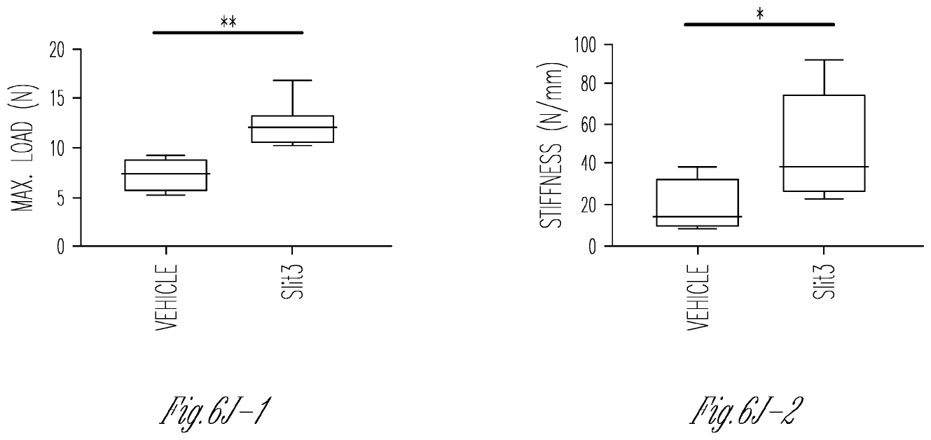

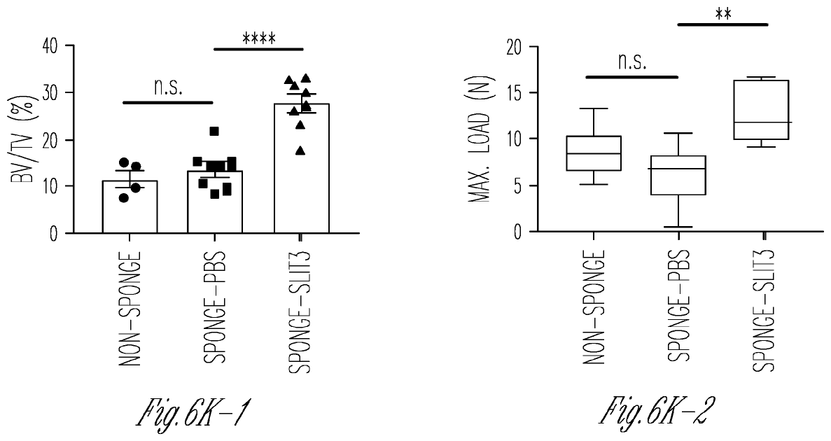

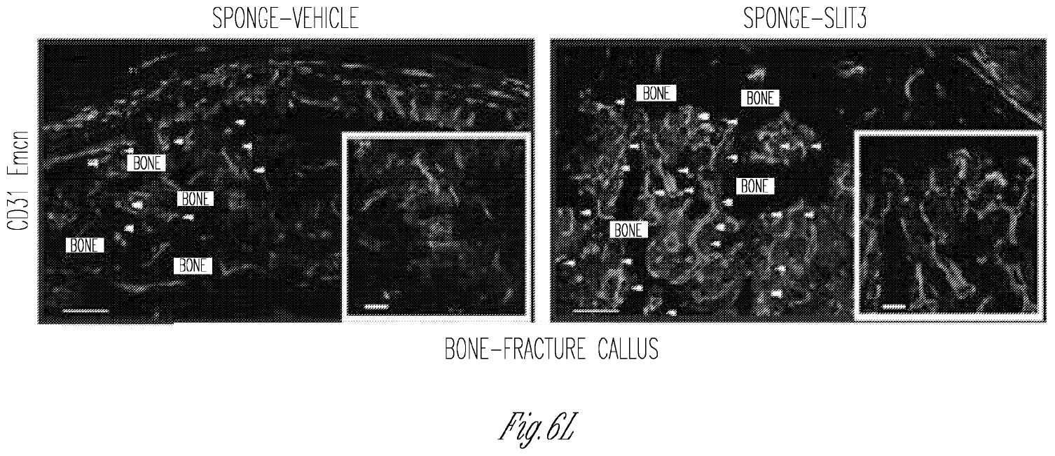

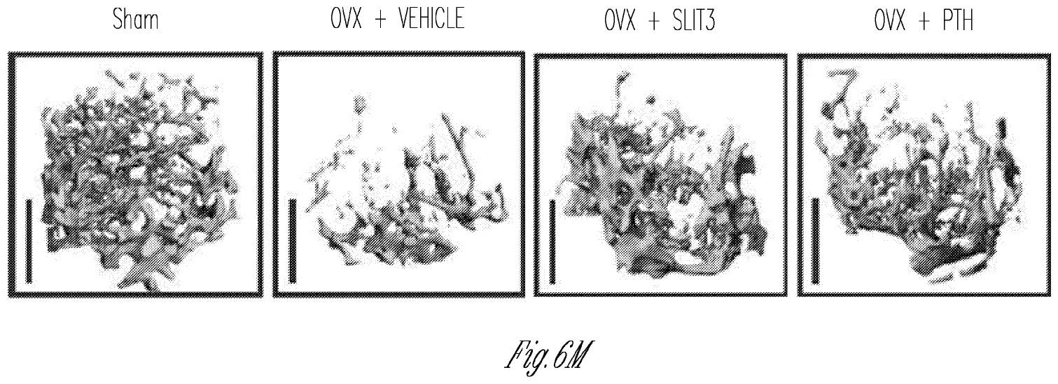

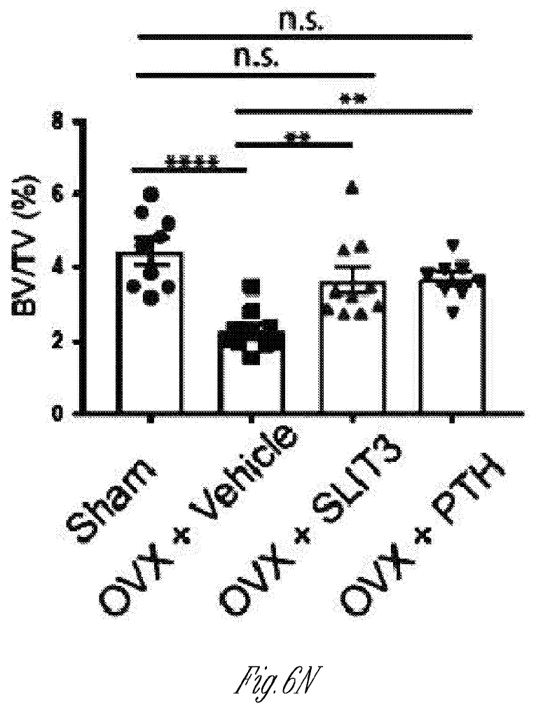

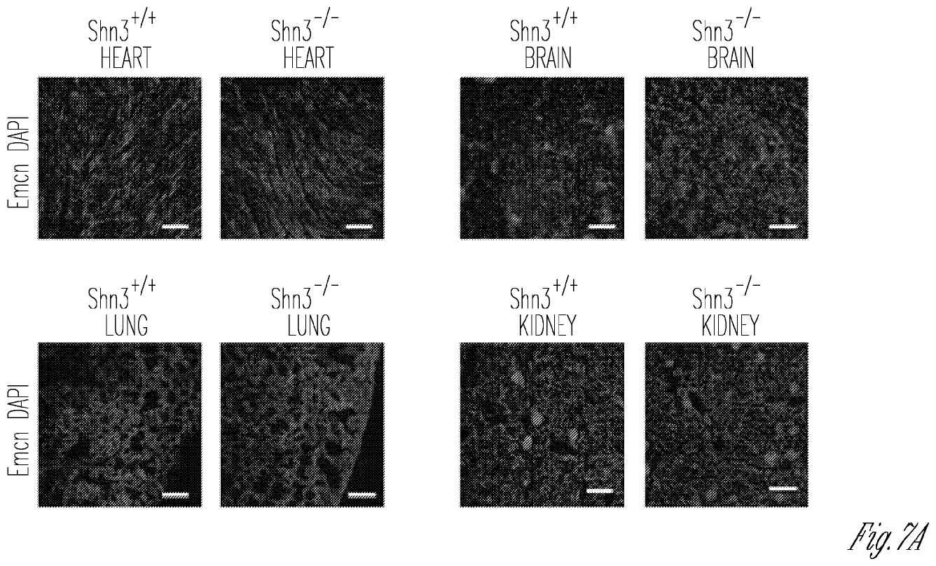

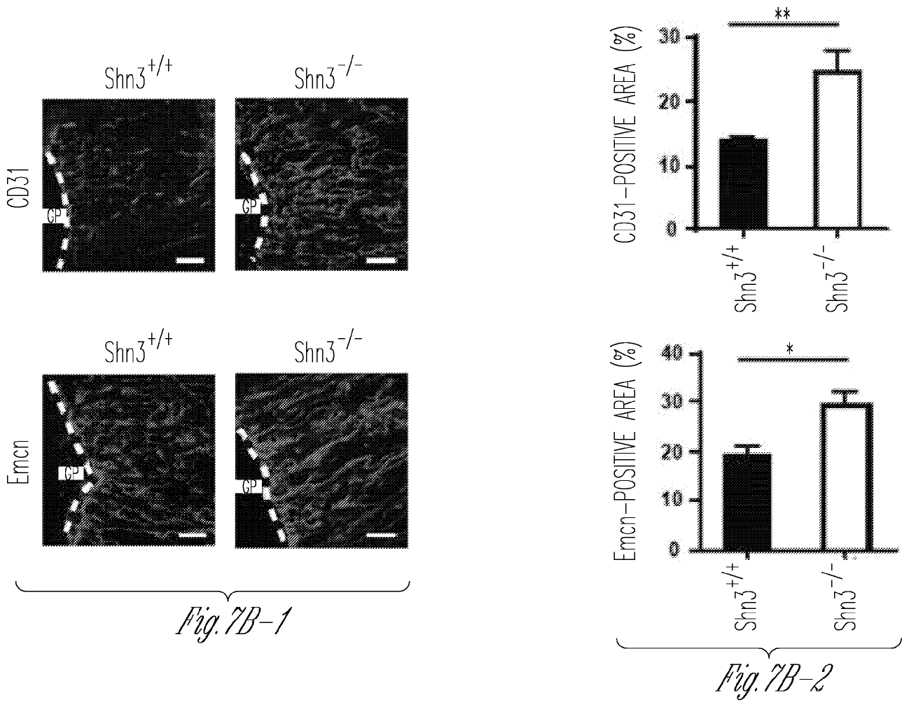

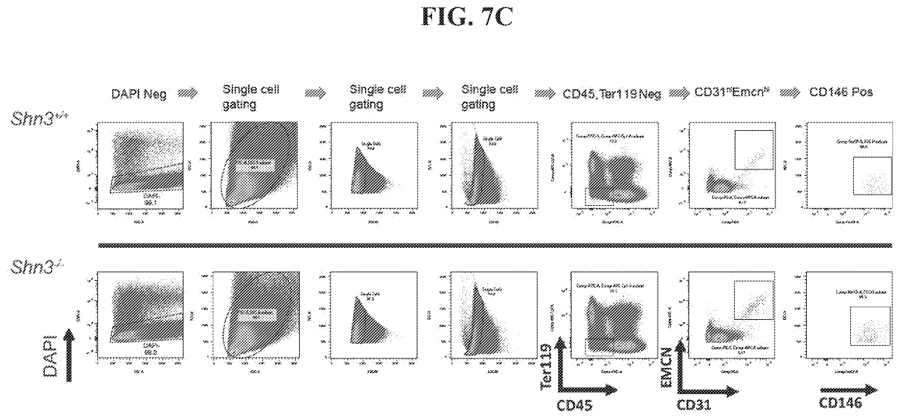

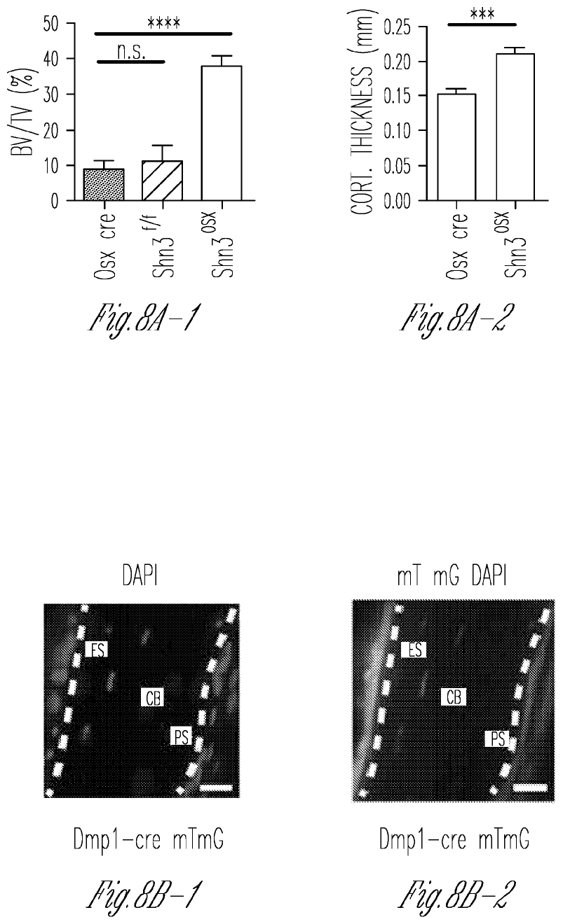

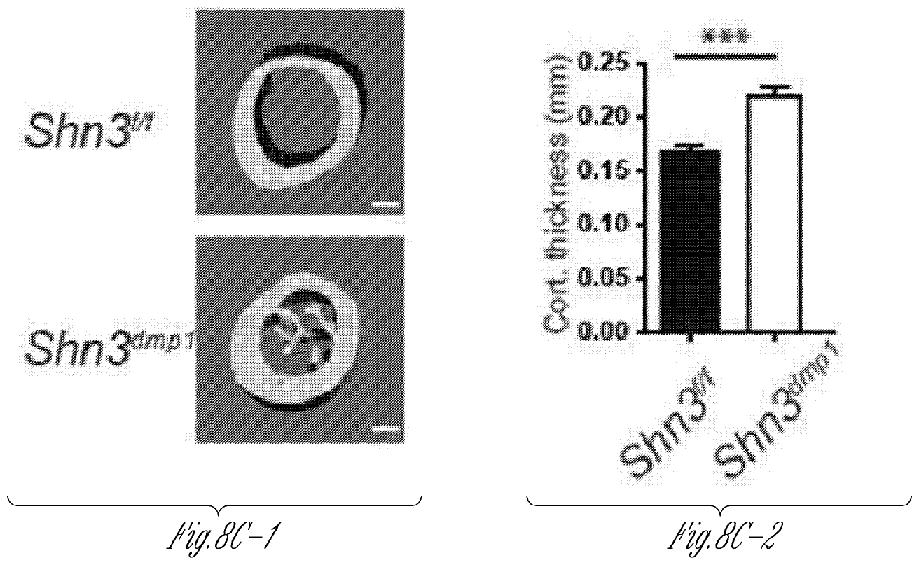

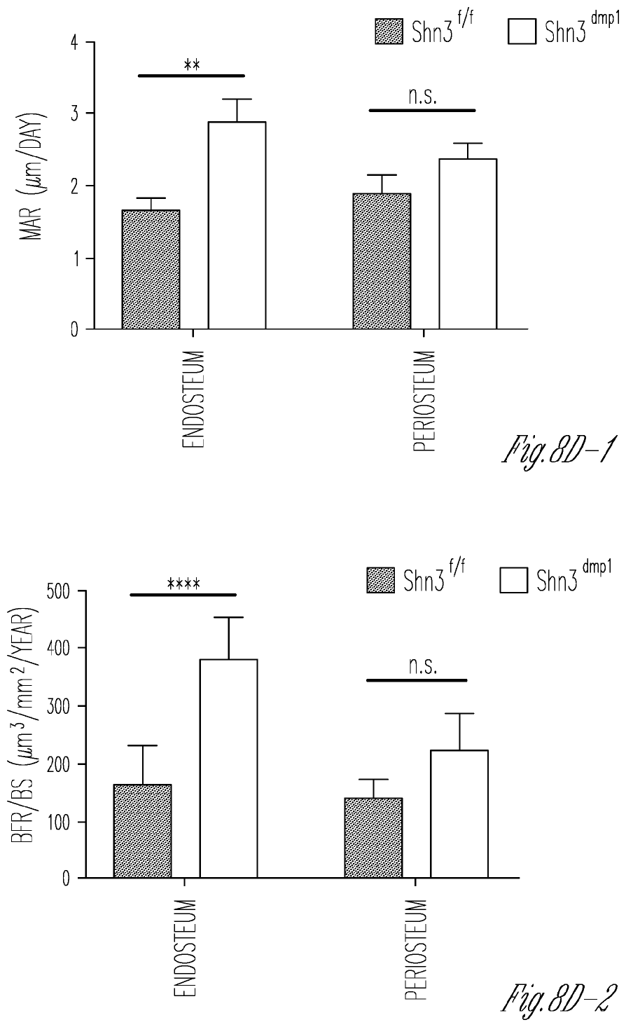

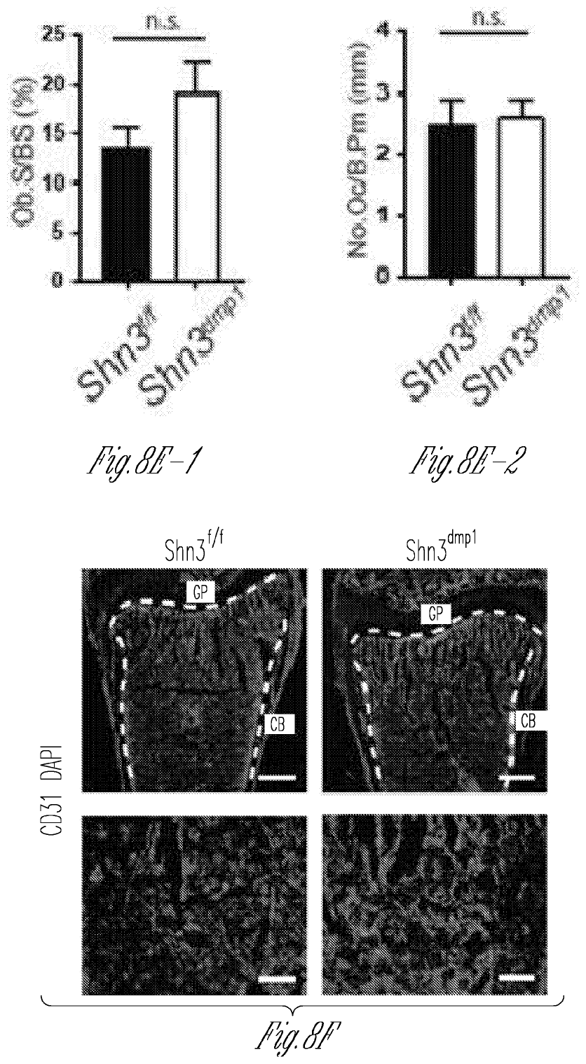

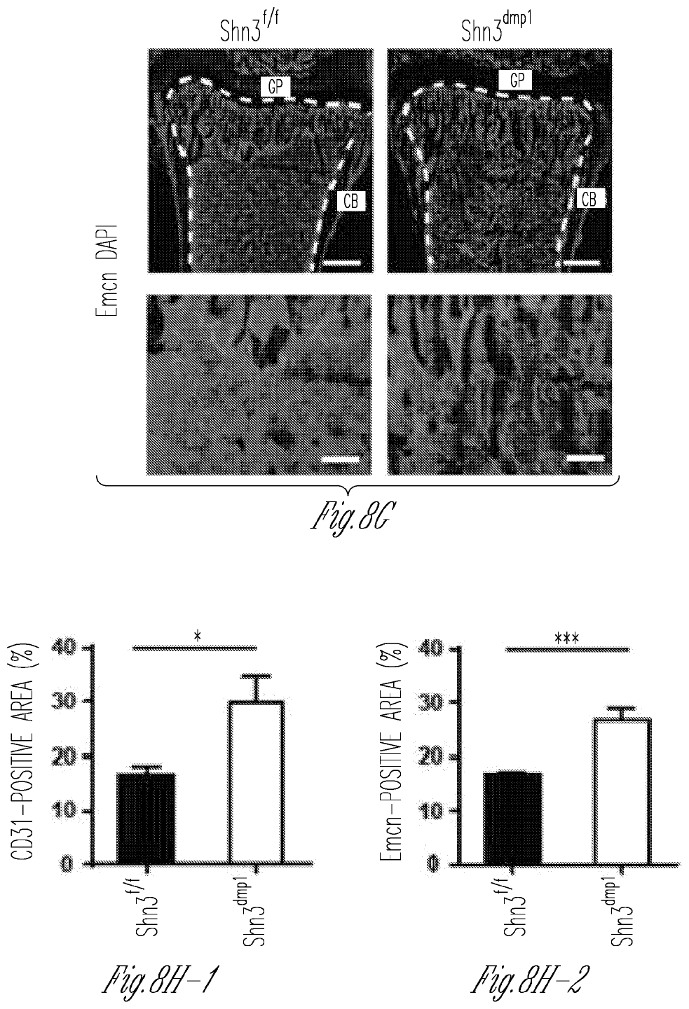

Femur and tibia were dissected from mutant mice and control groups after removing surrounding connective tissues. The metaphysis region and diaphysis regions of bone was crushed in Hanks Balanced Salt Solution (Life Technologies) containing 10 mM HOPES (pH 7.2) (CellGro) and enzymatically digested with 2.5 mg/mL Collagenase A (Roche) and 1 unit/mL Dispase II (Roche) for 15 minutes at 37° C. under gentle agitation. The resulting cell suspensions were filtered (40 μm), washed using PBS (pH 7.2) containing 0.5% BSA (Fraction V) and 2 mM EDTA. After washing, equal numbers of cells per mouse were blocked with Purified Rat Anti-Mouse CD16/CD32 (BD Biosciences) for 30 min on ice, then stained with APC-conjugated EMCN antibody (ebioscience 50-5851-80), PE-conjugated CD31 (ebioscience 12-0311-81). FITC-conjugated CD45 (Tonbo 35-0451), APC/Cy7-conjugated Ter119 (Biolegend 116223) and PerCP-Cy5.5-conjugated CD146 (BD Biosciences 562231) for 45 min on ice. After washing, cells were resuspended in PBS (pH 7.2) with 2 mM EDTA and 1 μg/mL 4-6,Diamidino-2-Phenylindole (DAPI) (live/dead exclusion) for analysis on an LSRII flow cytometer system (BD Biosciences) cytometer and analyzed using FlowJo software (TreeStar). Cell sorting was performed with a FACS Aria II SORP cell sorter (Becton Dickinson) at Weill Cornell Medical College, with exclusion of DAPI⁺ cells and doublets. The strategy to sort CD31^{hi}EMCN^{hi} endothelial cells is diagrammed in extended FIG. 7C.

### Osteoblast Culture and Differentiation Assays.

Primary calvarial osteoblasts were isolated from five-day-old mice by triple collagenase/dispase II digestion. Cells were cultured in α-MEM medium (Gibco) containing 10% FBS, 2 mM L-glutamine, 1% penicillin/streptomycin, 1% Hepes, and 1% nonessential amino acids and differentiated with ascorbic acid and β-glycerophosphate. Conditioned medium was collected from culture of primary osteoblasts and stocked at −80° C. hMSCs were cultured and differentiated into osteoblasts using a commercial kit (Cyagen). All cells were routinely tested to be mycoplasma negative.

For staining of extracellular matrix mineralization, cells were fixed with 10% neutral buffered formalin and stained with alizarin red. Mineralization activity was measured by colorimetric analysis. For alkaline phosphatase (ALP) activity, osteoblasts were fixed with 10% neutral formalin buffer and stained with the solution containing Fast Blue and Naphthol (Sigma-Aldrich). Alternatively, osteoblasts were incubated with 10-fold diluted Alamar Blue solution, washed, and incubated with a solution containing 6.5 mM Na₂CO₃, 18.5 mM NaHCO₃, 2 mM MgCl₂, and phosphatase substrate (Sigma-Aldrich). ALP activity was measured by a spectrophotometer (Thermo).

### Osteoclast Culture and Differentiation.

Murine bone marrow cells were flushed from the femur and tibia of mice and cultured in petri dishes in α-MEM medium with 10% FBS and 20 ng/ml of rM-CSF. Nonadherent cells were replated into tissue culture dishes and cultured in the same medium for 3 d to obtain osteoclast precursors. The osteoclast precursors then differentiated into osteoclasts in the presence of human RAMKL (50 ng/ml; PeproTech) and M-CSF for 3 days. Peripheral blood mononuclear cells from the whole blood of healthy volunteers were isolated by density gradient centrifugation using Ficoll (Invitrogen, Carlsbad, Calif.). CD14-positive cells were purified from fresh PBMCs using anti-CD14 magnetic beads (Miltenyi Biotec, Auburn, Calif.), as recommended by the manufacturer. Human monocytes were cultured in a-MEM with 10% FBS in the presence of M-CSF (20 ng/ml; PeproTech, Rocky Hill, N.J.) for 2 d to obtain monocyte-derived macrophages. Experiments with human cells were approved by the Hospital for Special Surgery Institutional Review Board.

### Endothelial Cell Culture and Functional Assays.

Mouse bone marrow derived late-stage endothelial progenitor outgrowth cells (EPOCs) were obtained from BioChain (7030031) and cultured in growth medium (BioChain Z7030035) as described previously (Xie et al. 2014). Endothelial cell migration assay was set up in Trans well-24 well plates with 8-um pore filters. Briefly, 1×10⁵ cells/well after 1-hour serum starvation were seeded in the upper chamber, then incubated with conditioned medium from osteoblasts and control in the lower chambers for a further 3 h. The cells in the upper surface of each filter were removed with cotton swabs. The cells that migrated into the lower surface were fixed with 4% PFA for 30 min and then stained with crystal violet. The cell numbers were quantified by counting a centered microscope field per each filter (5 wells for each condition). Endothelial cell wound healing assays were conducted in 12-well plates precoated with gelatin (Stemcell Technologies). 3×10⁵ cells/well were plated overnight and stimulated with a wound in the form of a single linear scratch made with a yellow pipette tip. After gently washing the well twice, cells were cultured in medium with SLIT3 or vehicle. At 6 and 12 hours after injury, cells were stained by 0.5% crystal violet and photographed. The width of the wound area was quantitatively evaluated using ImageJ (see website at rsb.info.uih.gov/ij/download.html). Endothelial cell proliferation assays were conducted in 96-well plates pre-coated with gelatin (Stemcell Technologies). EPOCs (3×10⁴ cells/well) were seeded in the medium with a serial dilution of SLIT3 protein or vehicle in plates. At 0, 24, 48 hours after seeding, cells were incubated with 10-fold diluted Alamar Blue solution (Thermo Fisher) and the supernatant was evaluated with a spectrophotometer (Thermo) (5 wells for each condition). Endothelial cell tube formation assay was conducted in 96-well plates pre-coated with Matrigel (BD). After 1 h serum starvation, EPOCs (3×10⁴ cells/well) were seeded in conditioned medium dilution or control medium on polymerized Matrigel in plates. Alter 5 hours incubation at 37° C., the number of tube branches each well was observed and quantified by counting four random fields per well with microscopy (5 wells for each condition).

### Quantitative Real-Time PCR Analysis

Total RNA was extracted using TRIzol reagent (Invitrogen) or RNeasy Mini Kit (Qiagen), and reverse transcription was performed with the High-Capacity cDNA Reverse Transcription Kit from Applied Biosystems according to the manufacturer's instructions. We performed quantitative analysis of gene expression using SYBR® Green PCR Master Mix (Applied Biosystems) with the Mx3000P real-time PCR system (Agilent Technologies). Hprt expression was used as an internal control.

### RNA Sequencing and Analysis.

Reads were aligned to the mm9 mouse transcripts using STAR (version 2.3.0e) (Dobin et al. 2013) using default parameters and resulting bam files were sorted and indexed using samtools. Gene counts were obtained by applying feature counts (version 1.4.3) (Liao, Smyth, and Shi 2014) to sorted bam files, and only unique-mapping reads were used. Genes without any expression counts in any sample were discarded. The DESeq2 (version 1.4.5) R package (Love, Huber, and Anders 2014) was employed to normalize gene count data, and then detect differentially expressed genes (DEG) between mutant mice and control groups with (FDR<0.1 and absolute log 2 fold-change>0.5). Mosaic version 1.1 was used to retrieve gene ontology (GO) information for all genes of the mouse genome (Zhang, Hanspers, et al. 2012). Functional analysis was performed on DEG with DAVID (Huang da. Sherman, and Lempicki 2009) (version 6.7) and biological process GO terms with enrichment p<0.05 were selected as overrepresented functions

### Western Blot Analysis.

Western blot analysis was performed according to a previously described standard protocol. (Greenblatt et al. 2016) Primary antibodies were specific for SLIT3 (1:500; R&D Systems, AF3629), ROBO1 (1:500; Abeam, ab7279), ROBO2 (1:1000; Abcam, ab75014), YAP (1:1,000; Cell Signaling, 4912), p-YAP (1:1,000; Cell signaling, 4911), AKT (1:1,000; Cell Signaling, 4691), p-AKT (1:1,000; Cell Signaling, 4060), ERK (1:1,000; Cell Signaling, 9102), p-ERK (1:1,000; Cell Signaling, 9101) and beta-actin (1:5,000, sc-47778, Santa Cruz) or Hsp90 (1:1000; sc-13119, Santa Cruz). Secondary anti-mouse/rabbit HRP-conjugated antibodies were subsequently applied.

### ELISA Analysis.

SLIT3 ELISA (Lifespan LS-F7173) and CTX ELISA (Lifespan LS-F21349) analysis was performed by using a kit. All ELISA assays were run according to the manufacturer's instructions.

### Bone Fracture Model

All surgical procedures were performed under isoflurane (1-4%) anesthesia via nosecone. Surgical sites were sterilized using a betadine/iodide/isopropanol prep after hair removal by a clipper with a #40 blade and depilatory cream (Nair). After surgery, the visceral lining or muscle was sutured with absorbable Ethicon vicryl sutures (VWR, Cat #95057-014) prior to closing the skin with wound clips that were then removed 2 weeks post-operatively. Animals received intraperitoneal Buprenex (0.5 mg/kg) and oral Meloxicam (2.0 mg/kg) as analgesia prior to surgery and once every 24 hours post-surgery for 3 days. All surgical procedures are approved by the IACUC of Weill Cornell Medical College (Protocol #2012-0005).

Bone fracture was done following previously described protocols with modifications (Bradaschia-Correa et al. 2017). In brief, after anesthesia and surgical site sterilization, an incision above the right anterolateral femur was made. The femur and patella were then exposed, and a 27-gauge syringe needle was inserted parallel with the long axis of the femur through the patellar groove into the marrow cavity. The needle was then removed, and a single cut was made in the middle of the femoral diaphysis using a dremel saw with a diamond thin cutting wheel (VWR, Cat #100230-724). A blunt 25-gauge needle was then inserted into the marrow space through the hole made in the patella to stabilize the fracture. The needle was then trimmed to avoid it from projecting into the patella-femoral joint space. Muscle was then placed over the osteotomy site and stitched with absorbable sutures prior to closing the skin with wound clips.

When assessing the therapeutic effects of SLIT3, 1 mg/kg body weight of SLIT3 recombinant protein (R&D) or vehicle was intravenous injected to the mice twice per week for 3 weeks after the surgery. Alternatively, the gelatin sponge was manually soaked with SLITS (300 ug/ml in PBS) or vehicle for 1 hour on ice and immediately placed to the surgical fracture area. All animals were euthanized by CO2 at time points indicated.

### Ovariectomy-Induced Bone Loss

For the prophylactic model, 12-week-old female mice (JAX) were anesthetized and bilaterally ovariectomized or sham operated. Ovariectomized mice were given twice weekly intravenous injections of 1 mg per kg body weight of SLITS or vehicle or daily subcutaneous (sc) injections of 80 ug/kg PTH (1-34) for 6 weeks starting 2 weeks after ovariectomy. For the model where SLITS was delivered in a therapeutic manner after osteopenia onset, OVX was performed in 12-week old mice, mice were observed for 8 weeks post-OVX to allow for onset of osteopenia, and then mice were treated with SLITS (1 mg/kg) or vehicle for 6 further weeks. All mice were then randomly assigned to one of four groups: Sham, OVX+vehicle, OVX+SLITS and OVX+PTH. Three days after the last injection, all of the mice were euthanized and subjected to bone analysis as described earlier.

### Biomechanical Analysis

All bones were tested to failure using four-point bending on a precision electromagnetic-based load frame (EnduraTEC ELF 3200, Bose Corporation, Minnetonka, MC). Femurs were placed with the posterior surface on the lower supports, spaced 9.9 mm apart. The upper supports were spaced 3.3 ram. Load was applied at a rate of 0.1 mm/s until failure occurred. The failure load (N) and bending stiffness (N/mm²) within the elastic range were calculated from the force-displacement curves and the four-point dimensions.

### Human Bone Callus Collection

The project was approved by the Ethics Committee of Shaoxing People's Hospital (No. 080) and the protocol was carried out in accordance with approved guidelines. Preoperative informed consent was obtained from each patient From January 2010 to June 2014, bone callus samples were obtained from patients undergoing surgical treatment in the Department of Orthopedics of Shaoxing People's Hospital. Callus was collected from patients who required surgical treatment for failure of skeletal traction.

Inclusion criteria were as follows: (1) surgeries after failure of conservative treatment or external fixation were applied temporarily before open reduction and plate fixation for long bone fractures; (2) secondary surgeries after failure of internal fixation, including loosened or broken plates or screws, bent or broken intramedullary nails, and fracture angulation and aversion abnormalities; and (3) secondary surgeries for hypertrophic nonunion. Exclusion criteria were as follows: (1) fracture complicated with microbial infection; (2) fracture complicated with brain injury; (3) bone tumors; (4) systemic bone-related diseases; and (5) patients treated with hormones, steroids, vitamin D, or calcium.

### Statistical Methods

All data were presented as the mean±s.e.m. Sample sizes were calculated on the assumption that a 30% difference in the parameters measured would be considered biologically significant with an estimate of sigma of 10-20% of the expected mean. Alpha and Beta were set to the standard values of 0.05 and 0.8, respectively. No animals or samples were excluded from analysis, and, where applicable, animals were randomized to treatment versus control groups. For data analysis, where relevant, we first performed the Shapiro-Wilk normality test for checking normal distributions of the groups. If normality tests passed, two-tailed, unpaired Student's t-test and if normality tests failed, and Mann-Whitney tests were used for the comparisons between two groups. For the comparisons of three or four groups, we used one-way ANOVA if normality tests passed, followed by Tukey's multiple comparison test for all pairs of groups. If normality tests failed, Kruskal-Wallis test was performed and was followed by Dunn's multiple comparison test. The GraphPad PRISM software (v6.0a, La Jolla Calif.) was used for statistical analysis. P<0.05 was considered statistically significant. *P<0.05, **P<0.01, ***P<0.001. **** P<0.0001.

### Results

Shn3^{-/-} Mice Exhibit Increases in CD31^{hi}EMCN^{hi} Endothelium

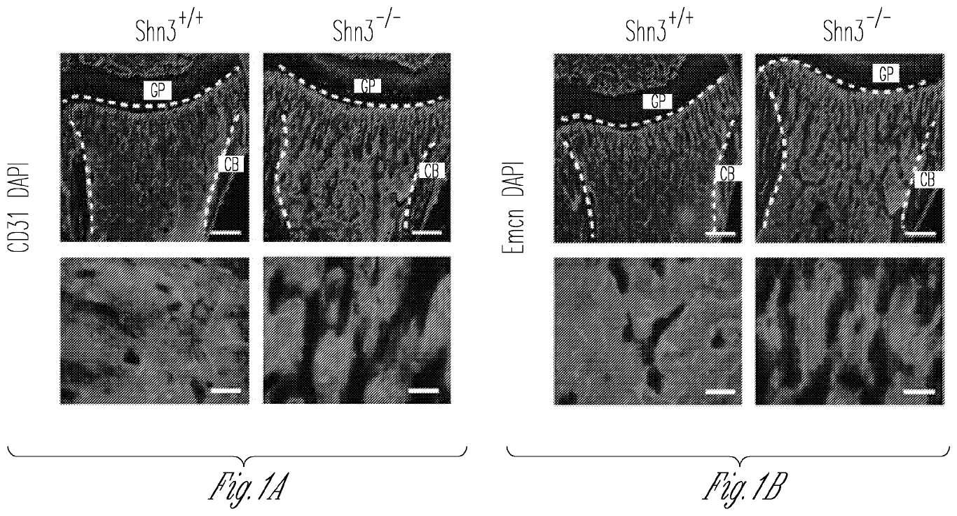

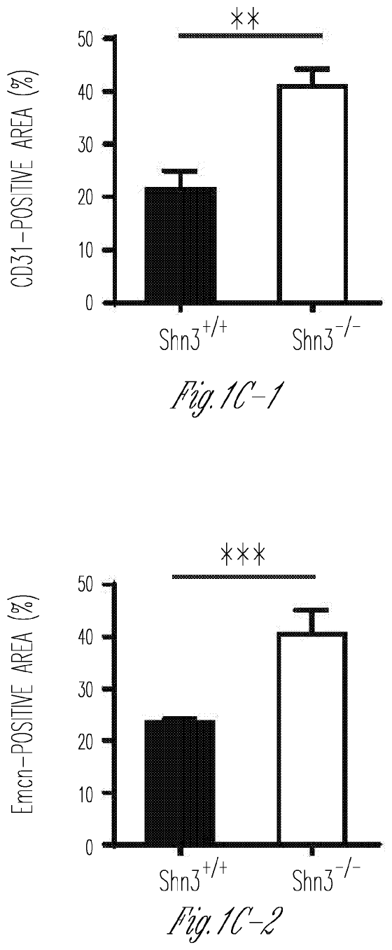

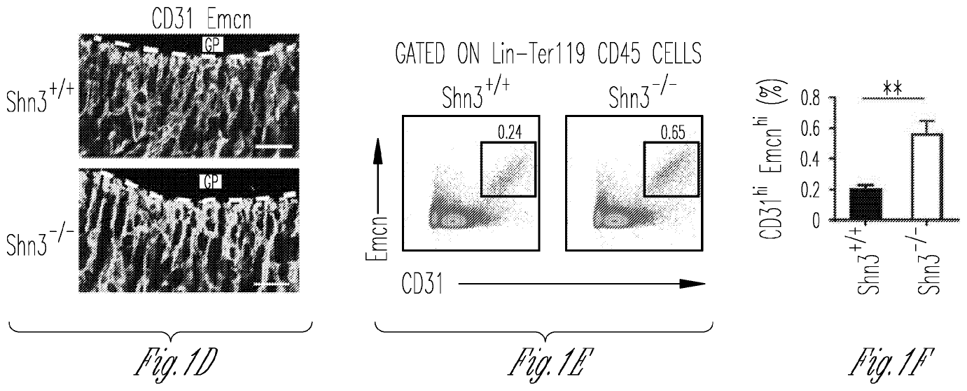

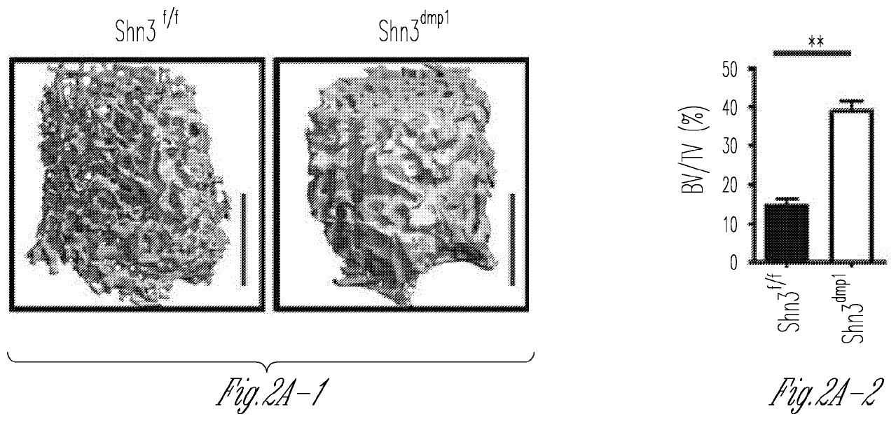

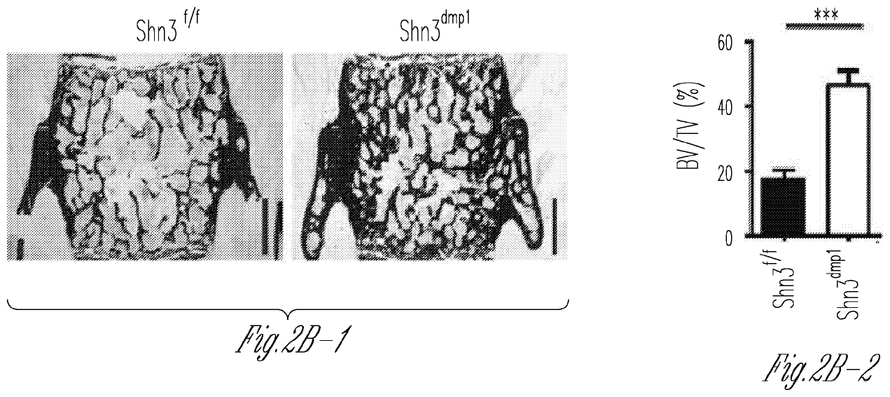

To address our hypothesis that osteoblasts are able to coordinate levels of osteogenic CD31^{hi}EMCN^{hi} endothelium to maintain bone formation capacity, CD31^{hi}EMCN^{hi} endothelium levels were assessed in a mouse strain displaying augmented postnatal bone formation, Shn3^{-/-} mice. CD31, EMCN-double positive endothelium was present in the marrow immediately beneath the growth plate and was significantly increased in Shn3^{-/-} mice (FIG. 1A-1D). This increase was bone-specific as Shn3^{-/-} mice displayed normal levels of EMCN-positive vessels in other organs including heart, brain, lung and kidney (FIG. 7A). This vessel phenotype was present in neonatal Shn3^{-/-} mice and thus preceded the appearance of the high bone mass phenotype (Jones et al. 2006) (FIG. 7B). Flow cytometry also confirmed an increase in CD31^{hi}EMCN^{hi} vascular endothelium in the bones of Shn3^{-/-} mice (FIG. 1E-1F and FIG. 7C). Taken together, these observations indicate that SHN3 regulates levels of skeletal CD31^{hi}EMCN^{hi} endothelium in addition to its role in regulating osteoblast activity.

Osteoblasts Regulate Levels of CD31^{hi}EMCN^{hi} Endothelium

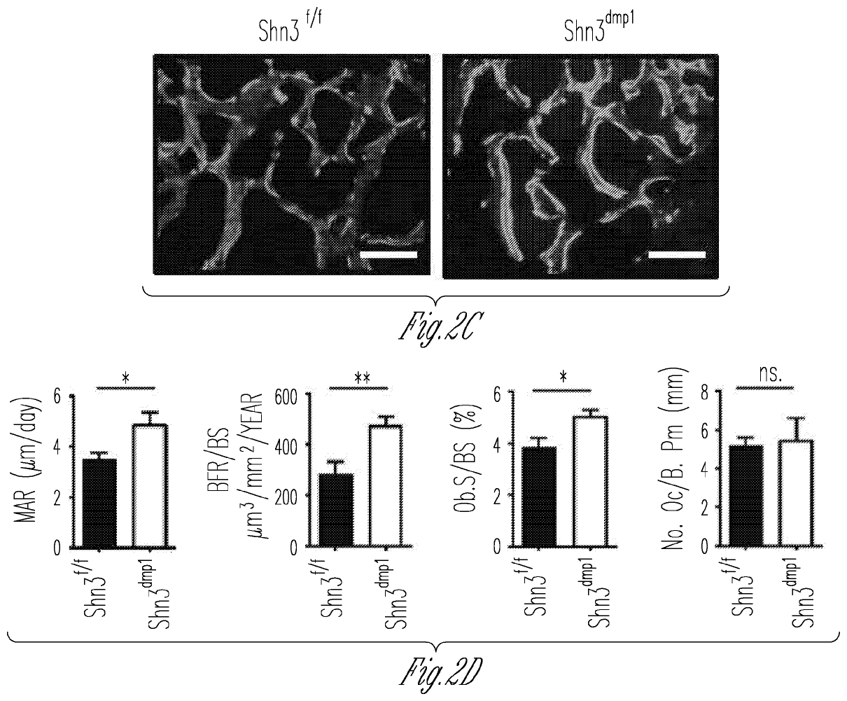

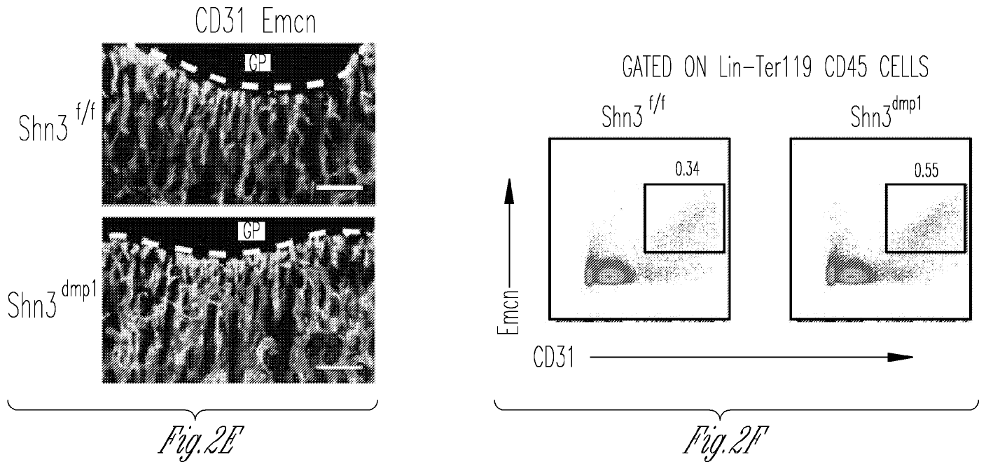

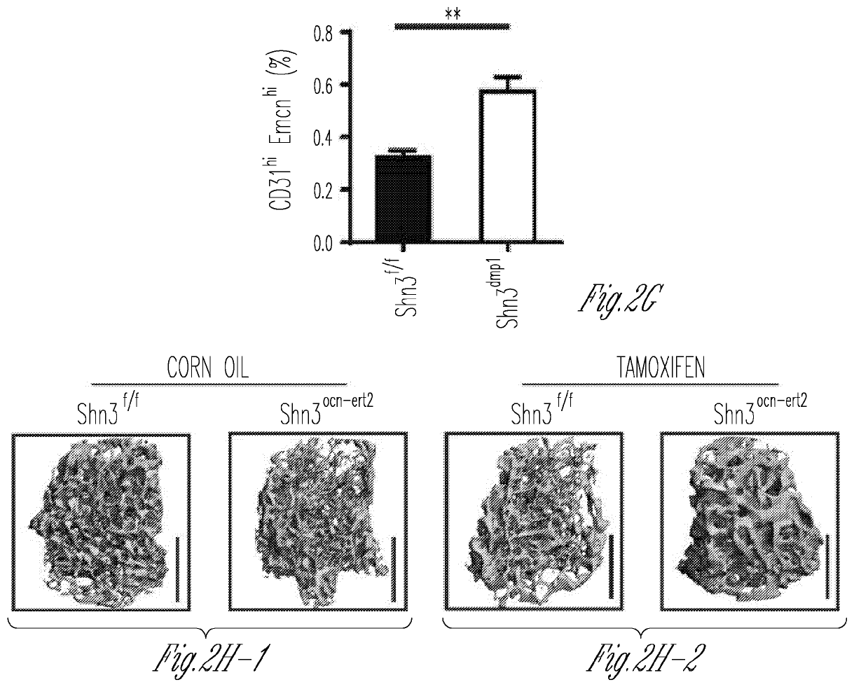

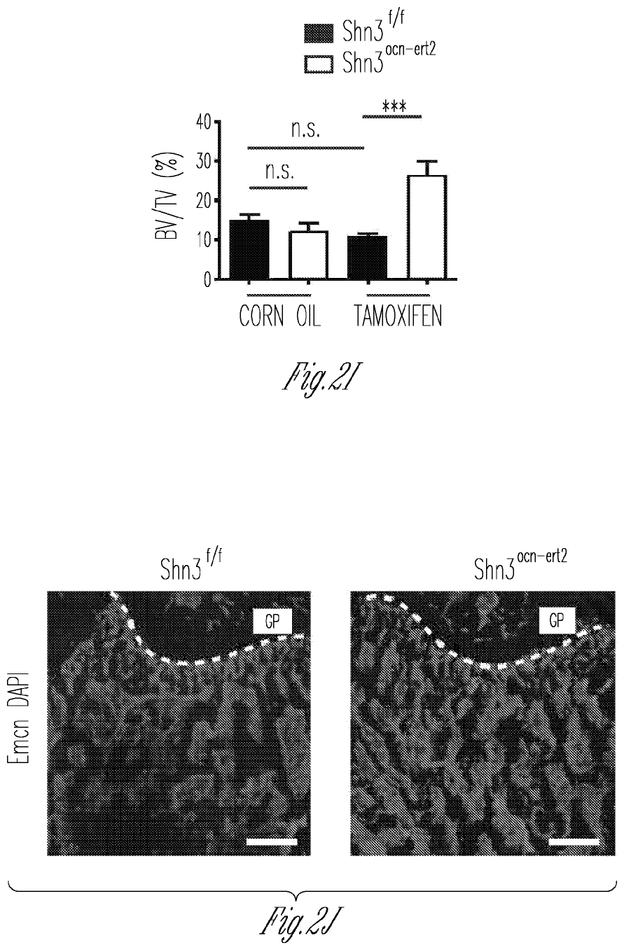

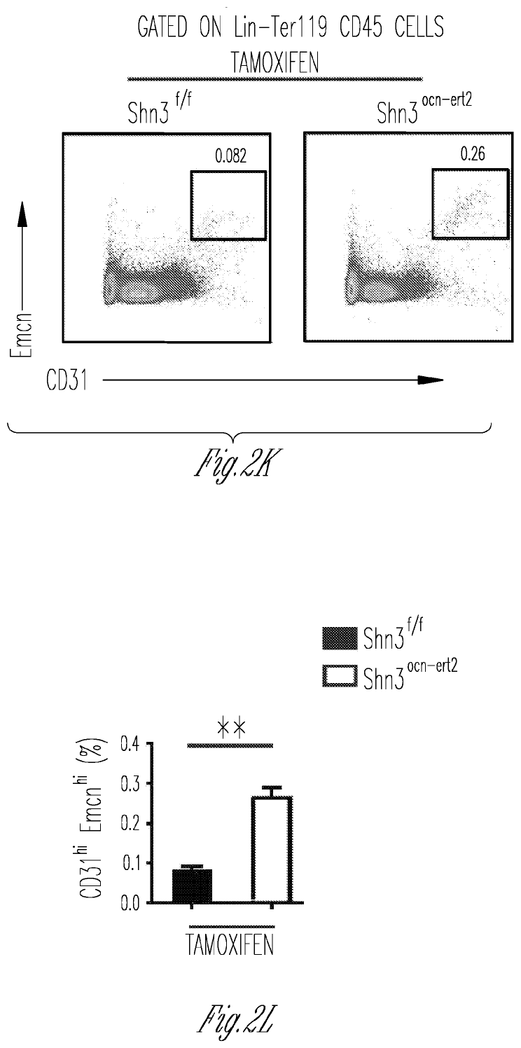

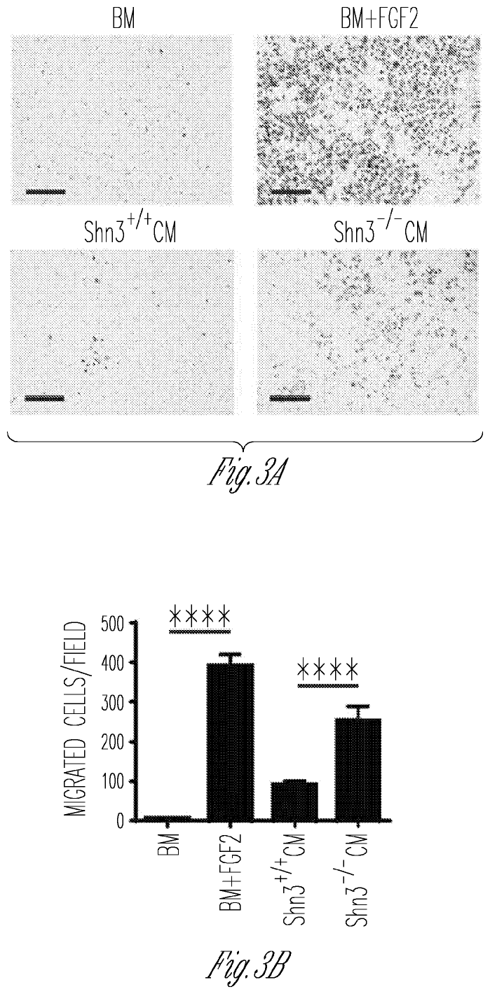

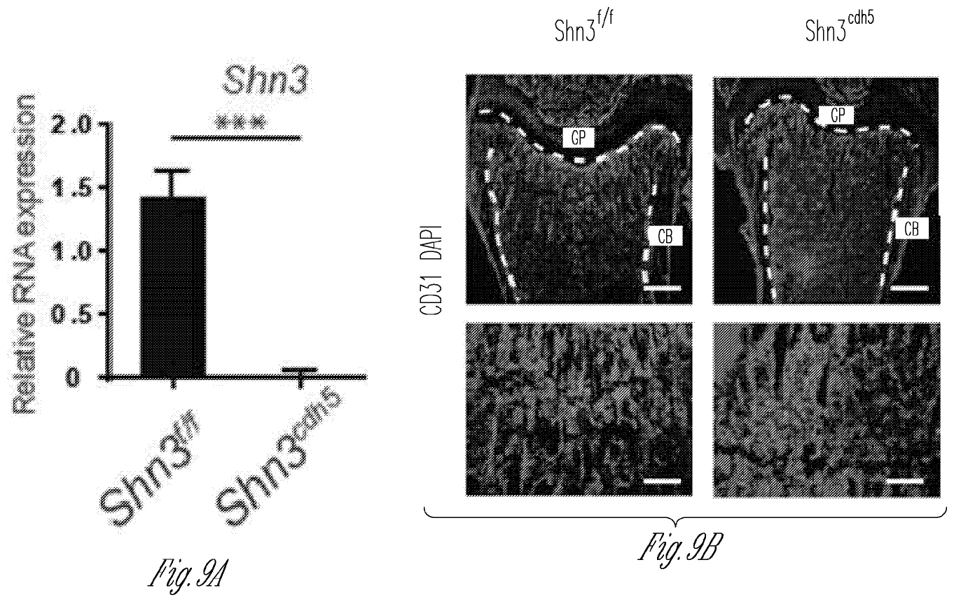

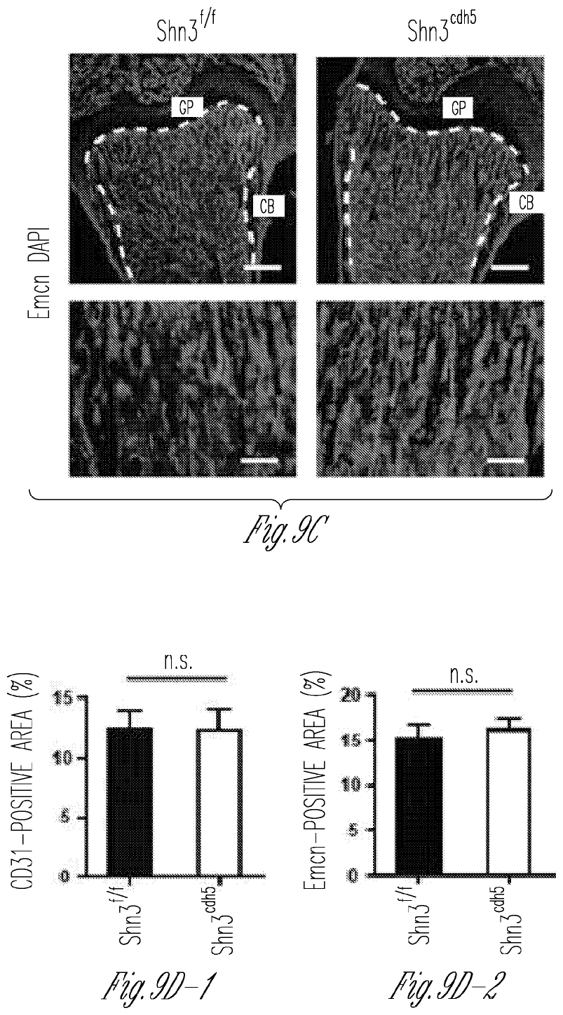

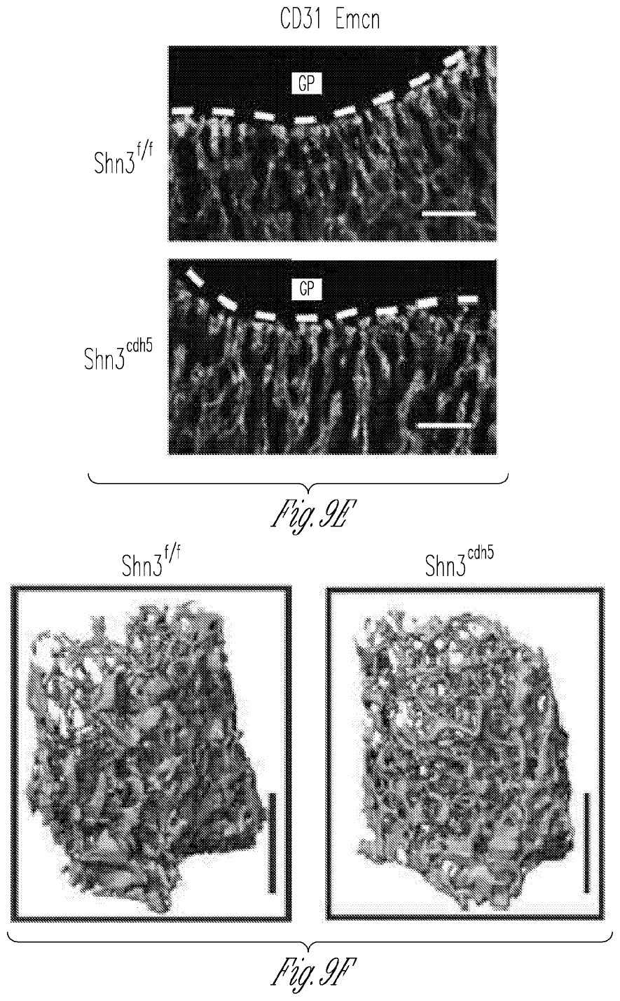

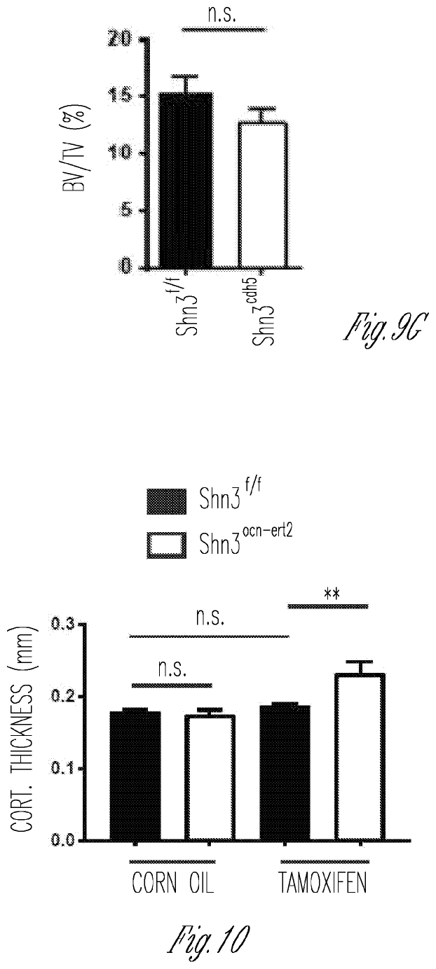

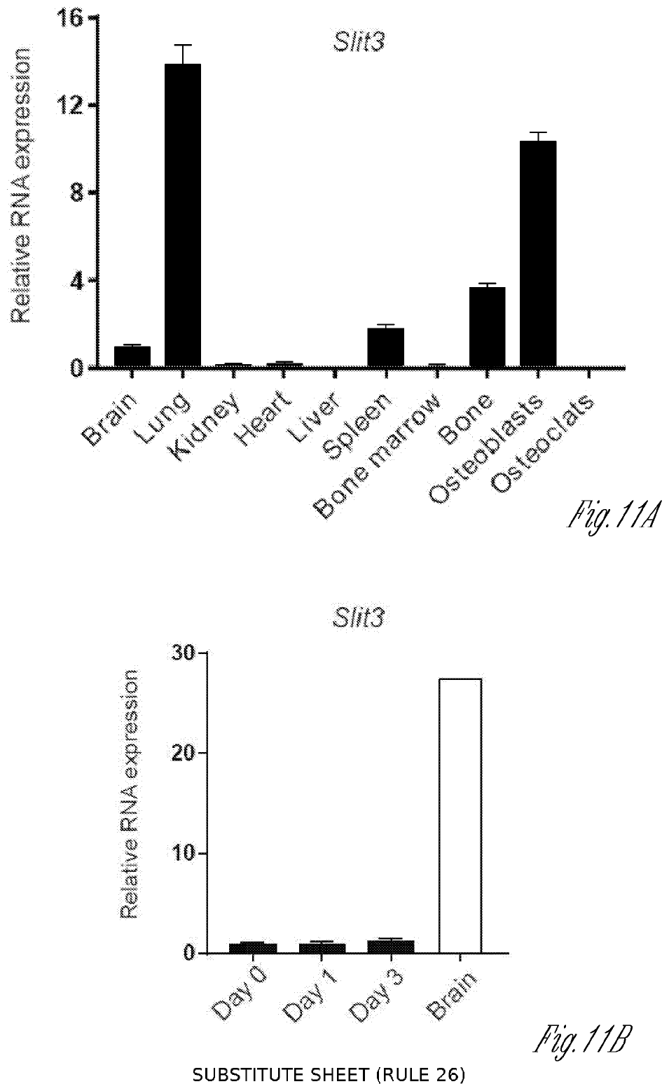

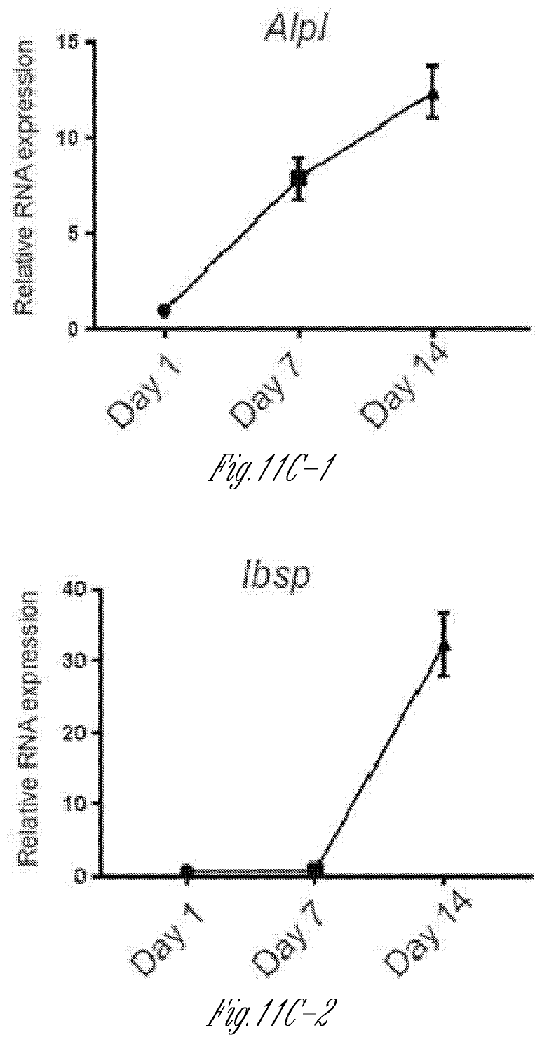

As SHN3 acts in a cell intrinsic manner to regulate bone formation by osteoblasts, we reasoned that SHN3 also acts in osteoblasts to control levels of CD31^{hi}EMCN^{hi} endothelium. To test this directly, mice harboring a Shn3 allele in which exon 4 is flanked by loxP sites (called Shn3^{f/f} mice) were bred to a cre-deleter strain targeting osteoblast progenitors, OSX-cre, and to a cre-deleter strain targeting mature osteoblasts, DMP1-cre. Both Shn3^{osx} and Shn3^{dmp1} mice exhibited a similar degree of increased bone mass, including increased cortical bone thickness, largely recapitulating the characteristic bone phenotype of Shn3^{-/-} mice (FIG. 2A-2B and FIG. 8A-8C). Based on this, the Shn3^{dmp1} strain was selected far further study as it implicates a more restricted subpopulation in any phenotypes observed (Zhang and Link 2016, Chen et al. 2014). Histomorphometric analysis confirmed that the high bone mass phenotype of Shn3^{dmp1} mice was due to increased osteoblast-mediated bone formation predominantly on the endosteal surface (FIG. 2C-2D and FIG. 8D-8E). Analysis of skeletal CD31^{hi}EMCN^{hi} endothelium by both immunostaining and flow cytometry demonstrated that Shn3^{dmp1} mice displayed a similar increase in this subset of endothelial cells as that observed in Shn3^{-/-} mice (FIG. 2E-2G and FIG. 8F-8H).

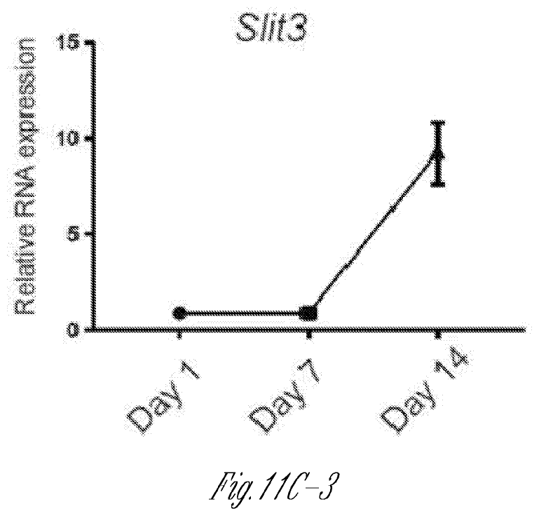

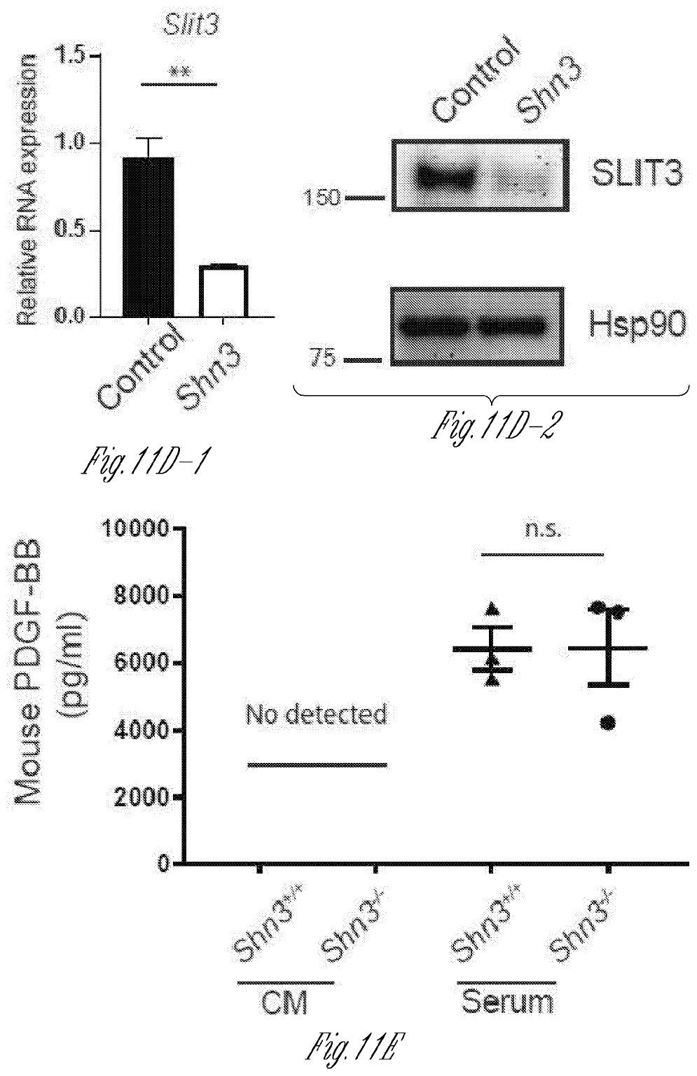

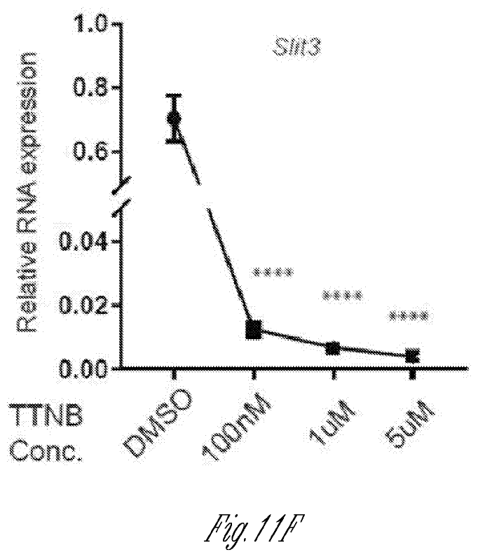

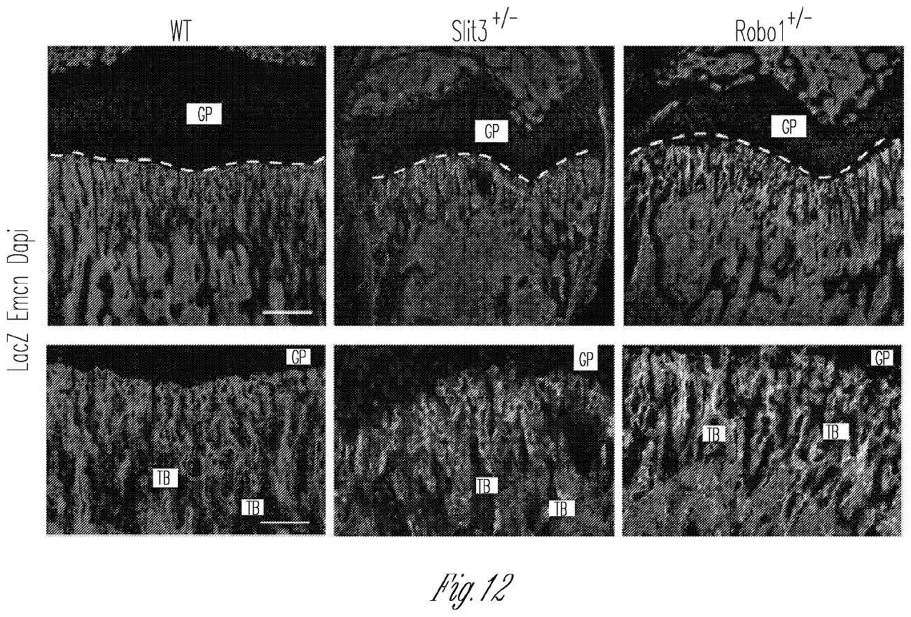

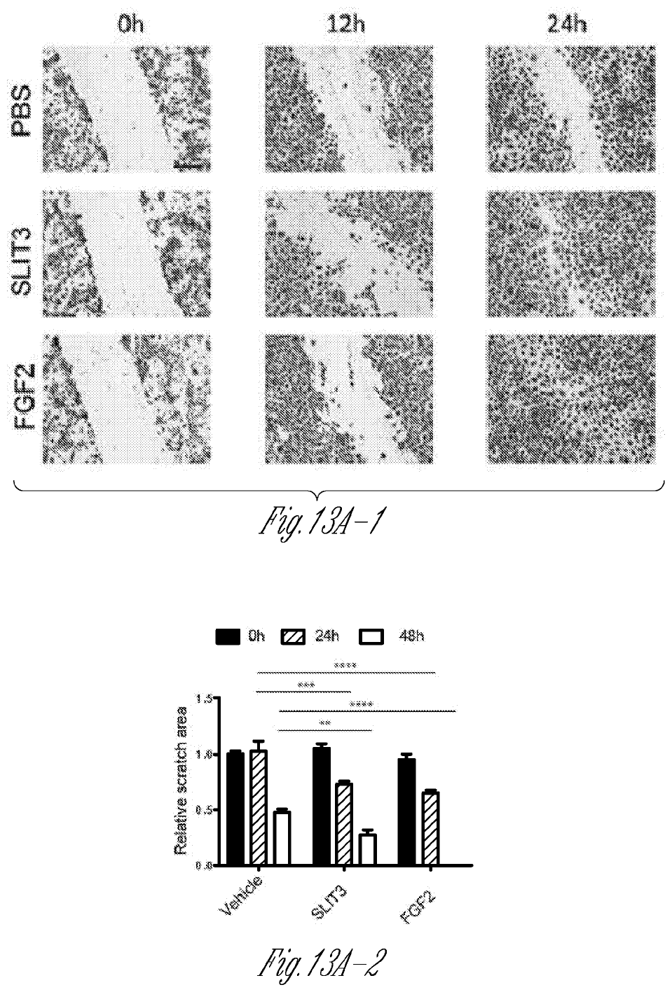

To further confirm that the function of SHN3 to regulate skeletal CD31^{hi}EMCN^{hi} vascular endothelium maps to osteoblasts, endothelial cell-specific Shn3-deficient mice (Shn3^{cdh5} mice) were generated using the Cdh5 (VE-cadherin)-Cre. Despite observing efficient deletion of Shn3 in bone marrow endothelial cells (FIG. 9A), levels of CD31^{hi}EMCN^{hi} endothelial cells were unchanged in Shn3^{cdh5} mice (FIG. 9B-9E). Accordingly, Shn3^{cdh5} mice also displayed normal bone mass (FIG. 9F-9G). Taken together with the Shn3^{-/-} and Shn3^{dmp1} phenotype, we conclude that osteoblasts regulate skeletal CD31^{hi}EMCN^{hi} vascular endothelium in a SHN3-dependent manner, and that SHN3 does not act directly in endothelial cells to regulate skeletal phenotypes.

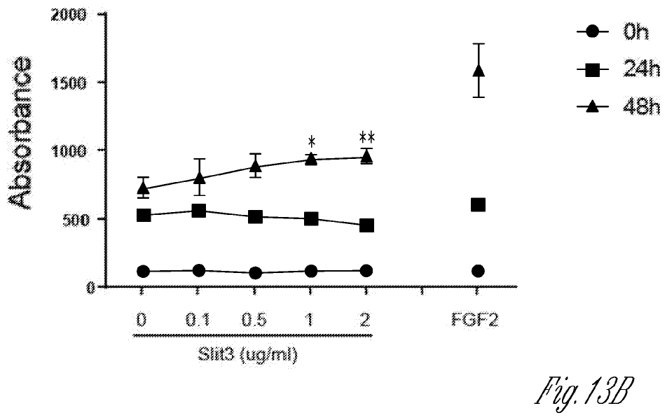

To determine if osteoblasts continuously participate in this regulation or if this process is limited to embryonic development, Shn3^{f/f} mice were intercrossed with osteocalcin-CreERT mice expressing a tamoxifen-activated Cre recombinase in mature osteoblasts under the control of the osteocalcin promoter (Shn3^{ocn-en} mice). Cre-mediated deletion was induced with tamoxifen, generating tamoxifen-inducible inhibition of Shn3 (Shn3^{ocn-ert}) in these mice, and the resulting skeletal and vascular phenotypes exhibited by the mice were analyzed. Tamoxifen treatment increased CD31^{hi}EMCN^{hi} endothelial levels and both trabecular bone mass and cortical bone thickness, with the increase in CD31^{hi}EMCN^{hi} endothelium preceding the increase in bone mass (FIG. 2H-2L and FIG. 10). Thus, mature osteoblasts continuously participate in tuning the levels of CD31^{hi}EMCN^{hi} endothelium in bone, and this coupling is not solely a developmental phenomenon.

### SHN3 Regulates Slit3 Expression in Osteoblasts

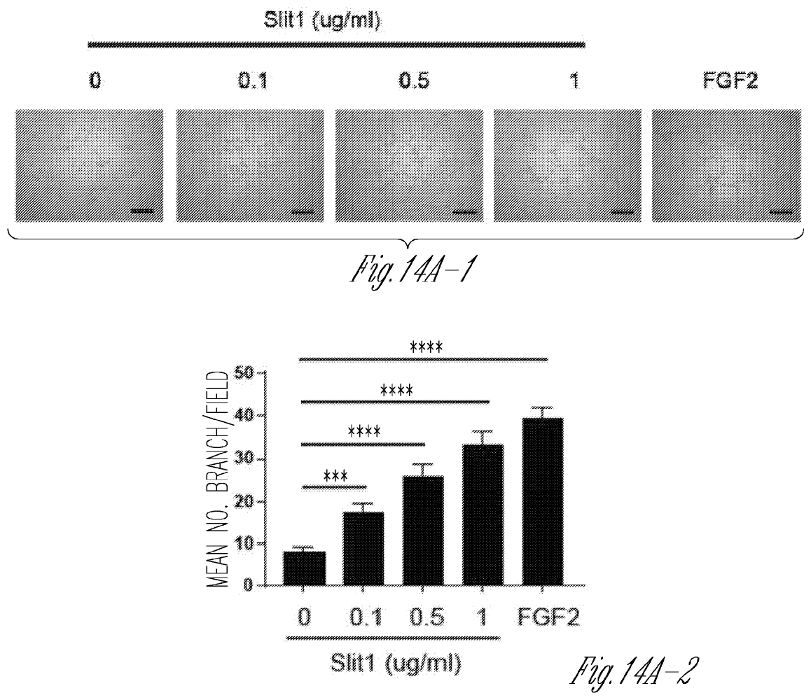

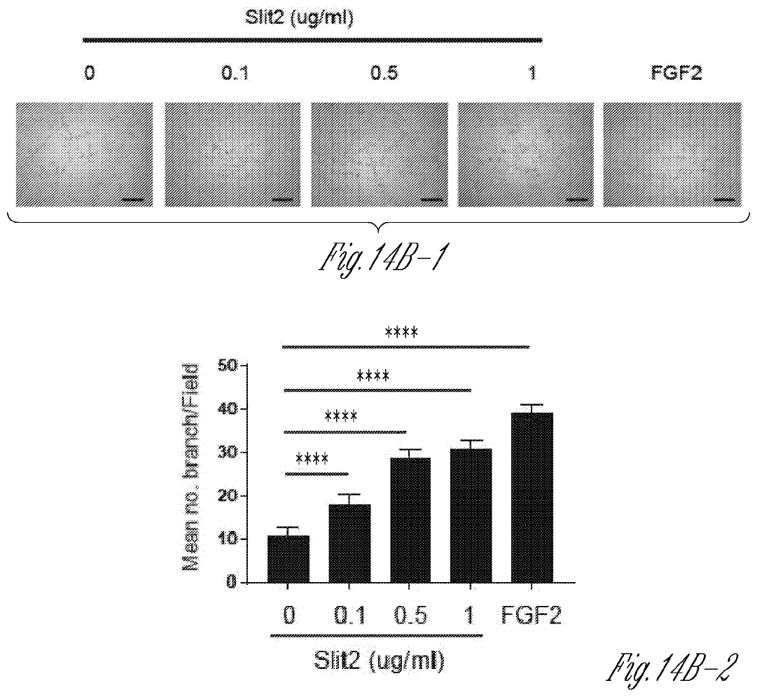

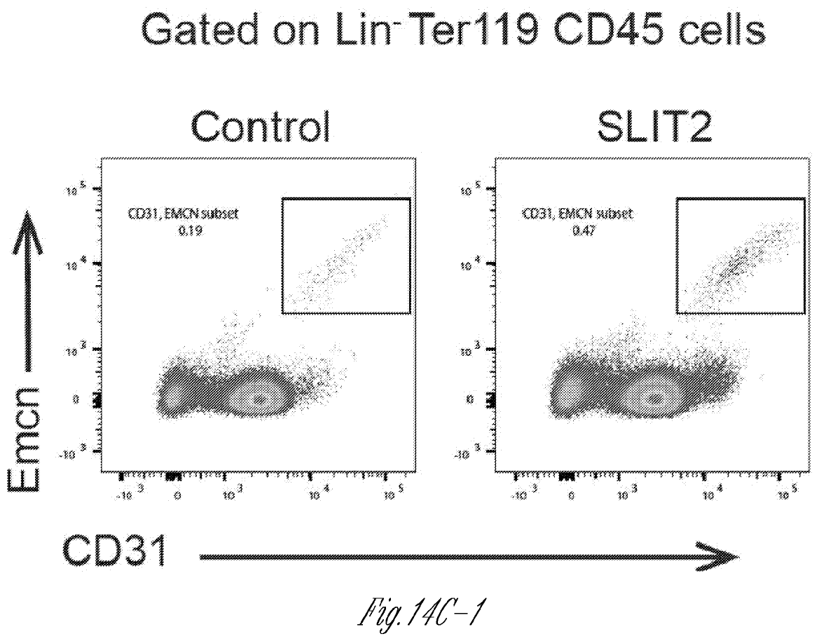

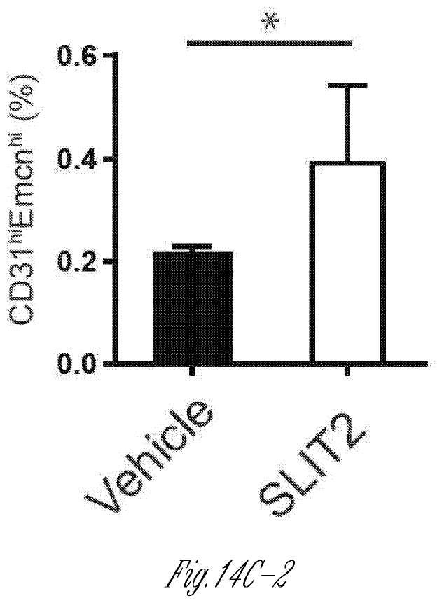

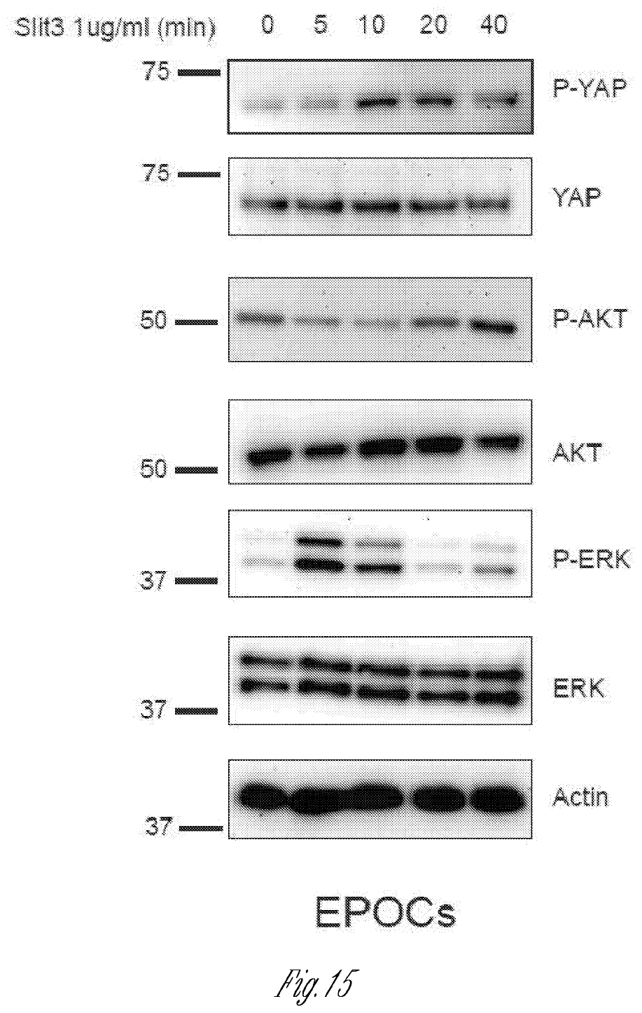

To investigate if the regulation of marrow angiogenesis by SHN3 is a direct effect of osteoblasts on endothelial cells, conditioned medium was harvested from WT and Shn3^{-/-} primary osteoblasts and introduced to cultures of primary bone-marrow derived endothelial cells. Conditioned medium from Shn3^{-/-} primary osteoblasts displayed an enhanced ability to induce endothelial migration and capillary tube formation, indicating that the osteoblasts secrete a soluble mediator that can induce endothelial migration and capillary tube formation (FIG. 3A-3D). To identify potential proangiogenic factors regulated by SHN3 in osteoblasts, RNA sequencing and transcriptional profiling was performed. Gene ontology (GO) analysis demonstrated that the set of differentially expressed genes in Shn3^{-/-} versus WT osteoblasts is enriched for genes mediating angiogenesis in addition to the expected enrichment for genes involved with bone development and mineralization (FIG. 3E). Examination of the expression of a number of proangiogenic factors revealed that only SLIT3, a soluble axonal chemorepellent recently shown to have angiogenic functions, showed a substantial increase in Shn3^{-/-} osteoblasts (FIG. 3F) (Zhang et al. 2009, Xie et al. 2014). Further expression analysis confirmed robust Slit3 expression in osteoblasts and demonstrated negligible Slit3 expression in osteoclasts (FIG. 11A-11B)(Su et al. 2004). Similarly, immunofluorescence for beta-galactosidase in Slit3^{+/-} mice (Yuan et al. 2003) bearing a targeted insertion of a LacZ cassette into the Slit3 locus identified SLIT3 expression in cells adjacent to the bone surface consistent with osteoblasts (FIG. 12). Slit3 was also found to be expressed in human mesenchymal stromal cell-derived osteoblasts (FIG. 11C). To verify that Slit3 expression is increased in Shn3^{-/-} osteoblasts, three complimentary approaches were utilized. First, real-time PCR was used to validate the increase in Slit3 observed in Shn3^{-/-} osteoblasts by RNA sequencing (FIG. 3G). Second, overexpression or knockdown of Shn3 in human mesenchymal stem cell (hMSC)-derived osteoblasts demonstrated, respectively, a suppression or an enhancement of both mRNA and protein levels of Slit3 (FIG. 3H-3I). Moreover, overexpression of Shn3 in murine primary osteoblasts also dramatically reduced SLIT3 expression (FIG. 11D). Lastly, ELISA demonstrated a 3-fold increase in SLIT3 secretion in conditioned medium from Shn3^{-/-} osteoblasts relative to controls (FIG. 3J). Thus, SHN3 is a negative regulator of SLIT3 expression in osteoblasts.

Recent studies have shown that preosteoclast-derived PDGF-BB is able to induce CD31^{hi}EMCN^{hi} endothelium in bone (Xie et al. 2014). PDGF-BB secretion by osteoblasts was not detected, and negligible Pdgfb mRNA was observed in both WT and Shn3^{-/-} osteoblasts (FIG. 3F and FIG. 11E). In addition, scrum PDGF-BB levels are unaltered in Shn3^{-/-} mice (FIG. 11E). Thus, we do not observe evidence of regulation of PDGF-BB by SHN3.

SHN3 acts predominantly by regulating ERK activity, as mice bearing a knock-in of a mutation in 3 amino acids within the ERK interacting motif (Shn3^{KI/KI} mice) in SHN3 largely recapitulate the high bone mass phenotype of Shn3^{-/-} mice. Slit3 levels demonstrated a similar increase in primary Shn3^{KI/KI} osteoblasts as that seen in Shn3^{-/-} osteoblasts (FIG. 3K). Similarly, treatment with the ERK pathway inhibitor trametinib (TTNB) reduced Slit3 expression in both hMSC-derived osteoblasts and murine primary calvarial osteoblasts (FIG. 3L and FIG. 11F). Consistent with these observations, Shn3^{KI/KI} mice displayed an increase in CD31^{hi}EMCN^{hi} skeletal endothelium similar to that seen in Shn3^{-/-} mice (FIG. 3M). Thus, SHN3 regulates SLIT3 expression and CD31^{hi}EMCN^{hi} endothelium levels via its ability to bind and regulate ERK.

SLIT3 Promotes CD31^{hi}EMCN^{hi} Endothelium Formation and Bone Formation In Vivo

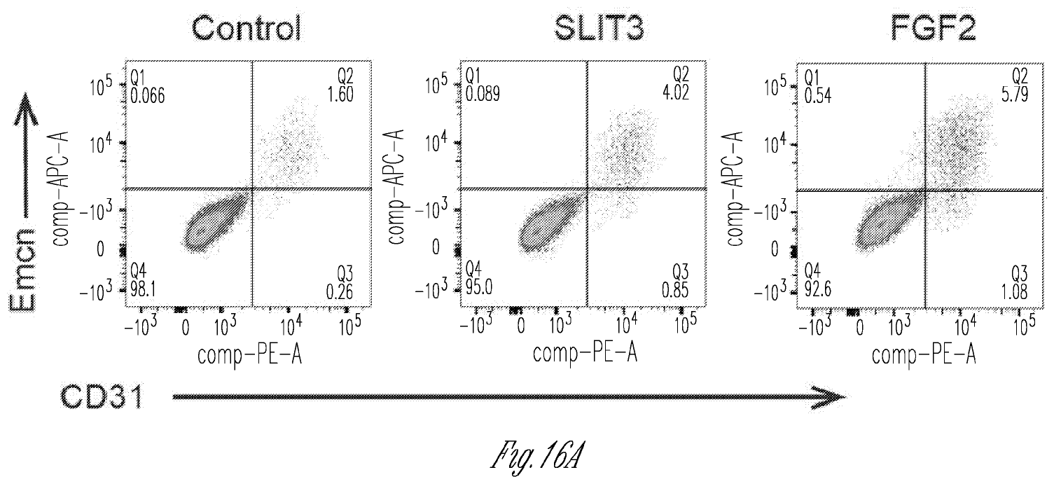

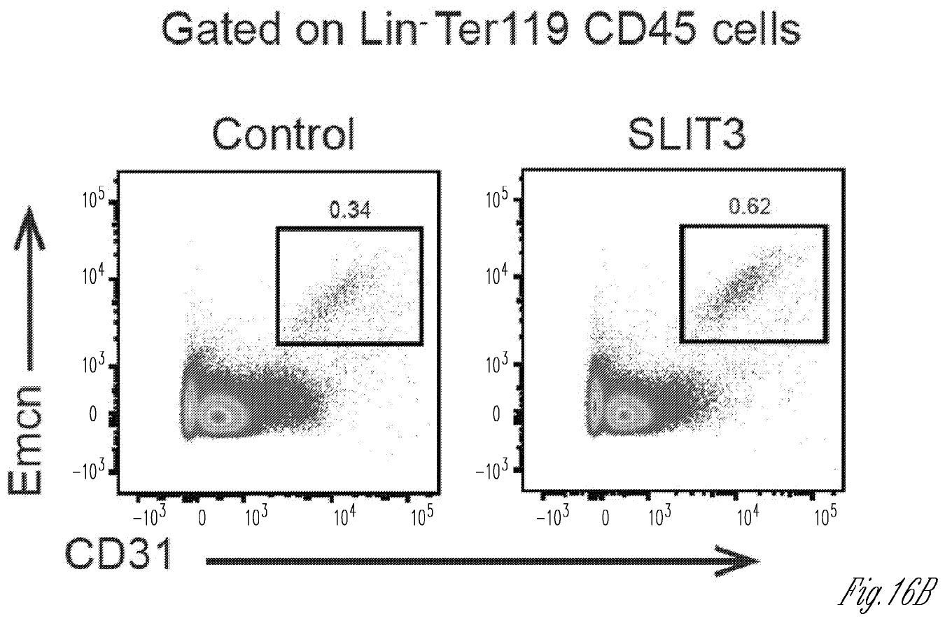

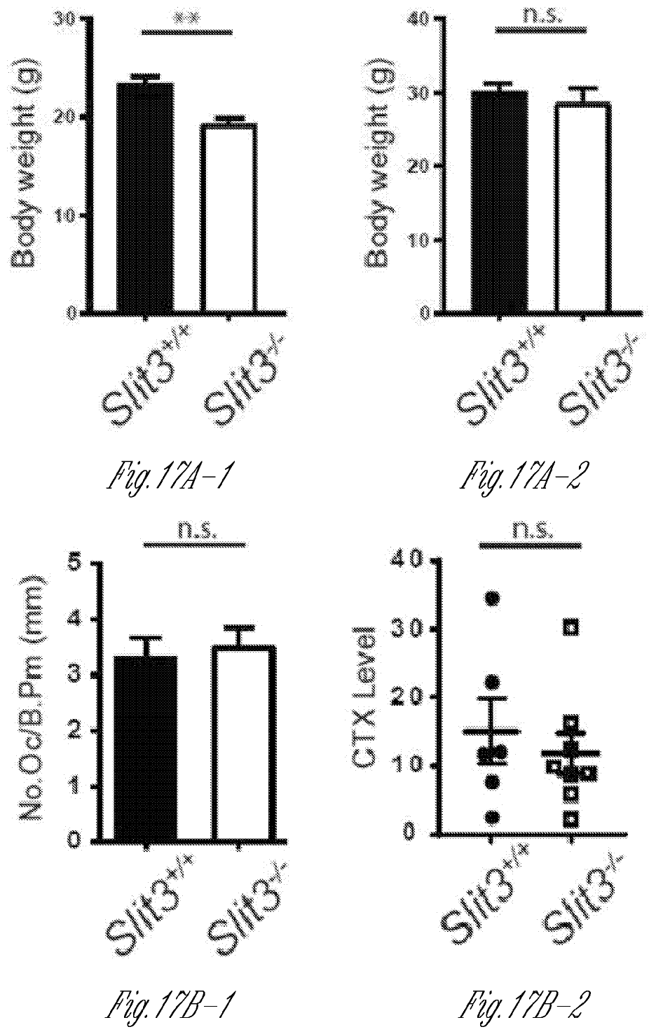

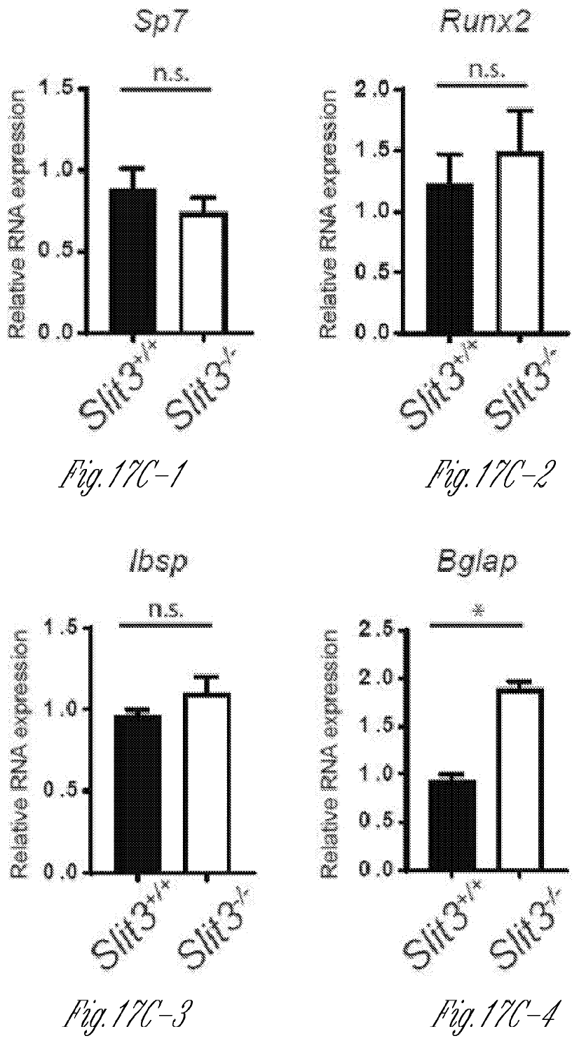

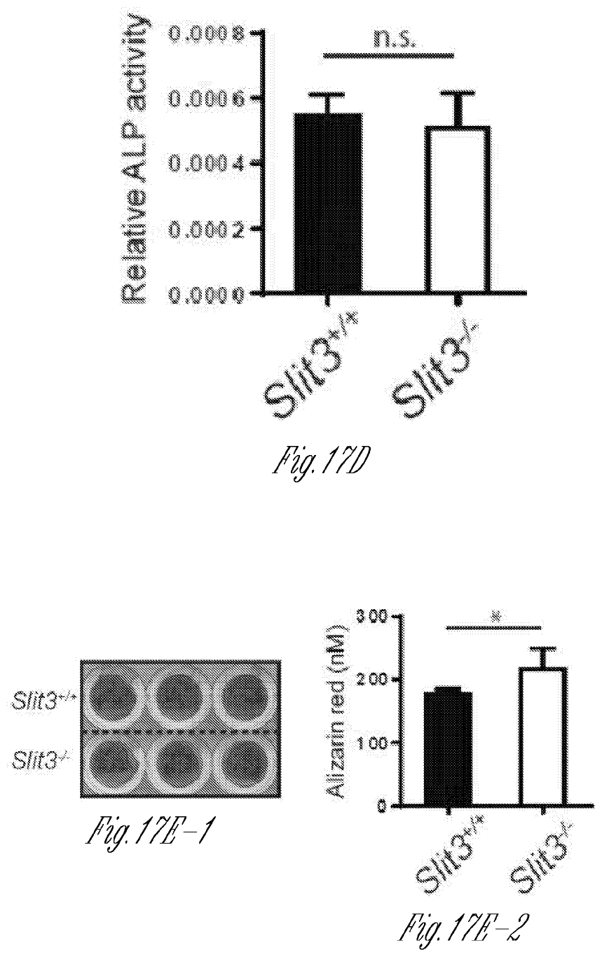

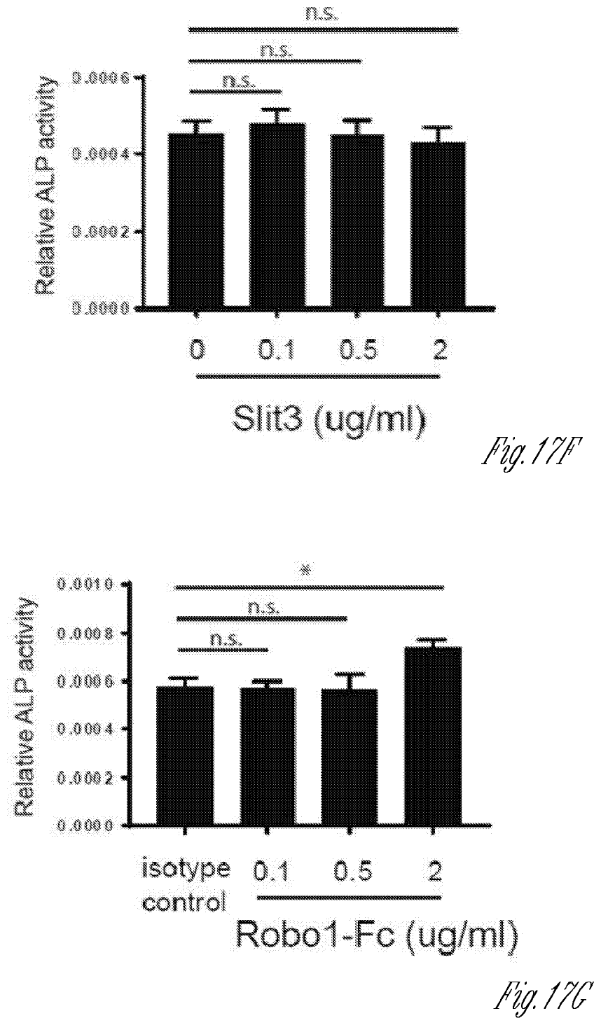

To determine if SLIT3 contributes to the regulation of bone marrow endothelium by osteoblasts in vitro, a dose response curve for SLIT3 treatment was conducted as described in prior studies (Zhang et al. 2009, Geutskens, Hordijk, and van Hennik 2010, Naska et al. 2010). Bone marrow endothelial progenitor outgrowth cells (EPOCs) treated with recombinant SLIT3 displayed enhanced migration and tube formation (FIG. 4A-4B and FIG. 13A). Additionally, SLIT3 modestly increased the proliferation of bone marrow derived EPOCs, consistent with the activity of SLIT3 observed in non-skeletal vascular endothelium (Zhang et al. 2009) (FIG. 13B). SLIT1 and SLIT2 displayed a similar ability to promote tube formation, consistent with observations that each of the SLITs displays a similar capacity far ROBO binding and activation (Howitt, Clout, and Hohenester 2004, Liu et al. 2004, Rama et al. 2015) (FIG. 14A-14B). Next, conditioned medium was collected from WT and Shn3^{-/-} osteoblasts and placed on bone marrow derived endothelial cells together with an anti-SLIT3 blocking antibody or an isotype control. Enhanced tube formation was seen with conditioned medium from SHN3-deficient osteoblasts, and treatment with an anti-SLIT3 blocking antibody abrogated this effect (FIG. 4C-4D). Investigation of the signaling pathways downstream of SLIT3 in bone marrow endothelial cells demonstrated activation of ERK MAPK and Hippo signaling, pathways known to participate in angiogenesis (FIG. 15) (Shin et al. 2016, Choi et al. 2015). Interestingly, we also noted that treatment of bone marrow-derived endothelial cells with SLIT3 enhanced the acquisition of a CD31^{hi}EMCN^{hi} surface immunophenotype in culture (FIG. 16A).

### SLIT2 and SLIT3 Unexpectedly Display Divergent Functions in the Skeletal System

Current biochemical evidence shows that the ROBO receptors are largely unable to discriminate among SLIT ligands. Thus, based on current theories, one would expect that SLIT2 would show a similar ability as SLIT3 to promote fracture healing.

Systemic administration of SLIT3 increased levels of skeletal CD31^{hi}EMCN^{hi} endothelium production in vivo (FIG. 16B). In addition, SLIT2 increased formation of CD31^{hi}EMCN^{hi} endothelium in vivo. (FIG. 14C).

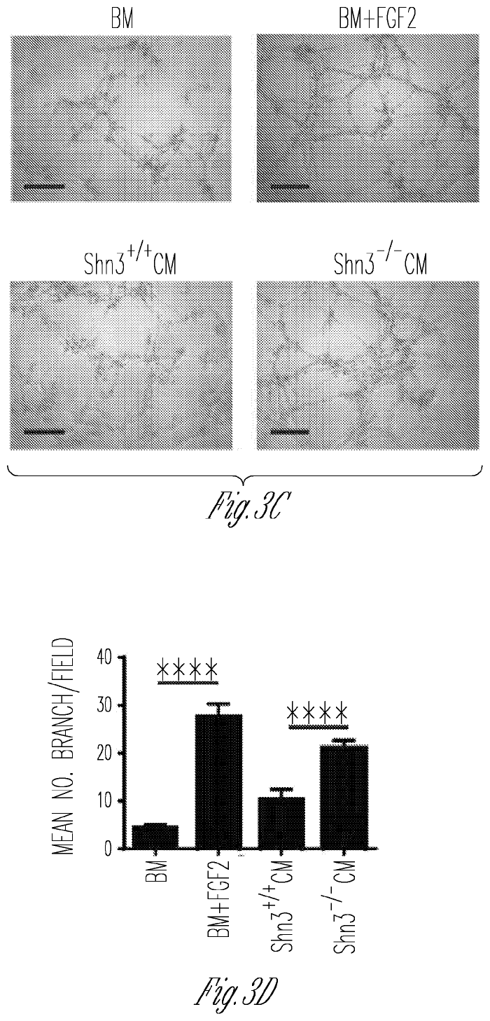

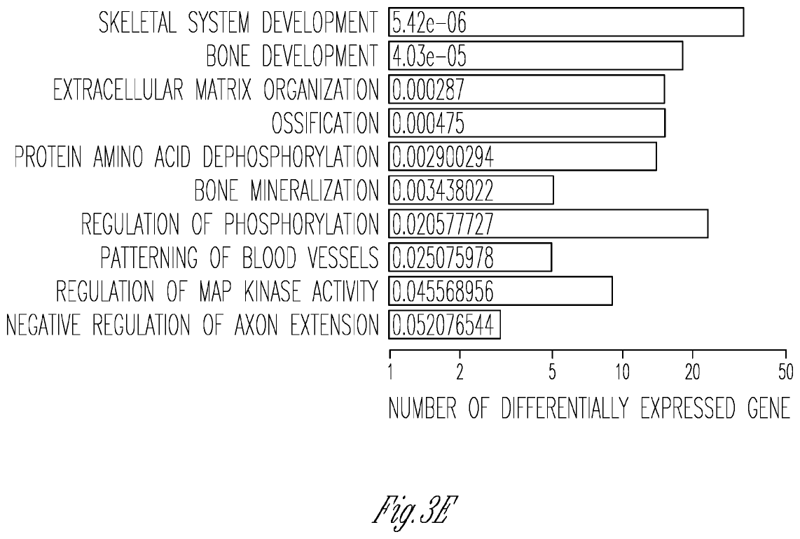

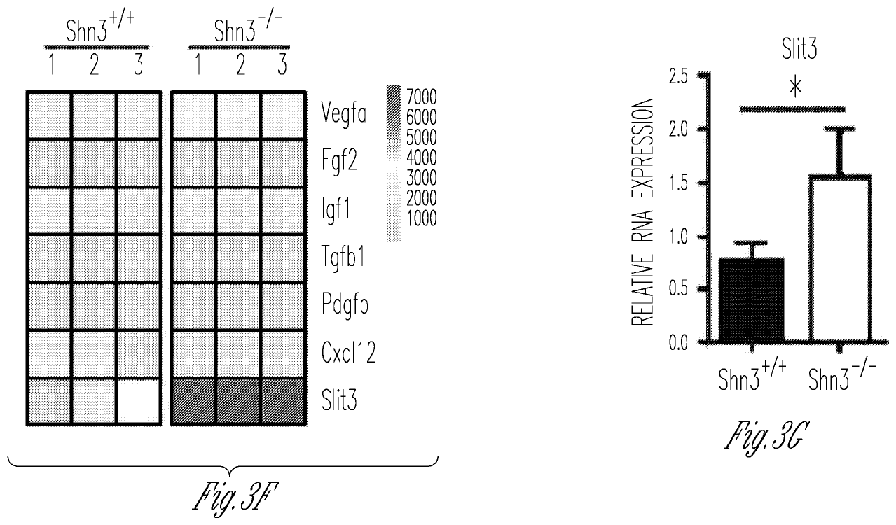

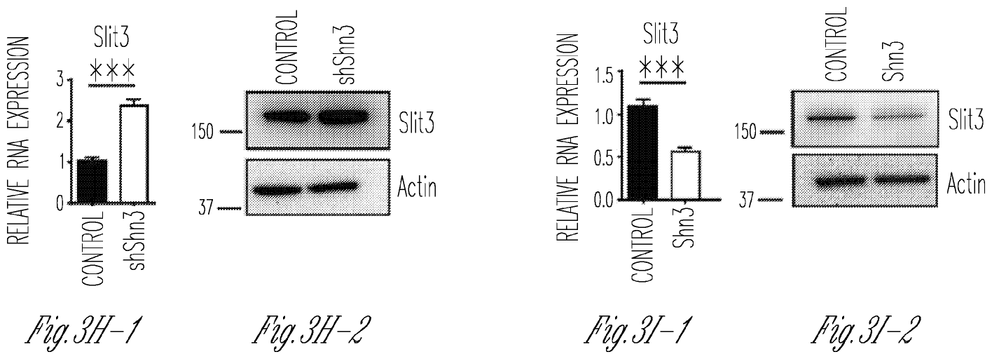

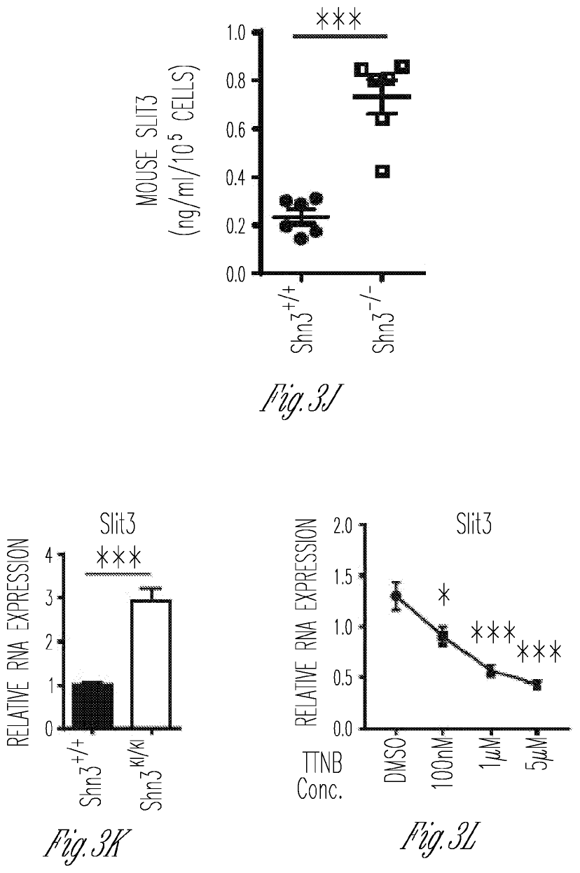

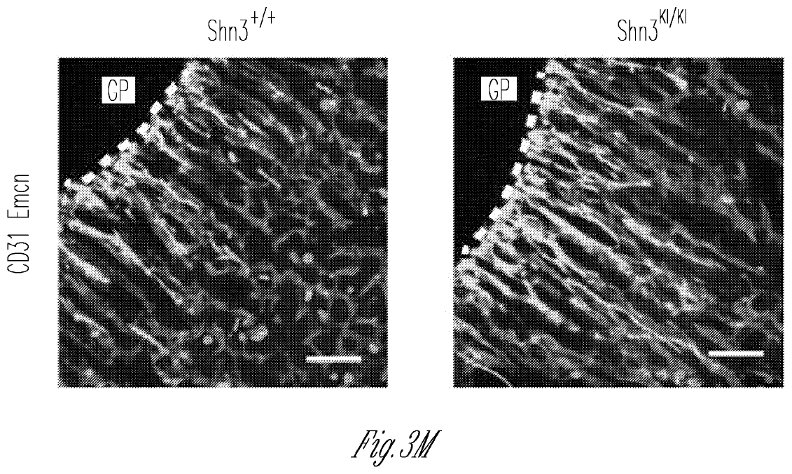

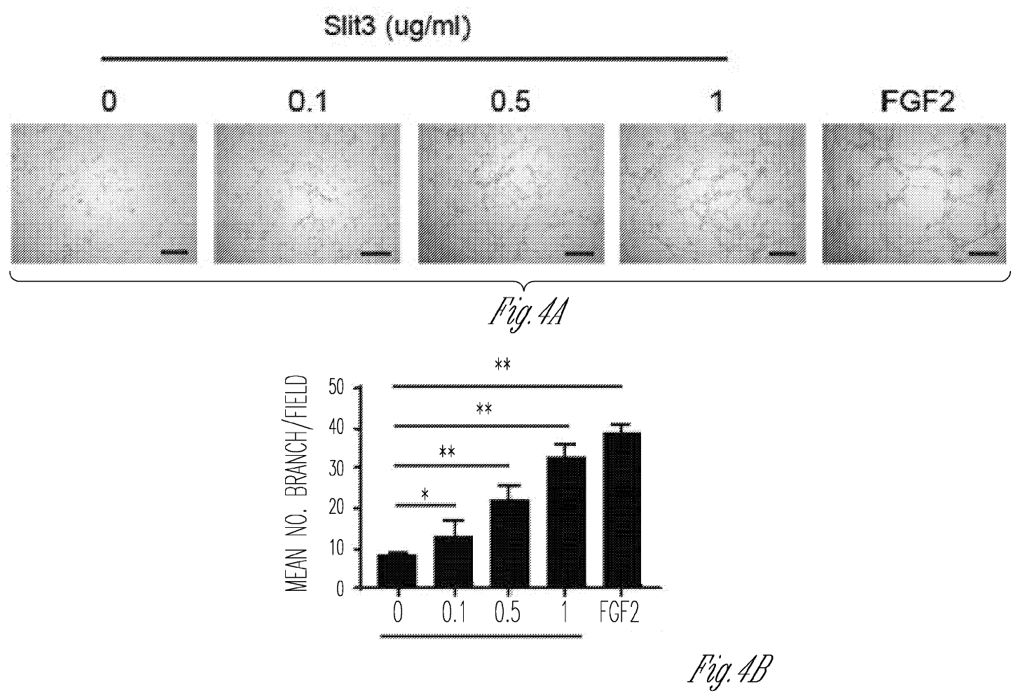

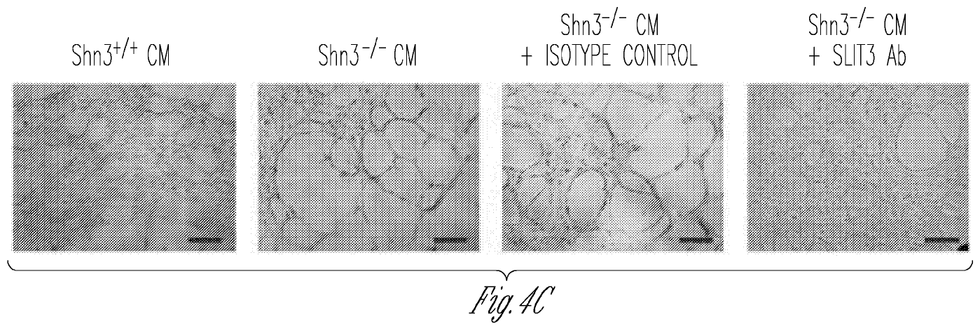

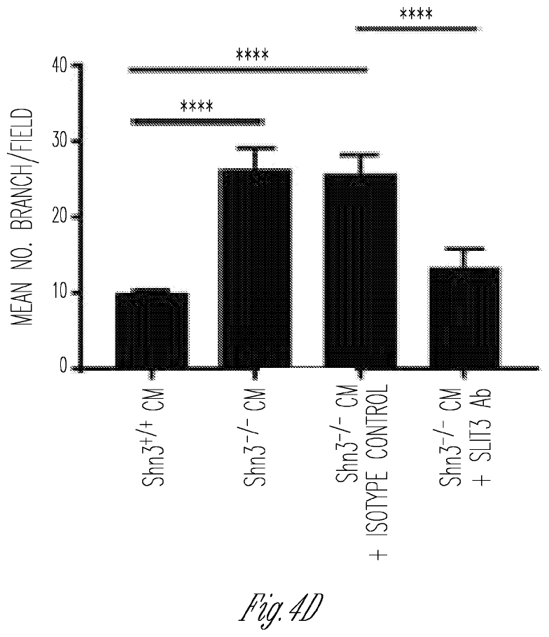

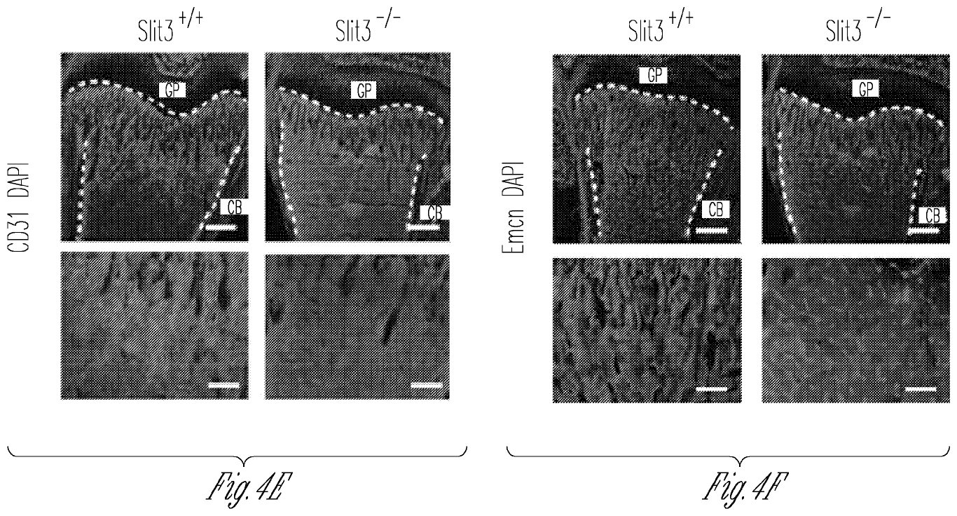

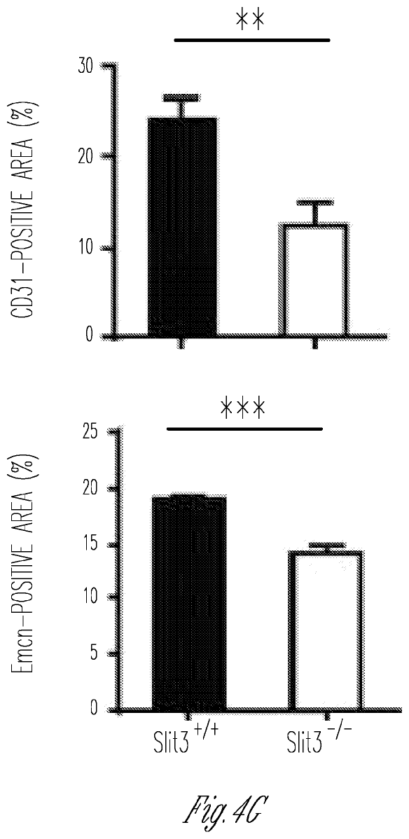

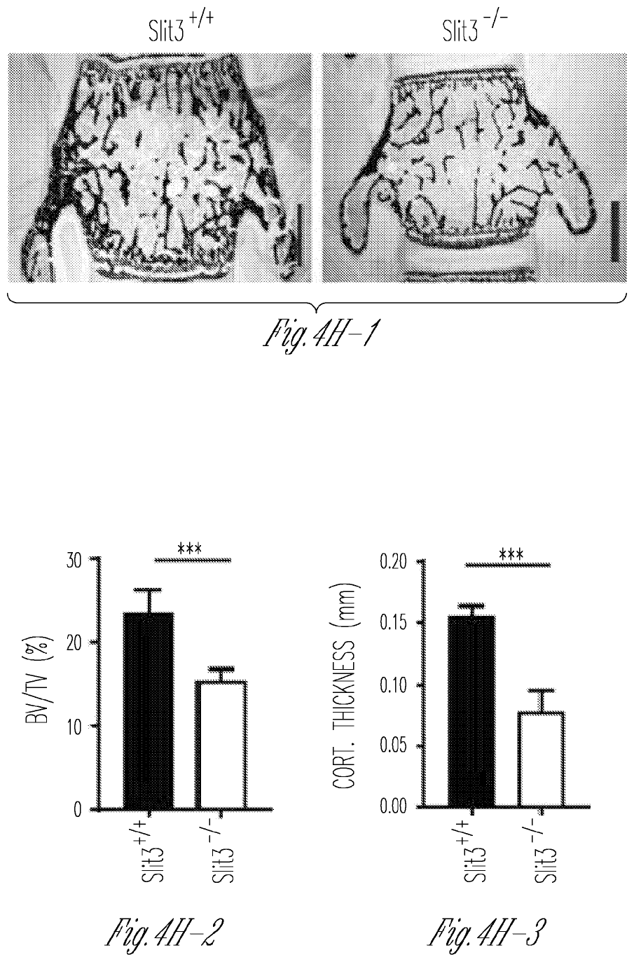

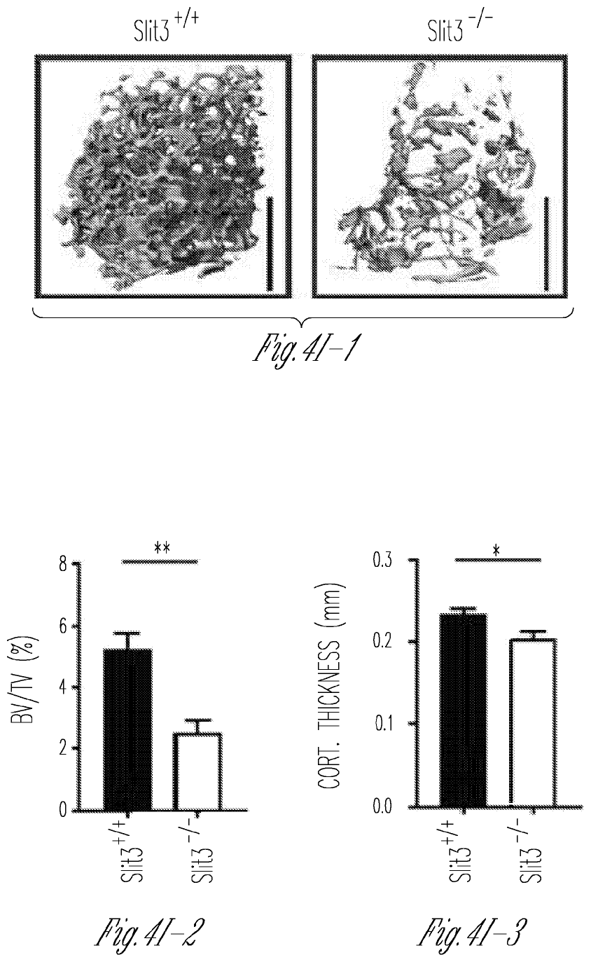

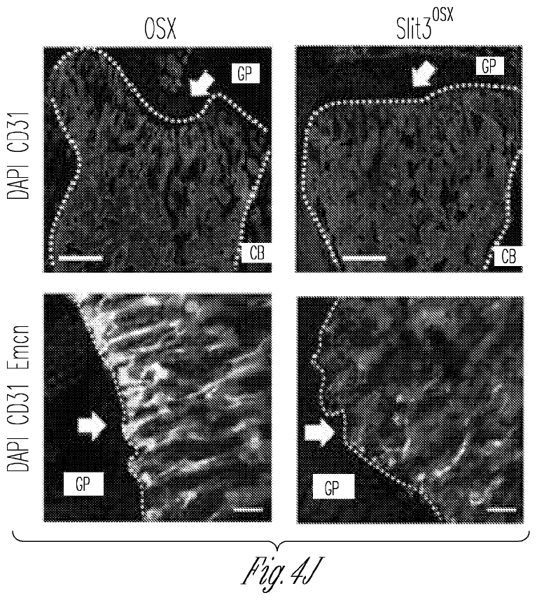

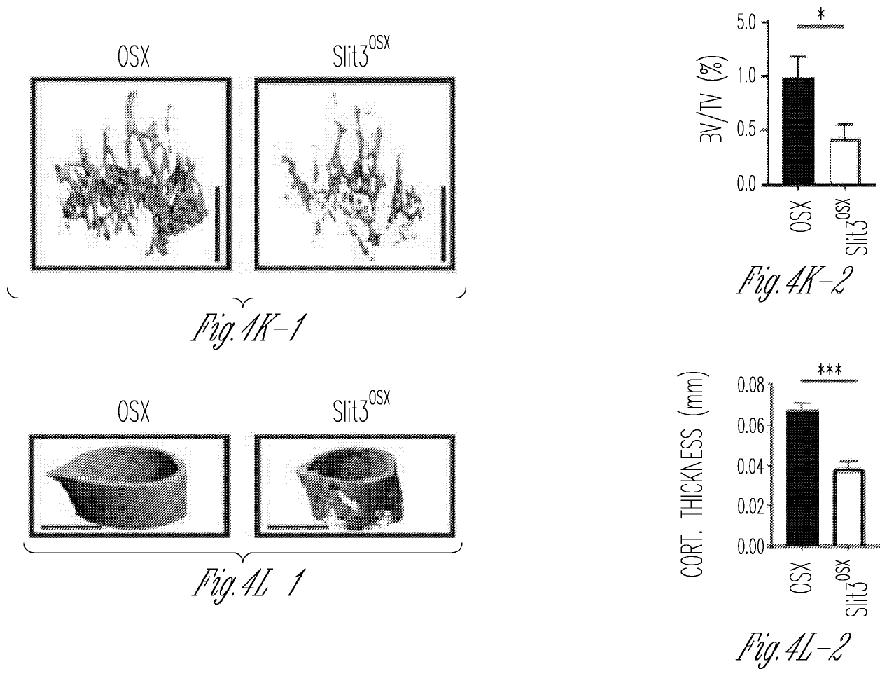

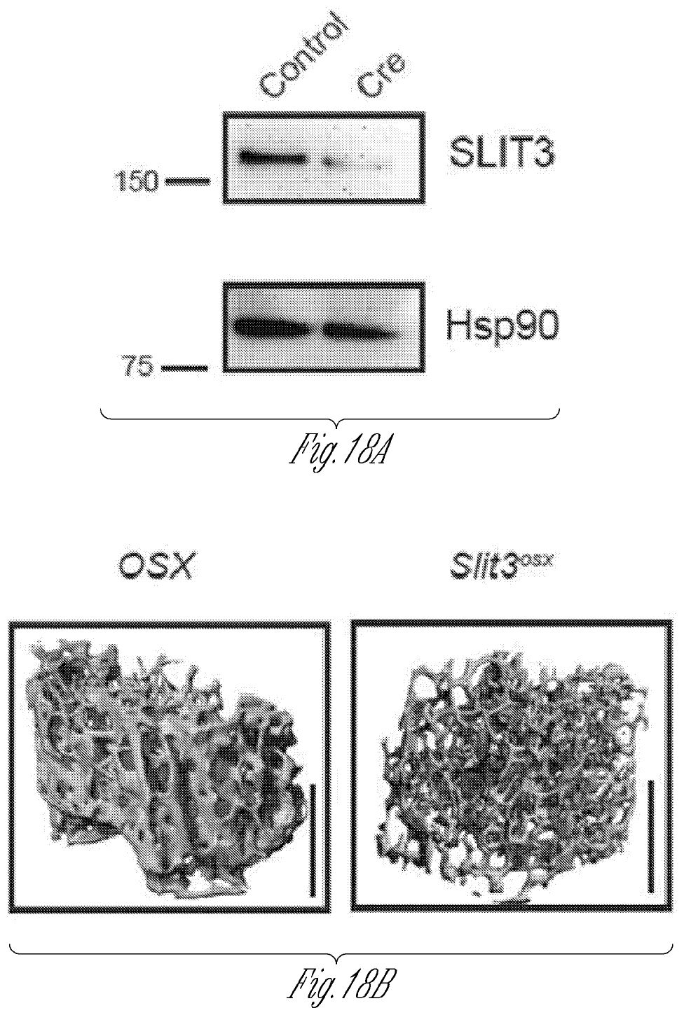

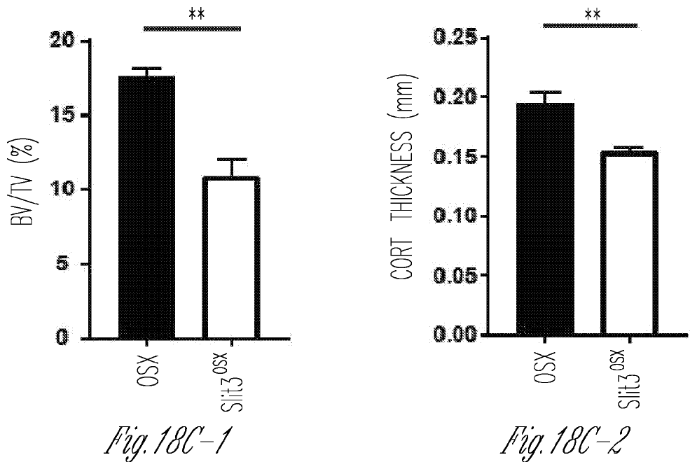

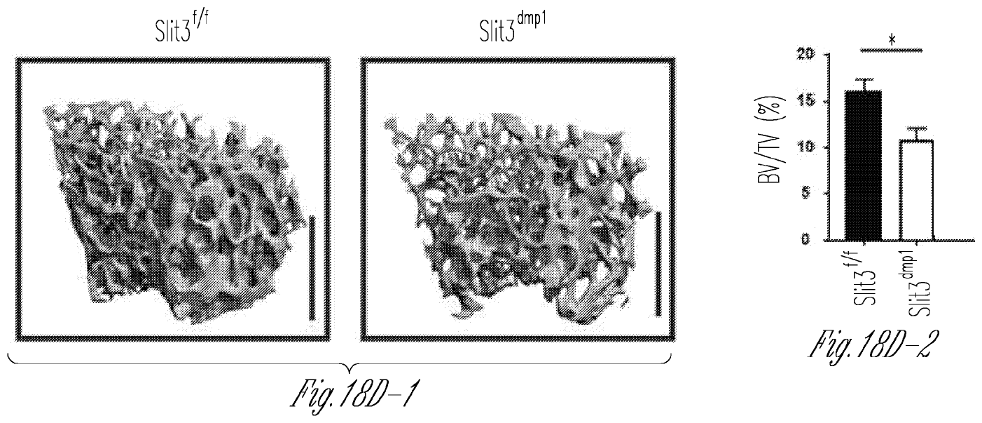

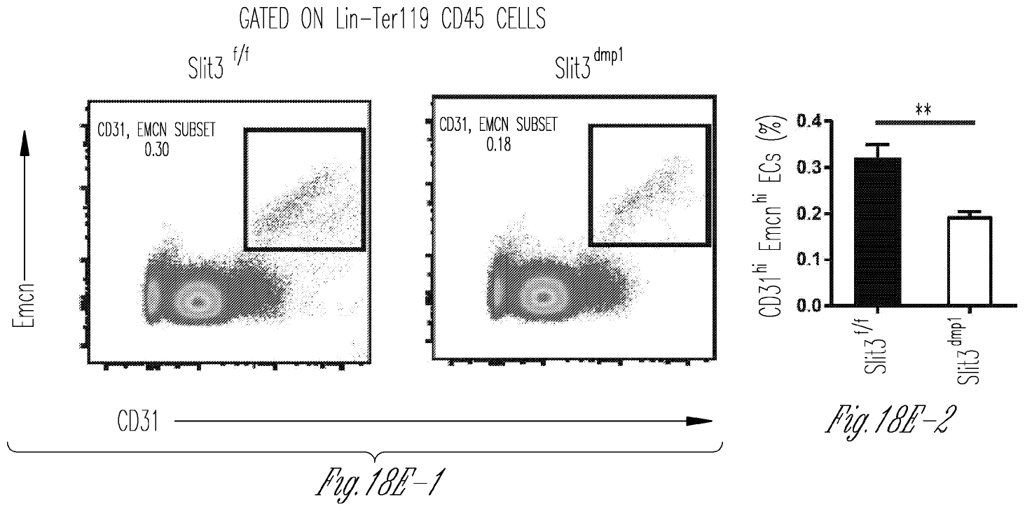

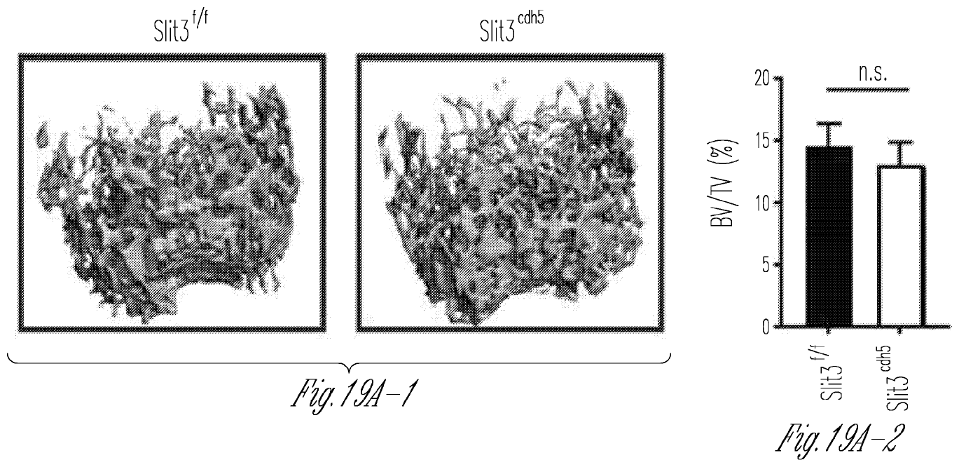

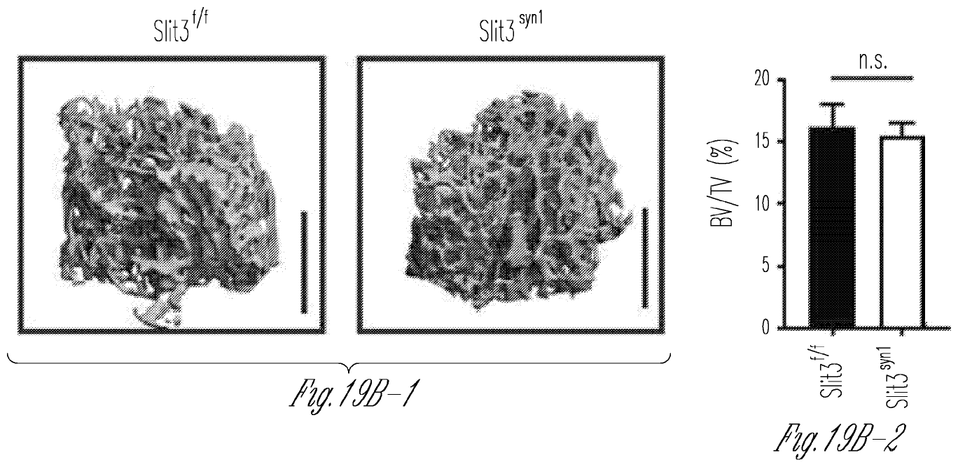

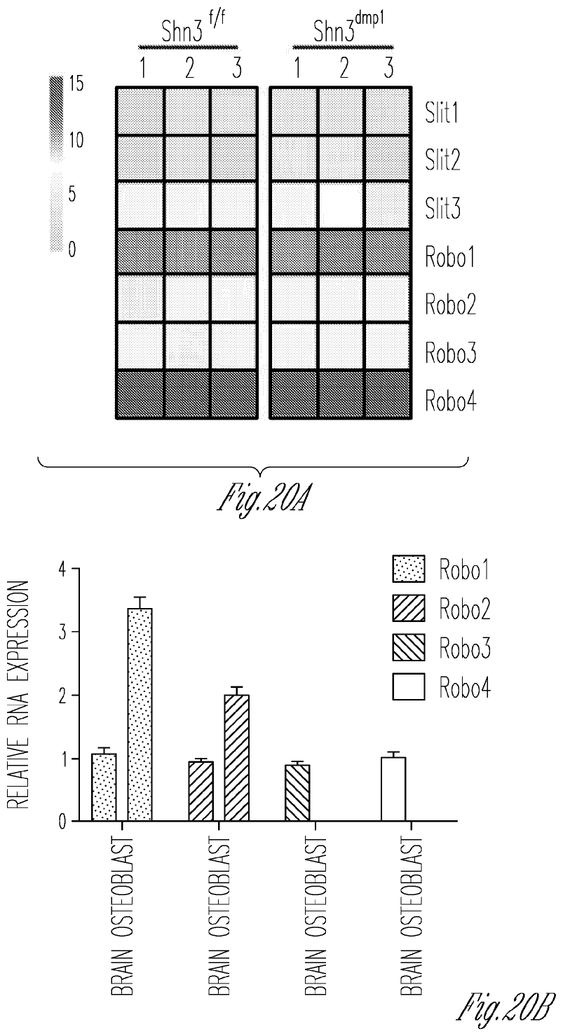

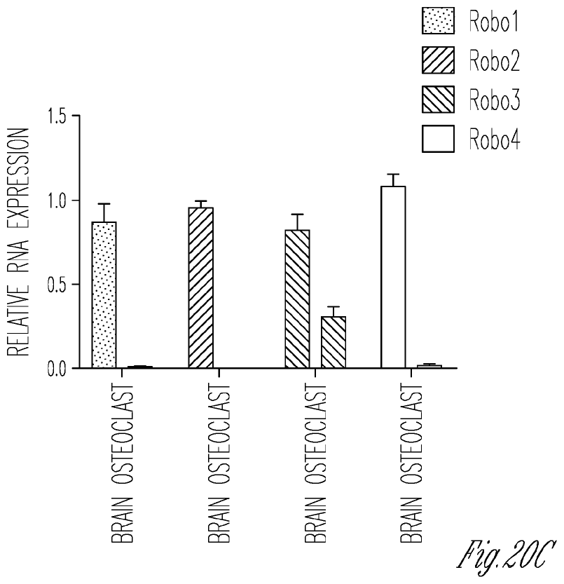

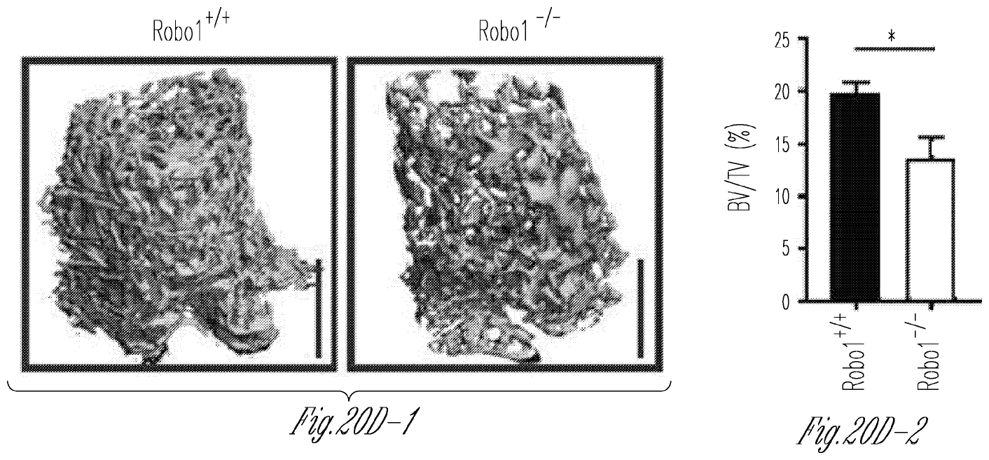

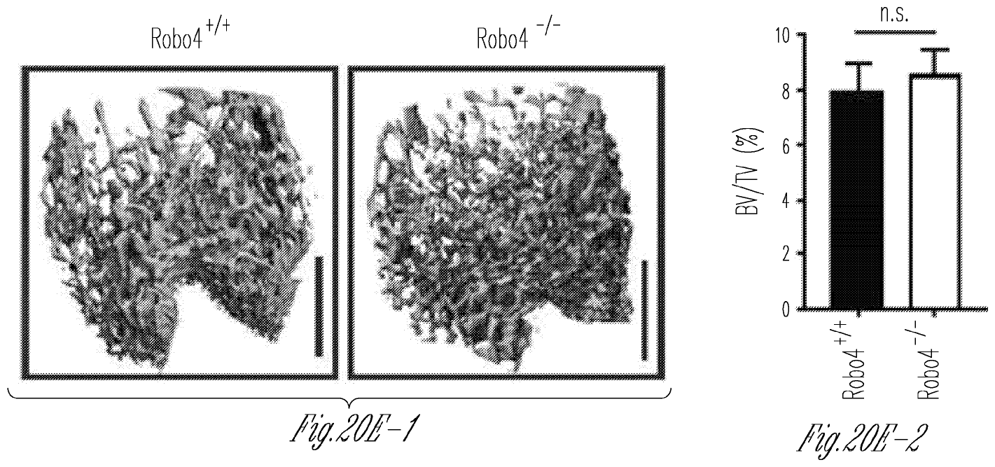

To assess if SLIT3 regulates CD31^{hi}EMCN^{hi} endothelium under physiologic conditions, immunofluorescence and flow cytometry were performed on Slit3^{-/-} mice, with both approaches revealing a reduction in CD31^{hi}EMCN^{hi} skeletal endothelium (FIG. 4E-14G and FIG. 5A-5B). Concurrent with this reduction in CD31^{hi}EMCN^{hi} endothelium, total bone mass and cortical bone thickness were also substantially reduced in Slit3^{-/-} mice (FIG. 4H-4I; FIG. 5C). Consistent with prior reports (Yuan et al. 2003). Slit3^{-/-} mice displayed a decrease in body weight at 1 month of age, with body weight normalization occurring by 3 months of age (FIG. 17A). Thus, osetopenia could be observed even at ages where the weight of Slit3^{-/-} mice was indistinguishable from that of littermate controls. Such osteopenia was due to reduced osteoblast activity, as bone formation was reduced without significant alterations in osteoclast numbers or scrum CTX, a marker of osteoclast activity (FIG. 5D-5F and FIG. 17B). To evaluate if this decrease in bone formation was due to cell intrinsic defects in osteoblast activity, the differentiation of SLIT3-deficient osteoblasts was examined. Mineralization activity, ALP induction, and induction of characteristic osteoblast transcripts were all intact or even slightly enhanced in the absence of SLIT3 (FIG. 17C-17E). Furthermore, treatment of osteoblasts with recombinant SLIT3 did not enhance osteoblast activation, nor did treatment with a ROBO1-Fc fusion that blocks SLIT signaling impair osteoblast differentiation (FIG. 17F-17G). Thus, the ability of SLIT3 to enhance bone formation in vivo is not attributable to the direct effects of SLIT3 on osteoblasts, consistent with the model that SLIT3 directly increases levels of CD31^{hi}EMCN^{hi} endothelium to augment bone formation. To confirm if osteoblasts represent the key cellular source of SLIT3 controlling levels of skeletal CD31^{hi}EMCN^{hi} endothelium and bone mass accrual in vivo, mice bearing a conditional Slit3 floxed allele (Slit3^{f/f} mice) were generated, and their deletion capacity was validated (FIG. 18A). Slit3^{f/f} mice were bred to cre-deleter strains targeting either osteoblast progenitors, OSX-cre, or mature osteoblasts, DMP1-cre. Osteoblast-specific deletion of Slit3 in vivo recapitulated both the impaired CD3^{hi}EMCN^{hi} endothelium levels and the osteopenia phenotypes observed in Slit3^{-/-} mice (FIG. 4J-4L and FIG. 18B-18E). Furthermore, neither neuron-specific deletion of Slit3 by using Synapsin-cre nor specific deletion of Slit3 in endothelial cells by using Cdh5-cre in vivo led to detectable bone loss (FIG. 19A-19B). Thus, osteoblasts are the key functional source of SLIT3 influencing skeletal biology.

### ROBO1 is a Key Receptor for SLIT3 on Marrow Endothelial Cells.

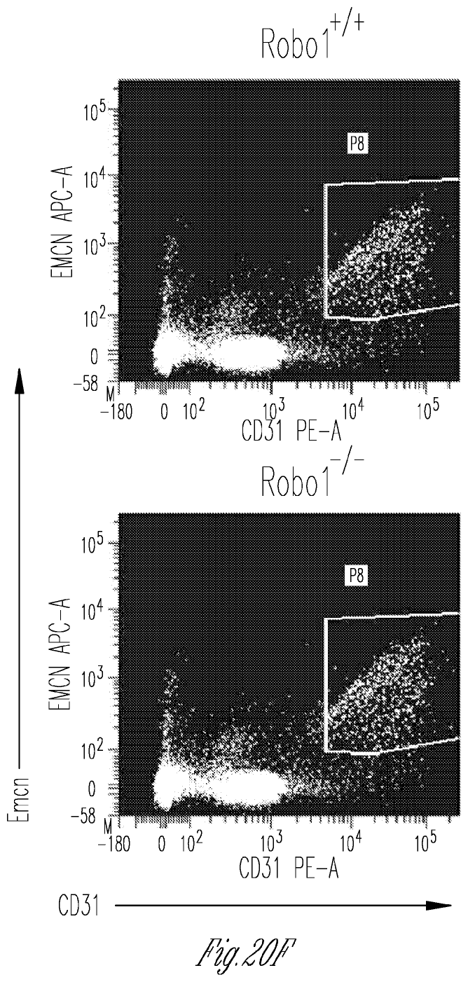

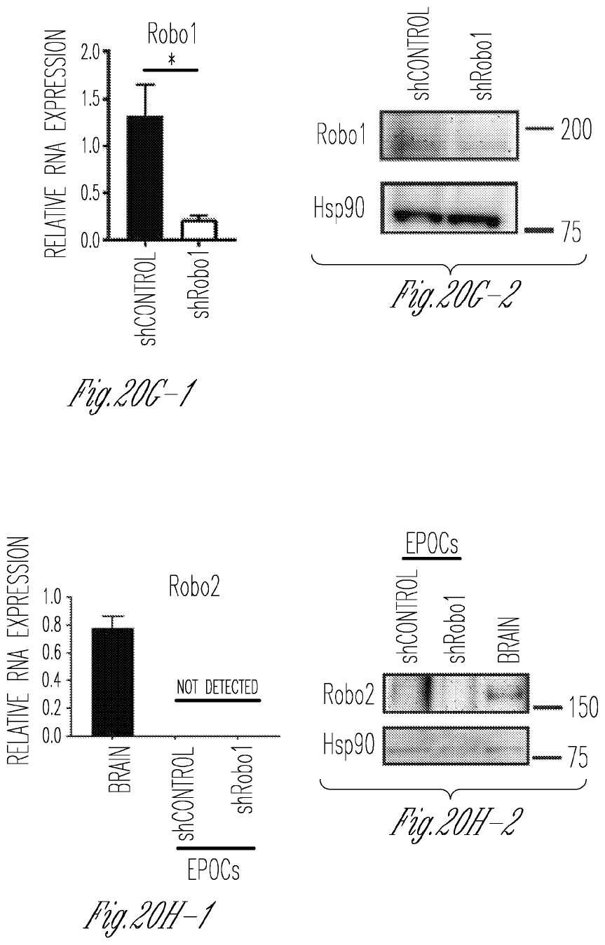

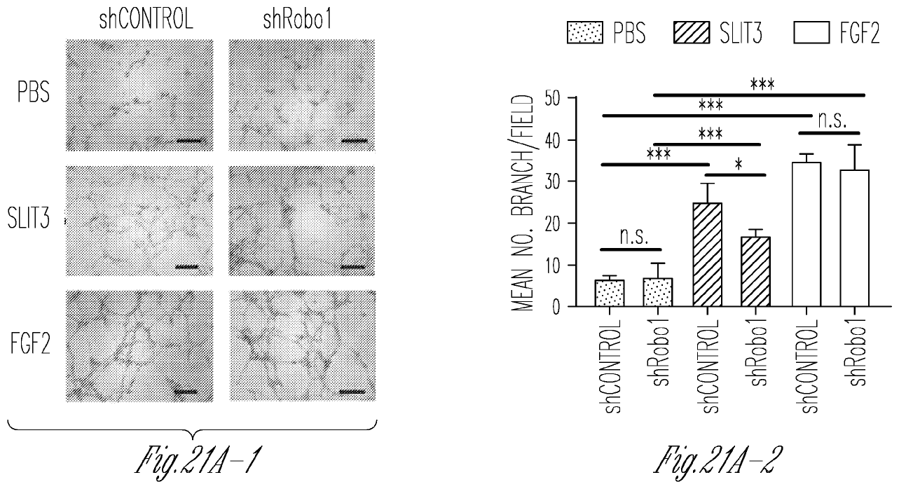

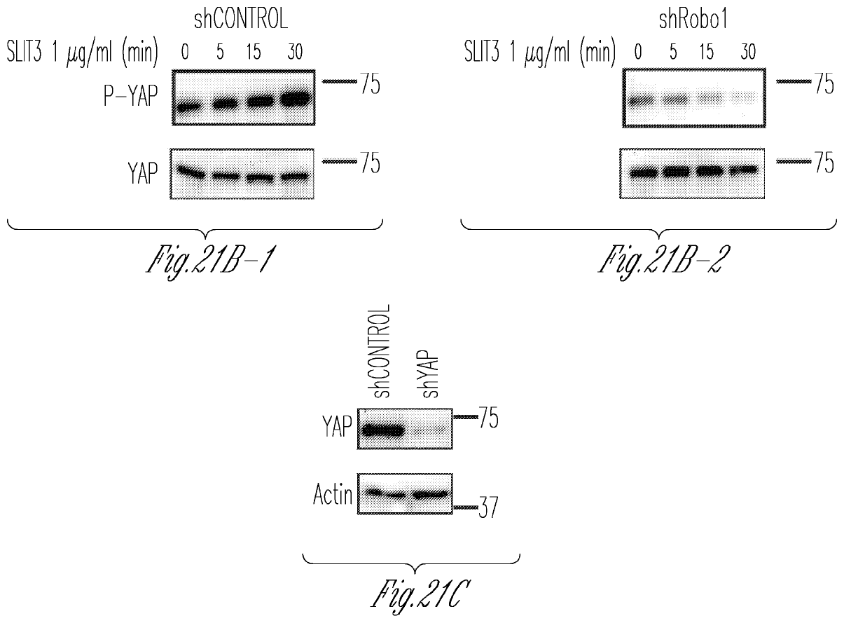

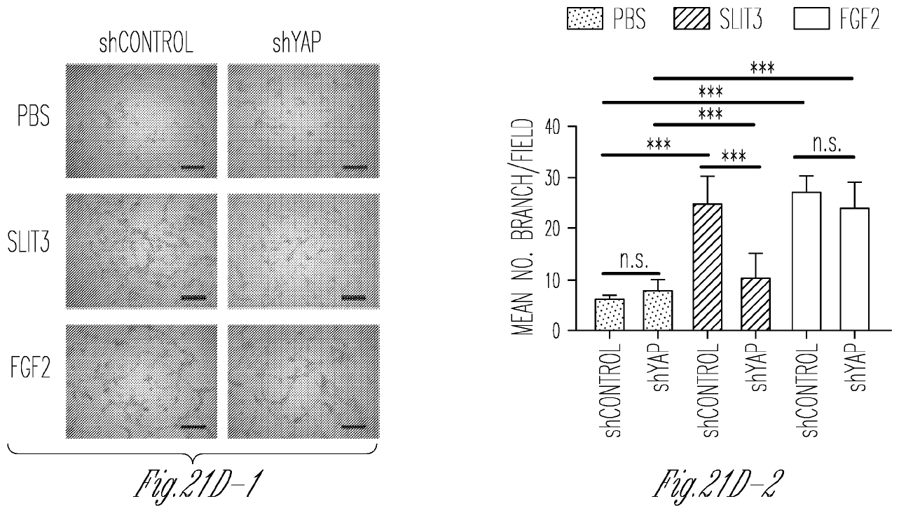

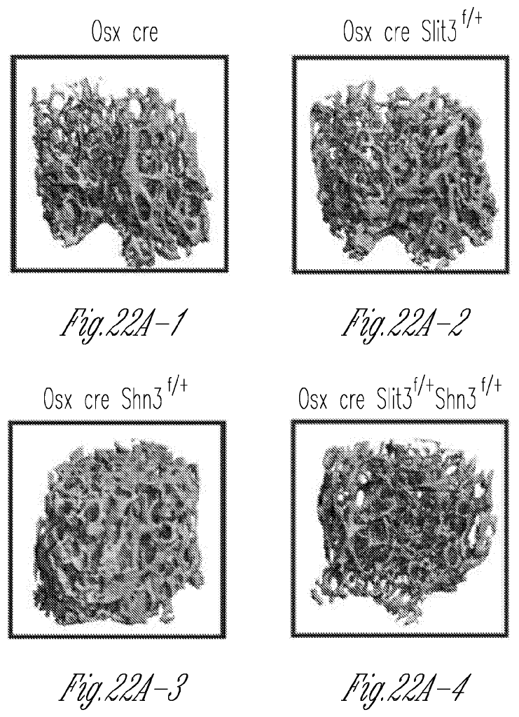

As SLITs are known to signal through the Roundabout family (ROBO1-4) of receptors, we next explored which ROBO receptors might be acting in endothelial cells to mediate the response to SLIT3 (Blockus and Chedotal 2016). CD31^{hi}EMCN^{hi} skeletal endothelial cells isolated by FACS and subjected to RNA-seq transcriptome analysis, revealed that Robo1 and Robo4 are the predominant ROBO family receptors expressed (FIG. 20A). Immunofluorescence for beta-galactosidase in the bones of Robo13^{+/-} mice bearing a knock-in of a LacZ cassette into the Robo1 locus confirmed that Robo1 expression is present in endothelium near the growth plate, consistent with CD31^{hi}EMCN^{hi} endothelial cells (FIG. 12). ROBO expression was also characterized in other skeletal cell types, indicating that osteoblasts express Robo1 and Robo2 whereas osteoclasts show negligible expression of ROBO family members. (FIG. 20B-20C). Given the expression of Robo1 and Robo1 in CD31^{hi}EMCN^{hi} endothelial cells, the skeletal phenotype of Robo1^{-/-} and Robo4^{-/-} mice was examined. Robo1^{-/-} but not Robo4^{-/-} mice display low trabecular bone mass in long bones (FIG. 5G and FIG. 18D-18E), and Robo1^{-/-} mice also showed decreased levels of CD31^{hi}EMCN^{hi} skeletal endothelium (FIG. 5H). Robo2 expression was not detected in either sorted CD31^{hi}EMCN^{hi} skeletal endothelial cells or cultured bone marrow derived endothelial cells, and Robo2 expression was not induced by either germline Robo1-deficiency or shRNA-mediated Robo1 knockdown. (FIG. 20F-20H). Thus, no evidence of ROBO2-mediated compensation for ROBO1 loss-of-function was observed.

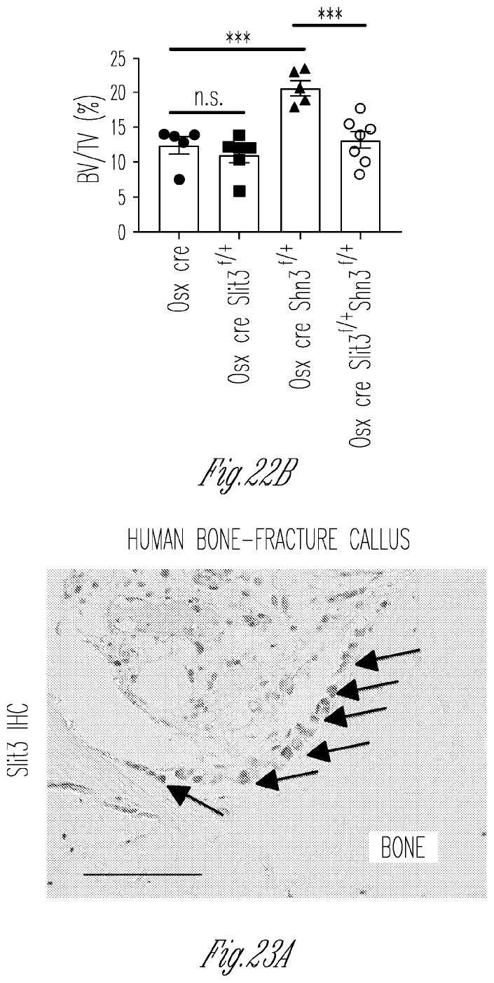

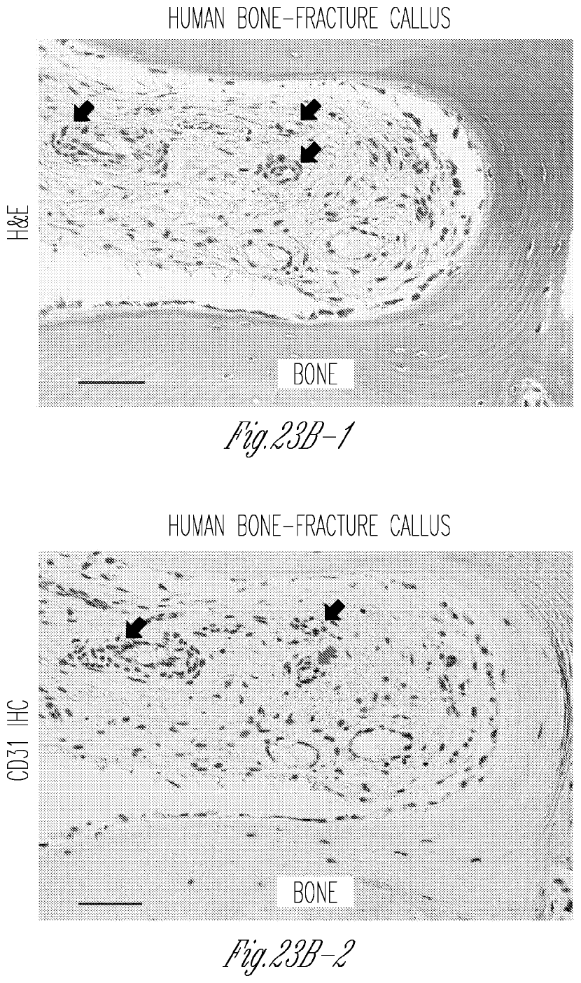

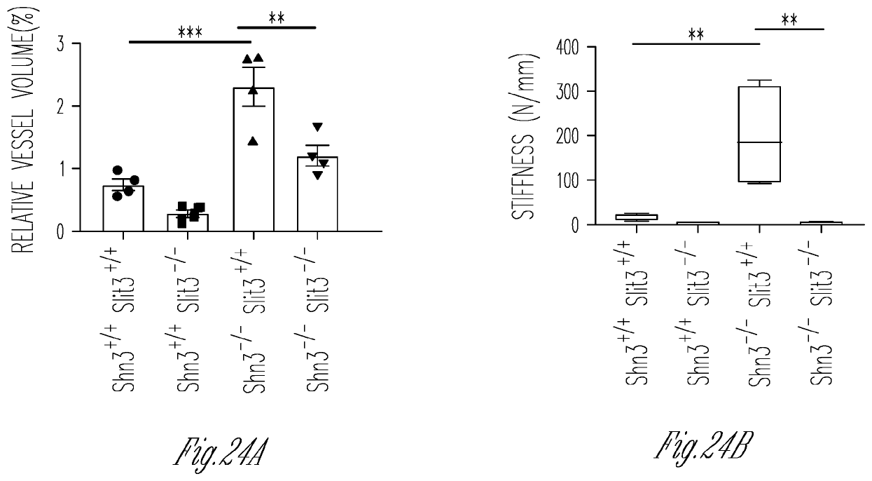

Next, the mechanism of SLIT3 mediated effects on bone marrow endothelial cells was investigated. Knockdown of Robo1 in bone marrow derived endothelial cells impaired their response to SLIT3 as determined by both their tube formation capacity and phosphorylation of the hippo pathway signaling intermediate YAP (FIG. 21A-21B). YAP has been reported to play a crucial role endothelial cell migration and tube formation (Choi et al. 2015, Sakabe et al. 2017). Consistent with this, shRNA-mediated knockdown of YAP significantly reduced the ability of SLIT3 but not FGF2 to induce tube formation in bone marrow-derived endothelial cells, indicating that YAP acts downstream of the SLIT3/ROBO1 pathway to control tube formation (FIG. 21C-21D). Taken together, ROBO1 is a key receptor controlling both endothelial cell responses to SLIT3 and overall bone mass accrual.

### Slit3 Contributes to the Ability of Shn3 to Control Osteogenesis and Skeletal Endothelium In Vivo

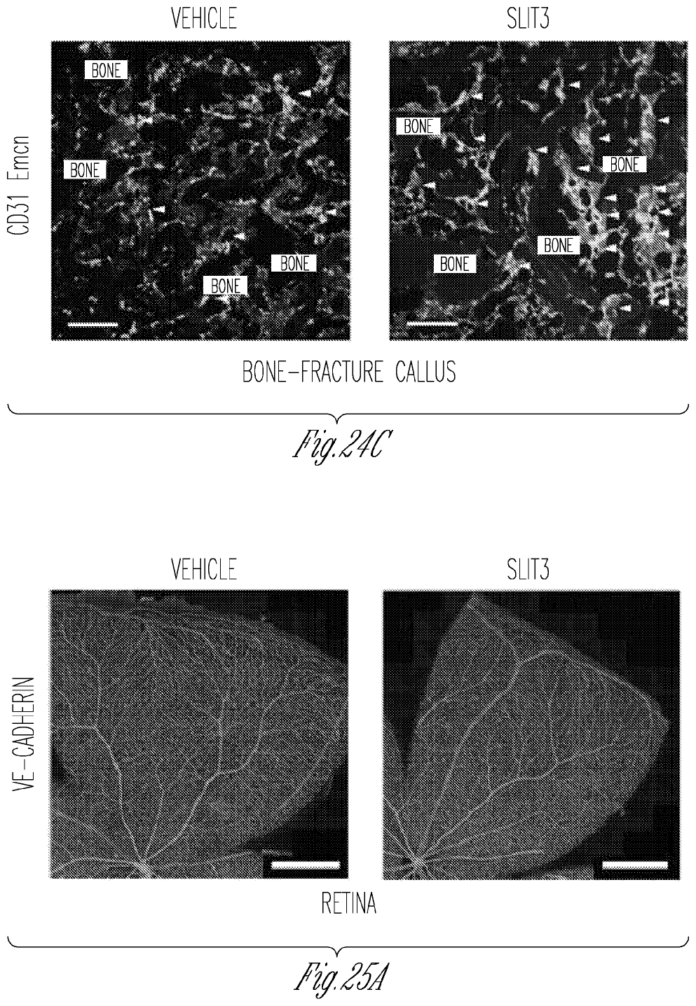

We next questioned whether the enhanced production of SLIT3 by SHN3-deficient osteoblasts contributes to the high bone mass phenotype of SHN3-deficient mice. To address this, a genetic interaction study was performed by intercrossing Shn3^{-/-} and Slit3^{-/-} mice. As shown in FIG. 5, an epistatic genetic interaction was observed between Shn3- and Slit3-null alleles, as the increases in bone formation, trabecular bone mass, and CD31^{hi}EMCN^{hi} endothelial cell levels in Shn3^{-/-} mice were partially reversed in Shn3^{-/-}Slit3^{-/-} mice. Thus, SLIT3 contributes to both the high bone mass phenotype and enhanced CD31^{hi}EMCN^{hi} endothelium phenotype of SHN3-deficient mice in vivo. In addition, a genetic interaction similar to that observed with the Shn3 and Slit3 germline null alleles was recapitulated using conditional deletion of Shn3 and Slit3 alleles in osteoblasts using Osx-cre (FIG. 22A-22B). Thus, the interaction between Shn3 and Slit3 alleles to regulate bone mass is intrinsic to osteoblasts. Taken together, these results imply that SLIT3 production is utilized by osteoblasts to condition their environment through angiogenesis to be conducive for bone formation.

### A SHN3/SLIT3 Osteoblast:Endothelium Coupling Pathway Plays an Essential Role in Fracture Healing

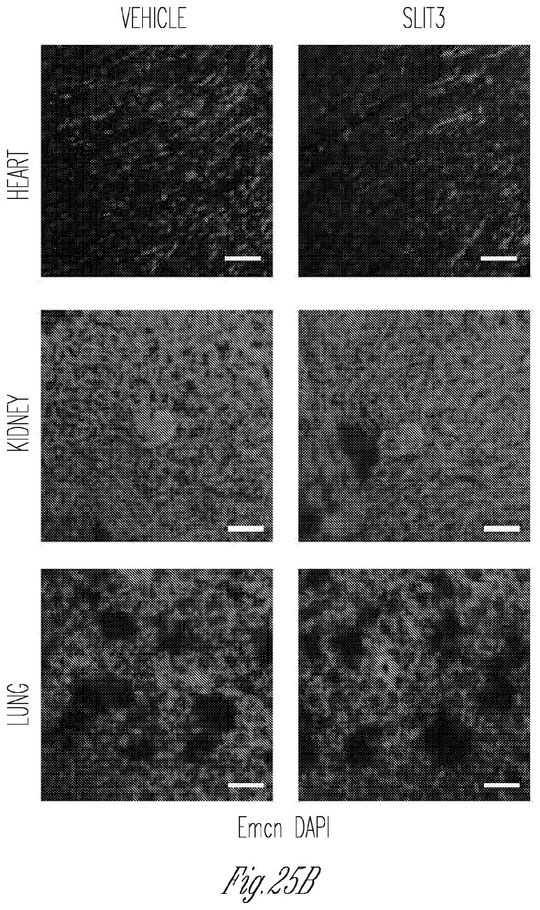

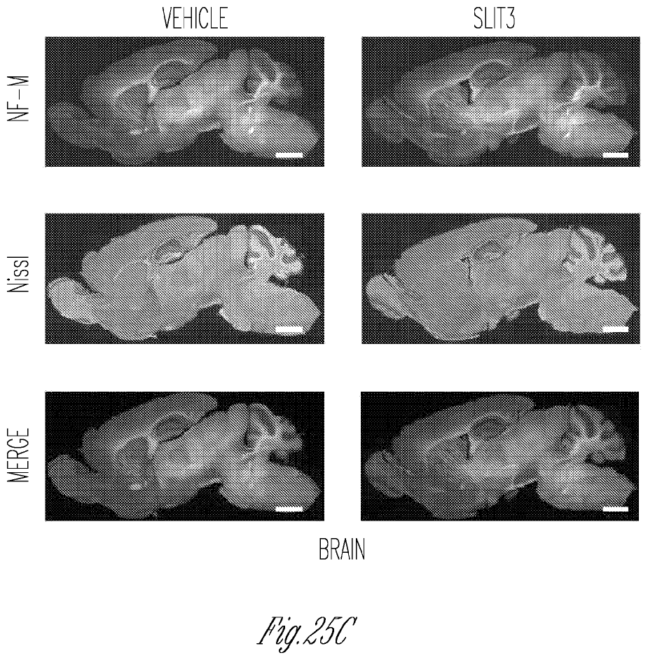

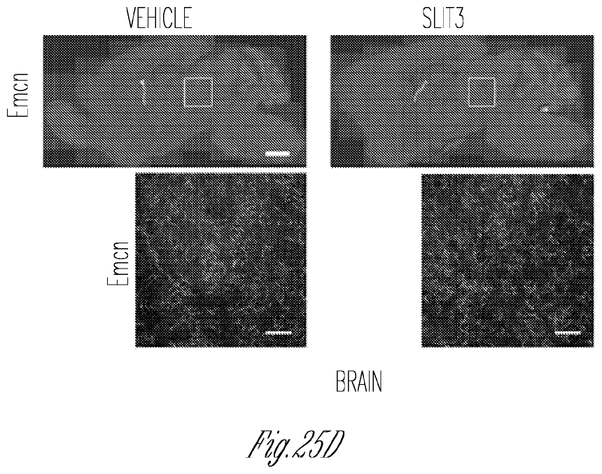

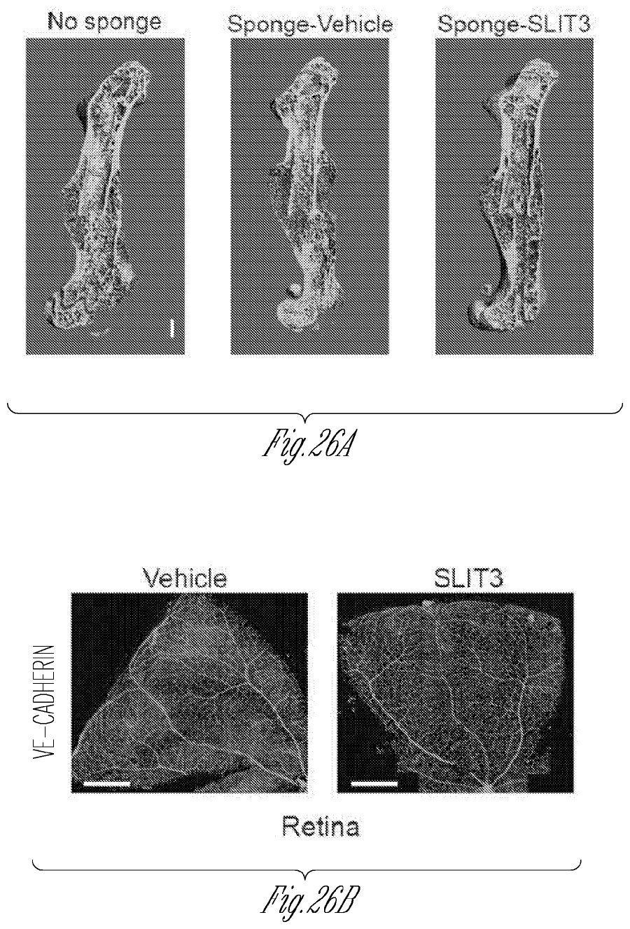

Given that bone repair is accompanied by extensive elaboration of new blood vessels, we hypothesized that SHN3/SLIT3 pathway mediated communication between osteoblasts and endothelial cells may be vital for bone fracture healing. In support of this hypothesis, immunohistochemical analysis of human fracture callus tissue demonstrated robust expression of SLIT3 in osteoblasts and the presence of CD31⁺ endothelium in physical proximity to osteoblasts (FIG. 23A-23B). An open femoral midshaft fracture model was established and used to study bone healing in Shn3^{-/-}, Slit3^{-/-}, Shn3^{-/-}Slit3^{-/-} and WT control mice. 21 days after fracture, μCT and histology analysis showed that fracture healing was enhanced in Shn3^{-/-} mice. The fracture site in Shn3^{-/-} mice displayed extensive bridging with mature lamellar bone that had already remodeled to contain marrow elements within the newly formed callus tissue. In contrast, deletion of Slit3 led to complete non-union and arrest of fracture healing with only trace amounts of cartilaginous callus present (FIG. 6A-6C). Consistent with the results of the bone mass genetic interaction studies, SLIT3 deficiency largely reversed the enhanced healing phenotype of SHN3-deficient mice. These observations of enhanced and impaired healing in SHN3-deficient and SLIT3-deficient mice, respectively, translated into improved or impaired biomechanical properties of the callus, as shown by the load to failure or maximum load sustained across the fracture site. Consistent with observations in basal bone mass phenotypes, Shn3^{-/-} mice displayed enhanced angiogenesis throughout the callus, and this phenotype was reversed by SLIT3 deficiency (FIG. 6D-6E and FIG. 24A-24B). Thus, the coupling between osteoblasts and vascular endothelium mediated by the SHN3/SLIT3 pathway is essential for fracture healing.

### SLIT3 has Therapeutic Effects in Models of Fracture Healing and Postmenopausal Osteoporosis

Due to evidence that SLIT3 mediated crosstalk between osteoblasts and CD31^{hi}EMCN^{hi} endothelium is an important regulator of bone mass accrual and bone-fracture healing, we hypothesized that administration of exogenous SLIT3 may have therapeutic effects to promote bone formation and regeneration. To examine this, recombinant SLIT3 was administered twice weekly via IV injection in 5-week-old male mice concurrent with performing an open femoral midshaft fracture. After 21 days of treatment, μCT and histological analysis showed an enhancement of bone fracture healing in SLIT3-treated mice (FIG. 6F-6G). The bone volume in the callus area was increased almost 2-fold relative to controls (FIG. 6H). Furthermore, vascularization of fracture area including CD31^{hi}EMCN^{hi} endothelium was also increased by SLIT3 administration (FIG. 6I and FIG. 24C). Finally, biomechanical testing demonstrated that SLIT3 treatment significantly enhanced the maximum load sustained and stiffness of the fracture callus, demonstrating improvements in clinically meaningful endpoints (FIG. 6J).

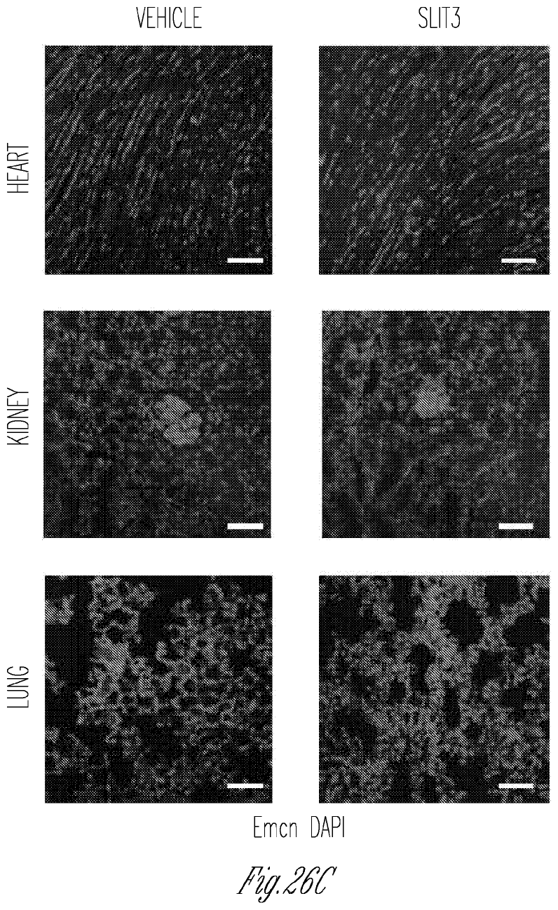

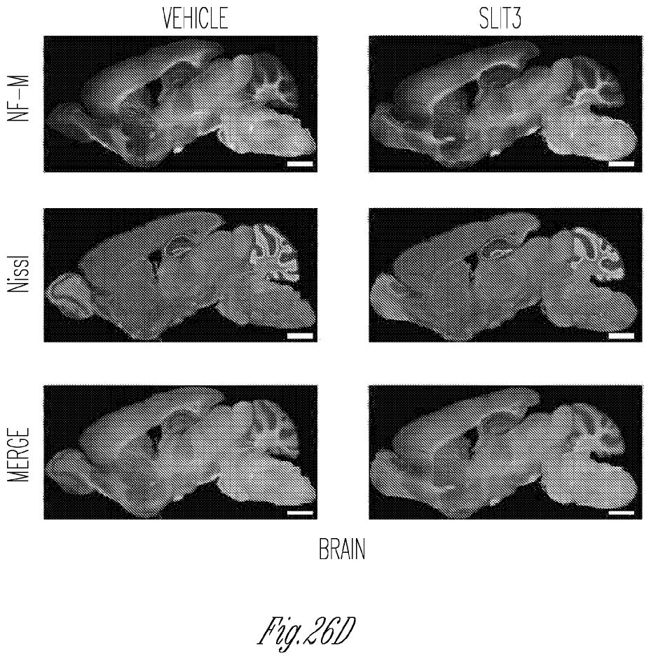

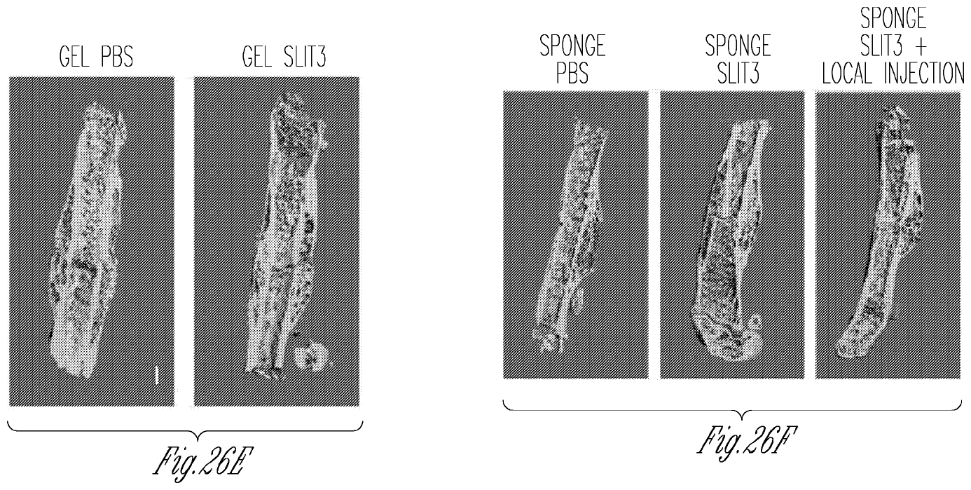

However, it is currently unclear if systemic treatment with SLITs will result in systemic toxicity (Kruszka et al. 2017; Morin-Poulard et al. 2016; Jaworski and Tessier-Lavigne 2012; Kidd T et al. 1998). To investigate this, mice underwent systemic IV treatment with SLIT3 and were examined for unexpected toxicity. Notably, examination of vascular morphology did not detect alterations in lung, heart, kidney or retina, and no changes in brain ultrastructure were present after SLIT3 administration (FIG. 25A-25D). Thus, under this dosing strategy and with regards to these endpoints, the effects of SLIT3 were specific to bone. However, while SLIT3 did not alter retinal vascular morphogenesis, some mice treated with SLIT3 unexpectedly displayed increased retinal vascular permeability (FIG. 31, arrowheads). As increased retinal vascular permeability is known to result in vision loss as seen in conditions such as diabetic retinopathy or exudative macular degeneration, this toxicity may preclude systemic therapeutic administration of SLIT3.

### Local Delivery

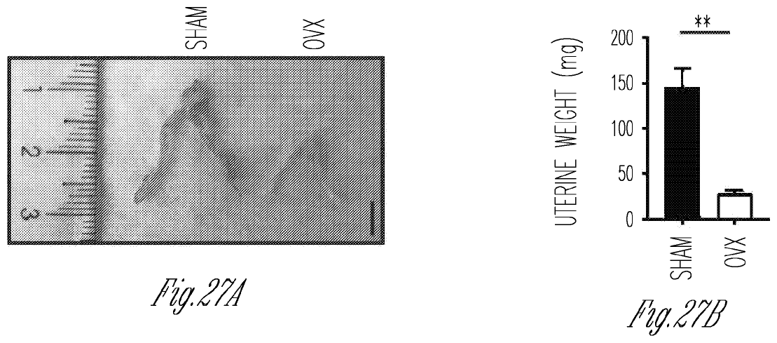

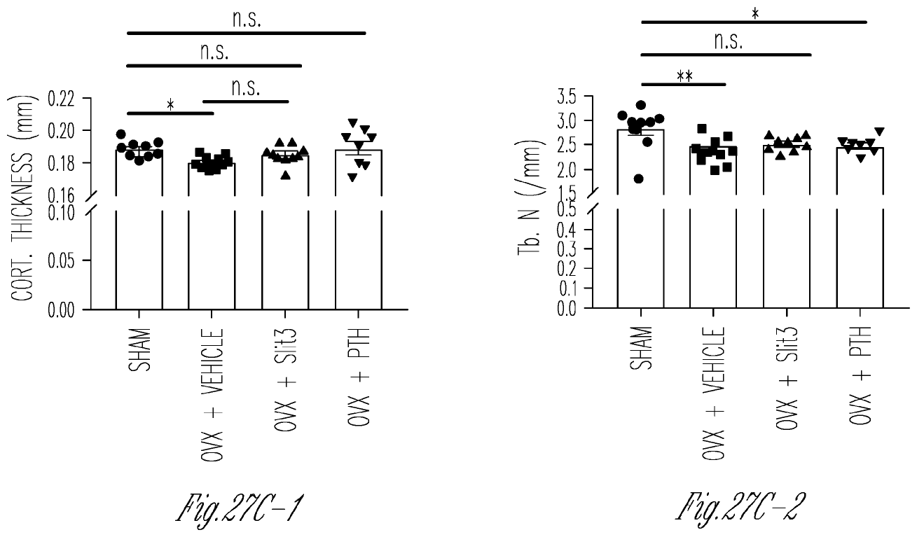

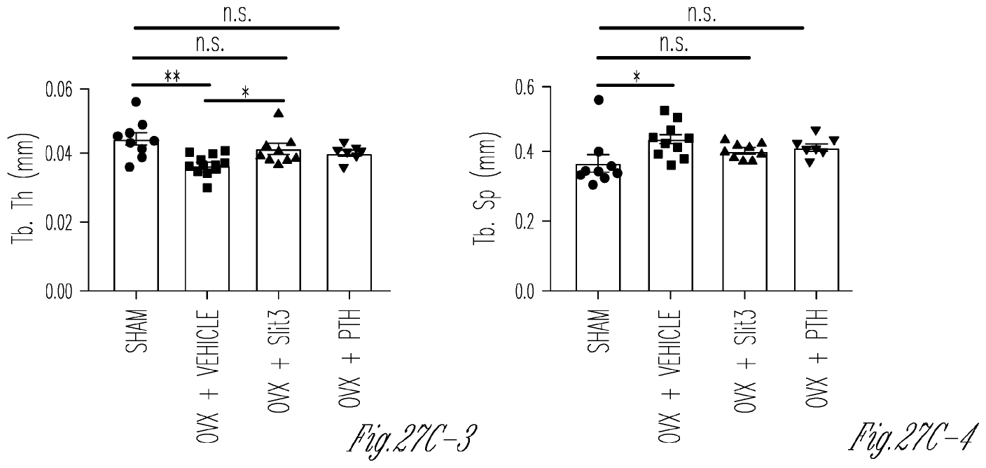

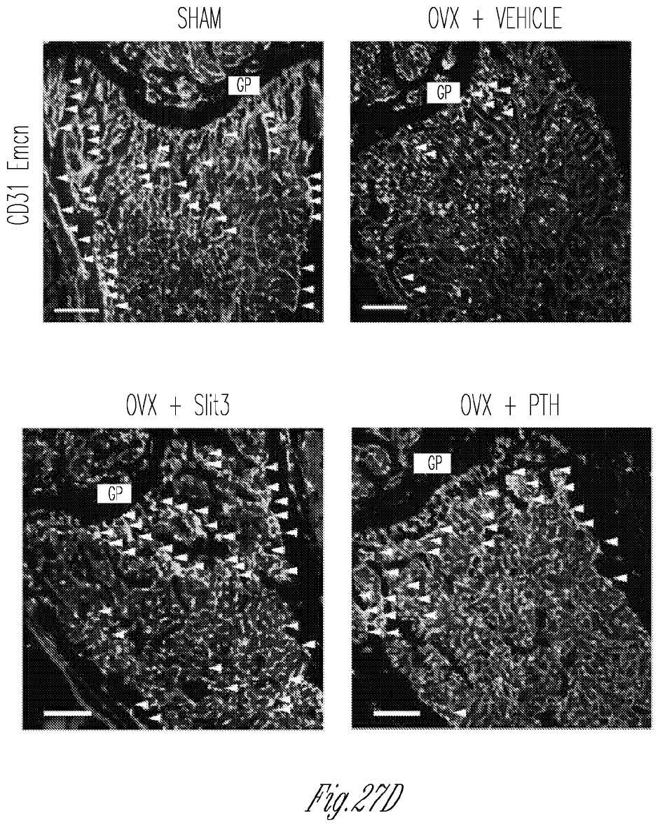

Based on these observations, local delivery of SLIT3 would avoid the risk of an undesired effect of SLIT3 to promote retinal vascular permeability. As a proof-of-principle strategy to avoid the potential extra-skeletal toxicities that were identified as described herein, local delivery of SLIT3 into a fracture site was achieved with a SLIT3-loaded collagen sponge. A hydrogel containing SLIT3 has also been implanted. These approaches recapitulated the effects of systemic SLIT3 delivery in promoting fracture healing as judged by improved mineralization and biomechanical properties of the fracture callus and increased formation of CD31^{hi}EMCN^{hi} endothelium (FIG. 6K-6L and FIG. 26). Local delivery of SLIT3 also did not impact non-skeletal vascular abundance or morphology or brain ultrastructure (FIG. 26B-26D).

Thus, local delivery of SLIT3 using a SLIT3-containing construct is effective in preclinical models to enhance skeletal healing. This approach is expected to be broadly applicable for the treatment of skeletal diseases and conditions, including the treatment of osteoporosis.

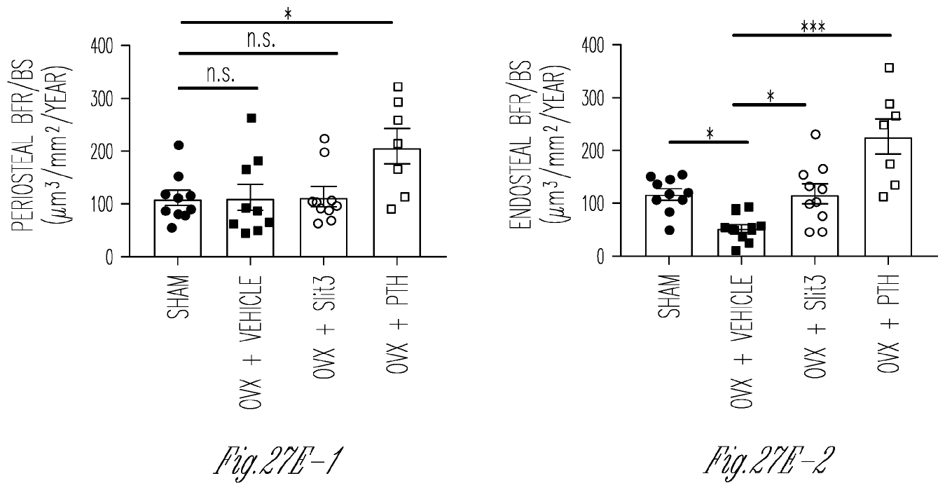

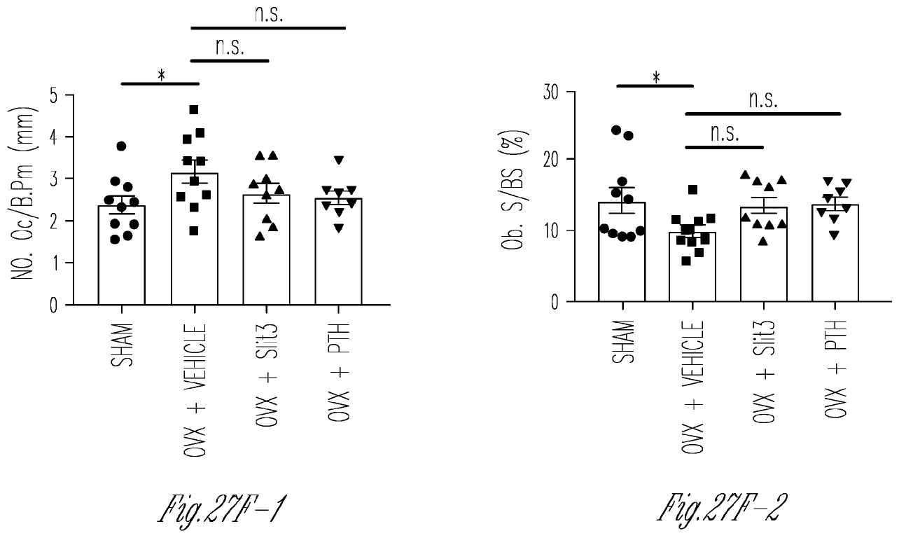

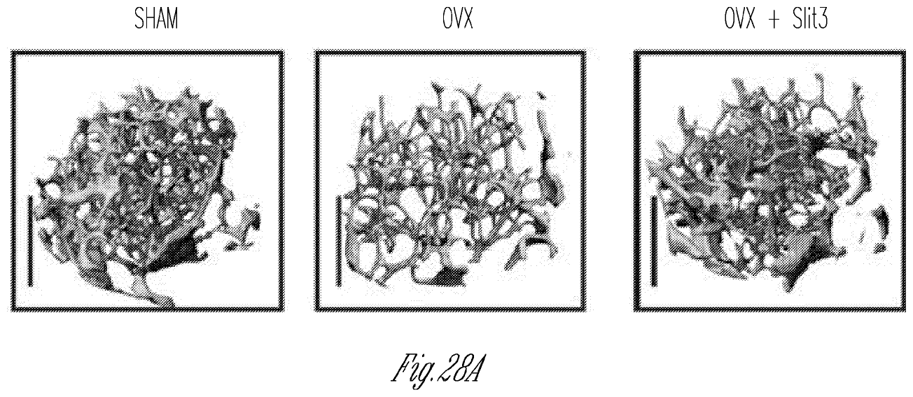

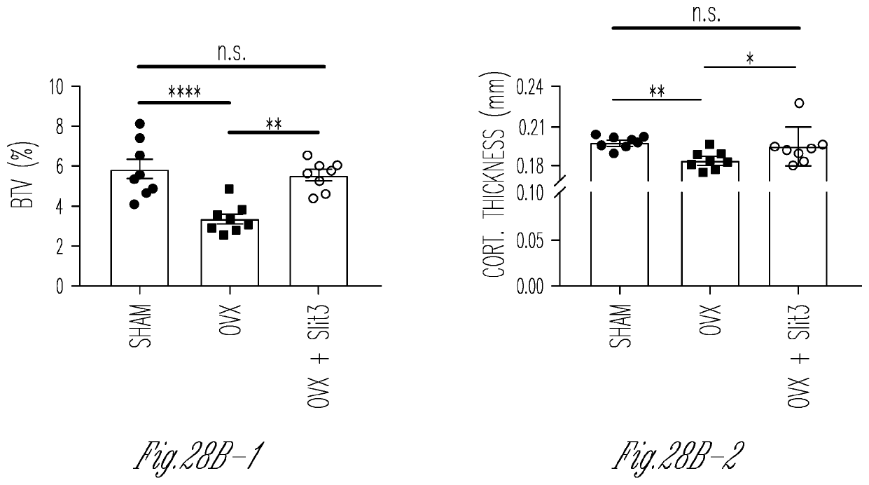

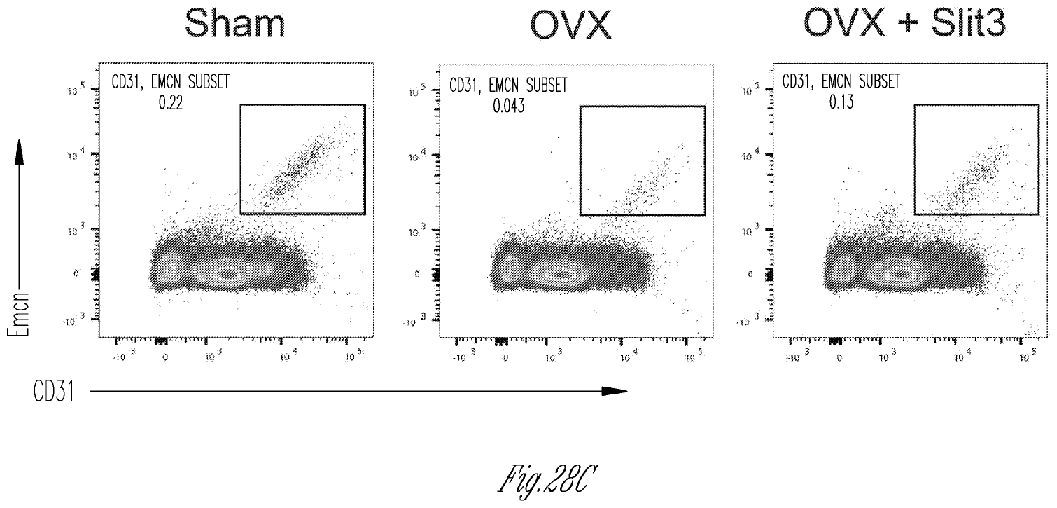

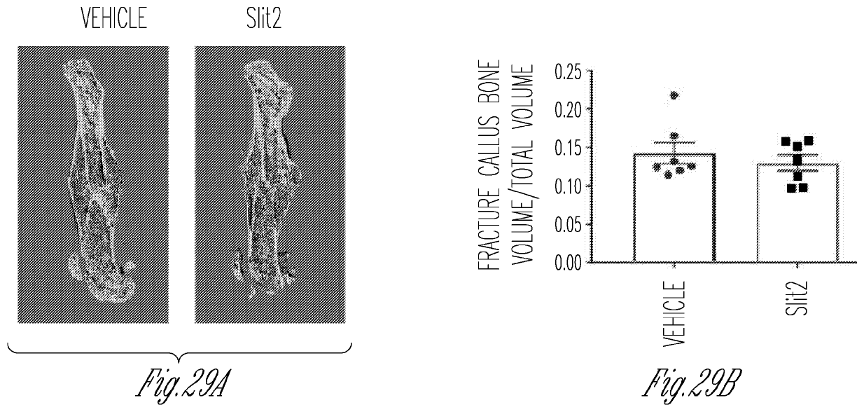

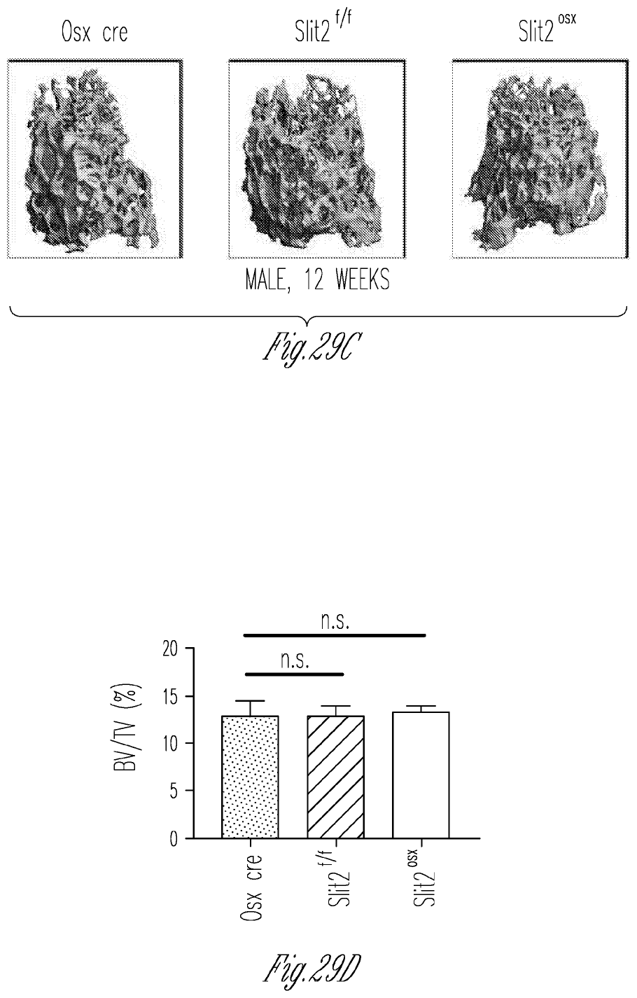

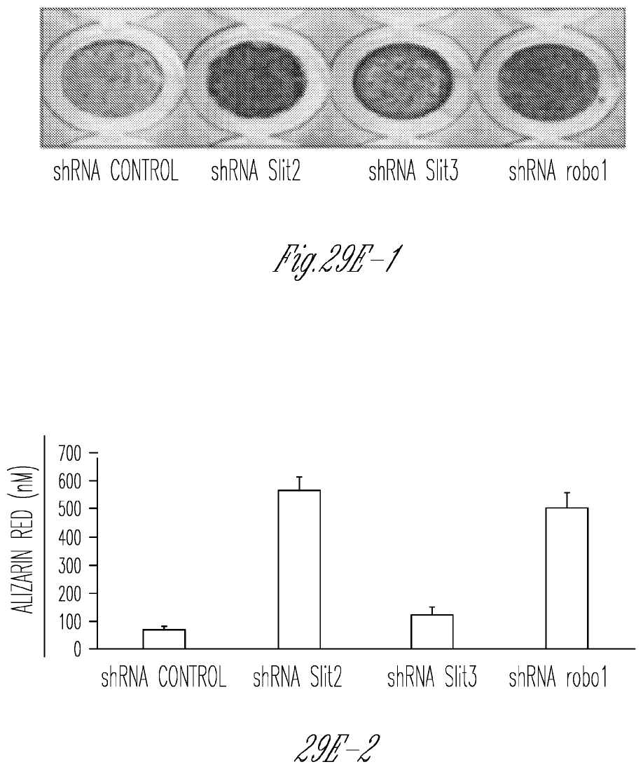

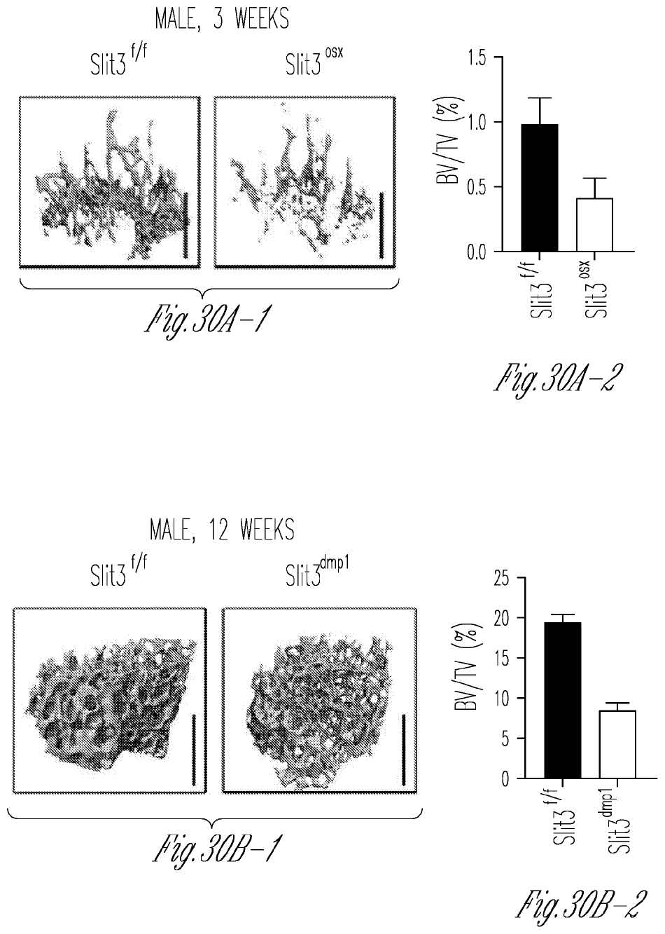

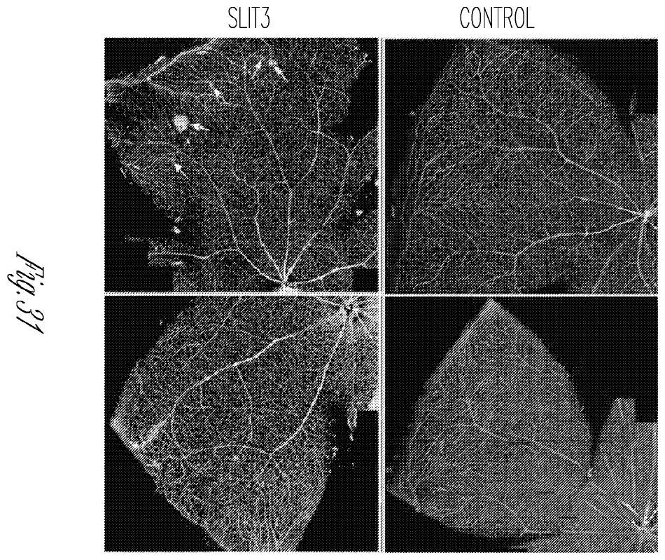

Given the therapeutic effects of SLIT3 in a fracture model, we next examined whether systemic SLIT3 administration can protect from bone loss in the murine ovariectomy (OVX) model of postmenopausal osteoporosis (Bouxsein et al. 2005). Successful OVX was confirmed two months after OVX by the presence of both osteopenia and uterine atrophy (FIG. 6M and FIG. 27A-27B). First, the ability of SLIT3 to prevent bone loss was examined in this model. Mice were treated with IV injection of SLIT3 or vehicle twice weekly initiated two weeks after OVX. Micro-CT analysis showed that SLIT3 administration significantly countered OVX-induced bone loss as shown by increased trabecular BV/TV and cortical thickness (FIG. 6M and FIG. 27C). As the effects of SLIT3 were comparable to the effects of PTH treatment, this indicates that the magnitude of SLIT3 effect is clinically significant. Moreover, both SLIT3 and PTH administration reversed the attenuation of endosteal CD31^{hi}EMCN^{hi} endothelium occurring after OVX and similarly rescued endosteal bone formation (FIG. 27D-27F). To further investigate the therapeutic activity of SLIT3, the ability of SLIT3 to promote bone formation after the onset of OVX-induced osteopenia was examined. Mice were treated with IV injection of SLIT3 or vehicle twice weekly initiated eight weeks after OVX surgery. SLIT3 administration significantly reversed bone loss as shown by increased trabecular BV/TV and cortical thickness (FIG. 28A-28B). Furthermore, SLIT3 administration significantly increased CD31^{hi}EMCN^{hi} endothelial cells in this context (FIG. 28C). Taken together, these results provide proof-of-principle that SLIT3 may have clinical utility to enhance fracture healing and to treat disorders of low bone mass such as postmenopausal osteoporosis.

### Discussion

Though bone formation is mediated solely by osteoblasts, it is likely that many other tissue types present in bone, such as vascular endothelium or autonomic and sensory nerves, contribute to creating a conducive milieu for bone formation (Fukuda et al. 2013, Kusumbe, Ramasamy, and Adams 2014, Ramasamy et al. 2014, Zhang et al. 2016, Xu 2014). To the degree that the creation of a local osteogenic milieu should be coordinated with the cell intrinsic matrix production capacity of osteoblasts, it would be mechanistically attractive for osteoblasts to regulate their own matrix production alongside the activities of these supporting cell types. However, this remains a poorly understood facet of bone physiology. Here, the inventors hypothesized that mice with extreme increases in bone formation represent an opportunity to identify how osteoblasts regulate supporting tissue types in bone to create a pro-osteogenic milieu. In particular, the greatly enhanced bone formation phenotype of mice lacking the adaptor protein SHN3 was utilized to identify that osteoblast-derived SLIT3 enhances levels of an osteogenic subtype of vascular endothelium in bone. CD31^{hi}EMCN^{hi} endothelium. Accordingly, mice lacking SLIT3 or the known SLIT receptor ROBO1 display reduction in both the levels of marrow CD31^{hi}EMCN^{hi} endothelium and basal bone mass.

Support for osteoblasts being a key source of SLIT3 in bone include the observation of robust and specific SLIT3 expression in osteoblasts, without appreciable SLIT3 expression in osteoclasts or CD31^{hi}EMCN^{hi} endothelium (FIG. 11A-11B and FIG. 20A). Conditional deletion of Shn3 in late-stage osteoblasts enhanced SLIT3 expression and CD31^{hi}EMCN^{hi} endothelium levels, and osteoblast derived conditioned medium enhanced bone marrow endothelial tube formation in a SLIT3-dependent manner. Moreover, genetic interaction studies demonstrated that the CD31^{hi}EMCN^{hi} endothelium-promoting and enhanced bone formation effects of SHN3 deficiency are partially SLIT3 dependent. This rescue of the SHN3 phenotype is not a generic property of crossing SHN3 to a mouse with low bone mass, as crossing the Shn3^{-/-} mouse strain to the osteopenic Rsk2^{-/-} strain did not significantly alter the Shn3^{-/-} phenotype (Shim, J. H., et al. 2013). Thus, the epistatic interaction between Shn3 and Slit3 alleles in both regulation of bone mass and CD31^{hi}EMCN^{hi} endothelium levels provides in vivo genetic evidence that SLIT3 is a critical effector downstream of SHN3, though it does not preclude the existence of additional effectors.

Additionally, ROBO1-deficient but not ROBO4-deficient mice show an osteopenic phenotype, and accordingly ROBO1 knockdown partially blocks bone marrow endothelial responses to SLIT3. Though the phenotype of ROBO1-deficient mice and the in vitro studies indicate that it is a key receptor of SLIT signals in the regulation of bone mass accrual, it cannot be excluded that other SLIT/ROBO members similarly contribute in either an independent or redundant manner (Blockus and Chedotal 2016).

Flow cytometry analysis of CD31^{hi}EMCN^{hi} endothelium indicates that this is a relatively rare population of cells, with only very limited numbers of cells present relative to other hematopoietic or mesenchymal lineages. This raises the question of how such a small population can exert such a large effect on organ physiology. One possible explanation is that the highest density of CD31^{hi}EMCN^{hi} endothelium is observed at very active sites of bone formation, such as the primary spongiosum immediately adjacent to the growth plate of an actively growing long bone, and within this site CD31^{hi}EMCN^{hi} endothelium is observed to be in close physical proximity with osteoblast-lineage cells. This physical proximity between CD31^{hi}EMCN^{hi} endothelium and the osteoblast lineage cells they support may act to amplify each other's physiologic effects. Further work is needed to clarify the nature and mediators of these interactions beyond SLIT3. Additionally, much remains to be learned about properties that define CD31^{hi}EMCN^{hi} endothelium, including how this population relates to other endothelial cell types present in bone (Ramalingam, Poulos, and Butler 2017).

Given the evidence that fracture healing is accompanied by extensive elaboration of new blood vessels, the role of SHN3/SLIT3 mediated coupling between osteogenesis and CD31^{hi}EMCN^{hi} endothelium was explored in bone regeneration and found to be critically important.

### Example 2: Osteoclasts are not a Source of Physiologically Relevant SLIT3

As described herein SLIT3 is of fundamental importance as an osteo-anabolic reagent. This Example describes further studies clarifying the cellular sources and targets of SLIT3.

### Materials and Methods

Animals. Slit3^{-/-} and CTSK-Cre mice were described previously. Slit3^{fl/fl} were generated by homologous recombination insertion of a floxed allele into the Slit3 locus. All experiments were performed according to protocols approved by the institutional animal care and use committee of Weill Cornell Medical College. All mice were maintained under specific pathogen free conditions, fed ad libitum chow and housed up to 4 animals per cage on a standard day-night cycle lighting.

Osteoclast culture, differentiation assays. Western Molting and qPCR. In vitro primary osteoclast cultures, quantitative PCR, Western blotting, and TRAP staining were performed as previously described. Primary antibodies were specific for SLIT3. (1:1,000; Cell Signaling) and HSP90 (1:2,000; Sigma).

Micro-CT Analysis histology, immunohistochemistry, and histomorphometry. Micro-CT analysis was conducted on a Scanco Medical Micro-CT 35 system by an investigator blinded to the genotypes of the animals under analysis. Paraffin embedding, sectioning and TRAP staining were performed as previously described. Static and dynamic histomorphometric analyses were performed using the Osteomeasure Analysis System (Osteometries) as previously described, also by an investigator blinded to the genotypes of the animals being analyzed. Immunofluorescence staining was performed according to a previously published protocol.

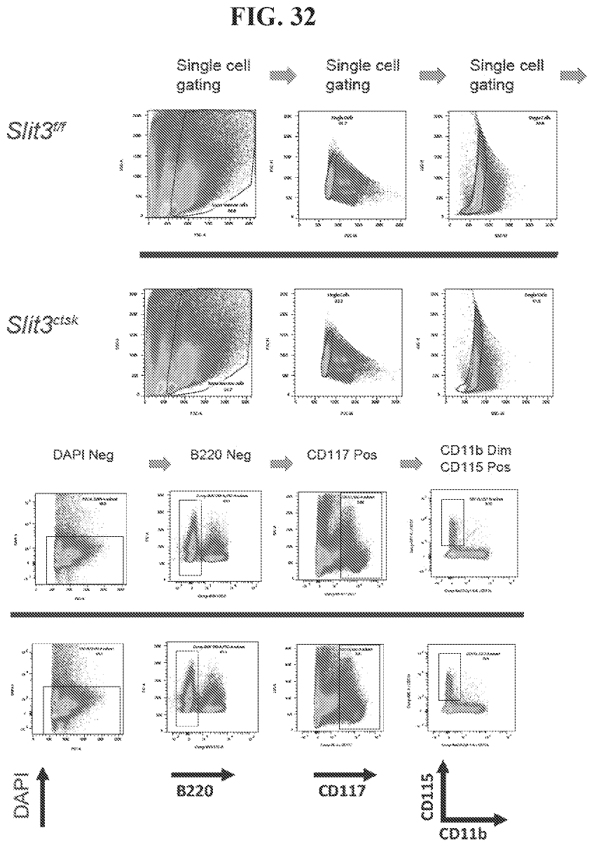

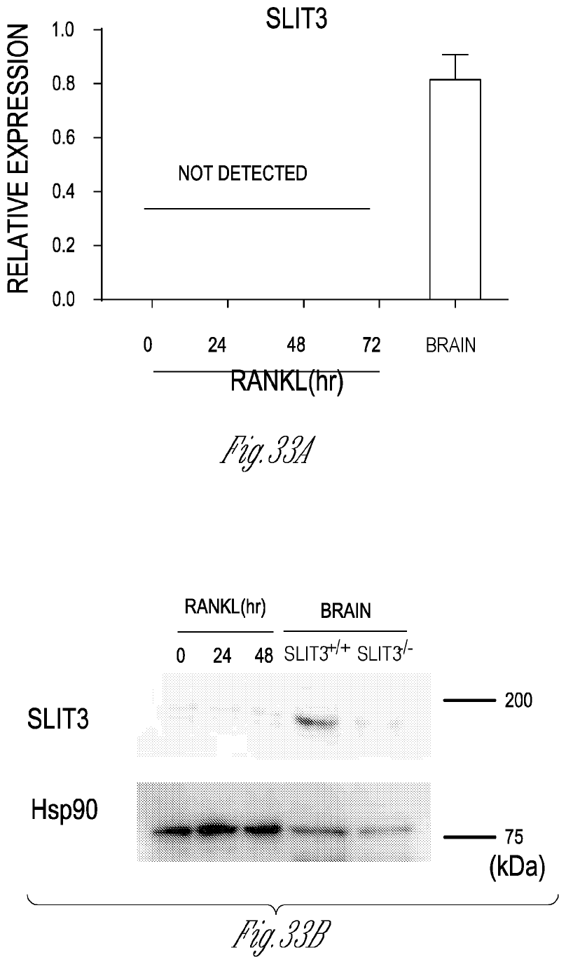

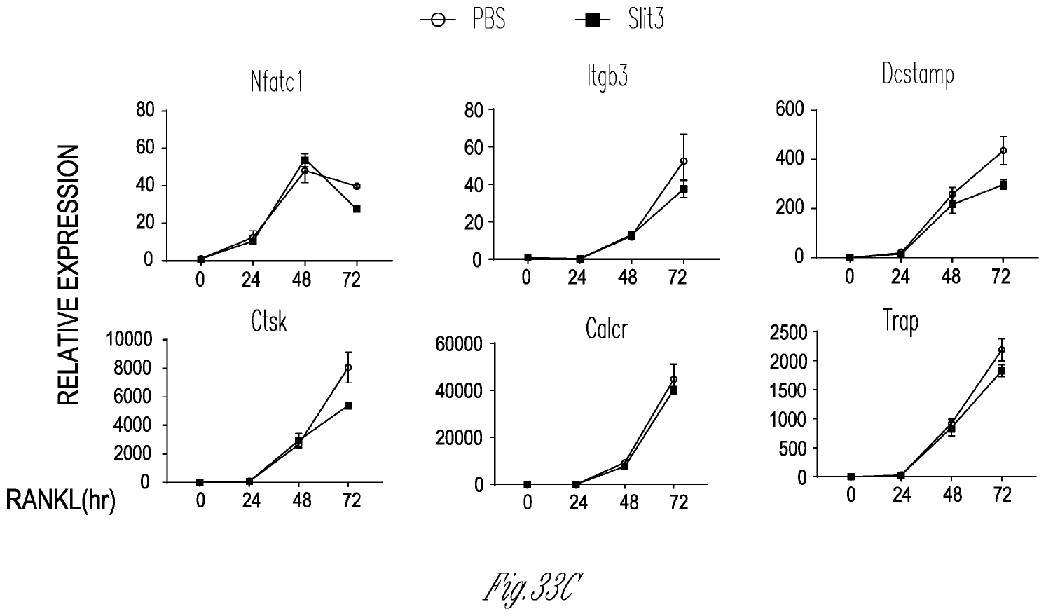

Flow cytometry analysis and cell sorting. Bone marrow cells collected from Slit3^{fl/fl} and Slit3^{ctsk} mice were filtered through a cell strainer (70 μm; BD Falcon) to remove debris. After washing, equal numbers of cells per mouse were blocked with Purified Rat Anti-Mouse CD16/CD32 (BD Biosciences) for 30 min on ice, then stained with BUV395-conjugated B220 antibody (BD), Percp-Cy5.5-conjugated CD11b (Biolegend), FITC-conjugated CD115 (Biolegend) and PE/Cy7-conjugated CD117 (BioLegend) for 45 min on ice. After washing, cells were resuspended in PBS (pH 7.2) with 2 mM EDTA and 1 μg mL-1 DAPI (live/dead exclusion) for analysis on an LSRII flow cytometer system (BD Biosciences) cytometer and analyzed using FlowJo software (Tree Star). Cell sorting was performed with a FACSAria II SORP cell sorter (Becton Dickinson), with exclusion of DAPI+ cells and doublets. The strategy to sort osteoclast progenitor cells (OCPs) is diagrammed in FIG. 32.

Bone marrow transplantation. We followed a published protocol for bone marrow transplantation (Zhao B, et al. Nature Medicine 2009). Briefly, Bone marrow cells from wild-type or KO Attenuate mice were harvested and 5 million of total bone marrow cells from each donor were transplanted by intravenous tail vein injection into each of the irradiated wild-type recipients. Recipient mice (6-week-old BALB/c mice) were lethally irradiated with a single dose of 875 rads 1 day prior to transplantation. Bone marrow-chimeric mice were sacrificed 16 weeks after bone marrow transplantation. The experiments using chimeric mice were approved by the Hospital for Special Surgery Institutional Animal Care and Use Committee.

Statistical methods. All data were presented as the mean±s.e.m. Sample sizes were calculated on the assumption that a 30% difference in the parameters measured would be considered biologically significant with an estimate of sigma of 10-20% of the expected mean. Alpha and Beta were set to the standard values of 0.05 and 0.8, respectively. No animals or samples were excluded from analysis, and where applicable, animals were randomized to treatment versus control groups. Statistical methods are indicated in the figure legends. The GraphPad PRISM software (v6.0a; GraphPad, La Jolla, Calif., USA) was used for statistical analysis. A P value<0.05 was considered statistically significant.

### Results

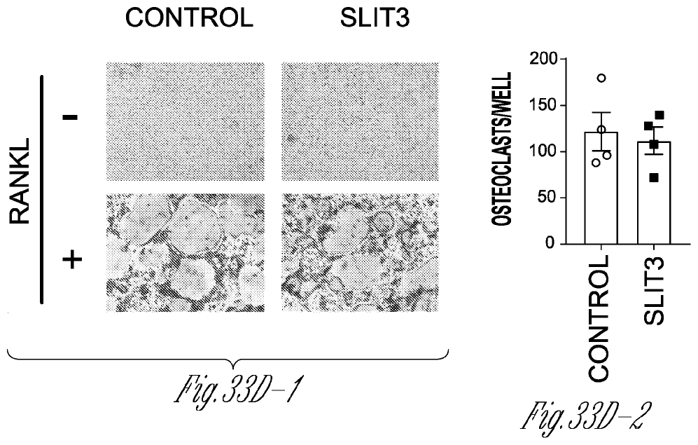

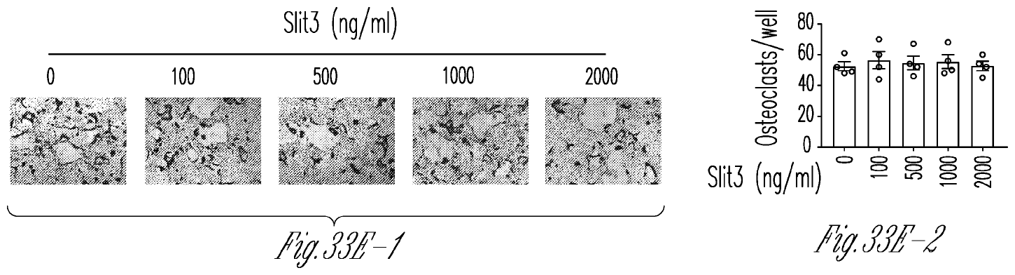

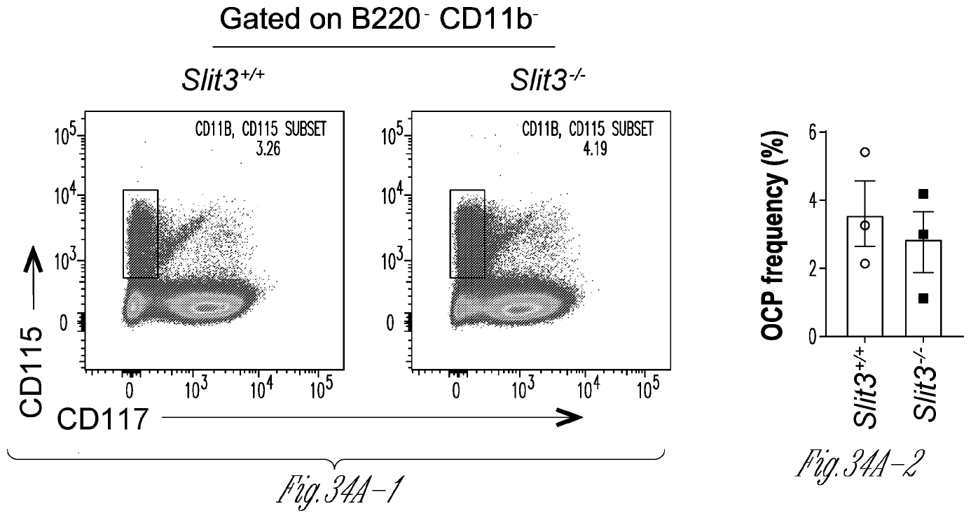

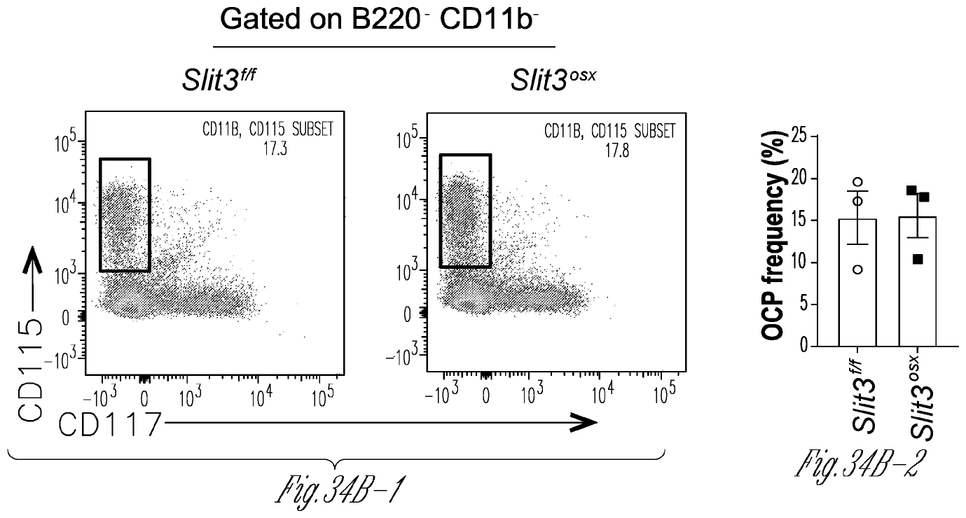

To measure SLIT3 expression during osteoclastogenesis, an in vitro osteoclast formation assay was first performed using wild type bone marrow macrophages (BMMs). Interestingly, compared with the robust SLIT3 expression seen in brain, SLIT3 expression was not detected during osteoclastogenesis at either the mRNA or protein level (FIG. 33A-33B). This finding is consistent with a previous Gene-Chip analysis of axon guidance cues in osteoclast and osteoblast differentiation and another independent expression study in mouse macrophages. In addition, these data indicate that osteoblasts but not osteoclasts are a major source of SLIT3 production in bone compartments, consistent with findings described above on severe osteopenia in Slit3^{dmp1} and Slit3^{osx} mice.

To assess the effects of exogenous SLIT3 on osteoclastogenesis, BMMs were treated with recombinant SLIT3 at levels showing bioactivity in multiple other cellular assays, including tube formation assays in endothelial cells. Although SLIT3 treatment modestly inhibited expression of osteoclast marker genes, no significant changes were observed in the number of tartrate-resistant acid phosphatase (TRAP) positive osteoclasts compared with controls (FIG. 33C-33D). Similar negative results were obtained during a dose-titration of SLIT3 during osteoclast differentiation (FIG. 33E). Consistent with these in vitro findings, treatment for six weeks with recombinant SLIT3 in vivo was only able to increase amounts of CD31^{hi}EMCN^{hi} vasculature but not osteoclast formation, indicating that SLIT3 has in vivo angiogenic but not anti-osteoclastogenic activity in vivo.

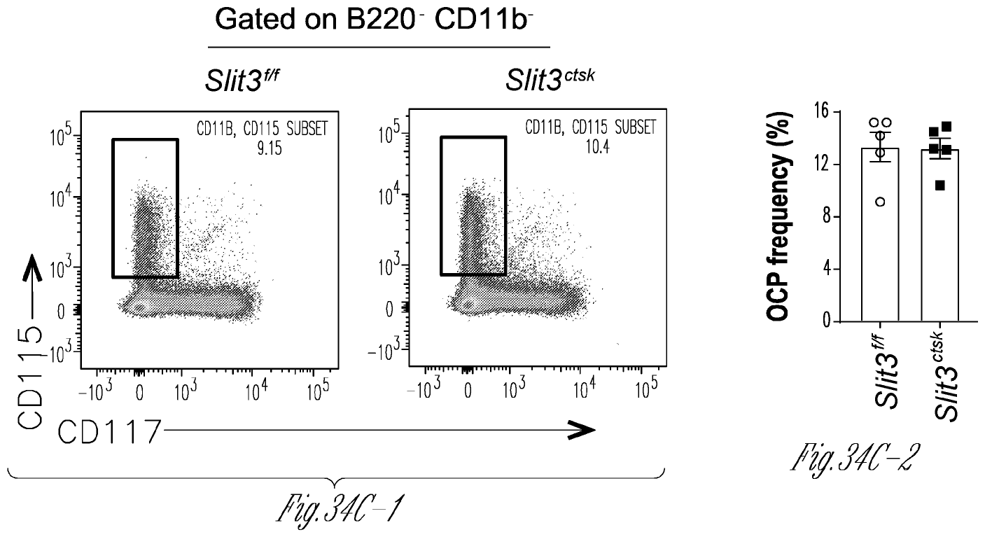

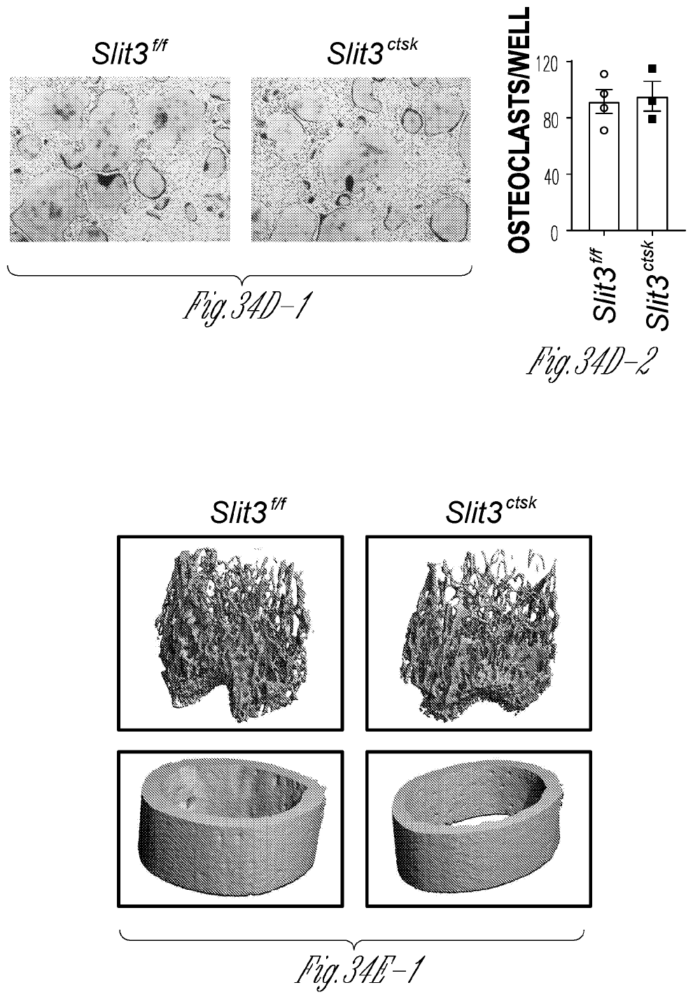

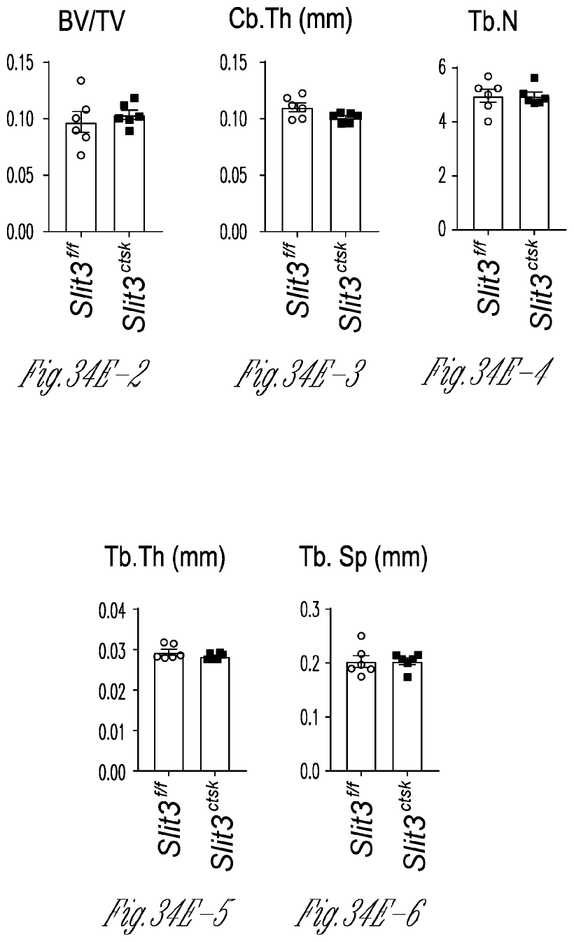

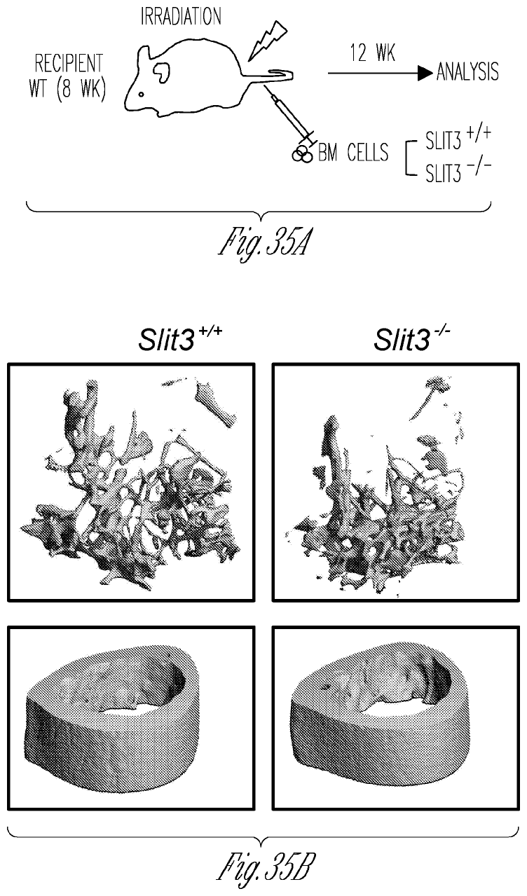

To further assess the role of SLIT3 in osteoclastogenesis in vivo, CD117⁺CD11b^{dim}CD115⁺ osteoclast precursors were first analyzed in Slit3^{-/-} mice using flow cytometry. No significant difference was observed in the overall abundance of osteoclast cells in Slit3^{-/-} mice, indicating that global deletion of SLIT3 is not able to disrupt osteoclast precursors (FIG. 34A). Next, as osteoblast-derived SLIT3 is crucial for bone mass accrual, the inventors designed tests to evaluate whether osteoblast derived SLIT3 would specifically affect osteoclast precursors. Slit3^{f/f} mice were bred to cre-deleter strains targeting osteoblasts via OSX-cre (Slit3^{osx}). Like the observation using animals with global deletion of SLIT3, numbers of osteoclast precursors were unaltered in Slit3^{osx} mice (FIG. 34B). Lastly, to confirm the role of SLIT3 in osteoclasts. Slit3^{f/f} mice were bred to a cre-deleter strain targeting mature osteoclasts, CTSK-cre (Slit3^{ctsk}). No apparent difference was observed in the overall abundance of osteoclast precursors in Slit3^{ctsk} mice, indicating that ablation of SLIT3 production by CTSK positive osteoclasts is not able to disrupt osteoclast precursors (FIG. 34C). Furthermore, osteoclast precursors derived from Slit3^{ctsk} mice and Slit3^{f/f} mice were sorted and an osteoclast formation assay was conducted. No differences in osteoclast production were detected (FIG. 34D). Consistent with this, Slit3^{ctsk} mice developed normally without any gross abnormality and display a normal bone mass relative to control mice at both 5 weeks (FIG. 34E). Consistent with this, histomorphometric analysis showed that mineralization rate, bone formation rate, osteoblast number and osteoclast number were not affected in Slit3^{ctsk} mice. Moreover, the skeletal vasculature was not altered in Slit3^{ctsk} mice relative to Slit3^{f/f} mice as well.

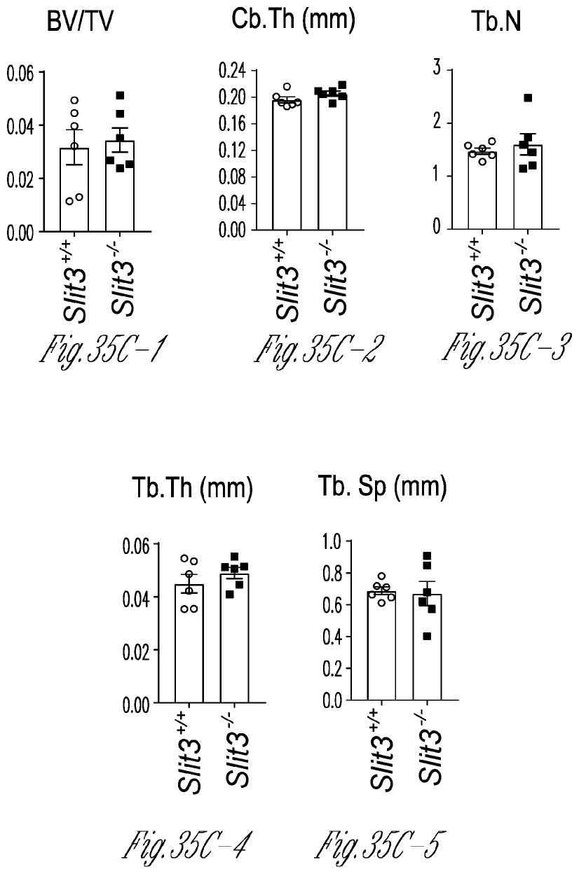

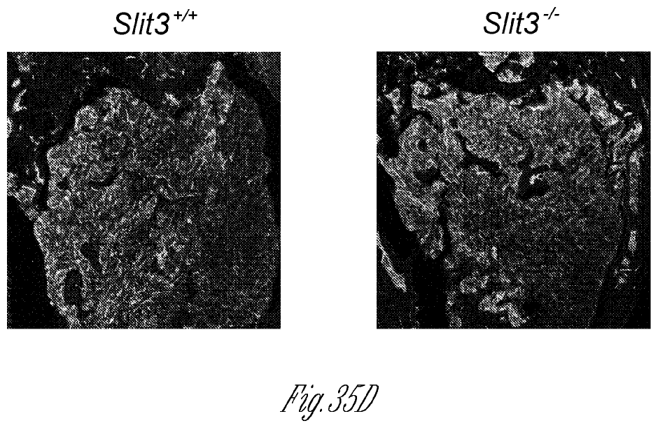

As CathepsinK-cre deletes relatively late during osteoclast differentiation, bone marrow chimeras from Slit3^{-/-} donors were created to assess the importance of SLIT3 production by early stage osteoclast-lineage cells. Bone marrow was collected from 8-week old Slit3^{+/+} mice and Slit3^{-/-} mice and then injected into lethally irradiated 8-week-old WT mice. The resulting chimeras were maintained for 12 weeks before analysis (the protocol is outlined in FIG. 35A). Micro-CT analysis showed that the mice reconstituted with bone marrow cells isolated from Slit3^{-/-} mice had normal bone mass compared with those reconstituted with WT bone marrow cells, as illustrated by the analysis of the trabecular bone volume/total volume and cortical bone thickness (FIG. 35B-35C). Further histomorphometry and immunofluorescence staining showed that neither osteoclast numbers nor bone vasculature were altered by bone marrow transplantation and the absence of SLIT3 from osteoclast lineage cells (FIG. 35D). In summary, both by using conditional deletion of Slit3 in mature osteoclasts and by creating bone marrow chimeras incapable of SLIT3 production in osteoclast-lineage cells, evidence for osteoclasts as a physiologically relevant source of SLIT3 was not observed.

The following statements are potential claims that may be converted to claims in a future application. No modifications of the following statements should be allowed to affect the interpretation of claims which may be drafted when this provisional application is converted into a regular utility application.

### Statements

1) A method for prevention of bone loss or for promoting bone growth, bone-strengthening, or bone-healing in a subject in need thereof, comprising administering a SLIT3 or SLIT2 agent to said subject.
2) The method of statement 1, wherein the SLIT3 or SLIT2 agent is a protein.
3) The method of statement 1, wherein the SLIT3 or SLIT2 agent is a gene transfer vector comprising a promoter operably linked to nucleic segment encoding a SLIT3 or SLIT2 protein.
4) The method of statement 1, 2 or 3, wherein the SLIT3 or SLIT2 is targeted to bone.
5) The method of statement 1-3 or 4, wherein the SLIT3 or SLIT2 is delivered locally.
6) The method of statement 5, wherein said local administration is by injection or via a combination with a medical device.
7) The method of statement 1-5 or 6, wherein bone formation comprises promoting the formation or growth of CD31^{hi}EMCN^{hi} endothelium.
8) The method of statement 1-6 or 7, further comprises administering an agent that inhibits Shn3 expression or SHN3 activity.
9) The method of statement 1-7 or 8, wherein administering is daily, thrice weekly, twice weekly, once weekly, twice monthly, or once monthly.
10) The method of statement 1-7 or 8, wherein administering is every 2 months, every three months, every four months, every five months, every six months, or once per year.
11) The method of statement 1-9 or 10, wherein the SLIT3 or SLIT2 agent is a gene transfer vector comprising a promoter operably linked to nucleic segment encoding a SLIT3 or SLIT2 protein, and administering is once.
12) A method of preventing bone growth in a subject in need thereof, comprising administering a SLIT3 or SLIT2 inhibiting agent to said subject.
13) The method of statement 12, wherein the SLIT3 or SLIT2 inhibiting agent is targeted to bone or delivered locally.
14) The method of statement 12 or 13, wherein the SLIT3 or SLIT2 inhibiting agent is a small interfering RNA.
15) The method of statement 12, 13 or 14 wherein the SLIT3 or SLIT2 inhibiting agent is an antibody.
16) A method comprising locally administering a composition comprising one or more types of SLIT2 or SLIT3 protein, or one or more types of gene transfer vector encoding a SLIT2 or SLIT3 protein to a site where enhanced bone growth or repair is desired in a subject.
17) The method of statement 16, wherein the subject is a mammal or bird.
18) The method of statement 16 or 17, wherein the subject is a human.
19) The method of statement 16, 17, or 18, wherein the subject has substantially no vascular leakage in retinal vasculature from locally administering the SLIT2 or SLIT3 protein, or the SLIT2 or SLIT3 gene transfer vector.
20) The method of statement 16-18 or 19, wherein the composition comprises a carrier or excipient.
21) The method of statement 16-19 or 20, wherein the composition comprises one or more crystalline materials, amorphous materials, liposomes, lipids, charged lipids, DNA-protein complexes, polymers, or a combination thereof.
22) The method of statement 16-20 or 21, wherein the composition comprises gelatin or collagen.
23) The method of statement 16-21 or 22, wherein the composition comprises a sponge.
24) The method of statement 16-22 or 23, wherein the composition coats or is covalently bound to an orthopedic implant.
25) The method of statement 16-23 or 24, wherein the composition is formulated for slow or reduced release.
26) The method of statement 16-24 or 25, which promotes bone growth, strengthens bone, or heals bone the site in the subject.
27) The method of statement 16-25 or 26, which promotes bone growth or strengthens bone at the site in the subject, where the bone growth or bone strength is increased by at least 20% compared to animal that was not administered the SLIT2 or SLIT3 protein, or the SLIT2 or SLIT3 gene transfer vector to a subject.
28) The method of statement 16-26 or 27, which heals bone by increasing maximal load tolerated by a bone in the animal by at least 20% compared to animal that was not administered the SLIT2 or SLIT3 protein, or the SLIT2 or SLIT3 gene transfer vector to a subject.
29) The method of statement 16-27 or 28, wherein the composition comprises about 0.01 mg/kg to about 500 to 750 mg/kg of each SLIT2 protein, SLIT3 protein, gene transfer vector encoding a SLIT2, or gene transfer vector encoding a SLIT3 protein.
30) A method of preventing bone growth in a subject in need thereof, comprising administering a SLIT3 or SLIT2 inhibiting agent to said subject.
31) The method of statement 30, wherein the SLIT3 or SLIT2 inhibiting agent is targeted to bone or delivered locally.
32) The method of statement 30 or 31, wherein the SLIT3 or SLIT2 inhibiting agent is a small interfering RNA that binds to an endogenous SLIT3 or SLIT2 nucleic acid under physiological conditions.
33) The method of statement 30, 31 or 32, wherein the SLIT3 or SLIT2 inhibiting agent is an antibody that binds to SLIT3 protein or SLIT2 protein.
34) The method of statement 30-32 or 33, wherein the SLIT3 or SLIT2 inhibiting agent is trametinib (TTNB).
35) A composition comprising at least one SLIT2 protein, SLIT3 protein, SLIT2 gene transfer vector or SLIT3 gene transfer vector and a carrier or excipient that reduces release or diffusion of the SLIT2 protein, SLIT3 protein, SLIT2 gene transfer vector, or SLIT3 gene transfer vector.
36) The composition of statement 35, wherein the carrier or excipient comprises one or more crystalline materials, amorphous materials, liposomes, lipids, charged lipids, DNA-protein complexes, polymers, or a combination thereof.
37) The composition of statement 35 or 36, wherein the carrier or excipient comprises gelatin or collagen.
38) The composition of statement 35, 36 or 37, wherein the carrier or excipient comprises a sponge.
39) An implant comprising at least one SLIT2 protein, SLIT3 protein, SLIT2 gene transfer vector or SLIT3 gene transfer.
40) The implant of statement 39 which is an orthopedic implant.
41) The implant of statement 39 or 40, wherein the at least one SLIT2 protein, SLIT3 protein, SLIT2 gene transfer vector or SLIT3 gene transfer coats the implant.
42) The implant of statement 39, 40, or 41, wherein the at least one SLIT2 protein, SLIT3 protein, SLIT2 gene transfer vector or SLIT3 gene transfer is covalently bond to the implant.

### REFERENCES

Blockus, H., and A. Chedotal. 2016. “Slit-Robo signaling.” Development 143 (17):3037-44. doi: 10.1242/dev.132829.
Bouxsein, M. L., K. S. Myers. K. L. Shultz, L. R. Donahue, C. J. Rosen, and W. G. Beamer. 2005. “Ovariectomy-induced bone loss varies among inbred strains of mice.” J Bone Miner Res 20 (7): 1085-92. doi: 10.1359/JBMR.050307.
Bradaschia-Correa. V., A. M. Josephson, D. Mehta, M. Mizrahi, S. S. Neibart, C. Liu, O. D. Kennedy, A. B. Castillo, K. A. Egol, and P. Leucht 2017. “The Selective Serotonin Reuptake Inhibitor Fluoxetine Directly Inhibits Osteoblast Differentiation and Mineralization During Fracture Healing in Mice.” J Bone Miner Res 32 (4):821-833. doi: 10.1002/jbmr.3045.
Buza, J. A., 3rd, and T. Einhom. 2016. “Bone healing in 2016.” Clin Cases Miner Bone Metab 13 (2):101-105. doi: 10.11138/ccmbm/2016.13.2.101.
Chen, J., Y. Shi, J. Regan, K. Karuppaiah, D. M. Omitz, and F. Long. 2014. “Osx-Cre targets multiple cell types besides osteoblast lineage in postnatal mice.” PLoS One 9 (1):e85161. doi: 10.1371/journal.pone.0085161.
Chen, M. J., T. Yokomizo, B. M. Zeigler, E. Dzierzak, and N. A. Speck. 2009. “Runx1 is required for the endothelial to haematopoietic cell transition but not thereafter.” Nature 457 (7231):887-91. doi: 10.1038/nature07619.

Choi, H. J., H. Zhang, H. Park, K. S. Choi, H. W. Lee, V. Agrawal, Y. M. Kim, and Y. G. Kwon. 2015. “Yes-associated protein regulates endothelial cell contact-mediated expression of angiopoietin-2.” Nat Commun 6:6943. doi: 10.1038/ncomms7943.
Dempster, D. W., J. E. Compston, M. K. Drezner, F. H. Glorieux, J. A. Kanis, H. Malluche, P. J. Meunier, S. M. Ott, R. R. Recker, and A. M. Parfitt. 2013. “Standardized nomenclature, symbols, and units for bone histomorphometry: a 2012 update of the report of the ASBMR Histomorphometry Nomenclature Committee.” J Bone Miner Res 28 (1):2-17. doi: 10.1002/jbmr.l805.
Dobin, A., C. A. Davis, F. Schlesinger, J. Drenkow, C. Zaleski, S. Jha, P. Batut, M. Chaisson, and T. R. Gingeras. 2013. “STAR: ultrafast universal RNA-seq aligner.” Bioinformatics 29 (1):15-21. doi: 10.1093/bioinformatics/bts635.
Friedlaender, G E; Lin, S; Solchaga, L A; Snel, L B; Lynch, S E (2013). “The role of recombinant human platelet-derived growth factor-BB (rhPDGF-BB) in orthopaedic bone repair and regeneration”. Current pharmaceutical design. 19 (19): 3384-90. PMID 23432673.
Fukuda, T., S. Takeda. R. Xu, H. Ochi, S. Sunamura, T. Sato, S. Shibata, Y. Yoshida, Z. Gu, A. Kimura, C. Ma, C. Xu, W. Bando, K. Fujita, K. Shinomiya, T. Hirai. Y. Asou, M. Enomoto, H. Okano, A. Okawa, and H. Itoh. 2013. “Sema3A regulates bone-mass accrual through sensory innervations.” Nature 497 (7450):490-3. doi: 10.1038/nature12115.
Geutskens, S. B., W. D. Andrews. A. M. van Stalborch, K. Brussen. S. E. Holtrop-de Haan. J. G. Parnavelas, P. L. Hordijk, and P. B. van Hennik. 2012. “Control of human hematopoietic stem/progenitor cell migration by the extracellular matrix protein Slit3.” Lab Invest 92 (8): 1129-39. doi: 10.1038/labinvest.2012.81.
Geutskens, S. B., P. L Hordijk, and P. B. van Hennik. 2010. “The chemorepellent Slit3 promotes monocyte migration.” J Immunol 185 (12):7691-8. doi: 10.4049/jimmunol.0903898.
Greenblatt, M. B., D. Y. Shin, H. Oh, K. Y. Lee, B. Zhai, S. P. Gygi, S. Lotinun, R. Baron, D. Liu, B. Su, L. H. Glimcher, and J. H. Shim. 2016. “MEKK2 mediates an alternative beta-catenin pathway that promotes bone formation.” Proc Natl Acad Sci USA 113 (9):E1226-35. doi: 10.1073/pnas.1600813113.
Guan, M., W. Yao, R. Liu, K. S. Lam, J. Nolta, J. Jia, B. Panganiban, L. Meng, P. Zhou, M. Shahnazari, R. O. Ritchie, and N. E. Lane. 2012. “Directing mesenchymal stem cells to bone to augment bone formation and increase bone mass.” Nat Med 18 (3):456-62. doi: 10.1038/nm.2665.
Howitt, J. A., N. J. Clout, and E. Hohenester. 2004. “Binding site for Robo receptors revealed by dissection of the leucine-rich repeat region of Slit.” EMBO J 23 (22):4406-12. doi: 10.1038/sj.emboj.7600446.
Huang da, W., B. T. Sherman, and R. A. Lempicki. 2009. “Systematic and integrative analysis of large gene lists using DAVID bioinformatics resources.” Nat Protoc 4 (1):44-57. doi: 10.1038/nprot.2008.211.
Jaworski, A., and M. Tessier-Lavigne. 2012. “Autocrine/juxtaparacrine regulation of axon fasciculation by Slit-Robo signaling.” Nat Neurosci 15 (3):367-9. doi: 10.1038/nn.3037.
Jones, C. A., N. R. London, H. Chen, K. W. Park, D. Sauvaget, R. A. Stockton, J. D. Wythe, W. Suh, F. Larrieu-Lahargue, Y. S. Mukouyama, P. Lindblom, P. Seth, A. Frias, N. Nishiya, M. H. Ginsberg, H. Gerhardt, K. Zhang, and D. Y. Li. 2008. “Robo4 stabilizes the vascular network by inhibiting pathologic angiogenesis and endothelial hyperpermeability.” Nat Med 14 (4):448-53. doi: 10.1038/nm1742.
Jones, D. C., M. N. Wein, M. Oukka, J. G. Hofstaetter, M. J. Glimcher, and L. H. Glimcher. 2006. “Regulation of adult bone mass by the zinc finger adapter protein Schnurri-3.” Science 312 (5777): 1223-7. doi: 10.1126/science.1126313.
Kusumbe, A. P., S. K. Ramasamy, and R. H. Adams. 2014. “Coupling of angiogenesis and osteogenesis by a specific vessel subtype in bone.” Nature 507 (7492):323-8. doi: 10.1038/nature13145.

Leder. B. Z., J. N. Tsai, A. V. Uihlein. P. M. Wallace, H. Lee, R. M. Neer, and S. A. Burnett-Bowie. 2015. “Denosumab and teriparatide transitions in postmenopausal osteoporosis (the DATA-Switch study): extension of a randomised controlled trial.” Lancet 386 (9999):1147-55. doi: 10.1016/S0140-6736(15)61120-5.
Liang, C., B. Guo, H. Wu, N. Shao, D. Li, J. Liu, L. Dang, C. Wang, H. Li, S. Li, W. K. Lau, Y. Cao, Z. Yang, C. Lu, X. He, D. W. Au, X. Pan, B. T. Zhang, C. Lu, H. Zhang, K. Yue, A. Qian, P. Shang, J. Xu, L Xiao, Z. Bian, W. Tan, Z. Liang, F. He, L. Zhang, A. Lu, and G. Zhang. 2015. “Aptamer-functionalized lipid nanoparticles targeting osteoblasts as a novel RNA interference-based bone anabolic strategy.” Nat Med 21 (3):288-94. doi: 10.1038/nm.3791.
Liao, Y., G. K. Smyth, and W. Shi. 2014. “featureCounts: an efficient general purpose program for assigning sequence reads to genomic features.” Bioinformatics 30 (7):923-30. doi: 10.1093/bioinformatics/btt656.
Liu, Z., K. Patel, H. Schmidt, W. Andrews, A. Pini, and V. Sundaresan. 2004. “Extracellular Ig domains 1 and 2 of Robo are important for ligand (Slit) binding.” Mol Cell Neurosci 26 (2):232-40. doi: 10.1016/j.mcn.2004.01.002.
Love, M. I., W. Huber, and S. Anders. 2014. “Moderated estimation of fold change and dispersion for RNA-seq data with DESeq2.” Genome Biol 15 (12):550. doi: 10.1186/s13059-014-0550-8.
Lu. Y., Y. Xie, S. Zhang, V. Dusevich, L. F. Boncwald, and J. Q. Feng. 2007. “DMP1-targeted Cre expression in odontoblasts and osteocytes.” J Dent Res 86 (4):320-5. doi: 10.1177/154405910708600404.
Mehlen, P., C. Delloye-Bourgeois, and A. Chedotal. 2011. “Novel roles for Slits and netrins: axon guidance cues as anticancer targets?” Nat Rev Cancer 11 (3): 188-97. doi: 10.1038/nrc3005.
Naska, S., D. C. Lin, F. D. Miller, and D. R. Kaplan. 2010. “p75NTR is an obligate signaling receptor required for cues that cause sympathetic neuron growth cone collapse.” Mol Cell Neurosci 45 (2): 108-20. doi: 10.1016/j.men.2010.05.015.
Nguyen Ba-Charvet, K. T., K. Brose, V. Marillat, T. Kidd, C. S. Goodman, M. Tessier-Lavigne, C. Sotelo, and A. Chedotal. 1999. “Slit2-Mediated chemorepulsion and collapse of developing forebrain axons.” Neuron 22 (3):463-73.
Park, D., J. A. Spencer, B. I. Koh, T. Kobayashi, J. Fujisaki, T. L Clemens, C. P. Lin, H. M. Kronenberg, and D. T. Scadden. 2012. “Endogenous bone marrow MSCs are dynamic, fate-restricted participants in bone maintenance and regeneration.” Cell Stem Cell 10 (3):259-72. doi: 10.1016/j.stem.2012.02.003.
Paul, J. D., K. L K. Coulombe, P. T. Toth, Y. Zhang, G. Marsboom, V. P. Bindokas, D. W. Smith, C. E. Murry, and J. Rehman. 2013. “SLIT3-R0B04 activation promotes vascular network formation in human engineered tissue and angiogenesis in vivo.” J Mol Cell Cardiol 64:124-31. doi: 10.1016/j.yjmcc.2013.09.005.
Rama, N., A. Dubrac, T. Mathivet, R. A. Ni Charthaigh, G. Genet, B. Cristofaro, L. Pibouin-Fragner, L. Ma, A. Eichmann, and A. Chedotal. 2015. “Slit2 signaling through Robo1 and Robo2 is required for retinal neovascularization.” Nat Med 21 (5):483-91. doi: 10.1038/nm.3849.
Ramalingam, P., M. G. Poulos, and J. M. Butler. 2017. “Regulation of the hematopoietic stem cell lifecycle by the endothelial niche.” Curr Opin Hematol 24 (4):289-299. doi: 10.1097/MGH.0000000000000350.
Ramasamy, S. K., A. P. Kusumbe, L. Wang, and R. H. Adams. 2014. “Endothelial Notch activity promotes angiogenesis and osteogenesis in bone.” Nature 507 (7492):376-80. doi: 10.1038/nature13146.
Rodda, S. J., and A. P. McMahon. 2006. “Distinct roles for Hedgehog and canonical Wnt signaling in specification, differentiation and maintenance of osteoblast progenitors.” Development 133 (16):3231-44. doi: 10.1242/dev.02480.
Sakabe, M., J. Fan, Y. Odaka, N. Liu, A. Hasson, X. Duan, P. Stump, L. Byerly, M. Donaldson, J. Hao, M. Fruttiger, Q. R. Lu, Y. Zheng, R. A. Lang, and M. Xin. 2017. “YAP/TAZ-CDC42 signaling regulates vascular tip cell migration.” Proc Natl Acad Sci US A 114 (41):10918-10923. doi: 10.1073/pnas.1704030114.
Shah, P; Keppler, L; Rutkowski, J (December 2012). “Bone morphogenic protein: an elixir for bone grafting—a review”. The Journal of oral implantology. 38 (6): 767-78. doi: 10.1563/AAID-JOI-D-10-00196. PMID 21574851.
Shim, J. H., M. B. Greenblatt, W. Zou, Z. Huang, M. N. Wein, N. Brady, D. Hu, J. Charron, H. R. Brodkin, G. A. Petsko, D. Zaller, B. Zhai, S. Gygi, L. H. Glimcher, and D. C. Jones. 2013. “Schnurri-3 regulates ERK downstream of WNT signaling in osteoblasts.” J Clin Invest 123 (9):4010-22. doi: 10.1172/JCI69443.
Shin, M., T. J. Beane, A. Quillien, I. Male, L. J. Zhu, and N. D. Lawson. 2016. “Vegfa signals through ERK to promote angiogenesis, but not artery differentiation.” Development 143 (20):3796-3805. doi: 10.1242/dev.137919.
Su, A. I., T. Wiltshire, S. Batalov, H. Lapp, K. A. Ching, D. Block. J. Zhang, R. Soden, M. Hayakawa, G. Kreiman, M. P. Cooke, J. R. Walker, and J. B. Hogencsch. 2004. “A gene atlas of the mouse and human protein-encoding transcriptomes.” Proc Natl Acad Sri USA 101 (16):6062-7. doi: 10.1073/pnas.0400782101.
Sun, H., K. Dai, T. Tang, and X. Zhang. 2009. “Regulation of osteoblast differentiation by slit2 in osteoblastic cells.” Cells Tissues Organs 190 (2):69-80. doi: 10.1159/000178020.
Vasam, G., S. Joshi, S. E. Thatcher, S. H. Bartelmez, L. A. Cassis, and Y. P. Jarajapu. 2017. “Reversal of Bone Marrow Mobilopathy and Enhanced Vascular Repair by Angiotensin-(1-7) in Diabetes.” Diabetes 66 (2):505-518. doi: 10.2337/db16-1039.
Wein, M. N., D. C. Jones, J. H. Shim, A. O. Aliprantis, R. Sulyanto, V. Lazarevic, S. L. Poliadnk, T. S. Gross, and L. H. Glimcher. 2012. “Control of bone resorption in mice by Schnurri-3.” Proc Natl Acad Sri USA 109 (21):8173-8. doi: 10.1073/pnas.1205848109.
Xie, H., Z. Cui, L. Wang, Z. Xia, Y. Hu, L Xian, C. Li, L. Xie, J. Crane, M. Wan, G. Zhen, Q. Bian, B. Yu, W. Chang. T. Qiu, M. Pickarski, L. T. Duong. J. J. Windle, X. Luo, E. Liao, and X. Cao. 2014. “PDGF-BB secreted by preosteoclasts induces angiogenesis during coupling with osteogenesis.” Nat Med 20 (11): 1270-8. doi: 10.1038/nm.3668.
Xu, R. 2014. “Semaphorin 3A: A new player in bone remodeling.” Cell Adh Migr 8 (1):5-10. doi: 10.4161/cam.27752.
Xu, R., C. Zhang. D. Y. Shin, J. M. Kim. S. Lalani, N. Li, Y. S. Yang. Y. Liu, M. Eiseman, R. J. Davis, J. H. Shim, and M. B. Greenblatt. 2017a. “c-Jun N-Terminal Kinases (JNKs) Are Critical Mediators of Osteoblast Activity In Vivo.” J Bone Miner Res 32 (9):1811-1815. doi: 10.1002/jbmr.3184.
Xu, R., C. Zhang, D. Y. Shin, J. M. Kim, S. Lalani, N. Li, Y. S. Yang, Y. Liu, M. Eiseman, R. J. Davis, J. H. Shim, and M. B. Greenblatt. 2017b. “c-Jun N-terminal kinases (JNKs) are critical mediators of osteoblast activity in vivo.” J Bone Miner Res. doi: 10.1002/jbmr.3184.
Yao, W., M. Guan, J. Jia, W. Dai, Y. A. Lay, S. Amugongo, R. Liu, D. Olivos, M. Saunders, K. S. Lain, J. Nolta, D. Olvera, R. O. Ritchie, and N. E. Lane. 2013. “Reversing bone loss by directing mesenchymal stem cells to bone.” Stem Cells 31 (9):2003-14. doi: 10.1002/stem.1461.
Yuan, W., Y. Rao, R. P. Babiuk, J. J. Greer, J. Y. Wu, and D. M. Ornitz. 2003. “A genetic model for a central (septum transversum) congenital diaphragmatic hernia in mice lacking Slit3.” Proc Natl Acad Sci USA 100 (9):5217-22. doi: 10.1073/pnas.0730709100.
Zhang, B., U. M. Dietrich, J. G. Geng, R. Bicknell, J. D. Esko, and L. Wang. 2009. “Repulsive axon guidance molecule Slit3 is a novel angiogenic factor.” Blood 114 (19):4300-9. doi: 10.1182/blood-2008-12-193326.
Zhang, C., K. Hampers. A. Kuchinsky, N. Salomonis, D. Xu, and A. R. Pico. 2012. “Mosaic: making biological sense of complex networks.” Bioinformatics 28 (14): 1943-4. doi: 10.1093/bioinformatics/bts278.
Zhang, G., B. Guo, H. Wu, T. Tang, B. T. Zhang, L. Zheng, Y. He, Z. Yang, X. Pan, H. Chow, K. To, Y. Li, D. Li, X Wang, Y. Wang, K. Lee, Z. Hou, N. Dong, G. Li, K. Leung, L. Hung, F. He, L. Zhang, and L. Qin. 2012. “A delivery system targeting bone formation surfaces to facilitate RNAi-based anabolic therapy.” Nat Med 18 (2):307-14. doi: 10.1038/nm.2617.
Zhang, J., and D. C. Link. 2016. “Targeting of Mesenchymal Stromal Cells by Cre-Recombinase Transgenes Commonly Used to Target Osteoblast Lineage Cells.” J Bone Miner Res 31 (11):2001-2007. doi: 10.1002/jbmr.2877.
Zhang, Y., J. Xu. Y. C. Ruan, M. K. Yu, M. O'Laughlin, H. Wise, D. Chen, L. Tian, D. Shi, J. Wang. S. Chen, J. Q. Feng, D. H. Chow, X. Xie, L. Zheng, L. Huang, S. Huang, K. Leung, N. Lu, L Zhao, H. Li, D. Zhao, X. Guo, K. Chan, F. Witte, H. C. Chan, Y. Zheng, and L. Qin. 2016. “Implant-derived magnesium induces local neuronal production of CGRP to improve bone-fracture healing in rats.” Nat Med 22 (10): 1160-1169. doi: 10.1038/nm.4162.
Zhou, W. J., Z. H. Geng. J. R. Spence, and J. G. Geng. 2013. “Induction of intestinal stem cells by R-spondin 1 and Slit2 augments chemoradioprotection.” Nature 501 (7465): 107-11. doi: 10.1038/nature12416.
Kraszka P. Tanpaiboon P, Neas K. Crosby K, Berger S I, Martinez A F, et al. Loss of function in ROBO1 is associated with tetralogy of Fallot and septal defects. J Med Genet. 2017;
Mori 11-Poulard I, Sharma A, Louradour I, Vanzo N, Vincent A, Crozatier M. Vascular control of the Drosophila haematopoietic microenvironment by Slit/Robo signalling. Nat Commun. 2016; 7:11634.
Jaworski A. Tessier-Lavigne M. Autocrine/juxtaparacrine regulation of axon fasciculation by Slit-Robo signaling. Nat Neurosci [Internet]. 2012; 15:367-9. Available from: http://www.ncbi.nlm.nih.gov/pubmed/22306607
Kidd T, Brose K, Mitchell K J, Fetter R D, Tessier-Lavigne M, Goodman C S, et al. Roundabout controls axon crossing of the CNS midline and defines a novel subfamily of evolutionarily conserved guidance receptors. Cell. 1998:92:205-15.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific proteins, nucleic acids, methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential.

The specific proteins, nucleic acids, methods and compositions illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a protein,” “a nucleic add,” “a composition,” “a vector” or “a promoter” includes a plurality of such proteins, nucleic acids, compositions, vectors or promoters (for example, a solution of proteins, nucleic acids, compositions, vectors, or a series of promoters), and so forth.

The term “about”, as used herein, can allow for a degree of variability in a value or range, for example, within 10%, within 5%, or within 1% of a stated value or of a stated limit of a range.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

What is claimed:

1. A method comprising locally administering a composition comprising one or more types of SLIT2 or SLIT3 protein, or one or more types of gene transfer vector encoding a SLIT2 or SLIT3 protein to a site where enhanced bone growth or repair is desired in a subject.

2. The method of claim 1, wherein the subject is a mammal or bird.

3. The method of claim 1, wherein the subject is a human.

4. The method of claim 1, wherein the subject has substantially no vascular leakage in retinal vasculature from locally administering the SLIT2 or SLIT3 protein, or the SLIT2 or SLIT3 gene transfer vector.

5. The method of claim 1, wherein the composition comprises a carrier or excipient.

6. The method of claim 1, wherein the gene transfer vector comprises a viral vector.

7. The method of claim 1, wherein the composition comprises one or more crystalline materials, amorphous materials, liposomes, lipids, charged lipids, DNA-protein complexes, polymers, or a combination thereof.

8. The method of claim 1, wherein the composition comprises gelatin or collagen.

9. The method of claim 1, wherein the composition comprises a sponge.

10. The method of claim 1, wherein the composition coats or is covalently bound to an orthopedic implant.

11. The method of claim 1, wherein the composition is formulated for slow or reduced release.

12. The method of claim 1, which promotes bone growth, strengthens bone, or heals bone the site in the subject.

13. The method of claim 1, which promotes bone growth or strengthens bone at the site in the subject, where the bone growth or bone strength is increased by at least 20% compared to animal that was not administered the SLIT2 or SLIT3 protein, or the SLIT2 or SLIT3 gene transfer vector to a subject.

14. The method of claim 1, which heals bone by increasing maximal load tolerated by a bone in the animal by at least 20% compared to animal that was not administered the SLIT2 or SLIT3 protein, or the SLIT2 or SLIT3 gene transfer vector to a subject.

15. The method of claim 1, wherein the composition comprises about 0.01 mg/kg to about 500 to 750 mg/kg of each SLIT2 protein, SLIT3 protein, gene transfer vector encoding a SLIT2, or gene transfer vector encoding a SLIT3 protein.

16. A method of preventing bone growth in a subject in need thereof, comprising administering a SLIT3 or SLIT2 inhibiting agent to said subject.

17. The method of claim 16, wherein the SLIT3 or SLIT2 inhibiting agent is targeted to bone or delivered locally.

18. The method of claim 16, wherein the SLIT3 or SLIT2 inhibiting agent is a small interfering RNA that binds to an endogenous SLIT3 or SLIT2 nucleic acid under physiological conditions.

19. The method of claim 16, wherein the SLIT3 or SLIT2 inhibiting agent is an antibody that binds to SLIT3 protein or SLIT2 protein.

20. The method of claim 16, wherein the SLIT3 or SLIT2 inhibiting agent is trametinib (TTNB).


SEQUENCE LISTING (9 sequences)

SEQ ID NO: 1 - protein, 1523 aa, Homo sapiens [sequence omitted]

SEQ ID NO: 2 - DNA, 4842 bp, Homo sapiens
cgcgctccccgcgcgcctcctcgggctccacgcgtcttgccccgcagaggcagcctcctccaggagcggggccctgcacaccatggcccccgggtgggcaggggtcggcgccgccgtgcgcgcccgcctggcgctggccttggcgctggcgagcgtcctgagtgggcctccagccgtcgcctgccccaccaagtgtacctgctccgctgccagcgtggactgccacgggctgggcctccgcgcggttcctcggggcatcccccgcaacgctgagcgccttgacctggacagaaataatatcaccaggatcaccaagatggacttcgctgggctcaagaacctccgagtcttgcatctggaagacaaccaggtcagcgtcatcgagagaggcgccttccaggacctgaagcagctagagcgactgcgcctgaacaagaataagctgcaagtccttccagaattgcttttccagagcacgccgaagctcaccagactagatttgagtgaaaaccagatccaggggatcccgaggaaggcgttccgcggcatcaccgatgtgaagaacctgcaactggacaacaaccacatcagctgcattgaagatggagccttccgagcgctgcgcgatttggagatccttaccctcaacaacaacaacatcagtcgcatcctggtcaccagcttcaaccacatgccgaagatccgaactctgcgcctccactccaaccacctctactgcgactgccacctggcctggctctcggattggctgcgacagcgacggacagttggccagttcacactctgcatggctcctgtgcatttgaggggcttcaacgtggcggatgtgcagaagaaggagtacgtgtgcccagccccccactcggagcccccatcctgcaatgccaactccatctcctgcccttcgccctgcacgtgcagcaataacatcgtggactgtcgaggaaagggcttgatggagattcctgccaacttgccggagggcatcgtcgaaatacgcctagaacagaactccatcaaagccatccctgcaggagccttcacccagtacaagaaactgaagcgaatagacatcagcaagaatcagatatcggatattgctccagatgccttccagggcctgaaatcactcacatcgctggtcctgtatgggaacaagatcaccgagattgccaagggactgtttgatgggctggtgtccctacagctgctcctcctcaatgccaacaagatcaactgcctgcgggtgaacacgtttcaggacctgcagaacctcaacttgctctccctgtatgacaacaagctgcagaccatcagcaaggggctcttcgcccctctgcagtccatccagacactccacttagcccaaaacccatttgtgtgcgactgccacttgaagtggctggccgactacctccaggacaaccccatcgagacaagcggggcccgctgcagcagcccgcgccgactcgccaacaagcgcatcagccagatcaagagcaagaagttccgctgctcaggctccgaggattaccgcagcaggttcagcagcgagtgcttcatggacctcgtgtgccccgagaagtgtcgctgtgagggcacgattgtggactgctccaaccagaagctggtccgcatcccaagccacctccctgaatatgtcaccgacctgcgactgaatgacaatgaggtatctgttctggaggccactggcatcttcaagaagttgcccaacctgcggaaaataaatctgagtaacaataagatcaaggaggtgcgagagggagctttcgatggagcagccagcgtgcaggagctgatgctgacagggaaccagctggagaccgtgcacgggcgcgtgttccgtggcctcagtggcctcaaaaccttgatgctgaggagtaacttgatcagctgtgtgagtaatgacacctttgccggcctgagttcggtgagactgctgtccctctatgacaatcggatcaccaccatcacccctggggccttcaccacgcttgtctccctgtccaccataaacctcctgtccaaccccttcaactgcaactgccacctggcctggctcggcaagtggttgaggaagaggcggatcgtcagtgggaaccctaggtgccagaagccatttttcctcaaggagattcccatccaggatgtggccatccaggacttcacctgtgatggcaacgaggagagtagctgccagctgagcccgcgctgcccggagcagtgcacctgtatggagacagtggtgcgatgcagcaacaaggggctccgcgccctccccagaggcatgcccaaggatgtgaccgagctgtacctggaaggaaaccacctaacagccgtgcccagagagctgtccgccctccgacacctgacgcttattgacctgagcaacaacagcatcagcatgctgaccaattacaccttcagtaacatgtctcacctctccactctgatcctgagctacaaccggctgaggtgcatccccgtccacgccttcaacgggctgcggtccctgcgagtgctaaccctccatggcaatgacatttccagcgttcctgaaggctccttcaacgacctcacatctctttcccatctggcgctgggaaccaacccactccactgtgactgcagtcttcggtggctgtcggagtgggtgaaggcggggtacaaggagcctggcatcgcccgctgcagtagccctgagcccatggctgacaggctcctgctcaccaccccaacccaccgcttccagtgcaaagggccagtggacatcaacattgtggccaaatgcaatgcctgcctctccagcccgtgcaagaataacgggacatgcacccaggaccctgtggagctgtaccgctgtgcctgcccctacagctacaagggcaaggactgcactgtgcccatcaacacctgcatccagaacccctgtcagcatggaggcacctgccacctgagtgacagccacaaggatgggttcagctgctcctgccctctgggctttgaggggcagcggtgtgagatcaacccagatgactgtgaggacaacgactgcgaaaacaatgccacctgcgtggacgggatcaacaactacgtgtgtatctgtccgcctaactacacaggtgagctatgcgacgaggtgattgaccactgtgtgcctgagctgaacctctgtcagcatgaggccaagtgcatccccctggacaaaggattcagctgcgagtgtgtccctggctacagcgggaagctctgtgagacagacaatgatgactgtgtggcccacaagtgccgccacggggcccagtgcgtggacacaatcaatggctacacatgcacctgcccccagggcttcagtggacccttctgtgaacaccccccacccatggtcctactgcagaccagcccatgcgaccagtacgagtgccagaacggggcccagtgcatcgtggtgcagcaggagcccacctgccgctgcccaccaggcttcgccggccccagatgcgagaagctcatcactgtcaacttcgtgggcaaagactcctacgtggaactggcctccgccaaggtccgaccccaggccaacatctccctgcaggtggccactgacaaggacaacggcatccttctctacaaaggagacaatgaccccctggcactggagctgtaccagggccacgtgcggctggtctatgacagcctgagttcccctccaaccacagtgtacagtgtggagacagtgaatgatgggcagtttcacagtgtggagctggtgacgctaaaccagaccctgaacctagtagtggacaaaggaactccaaagagcctggggaagctccagaagcagccagcagtgggcatcaacagccccctctaccttggaggcatccccacctccaccggcctctccgccttgcgccagggcacggaccggcctctaggcggcttccacggatgcatccatgaggtgcgcatcaacaacgagctgcaggacttcaaggccctcccaccacagtccctgggggtgtcaccaggctgcaagtcctgcaccgtgtgcaagcacggcctgtgccgctccgtggagaaggacagcgtggtgtgcgagtgccgcccaggctggaccggcccactctgcgaccaggaggcccgggacccctgcctcggccacagatgccaccatggaaaatgtgtggcaactgggacctcatacatgtgcaagtgtgccgagggctatggaggggacttgtgtgacaacaagaatgactctgccaatgcctgctcagccttcaagtgtcaccatgggcagtgccacatctcagaccaaggggagccctactgcctgtgccagcccggctttagcggcgagcactgccaacaagagaatccgtgcctgggacaagtagtccgagaggtgatccgccgccagaaaggttatgcatcatgtgccacagcctccaaggtgcccatcatggaatgtcgtgggggctgtgggccccagtgctgccagcccacccgcagcaagcggcggaaatacgtcttccagtgcacggacggctcctcgtttgtagaagaggtggagagacacttagagtgcggctgcctcgcgtgttcctaagcccctgcccgcctgcctgccacctctcggactccagcttgatggagttgggacagccatgtgggaccccctggtgattcagcatgaaggaaatgaagctggagaggaaggtaaagaagaagagaatattaagtatattgtaaaataaacaaaaaatagaacttaaaaaaaaaaaaaaaaaaaaaaaa

SEQ ID NO: 3 - protein, 1523 aa, Homo sapiens
MAPGWAGVGAAVRARLALALALASVLSGPPAVACPTKCTCSAASVDCHGLGLRAVPRGIPRNAERLDLDRNNITRITKMDFAGLKNLRVLHLEDNQVSVIERGAFQDLKQLERLRLNKNKLQVLPELLFQSTPKLTRLDLSENQIQGIPRKAFRGITDVKNLQLDNNHISCIEDGAFRALRDLEILTLNNNNISRILVTSFNHMPKIRTLRLHSNHLYCDCHLAWLSDWLRQRRTVGQFTLCMAPVHLRGFNVADVQKKEYVCPAPHSEPPSCNANSISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGSEDYRSRFSSECFMDLVCPEKCRCEGTIVDCSNQKLVRIPSHLPEYVTDLRLNDNEVSVLEATGIFKKLPNLRKINLSNNKIKEVREGAFDGAASVQELMLTGNQLETVHGRVFRGLSGLKTLMLRSNLIGCVSNDTFAGLSSVRLLSLYDNRITTITPGAFTTLVSLSTINLLSNPFNCNCHLAWLGKWLRKRRIVSGNPRCQKPFFLKEIPIQDVAIQDFTCDGNEESSCQLSPRCPEQCTCMETVVRCSNKGLRALPRGMPKDVTELYLEGNHLTAVPRELSALRHLTLIDLSNNSISMLTNYTFSNMSHLSTLILSYNRLRCIPVHAFNGLRSLRVLTLHGNDISSVPEGSFNDLTSLSHLALGTNPLHCDCSLRWLSEWVKAGYKEPGIARCSSPEPMADRLLLTTPTHRFQCKGPVDINIVAKCNACLSSPCKNNGTCTQDPVELYRCACPYSYKGKDCTVPINTCIQNPCQHGGTCHLSDSHKDGFSCSCPLGFEGQRCEINPDDCEDNDCENNATCVDGINNYVCICPPNYTGELCDEVIDHCVPELNLCQHEAKCIPLDKGFSCECVPGYSGKLCETDNDDCVAHKCRHGAQCVDTINGYTCTCPQGFSGPFCEHPPPMVLLQTSPCDQYECQNGAQCIVVQQEPTCRCPPGFAGPRCEKLITVNFVGKDSYVELASAKVRPQANISLQVATDKDNGILLYKGDNDPLALELYQGHVRLVYDSLSSPPTTVYSVETVNDGQFHSVELVTLNQTLNLVVDKGTPKSLGKLQKQPAVGINSPLYLGGIPTSTGLSALRQGTDRPLGGFHGCIHEVRINNELQDFKALPPQSLGVSPGCKSCTVCKHGLCRSVEKDSVVCECRPGWTGPLCDQEARDPCLGHRCHHGKCVATGTSYMCKCAEGYGGDLCDNKNDSANACSAFKCHHGQCHISDQGEPYCLCQPGFSGEHCQQENPCLGQVVREVIRRQKGYASCATASKVPIMECRGGCGPQCCQPTRSKRRKYVFQCTDGSSFVEEVERHLECGCLACS

SEQ ID NO: 4 - protein, 1460 aa, Homo sapiens
MMGDLDRNNITRITKMDFAGLKNLRVLHLEDNQVSVIERGAFQDLKQLERLRLNKNKLQVLPELLFQSTPKLTRLDLSENQIQGIPRKAFRGITDVKNLQLDNNHISCIEDGAFRALRDLEILTLNNNNISRILVTSFNHMPKIRTLRLHSNHLYCDCHLAWLSDWLRQRRTVGQFTLCMAPVHLRGFNVADVQKKEYVCPAPHSEPPSCNANSISCPSPCTCSNNIVDCRGKGLMEIPANLPEGIVEIRLEQNSIKAIPAGAFTQYKKLKRIDISKNQISDIAPDAFQGLKSLTSLVLYGNKITEIVKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGSEDYRSRFSSECFMDLVCPEKCRCEGTIVDCSNQKLVRIPSHLPEYVTDLRLNDNEVSVLEATGIFKKLPNLRKINLSNNKIKEVREGAFDGAASVQELMLTGNQLETVHGRVFRGLSGLKTLMLRSNLIGCVSNDTFAGLSSVRLLSLYDNRITTITPGAFTTLVSLSTINLLSNPFNCNCHLAWLGKWLRKRRIVSGNPRCQKPFFLKEIPIQDVAIQDFTCDGNEESSCQLSPRCPEQCTCMETVVRCSNKGLRALPRGMPKDVTELYLEGNHLTAVPRELSALRHLTLIDLSNNSISMLTNYTFSNMSHLSTLILSYNRLRCIPVHAFNGLRSLRVLTLHGNDISSVPEGSFNDLTSLSHLALGTNPLHCDCSLRWLSEWVKAGYKEPGIARCSSPEPMADRLLLTTPTHRFQCKGPVDINIVAKCNACLSSPCKNNGTCTQDPVELYRCACPYSYKGKDCTVPINTCIQNPCQHGGTCHLSDSHKDGFSCSCPLGFEGQRCEINPDDCEDNDCENNATCVDGINNYVCICPPNYTGELCDEVIDHCVPELNLCQHEAKCIPLDKGFSCECVPGYSGKLCETDNDDCVAHKCRHGAQCVDTINGYTCTCPQGFSGPFCEHPPPMVLLQTSPCDQYECQNGAQCIVVQQEPTCRCPPGFAGPRCEKLITVNFVGKDSYVELASAKVRPQANISLQVATDKDNGILLYKGDNDPLALELYQGHVRLVYDSLSSPPTTVYSVETVNDGQFHSVELVTLNQTLNLVVDKGTPKSLGKLQKQPAVGINSPLYLGGIPTSTGLSALRQGTDRPLGGFHGCIHEVRINNELQDFKALPPQSLGVSPGCKSCTVCKHGLCRSVEKDSVVCECRPGWTGPLCDQEARDPCLGHRCHHGKCVATGTSYMCKCAEGYGGDLCDNKNDSANACSAFKCHHGQCHISDQGEPYCLCQPGFSGEHCQQENPCLGQVVREVIRRQKGYASCATASKVPIMECRGGCGPQCCQPTRSKRRKYVFQCTDGSSFVEEVERHLECGCLACS

SEQ ID NO: 5 - protein, 1525 aa, Homo sapiens [sequence omitted]

SEQ ID NO: 6 - DNA, 4758 bp, Homo sapiens
atgcgcggcgttggctggcagatgctgtccctgtcgctggggttagtgctggcgatcctgaacaaggtggcaccgcaggcgtgcccggcgcagtgctcttgctcgggcagcacagtggactgtcacgggctggcgctgcgcagcgtgcccaggaatatcccccgcaacaccgagagactggatttaaatggaaataacatcacaagaattacgaagacagattttgctggtcttagacatctaagagttcttcagcttatggagaataagattagcaccattgaaagaggagcattccaggatcttaaagaactagagagactgcgtttaaacagaaatcaccttcagctgtttcctgagttgctgtttcttgggactgcgaagctatacaggcttgatctcagtgaaaaccaaattcaggcaatcccaaggaaagctttccgtggggcagttgacataaaaaatttgcaactggattacaaccagatcagctgtattgaagatggggcattcagggctctccgggacctggaagtgctcactctcaacaataacaacattactagactttctgtggcaagtttcaaccatatgcctaaacttaggacttttcgactgcattcaaacaacctgtattgtgactgccacctggcctggctctccgactggcttcgcaaaaggcctcgggttggtctgtacactcagtgtatgggcccctcccacctgagaggccataatgtagccgaggttcaaaaacgagaatttgtctgcagtgatgaggaagaaggtcaccagtcatttatggctccttcttgtagtgttttgcactgccctgccgcctgtacctgtagcaacaatatcgtagactgtcgtgggaaaggtctcactgagatccccacaaatcttccagagaccatcacagaaatacgtttggaacagaacacaatcaaagtcatccctcctggagctttctcaccatataaaaagcttagacgaattgacctgagcaataatcagatctctgaacttgcaccagatgctttccaaggactacgctctctgaattcacttgtcctctatggaaataaaatcacagaactccccaaaagtttatttgaaggactgttttccttacagctcctattattgaatgccaacaagataaactgccttcgggtagatgcttttcaggatctccacaacttgaaccttctctccctatatgacaacaagcttcagaccatcgccaaggggaccttttcacctcttcgggccattcaaactatgcatttggcccagaacccctttatttgtgactgccatctcaagtggctagcggattatctccataccaacccgattgagaccagtggtgcccgttgcaccagcccccgccgcctggcaaacaaaagaattggacagatcaaaagcaagaaattccgttgttcaggtacagaagattatcgatcaaaattaagtggagactgctttgcggatctggcttgccctgaaaagtgtcgctgtgaaggaaccacagtagattgctctaatcaaaagctcaacaaaatcccggagcacattccccagtacactgcagagttgcgtctcaataataatgaatttaccgtgttggaagccacaggaatctttaagaaacttcctcaattacgtaaaataaactttagcaacaataagatcacagatattgaggagggagcatttgaaggagcatctggtgtaaatgaaatacttcttacgagtaatcgtttggaaaatgtgcagcataagatgttcaagggattggaaagcctcaaaactttgatgttgagaagcaatcgaataacctgtgtggggaatgacagtttcataggactcagttctgtgcgtttgctttctttgtatgataatcaaattactacagttgcaccaggggcatttgatactctccattctttatctactctaaacctcttggccaatccttttaactgtaactgctacctggcttggttgggagagtggctgagaaagaagagaattgtcacgggaaatcctagatgtcaaaaaccatacttcctgaaagaaatacccatccaggatgtggccattcaggacttcacttgtgatgacggaaatgatgacaatagttgctccccactttctcgctgtcctactgaatgtacttgcttggatacagtcgtccgatgtagcaacaagggtttgaaggtcttgccgaaaggtattccaagagatgtcacagagttgtatctggatggaaaccaatttacactggttcccaaggaactctccaactacaaacatttaacacttatagacttaagtaacaacagaataagcacgctttctaatcagagcttcagcaacatgacccagctcctcaccttaattcttagttacaaccgtctgagatgtattcctcctcgcacctttgatggattaaagtctcttcgattactttctctacatggaaatgacatttctgttgtgcctgaaggtgctttcaatgatctttctgcattatcacatctagcaattggagccaaccctctttactgtgattgtaacatgcagtggttatccgactgggtgaagtcggaatataaggagcctggaattgctcgttgtgctggtcctggagaaatggcagataaacttttactcacaactccctccaaaaaatttacctgtcaaggtcctgtggatgtcaatattctagctaagtgtaacccctgcctatcaaatccgtgtaaaaatgatggcacatgtaatagtgatccagttgacttttaccgatgcacctgtccatatggtttcaaggggcaggactgtgatgtcccaattcatgcctgcatcagtaacccatgtaaacatggaggaacttgccacttaaaggaaggagaagaagatggattctggtgtatttgtgctgatggatttgaaggagaaaattgtgaagtcaacgttgatgattgtgaagataatgactgtgaaaataattctacatgtgtcgatggcattaataactacacatgcctttgcccacctgagtatacaggtgagttgtgtgaggagaagctggacttctgtgcccaggacctgaacccctgccagcacgattcaaagtgcatcctaactccaaagggattcaaatgtgactgcacaccagggtacgtaggtgaacactgcgacatcgattttgacgactgccaagacaacaagtgtaaaaacggagcccactgcacagatgcagtgaacggctatacgtgcatatgccccgaaggttacagtggcttgttctgtgagttttctccacccatggtcctccctcgtaccagcccctgtgataattttgattgtcagaatggagctcagtgtatcgtcagaataaatgagccaatatgtcagtgtttgcctggctatcagggagaaaagtgtgaaaaattggttagtgtgaattttataaacaaagagtcttatcttcagattccttcagccaaggttcggcctcagacgaacataacacttcagattgccacagatgaagacagcggaatcctcctgtataagggtgacaaagaccatatcgcggtagaactctatcgggggcgtgttcgtgccagctatgacaccggctctcatccagcttctgccatttacagtgtggagacaatcaatgatggaaacttccacattgtggaactacttgccttggatcagagtctctctttgtccgtggatggtgggaaccccaaaatcatcactaacttgtcaaagcagtccactctgaattttgactctccactctatgtaggaggcatgccagggaagagtaacgtggcatctctgcgccaggcccctgggcagaacggaaccagcttccacggctgcatccggaacctttacatcaacagtgagctgcaggacttccagaaggtgccgatgcaaacaggcattttgcctggctgtgagccatgccacaagaaggtgtgtgcccatggcacatgccagcccagcagccaggcaggcttcacctgcgagtgccaggaaggatggatggggcccctctgtgaccaacggaccaatgacccttgccttggaaataaatgcgtacatggcacctgcttgcccatcaatgcgttctcctacagctgtaagtgcttggagggccatggaggtgtcctctgtgatgaagaggaggatctgtttaacccatgccaggcgatcaagtgcaagcatgggaagtgcaggctttcaggtctggggcagccctactgtgaatgcagcagtggatacacgggggacagctgtgatcgagaaatctcttgtcgaggggaaaggataagagattattaccaaaagcagcagggctatgctgcttgccaaacaaccaagaaggtgtcccgattagagtgcagaggtgggtgtgcaggagggcagtgctgtggaccgctgaggagcaagcggcggaaatactctttcgaatgcactgacggctcctcctttgtggacgaggttgagaaagtggtgaagtgcggctgtacgaggtgtgtgtcctaaacacactcccggcagctctgtctttggaaaaggttgtatacttcttgaccatgtgggactaatgaatgcttcatagtggaaatatttgaaatatattgtaaaatacagaacagacttatttttattatgagaataaagactttttttctgcatttggaaaaaaaaaaaaaaaaaactcga

SEQ ID NO: 7 - protein, 1521 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGCTRCVS

SEQ ID NO: 8 - protein, 1521 aa, Homo sapiens; identical to SEQ ID NO: 7

SEQ ID NO: 9 - protein, 1523 aa, Homo sapiens
MAPGWAGVGAAVRARLALALALASVLSGPPAVACPTKCTCSAASVDCHGLGLRAVPRGIPRNAERLDLDRNNITRITKMDFAGLKNLRVLHLEDNQVSVIERGAFQDLKQLERLRLNKNKLQVLPELLFQSTPKLTRLDLSENQIQGIPRKAFRGITDVKNLQLDNNHISCIEDGAFRALRDLEILTLNNNNISRILVTSFNHMPKIRTLRLHSNHLYCDCHLAWLSDWLRQRRTVGQFTLCMAPVHLRGFNVADVQKKEYVCPAPHSEPPSCNANSISCPSPCTCSNNIVDCRGKGLMEIPANLPEGIVEIRLEQNSIKAIPAGAFTQYKKLKRIDISKNQISDIAPDAFQGLKSLTSLVLYGNKITEIVKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGSEDYRSRFSSECFMDLVCPEKCRCEGTIVDCSNQKLVRIPSHLPEYVTDLRLNDNEVSVLEATGIFKKLPNLRKINLSNNKIKEVREGAFDGAASVQELMLTGNQLETVHGRVFRGLSGLKTLMLRSNLIGCVSNDTFAGLSSVRLLSLYDNRITTITPGAFTTLVSLSTINLLSNPFNCNCHLAWLGKWLRKRRIVSGNPRCQKPFFLKEIPIQDVAIQDFTCDGNEESSCQLSPRCPEQCTCMETVVRCSNKGLRALPRGMPKDVTELYLEGNHLTAVPRELSALRHLTLIDLSNNSISMLTNYTFSNMSHLSTLILSYNRLRCIPVHAFNGLRSLRVLTLHGNDISSVPEGSFNDLTSLSHLALGTNPLHCDCSLRWLSEWVKAGYKEPGIARCSSPEPMADRLLLTTPTHRFQCKGPVDINIVAKCNACLSSPCKNNGTCTQDPVELYRCACPYSYKGKDCTVPINTCIQNPCQHGGTCHLSDSHKDGFSCSCPLGFEGQRCEINPDDCEDNDCENNATCVDGINNYVCICPPNYTGELCDEVIDHCVPELNLCQHEAKCIPLDKGFSCECVPGYSGKLCETDNDDCVAHKCRHGAQCVDTINGYTCTCPQGFSGPFCEHPPPMVLLQTSPCDQYECQNGAQCIVVQQEPTCRCPPGFAGPRCEKLITVNFVGKDSYVELASAKVRPQANISLQVATDKDNGILLYKGDNDPLALELYQGHVRLVYDSLSSPPTTVYSVETVNDGQFHSVELVTLNQTLNLVVDKGTPKSLGKLQKQPAVGINSPLYLGGIPTSTGLSALRQGTDRPLGGFHGCIHEVRINNELQDFKALPPQSLGVSPGCKSCTVCKHGLCRSVEKDSVVCECRPGWTGPLCDQEARDPCLGHRCHHGKCVATGTSYMCKCAEGYGGDLCDNKNDSANACSAFKCHHGQCHISDQGEPYCLCQPGFSGEHCQQENPCLGQVVREVIRRQKGYASCATASKVPIMECRGGCGPQCCQPTRSKRRKYVFQCTDGSSFVEEVERHLECGCLACS